SEQUENCE LISTING (31 sequences)

SEQ ID NO: 1 - protein, 4 aa, Artificial Sequence
  Synthetic
  Misc_feature (1)..(1): May be modified by Decanoyl
  Misc_feature (4)..(4): May be modified by CMK
RVKR

SEQ ID NO: 2 - protein, 4 aa, Artificial Sequence
  Synthetic
  Misc_feature (1)..(1): May be modified by pIRES
  Misc_feature (4)..(4): May be modified by vector V5
EGFP

SEQ ID NO: 3 - protein, 11 aa, Artificial Sequence
  Synthetic
  Misc_feature (1)..(1): May be modified by FAM
  Misc_feature (11)..(11): May be modified by TAMRA
QRVRRAVGIDK

SEQ ID NO: 4 - protein, 13 aa, Artificial Sequence
  Synthetic
  Misc_feature (1)..(1): May be modified by GalNAc-T2
GGGGSDSTARIRR

SEQ ID NO: 5 - protein, 9 aa, Artificial Sequence
  Synthetic
  Misc_feature (9)..(9): May be modified by GFP
NAKGGGGGS

SEQ ID NO: 6 - protein, 8 aa, Artificial Sequence
  Synthetic
  Misc_feature (1)..(1): May be modified by DABSYL/Glu
TNSPRRAR

SEQ ID NO: 7 - protein, 4 aa, Artificial Sequence
  Synthetic
  Misc_feature (4)..(4): May be modified by EDANS
SVAS

SEQ ID NO: 8 - protein, 8 aa, Artificial Sequence
  Synthetic
YHTVSLLR

SEQ ID NO: 9 - protein, 4 aa, Artificial Sequence
  Synthetic
STSQ

SEQ ID NO: 10 - protein, 8 aa, Artificial Sequence
  Synthetic
TLTPRSVR

SEQ ID NO: 11 - protein, 4 aa, Artificial Sequence
  Synthetic
SVPG

SEQ ID NO: 12 - protein, 12 aa, Artificial Sequence
  Synthetic
TNSPRRAASVAS

SEQ ID NO: 13 - protein, 12 aa, Artificial Sequence
  Synthetic
TNSPARAASVAS

SEQ ID NO: 14 - protein, 12 aa, Artificial Sequence
  Synthetic
TNSPARARSVAS

SEQ ID NO: 15 - protein, 6 aa, Artificial Sequence
  Synthetic
GSRSAR

SEQ ID NO: 16 - protein, 4 aa, Artificial Sequence
  Synthetic
SAIE

SEQ ID NO: 17 - protein, 6 aa, Artificial Sequence
  Synthetic
SKPSKR

SEQ ID NO: 18 - protein, 4 aa, Artificial Sequence
  Synthetic
SFIE

SEQ ID NO: 19 - protein, 5 aa, Artificial Sequence
  Synthetic
PRRAA

SEQ ID NO: 20 - protein, 5 aa, Artificial Sequence
  Synthetic
PARAR

SEQ ID NO: 21 - protein, 5 aa, Artificial Sequence
  Synthetic
PARAA

SEQ ID NO: 22 - protein, 4 aa, Artificial Sequence
  Synthetic
RRAR

SEQ ID NO: 23 - protein, 4 aa, Artificial Sequence
  Synthetic
RRAA

SEQ ID NO: 24 - protein, 4 aa, Artificial Sequence
  Synthetic
ARAR

SEQ ID NO: 25 - protein, 4 aa, Artificial Sequence
  Synthetic
ARAA

SEQ ID NO: 26 - protein, 5 aa, Artificial Sequence
  Synthetic
PRRAR

SEQ ID NO: 27 - protein, 4 aa, Artificial Sequence
  Synthetic
RQSR

SEQ ID NO: 28 - protein, 22 aa, Artificial Sequence
  Synthetic
  Misc_feature (1)..(1): May be modified by GalNAc-T2
  Misc_feature (22)..(22): May be modified by GFP
GGGGSDSTARIRRNAKGGGGGS

SEQ ID NO: 29 - protein, 5 aa, Artificial Sequence
  Synthetic
KPSKR

SEQ ID NO: 30 - protein, 12 aa, Artificial Sequence
  Synthetic
SFIEDLLFNKVT

SEQ ID NO: 31 - protein, 4 aa, Artificial Sequence
  Synthetic
AVAS

(19) United States
(12) Patent Application Publication
(10) Pub. No.: US 2023/0149401 A1
(43) Pub. Date: May 18, 2023

(54) FURIN INHIBITORS FOR TREATING CORONAVIRUS INFECTIONS

(71) Applicant: BP Asset V, Inc., Cambridge, MA (US)

(72) Inventors: Keith Wilcoxen, Cambridge, MA (US); Claudine Mapa, Cambridge, MA (US); Tatjana Odrljin, Cambridge, MA (US); Nabil G. Seidah, Montreal (CA)

(73) Assignee: BP Asset V, Inc., Cambridge, MA (US)

(21) Appl. No.: 17/915,878

(22) PCT Filed: Apr. 1, 2021
(86) PCT No.: PCT/US2021/025382

§ 371 Date: Sep. 29, 2022

Related U.S. Application Data
(60) Provisional application No. 63/156,058, filed on Mar. 3, 2021.
(60) Provisional application No. 63/013,382, filed on Apr. 21, 2020.
(60) Provisional application No. 63/004,365, filed on Apr. 2, 2020.

(51) Int. Cl. A61K 31/506; A61P 31/14; A61K 31/551; A61K 31/55; A61K 31/496; A61K 31/4995; A61K 31/4545; G01N 33/50; A61K 31/553; A61K 31/675; A61K 31/69; A61K 31/537

(52) U.S. Cl. CPC A61K 31/506; A61P 31/14; A61K 31/551; A61K 31/55; A61K 31/496; A61K 31/4995; A61K 31/4545; G01N 33/5038; A61K 31/553; A61K 31/675; A61K 31/69; A61K 31/537

(57) ABSTRACT

Provided herein are methods, pharmaceutical compositions, and kits for treating and/or preventing a viral infection resulting from a coronaviridae family virus in a subject in need thereof, comprising administering to the subject a compound of Formula (I), or a pharmaceutical composition comprising a compound of Formula (I). Further provided herein are methods for inhibiting the viral entry into a cell of a coronaviridae family virus (e.g., alpha-coronavirus (e.g., HCoV-NL63, HCoV-229E), betacoronavirus (e.g., SARS-CoV, SARS-CoV-2, MERS-CoV, HCoV-OC43, HCoV-HKU1)) in a subject in need thereof, comprising administering to the subject a therapeutically effective amount of a compound of Formula (I), or a pharmaceutically acceptable salt thereof, or a pharmaceutical composition comprising a compound of Formula (I) as described herein. Also provided are pharmaceutical compositions and kits comprising a compound of Formula (I), or a pharmaceutically acceptable salt thereof, for use in the treatment and/or prevention of a viral infection resulting from a coronaviridae family virus in a subject in need thereof.

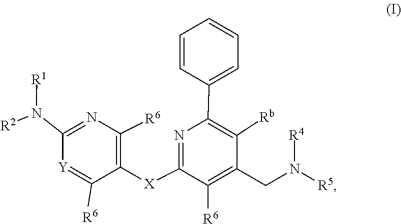

### RELATED APPLICATIONS

The present application claims priority under 35 U.S.C. § 119(e) to U.S. provisional applications, U.S. Ser. No. 63/004,365, filed Apr. 2, 2020, U.S. Ser. No. 63/013,382, filed Apr. 21, 2020, and U.S. Ser. No. 63/156,058, filed Mar. 3, 2021, which are incorporated herein by reference.

### BACKGROUND OF THE INVENTION

The human genome encodes more than 550 proteases. These molecular scissors play important roles in essentially all physiological processes. Proteolytic cleavage is certainly one of the most important post-translational modifications, generating a plethora of bioactive proteins and peptides with key roles in cell proliferation, immunity and inflammation. Not surprisingly, mutations in proteases and/or aberrant protease activity are associated with numerous pathological processes including cancer, cardiovascular disorders, and autoimmune diseases (Chakraborti S, Dhalla N S. Pathophysiological Aspects of Proteases. Berlin, Germany: Springer, 2017). Intriguingly, also many viral pathogens exploit cellular proteases for the proteolytic processing and maturation of their own proteins. Similarly, activation of bacterial toxins frequently requires cleavage by proteases of the infected or intoxicated host.

In recent years, modulation of protease activity has therefore emerged as a potential therapeutic approach in a variety of infectious and noninfectious diseases. One particularly promising target for therapeutic intervention is the cellular protease furin. This protease most likely cleaves and activates more than 150 mammalian, viral, and bacterial substrates (Tian S, Huang Q, Fang Y et al. FurinDB: a database of 20-residue furin cleavage site motifs, substrates and their associated drugs. Int J Mol Sci 2011; 12: 1060-1065.) Among them are viral envelope glycoproteins and bacterial toxins, as well as cellular factors that promote tumor development and growth if they are hyperactivated.

Furin is a member of the evolutionarily ancient family of proprotein convertases. Their similarity with bacterial subtilisin and yeast kexin proteases has led to the abbreviation PCSK (proprotein convertase subtilisin/kexin type). Humans encode nine members of this protease family (PCSK1-9), with PCSK3 representing furin. PCSKs are well known for their ability to activate other cellular proteins. The proteolytic conversion of inactive precursor proteins into bioactive molecules has already been described in the 1960s (Steiner DF, Cunningham D, Spigelman L et al. Insulin biosynthesis: evidence for a precursor. Science 1967; 157: 697-700). However, it took more than 20 years until furin was identified as the first mammalian proprotein convertase (van de Ven W J, Voorberg J, Fontijn R et al Furin is a subtilisin-like proprotein processing enzyme in higher eukaryotes. Mol Biol Rep 1990; 14: 265-275). To date, more than 200 cellular substrates of PCSKs have been described, including hormones, receptors, growth factors and adhesion molecules.

A potent peptidic furin inhibitor was identified by incorporating a reactive chloromethyl ketone (CMK) moiety (WO 2009/023306 A2; Garten W, Hallenberger S, Ortmann D, Schafer W, Vey M, Angliker H, et al. Biochimie 1994, 76(3-4), 217-225). This non-selective CMK peptide (Decanoyl-Arg-Val-Lys-Arg-CMK) engages the active site of furin at the catalytic Ser368 residue to give a tetrahedral hemiketal that irreversibly alkylates the His194 residue. This well-known irreversible protease inhibition mechanism of a halomethylketone provides very high and durable potency, however also can account for non-selective protease inhibition, particularly against other PCSK family members. Furin plays a diverse biological role in health and diseases with high unmet medical need. Therefore, potent and selective small molecule furin inhibitors with drug-like properties are desirable as an attractive approach to provide therapeutic benefit in many diseases, such as infectious diseases.

### SUMMARY OF THE INVENTION

Infectious diseases may be spread from one person to another and are caused by pathogenic microorganisms such as bacteria, viruses, parasites, or fungi. Pathogenicity is the ability of a microbial agent to cause disease and virulence is the degree to which an organism is pathogenic. In order for viruses to enter host cells and replicate, the envelope glycoproteins must be proteolytically activated (Nakayama K. Biochem. J. 1997, 327(3), 625-635). The processing of envelope glycoproteins may in some cases impact viral pathogenicity (Nakayama K. Biochem. J. 1997, 327(3), 625-635). The glycoprotein precursors of many virulent viruses, such as human immunodeficiency virus (HIV), avian influenza virus, measles virus, respiratory syncytial virus (RSV), Ebola virus, anthrax, and Zika virus (ZIKV), are cleaved at a site marked by a consensus sequence consistent with furin recognition (Thomas G. Nat. Rev. Mol. Cell. Biol. 2002, 3(10), 753-766; 2, 36-38). The cleavage of HIV glycoprotein160 and infectious virus production are blocked when the furin inhibitor al-PDX is expressed in cells (Nakayama K. Biochem. J. 1997, 327(3), 625-635). It is thus conceivable for the therapeutic use of furin inhibitor in a pandemic situation or biological warfare.

Provided herein are methods, pharmaceutical compositions, and kits for treating a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) comprising administering to the subject a therapeutically effective amount of a compound of Formula (I), or a pharmaceutically acceptable salt thereof, or a pharmaceutical composition comprising a compound of Formula (I) as described herein.

Further provided herein are methods, pharmaceutical compositions, and kits for preventing a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus), comprising administering to the subject a prophylactically effective amount of a compound of Formula (I), or a pharmaceutically acceptable salt thereof, or a pharmaceutical composition comprising a compound of Formula (I) as described herein.

In some aspects, the methods disclosed herein further comprise administering to a subject in need thereof an additional pharmaceutical agent (e.g., an antiviral, antibacterial, anti-inflammatory).

In another aspect, the present disclosure provides methods, pharmaceutical compositions, and kits for decreasing the viral infectivity of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject, the method comprising administering to the subject an effective amount of a compound of Formula (I), or pharmaceutically acceptable salt thereof, or a pharmaceutical composition comprising Formula (I) as described herein.

In another aspect, the pharmaceutical compositions and kits useful in the present disclosure comprise a compound of Formula (I), or a pharmaceutically acceptable salt thereof, as described herein, and optionally a pharmaceutically acceptable excipient.

In another aspect, the pharmaceutical compositions and kits useful in the present disclosure comprise a compound of Formula (I), or a pharmaceutically acceptable salt thereof, as described herein, and optionally an additional pharmaceutical agent (e.g., an antiviral, antibacterial, anti-inflammatory, an antifibrotic agent).

In yet another aspect, the present invention provides compounds of Formula (I), and pharmaceutical compositions thereof, for use in the treatment of a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In yet another aspect, the present invention provides compounds of Formula (I), and pharmaceutical compositions thereof, for use in the prevention of a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In another aspect, the present disclosure provides uses of compounds of Formula (I), and pharmaceutical compositions thereof, in the manufacture of a medicament for treating viral infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In another aspect, the present disclosure provides uses of compounds of Formula (I), and pharmaceutical compositions thereof, in the manufacture of a medicament for preventing viral infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In certain embodiments, the compounds useful in the present disclosure are of the Formula (I):

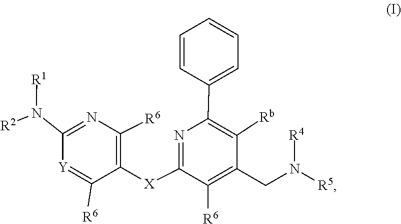

or a pharmaceutically acceptable salt thereof.

In certain embodiments, the compound of Formula (I) is of the Formula (II):

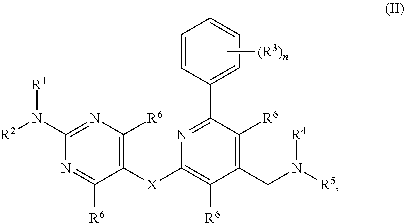

or a pharmaceutically acceptable salt thereof, wherein:

In certain embodiments, the compound of Formula (II) useful in the present disclosure is of the formula (Table 1, #192):

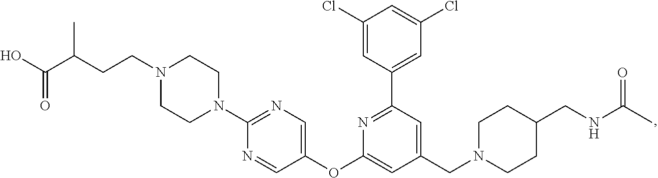 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]

or a pharmaceutically acceptable salt thereof.

In certain embodiments, the compound of Formula (I) is of the Formula (III):

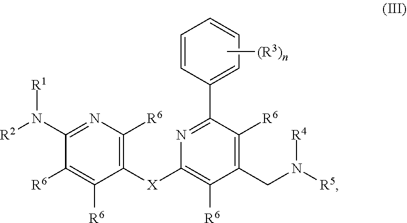

or a pharmaceutically acceptable salt thereof.

In certain embodiments, the compound of Formula (III) useful in the present disclosure is of the formula (Table 2, #219):

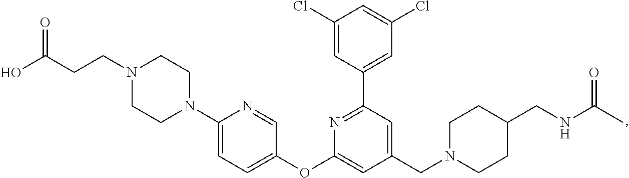 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

or a pharmaceutically acceptable salt thereof.

Another aspect of the present disclosure relates to kits comprising a container with a compound of Formula (I), or a pharmaceutical composition comprising a compound of Formula (I), as described herein. The kits described herein may include a single dose or multiple doses of the compound or pharmaceutical composition. The kits may be useful in a method of the disclosure. In certain embodiments, the kit further includes an additional pharmaceutical agent. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of certain embodiments of the disclosure are set forth in the Detailed Description of Certain Embodiments, as described below. Other features, objects, and advantages of the disclosure will be apparent from the Definitions, Examples, Figures, and Claims.

### Definitions

Terms are used within their ordinary and accepted meanings. The following definitions are meant to clarify, but not limit, the terms defined herein.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). This disclosure also encompasses compounds as individual isomers substantially free of other isomers, and alternatively, as mixtures of various isomers.

In a formula, the bond [symbol] is a single bond, the dashed line - - - is a single bond or absent, and the bond [symbol] or [symbol] is a single or double bond.

Unless otherwise provided, a formula includes compounds that do not include isotopically enriched atoms and also compounds that include isotopically enriched atoms.

Compounds that include isotopically enriched atoms may be useful, for example, as analytical tools and/or probes in biological assays.

When a range of values (“range”) is listed, it is intended to encompass each value and sub-range within the range. A range is inclusive of the values at the two ends of the range unless otherwise provided. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups. The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

“Alkoxy” refers to a group containing an alkyl radical, attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups include, without limitation, methoxy, ethoxy, n-propoxy, isopropoxy, n-butoxy, s-butoxy, isobutoxy, and t-butoxy.

When the term “alkyl” is used in combination with other substituent groups, such as “halo(C₁-C₆)alkyl”, “(C₃-C₆)cycloalkyl(C₁-C₄)alkyl-”, or “(C₁-C₄)alkoxy(C₂-C₄)alkyl-”, the term “alkyl” is intended to encompass a divalent straight or branched-chain hydrocarbon radical, wherein the point of attachment is through the alkyl moiety. The term “halo(C₁-C₆)alkyl” is intended to mean a radical having one or more halogen atoms, which may be the same or different, at one or more carbon atoms of an alkyl moiety containing from 1 to 6 carbon atoms, which is a straight or branched-chain carbon radical. Examples of “halo(C₁-C₆)alkyl” groups include, but are not limited to, —CH₂F (fluoromethyl), —CHF₂ (difluoromethyl), —CF₃ (trifluoromethyl), —CCl₃ (trichloromethyl), 1,1-difluoroethyl, 2-fluoro-2-methylpropyl, 2,2-difluoropropyl, 2,2,2-trifluoroethyl, and hexafluoroisopropyl. Examples of “(C₃-C₆)cycloalkyl(C₁-C₄)alkyl-” groups include, but are not limited to, cyclobutylmethyl, cyclopentylmethyl, cyclohexylmethyl, cyclobutylethyl, cyclopentylethyl, and cyclohexylethyl. Examples of “(C₁-C₄)alkoxy(C₂-C₄)alkyl-” groups include, but are not limited to, methoxyethyl, methoxyisopropyl, ethoxyethyl, ethoxyisopropyl, isopropoxyethyl, isopropoxyisopropyl, t-butoxyethyl, and t-butoxyisopropyl.

The term “haloalkyl” is a substituted alkyl group, wherein one or more of the —H atoms are independently replaced by a halogen, e.g., fluoro, bromo, chloro, or iodo. “Perhaloalkyl” is a subset of haloalkyl, and refers to an alkyl group wherein all of the —H atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₈ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl —H atoms are independently replaced with fluoro to provide a “perfluoroalkyl” group. In some embodiments, all of the haloalkyl —H atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms), such as oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl).

Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

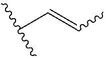

) may be in the (E)- or (Z)-configuration.
The term “heteroalkenyl” refers to an alkenyl group, which further includes at least one heteroatom (e.g., 1, 2, 3, or 4 heteroatoms) such as oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a non-aromatic, monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits. Exemplary “(C₃-C₆)cycloalkyl” groups include cyclopropyl, cyclobutyl, cyclopentyl, and cyclohexyl.

The term “heterocyclyl” or “heterocyclic” refers to a radical of a 3- to 14-membered non-aromatic ring system having ring carbon atoms and 1 to 4 ring heteroatoms, wherein each heteroatom is independently nitrogen, oxygen, or sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings.

“Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 4-11 membered heterocyclyl. In certain embodiments, the heterocyclyl group is a substituted 4-11 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently nitrogen, oxygen, or sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently nitrogen, oxygen, or sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently nitrogen, oxygen, or sulfur (“5-6 membered heterocyclyl”). In some embodiments, the 5-6 membered heterocyclyl group has 1-3 ring heteroatoms, such as nitrogen, oxygen, or sulfur. In some embodiments, the 5-6 membered heterocyclyl group has 1-2 ring heteroatoms such as nitrogen, oxygen, or sulfur. In some embodiments, the 5-6 membered heterocyclyl group has 1 ring heteroatom such as nitrogen, oxygen, or sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14× electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14× electrons shared in a cyclic array) having ring carbon atoms and 1-4 ring heteroatoms provided in the aromatic ring system, wherein each heteroatom is independently nitrogen, oxygen, or sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently nitrogen, oxygen, or sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently nitrogen, oxygen, or sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently nitrogen, oxygen, or sulfur (“5-6 membered heteroaryl”). In some embodiments, the 5-6 membered heteroaryl has 1-3 ring heteroatoms nitrogen, oxygen, or sulfur. In some embodiments, the 5-6 membered heteroaryl has 1-2 ring heteroatoms nitrogen, oxygen, or sulfur. In some embodiments, the 5-6 membered heteroaryl has 1 ring heteroatom nitrogen, oxygen, or sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one —H present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. Heteroatoms such as nitrogen may have —H substituents and/or any suitable substituent as described herein which satisfy the valencies of the heteroatoms and results in the formation of a stable moiety. This disclosure is not intended to be limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(ORC)₂, —P(R^{cc})₃⁺X⁻, —P(OR^{cc})₃⁺X⁻, —P(R_{cc})₄, —P(OR^{cc})₄, —OP(R^{cc})₂, —OP(R^{cc})₃⁺X⁻, —OP(OR^{cc})₂, —OP(OR_{cc})₃⁺X⁻, —OP(R^{cc})₄, —OP(OR_{cc})₄, —B(R^{aa})₂, —B(OR_{cc})₂, —BR^{aa}(OR_{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, —NNR^{bb}C(═O)R^{aa}, —NNR^{bb}C(═O)OR^{aa} ═NNR^{bb}S(═O)₂R^{aa}═NR^{bb}, or ═NOR^{cc}; each instance of R^{aa} is, independently, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, or 5-14 membered heteroaryl; or optionally, two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is independently —H, —OH, —OR^{aa}, —N(R^{cc})₂—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R_{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, or 5-14 membered heteroaryl; or optionally two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of RC is, independently, —H, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, or 5-14 membered heteroaryl; or optionally two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is independently halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁-10 alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, or 5-10 membered heteroaryl, wherein each alkyl, alkenyl, alkynyl, heteroalkyl, heteroalkenyl, heteroalkynyl, carbocyclyl, heterocyclyl, aryl, or heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 3-10 membered heteroaryl, wherein each alkyl, alkenyl, alkynyl, heteroalkyl, heteroalkenyl, heteroalkynyl, carbocyclyl, heterocyclyl, aryl, or heteroaryl is independently substituted with 0, 1, 2, 3, 4, or 5 R^{gg} groups; each instance of R^{ff} is independently —H, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl or 5-10 membered heteroaryl; or optionally two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{gg} is independently halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁—₆ alkyl), —NHC(═O)(C₁₋₆ alkyl), —N(C₁—₆ alkyl)C(═O)(C₁₋₆ alkyl), —NHCO₂(C₁₋₆ alkyl), —NHC(═O)N(C₁₋₆ alkyl)₂, —NHC(═O)NH(C₁—₆ alkyl), —NHC(═O)NH₂, —C(═NH)O(C₁₋₆ alkyl), —OC(═NH)(C₁—₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₄-alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form ═O or ═S; and each X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is —H, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently —H, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, the carbon atom substituents are independently halogen, substituted (e.g., substituted with one or more halogen) or unsubstituted C₁₋₆ alkyl, —OR^{aa}, —SR^{a}, —N(R^{bb})₂, —CN, —SCN, or —NO₂. In certain embodiments, the carbon atom substituents are independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is —H, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently —H, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, —H, fluorine, chlorine, bromine, iodine, oxygen, sulfur, nitrogen, and/or silicon atoms. In certain embodiments, a carbon atom substituent consists of carbon, —H, fluorine, chlorine, bromine, iodine, oxygen, sulfur, and/or nitrogen atoms. In certain embodiments, a carbon atom substituent consists of carbon, —H, fluorine, chlorine, bromine, and/or iodine atoms. In certain embodiments, a carbon atom substituent consists of carbon, —H, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than —H, and includes groups —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR_{cc})₂, or —OP(═O)(N(R^{bb}))₂, wherein X⁻, R^{aa}, R^{bb}, and R_{cc} are as defined herein.

“Oxo” represents a double-bonded oxygen moiety; for example, if attached directly to a carbon atom forms a carbonyl moiety (C═O).

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one —H and one group other than —H, and includes —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, or —NHP(═O)(N(R^{bb})₂)₂, wherein R^{aa}, R^{bb} and R^{cc} are as defined herein, and wherein R^{bb} of the group —NH(R^{bb}) is not —H.

The term “disubstituted amino” refers to an amino group wherein the nitrogen atom directly attached to the parent molecule is substituted with two groups other than —H, and includes groups —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂—NR^{bb}SO₂R^{aa}—NR^{bb}P(═O)(OR^{cc})₂, or —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with —H.

The term “trisubstituted amino” refers to an amino group wherein the nitrogen atom directly attached to the parent molecule is substituted with three groups, and includes —N(R^{bb})₃ or —N(R^{bb})₃⁺X⁻, wherein R^{bb} and X are as defined herein.

The term “sulfonyl” refers to —SO₂N(R^{bb})₂, —SO₂R^{aa}, or —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is —H; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), or imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “optionally” means that the subsequently described event(s) may or may not occur, and includes both event(s) that occur and event(s) that do not occur.

The terms “treatment,” “treat,” and “treating” refer to reversing, alleviating, delaying the onset of, or inhibiting the progress of a “pathological condition” (e.g., an infectious disease, or one or more signs or symptoms thereof) as described herein. In some embodiments, treatment may be administered after one or more signs or symptoms have developed or have been observed. In other embodiments, treatment may be administered in the absence of signs or symptoms of the disease or condition. In certain embodiments, treatment may be administered after a suspected exposure has occurred. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who is not and/or was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In certain embodiments, a prophylactic treatment may be administered after a suspected exposure has occurred to prevent viral infection. In some embodiments, a prophylactic treatment may be administered after a suspected exposure has occurred to lessen the severity of symptoms of the viral infection.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. For use in therapy, therapeutically effective amounts of a compound of Formula (I-a), as well as salts thereof, may be administered as the raw chemical. Additionally, the active ingredient may be presented as a pharmaceutical composition.

The term “inhibition,” “inhibiting,” “inhibit,” or “inhibitor” refer to the ability of a compound to reduce, slow, halt, or prevent activity of a particular biological process (e.g., furin activity, viral infectivity, viral entry into a cell, viral replication, toxin activation and/or activity) in a subject relative to vehicle.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)). In certain embodiments, the animal is a mammal. The animal may be a male or female and at any stage of development. In certain embodiments, the subject may have previously tested positive for infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus). In certain embodiments, the subject may have previously tested negative for infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus). In certain embodiments, the subject may be displaying symptoms of infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus), e.g., fever, cough, shortness of breath, tightness in the chest, loss of smell, loss of taste, diarrhea, and/or body aches. In certain embodiments, the subject may be not displaying any symptoms of infection from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus).

The terms “administer,” “administering,” or “administration,” refers to implanting, absorbing, ingesting, injecting, inhaling, or otherwise introducing a compound, or a pharmaceutical composition thereof to a subject.

### BRIEF DESCRIPTION OF THE DRAWINGS

FIG. 1 shows a model for the processing of S-protein and its blockade by furin and TMPRSS2 inhibitors. Viral infection is favored by the presence of a furin-like sites at S1/S2 and S2′. TMPRSS2 in target cells enhances infection by shedding ACE2 into soluble sACE2 (in bold) and is also enhanced by cleavage of S1 into S1′, which forms a secreted complex with sACE2. Optimal blockade of viral infection is achieved by a combination of furin and TMPRSS2 inhibitors. In the absence of a furin-like site at S1/S2 (μ5l/S2), high levels of TMPRSS2 can favor infection by cleaving S1 into S1′ and shedding ACE2 into soluble sACE2 complexed with S1′ (FIG. 1-2).

FIG. 2 shows inhibition of the processing of the pro-(S) protein, which was expressed with a V5 tag, to active (S)-protein by endogenous furin-like enzymes in VeroE6 (African green monkey kidney), BHK21 (Chinese hamster kidney), or A549 (Human pulmonary epithelial) cell lines when Compound 192 or Compound 219 are present at 0.3 μM, 1 μM, or 10 μM, or when decanoyl-RVKR-CMK (RVKR) is present at 50 μM.

FIG. 3 shows a schematic representation of the primary structure of proS and its domains and the furin-like S1/S2 site generating the S1- and S2-subunits, as well as the S2′ site preceding the fusion peptide (FIG. 3A). The signal peptide (SP), N-terminal domain (NTD), receptor binding domain (RBD) to ACE2, the two heptad repeats HR1 and HR2, the transmembrane Domain™, the cytosolic tail (CT) and the C-terminal V5-tag are indicated. In vitro furin and TMPRSS2 cleavage activity against the synthetic peptides is described in Table 1. Each substrate was tested at a final protease concentration of 2 and 100 nM (furin, enlarged box) and 50 nM (TMPRSS2) at pH 6 and 7.5, respectively. In vitro furin activity against the V5 tagged proS with the S1/S2 and S2′ cleavage site sequence of the spike protein from SARS-CoV-1, SARS-CoV-2, and MERS-CoV was measured (FIG. 3B). In vitro furin activity against WT and mutated proS proteins carrying substitutions in the furin-like cleavage site S1/S2 SARS-CoV-2 was measured and the results are shown in FIG. 3C. TMPRSS2 cleavage at both S1/S2 and S2′ was also measured (FIG. 3D). S1/S2 site is cleaved only in SARS-1 by TMPRSS2, while in SARS2, TMPESS2 cleaves at S2′ at pH6. Western blot analyses were conducted of the processing of WT proS into V5-tagged S2 and S2′ by the proprotein convertases furin, PC5A, PACE4, and PC7 following co-transfection of their cDNAs in HeLa cells (FIG. 3E). The migration positions of immature proSᵢₘ, S2 and S2′, as well as the actin loading control are marked. V=empty pIRES-EGFP-V5 vector. Western blot analyses were also conducted of HeLa cells following co-transfection with cDNAs coding for either WT S-protein or its double Ala-mutant [R685A+R682A] (μS1/S2) in the absence or presence of cDNAs coding for furin or TMPRSS2 at a ratio of 1:1 (FIG. 3F). *The estimated percent cleavages into S1/S2 and S2′ are shown, based on the ratio of the V5-immunoreactivity of the cleaved form to the sum of all forms (FIG. 3E, 3F). The data are representative of at least three independent experiments.

FIG. 4 shows comparative processing of proS and its S1/S2 mutants by endogenous proteases in HeLa cells and upon co-expression of furin or TMPRSS2. Hela cells were transiently co-transfected with cDNAs coding for an empty vector (V), including vectors encoding furin, TMPRSS2, and V5 tagged WT spike glycoprotein or its proprotein convertase (PC) cleavage site mutants at positions P4 (R682A), P1 (R685A), and P1′ (S686A). At 24 h post-transfection cell lysates were subjected to Western blotting using a V5-mAb (FIG. 4A). A Western blot showing the impact of ACE2 on the processing of spike glycoprotein by furin and TMPRSS2 is shown in FIG. 4B. The ratio of cDNAs used was S:ACE2:TMPRSS2=1:1:2. The percent processing shown under each lane was calculated from the ratio of the V5-imunoreactivity of each protein relative to the total V5-immunoreactivity. The data are representative of at least three independent experiments.

FIG. 5 shows inhibition of proprotein convertases (PCs) by representative compounds of the disclosure. FIG. 5A shows chemical motif of inhibitors. The structure of Compound 93 is shown in FIG. 5B. In vitro inhibition of the cleavage of the fluorogenic dibasic substrate FAM-QRVRRAVGIDK-TAMRA by each of the proprotein convertases furin, PC5A (PCSK5), PACE4 (PCSK6), and PC7 (PCSK7) is shown in FIG. 5C. All experiments were performed in 10 different wells, and the average pIC₅₀ (in nM) was calculated. For comparison, the inhibitory pIC₅₀ of the furin-like inhibitor RVKR-cmk performed >100 times is presented. Golgi assay: Last column represents the effects of the compounds of this disclosure on U2OS cells expressing each of furin, PC5A, PACE4, and PC7 simultaneously transduced with a BacMam-delivered construct containing a Golgi-targeting sequence followed by a 12-amino acid furin/PCSK cleavage site from Bone Morphogenic Protein 10 (BMP10) and then GFP at the C terminus (GalNAc-T2-GGGGS-DSTARIRR↓NAKG-GGGGS-GFP). Dibasic cleavage releases NAKG-GGGGS-GFP thereby reducing the Golgi-associated fluorescence estimated by imaging. In vitro inhibition of furin by Compounds 93, 192, and 219 (FIG. 5D). Furin (2 nM) was incubated with increasing concentration of the compounds, and its enzymatic activity against the synthetic peptides DABSYL/Glu-TNSPRRAR←SVAS-EDANS (5 μM) was determined at pH 7.5 (n=3). Furin-inhibitors abrogate endogenous processing of the spike glycoprotein (FIG. 5E). Hela cells were transiently transfected with a cDNA encoding an empty vector or with one expressing the spike (S) glycoprotein. Cells were treated 5 h post-transfection with no inhibitor (NT, duplicate) or with the furin-inhibitors at indicated concentrations, or RVKR-cmk at 50 μM. The media were then replaced with fresh ones not containing or containing the inhibitors for an additional 24 h, and finally the cells extracts were analyzed by Western blotting using a mAb-V5. The percent processing shown under each lane was calculated from the ratio of the V5-imunoreactivity of each protein relative to the total V5-immunoreactivity. All data are representative of at least three independent experiments.

FIG. 6 shows spike-induced cell-to-cell fusion relies on furin cleavage at S1/S2 using a luminescence-based assay HeLa TZM-bl reporter cells stably transfected with an HIV-1-based vector expressing luciferase under the control of the HIV-1 long terminal repeat (LTR), which can be activated by HIV Tat protein. Cell-to-cell fusion between donor cells (HeLa) expressing the fusogenic SARS-CoV-2 Spike protein along with the HIV trans-activator Tat, and acceptor cells (TZM-bl) that express ACE2 (FIG. 6A). Upon fusion, Tat is transferred from donor to acceptor cells, thereby inducing luciferase expression. Donor cells were transfected with vectors expressing either no protein (EV), PS1/S2, or WT Spike (S) in the absence (NT), or presence of vehicle (DMSO) and the furin-inhibitors RVKR (10 μM), Compound 93, Compound 192, and Compound 219 (300 nM) (FIG. 6B). Acceptor cells were transfected with a vector expressing ACE2. After 48 h, cells were co-cultured for 18 h. Luminescence was normalized to the EV value arbitrarily set to 1. Data are presented as mean values ±SD (n=3), One-Way Anova, Dunn-Sidák multiple comparison test. Donor HeLa cells were co-transfected with vectors (1:1 ratio) expressing WT Spike, μS1/S2 with EV or TMPRSS2 (FIG. 6C). Acceptor TZM-bl cells were transfected with ACE2. After 48 h, HeLa and TZM-bl were co-cultured for 18h and luciferase activity measured. The fusion is represented as ratio between the relative luminescence units (RLUs) measured for each condition and the RLU measured in the co-culture between donor cells transfected with EV and ACE2 acceptor cells. Data are presented as mean values ±SD (n=3), One-Way Anova, Bonferroni multiple comparison test. Donor HeLa cells express WT S or PS1/S2. Acceptor TZM-bl cells express EV only, EV+ACE2, EV+TMPRSS2, or ACE2+TMPRSS2 at a ratio 1:1 (FIG. 6D). The extent of fusion is represented as a ratio between the RLU measured for each condition and the RLU measured in the fusion between HeLa cells expressing EV with respective TZM-bl cells. The bar graph represents the average of 3 experiments performed in triplicates. Data are presented as mean values ±SEM (n=3), Two-Way Anova.

FIG. 7 shows processing of SARS-CoV-2 S by furin-like convertases is essential for viral entry in human lung epithelial cells but not in model HEK 293 cells stably expressing ACE2 (FIG. 7A). Furin cleavage of proS at the S1/S2 site is required for SARS-CoV-2 pseudoviral entry in Calu-3 but not 293T-ACE2. Cells were inoculated with luciferase-expressing HIV particles pseudotyped with SARS-CoV-2 wild-type Spike (WT S) or mutated S (μS1/S2). Each dot represents a different experiment with median luciferase activity calculated from three biological replicates. Three or four experiments were performed for each cell type. Error bars indicate standard deviation (SD). Inhibiting proS processing at S1/S2 by a novel furin-like inhibitor (Compound 93) during pseudovirion packaging prevents viral entry in Calu-3 but not in 293T-ACE2 (FIG. 7B). Each dot color depicts a different experiment and shown is mean f SD of two to three experiments (three biological replicates per experiment). Western blot analysis shows Compound 93 inhibits processing of proS at the S1/S2 site (FIG. 7C). Purified pseudovirions and cellular extracts of producing 293T17 cells treated or not with Compound 93 inhibitor were separated on SDS-PAGE gel and analyzed for HIV-1 p24 and V5-tagged S-protein (proSm or cleaved, S2) as indicated.

FIG. 8 shows furin-like inhibitors and camostat treatment decrease SARS-CoV-2 infection in Calu-3 Cells. Replication kinetics were studied at 12, 24, and 48 h post-infection by plaque assay to determine plaque-forming units (PFUs) of SARS-CoV-2 virus in the supernatant of infected Calu-3 cells treated or not with 1 μM Compound 93, 192, and 219 (FIG. 8A). A line graph represents results of the triplicate plaque assay results (mean f SD). The virus titers (expressed as plaque-forming units per milliliter (PFU/ml)) released in the supernatant (24h post-infection) of infected Calu-3 cells treated with indicated concentrations of Compound 93 were determined by plaque assay (mean f SD of triplicates, *, p<0.05; **, p<0.01; ***, p<0.001) (left panel) (FIG. 8B). The selectivity index (SI) of Compound 93 in Calu-3 cells as shown in top right panel was determined by selectivity index relation between IC50 and CC50 (CC₅₀/IC₅₀). The left y axis indicates the inhibition of virus titer (percent) relative to that of the untreated control group (red). The right y axis indicates the cell viability (percent) relative to that of the untreated control group (green). The CC₅₀ (50% cytotoxic concentration), IC₅₀ (half maximal inhibitory concentration), and SI (selectivity index) values for each inhibitor are as shown. Representative plaque images of infected Calu-3 cells treated with indicated doses of the compounds are shown in the bottom right panel. Immunoblots for the infected Calu-3 cells (right panel) and viral particles secreted in the supernatant (left panel) with and without treatment with the compounds indicate reduced viral protein levels (FIG. 8C). Immunoblots were probed for the full-length (proSm) and cleaved (S2) fragments of viral S protein and nucleocapsid (N) protein as indicated; i-Actin was included as the loading control for the cells. The virus titers (PFU per milliliter) released in the supernatant (24 h post-infection) of infected Calu-3 cells treated with Compound 192 and/or Camostat (Camo) were determined by plaque assay (mean f SD of duplicates, *, p<0.05; **, p<0.01; ***, p<0.001) (top panel) (FIG. 8D). Representative plaque images of infected Calu-3 cells are shown in the bottom panel.

FIG. 9 shows endo-F and Endo-H sensitivity of V5-tagged proS and its cleavage products by furin and TMPRSS2 in HeLa cells. Protein extracts from HeLa cells transiently expressing: V5-tagged spike protein alone, wild type (WT) or S1/S2 site mutant (μS1/S2) were treated with Endo-F and Endo-H or mock treated (NT) and analyzed by Western blotting using a V5-mAb (FIG. 9A). The spike protein WT alone or in combination with Furin or TMPRSS2 in the absence (NT) or presence of PC inhibitors RVKR (50 μM) or D6R (20 μM) treated in the same way is also shown (FIG. 9B). Spike protein, WT or R905A mutant, in combination with TMPRSS2, were treated with Endo-F and Endo-H or mock treated (NT) and analyzed by Western blotting using a V5-mAb (FIG. 9C). Consistently, endogenous and furin overexpression generated S2 fragment, was more pronounced when cells expressed PC inhibitors. TMPRSS2 expressing cells did not generate S2 fragment, nor was the TMPRSS2 cleavage of S impacted by these inhibitors.

FIG. 10 shows proteomic analysis of S2 and S2′. V5-tagged S-protein was immunoprecipitated from a Hela cell lysate using V5-agarose and subjected to SDS-PAGE electrophoresis. The bands corresponding to S2 and S2′ were excised and analyzed by mass spectrometry.

FIG. 11 shows that the compounds do not affect the generation of S2a and S2b by TMPRSS2. Hela cells were transiently transfected with cDNAs encoding either empty vector, S-protein in the presence or absence of human TMPRSS2, were either not-treated (NT) or treated with the compounds at indicated concentrations. At 48 h post-transfection the cells were collected, and their protein extracts were analyzed by Western blotting using a mAb-V5.

FIG. 12 shows TMPRSS2 cleaves proS into ER-retained fragments, sheds ACE2 into a soluble form (sACE2), and cleaves the spike protein S1-subunit into a shorter fragment (S1′) that forms a complex with sACE2. Camostat inhibits TMPRSS2 activities on proS, S1, and ACE2. HeLa-ACE2 cells were transiently transfected with empty vector (V) or increasing cDNA ratios of V5-tagged WT spike protein to TMPRSS2 (spike-V5:TMPRSS2) as indicated and 24 h later incubated for an additional 24 h in serum-free media containing 120 μM Camostat (+) or control DMSO (−) (FIG. 12A). Immunoblot of the 24 h conditioned media (concentrated 10-fold) was first probed for S1 and S1′ using an antibody against S1-subunit (GTX135356), stripped and next probed for sACE2 (ab108252) and mature TMPRSS2m (14437-1-AP) (upper panel). Cell lysates were immunoblotted for spike protein (V5-mAb), ACE2 (ab108252) and β-Actin (lower panel). HeLa-ACE2 cells were transiently transfected for 48 h with empty vector (V), TMPRSS2, and V5-tagged spike protein (S) alone or in combination with TMPRSS2 (S+TMPRSS2) in a ratio of 1:0.7 (FIG. 12B). For each condition, 1 ml of 24h serum free conditioned media was immunoprecipitated with 2 μg goat polyclonal ACE2 antibody (AF933, R&D) and 60 μl TrueBlot anti-goat Ig IP beads (00-8844-25; Rockland) and analyzed by western blot first for S1 and S1′ (GTX135356) and next for sACE2 (rabbit MAB; ab108252) (upper right panels). For reference, 50% of the media before immunoprecipitation (concentrated 10-fold) (input) was similarly analyzed (upper left panels). Immunoblots of the cell lysates (spike protein: V5-mAb) are also shown (lower panels).

FIG. 13 shows TMPRSS2-generates soluble ACE2 (sACE2) and enhances the production of S2′ in cells and S1′ in media. HeLa cells transfected for 48h with cDNAs coding for the empty vector (V), or V5-tagged spike protein, wild type (WT) or its μS1/S2 mutant, were incubated for the last 24 h with conditioned serum free media of HEK293 cells that were transfected with cDNAs encoding either empty vector (V), TMPRSS2, or full length ACE2 with TMPRSS2 in a 1:1 ratio (FIG. 13A). The HeLa cells media (concentrated 5-fold) (upper panel) and cell lysates (lower panel) were analyzed by immunoblotting as indicated. The condition pertaining to incubation of HeLa cells expressing WT spike protein with KEK293 media containing sACE2 and active mature TMPRSS2m is boxed in. dimer=sometimes observed dimer of proS; *ns=non-specific band. Media (concentrated 8-fold) and cell lysates from HeLa cells co-transfected for 48 h with cDNAs encoding V5-tagged spike protein (WT or μS1/S2)+TMPRSS2 f ACE2 were analyzed by Western blotting as indicated (FIG. 13B). The ratio of cDNAs used was S:TMPRSS2:ACE2=1:0.3:0.7. The following antibodies were used: S1 and S1′, GTX135356; TMPRSS2m, 14437-1-AP; ACE2, ab108252; S2, S2′ and S2a, V5-mAb.

FIG. 14 shows immunocytochemistry of the co-localization of ACE2 and S-protein or μS1/S2 in HeLa cells. Immunofluorescence of S-protein (S) and μS1/S2 (green) were revealed using the spike S1-antibody GTX632604 in non-permeabilized (NP) conditions or with anti-V5 in permeabilized (P) conditions (FIG. 14). ACE2 is labeled in red, and the cell nuclei are stained with DAPI (blue).

FIG. 15 shows immunofluorescence of S-protein (S) and μS1/S2 (green) in HeLa cells pre-incubated for 5 h before transfection and then for 24 h with 1 mM Compound 192. The confocal localizations were revealed as in FIG. 15A. Scale bar=10 μm. Cells expressing both S protein and ACE2 formed many syncytia associated with reduced cell surface expression of the S protein and even greater reduction of ACE2 (b). Cells expressing both μS1/S2 and ACE2 showed an accumulation of both S and ACE2 inside the cells and at the cell surface (c) however the syncytia formation was very reduced. When cells were preincubated with Compound 192 S-expressing HeLa cells phenocopy cells expressing μS1/S2.

FIG. 16 shows cell-to-cell fusion assay: correlation between syncytia formation and luciferase activity. The ACE2 expression in TZM-bl allowed fusion with cells expressing S in dose dependent manner (B) Expression of mS1/S2 in donor cells did not enhance the fusion with ACE2 expressing TZM-bl cells. Cell-to-cell fusion between donor cells (HeLa) and acceptor cells (TZM-bl) was evaluated using confocal microscopy (FIG. 16A). Hela cell transfected with and empty vector (EV), or expressing HIV-Env, SARS-CoV-2 Spike, or μS1/S2 were placed in co-culture for 18h and the number of syncytia was examined using CellMask™ to probe for the plasma membrane and Dapi to stain the nuclei. Donor cells were transfected with vectors expressing either no protein (EV), Tat, WT Spike (S), Tat and WT Spike (Tat+S), or Tat and HIV-Env (Tat+Env) (FIG. 16B). Acceptor cells were transfected with a vector expressing no protein (EV), with ACE2 or directly with Tat as appositive control. After 48 h, cells were co-cultured for 18 h. Luminescence was normalized to the EV value arbitrarily set to 1. Data are presented as mean values ±SD (n=3), and a representative experiment is shown. Donor cells were transfected with increasing amounts of plasmid expressing WT Spike, and acceptor cells were transfected with a vector expressing ACE2 (FIG. 16C). After 48 h, cells were co-cultured for 18 h, and prepared for luminescence or microscopy. Correlation between the number of syncytia counted by microscopy (n=10 per condition), and the luciferase activity was determined, and the correlation coefficient was calculated as shown. R²=0.87.

FIG. 17 shows the effects of target cells on TMPRSS2 and soluble ACE2 on cell-to-cell fusion. Donor HeLa cells were transfected with a plasmid vector expressing no protein EV, WT Spike, ρS1/S2 or WT-Spike together with soluble ACE2 (hACE2 707X). Acceptor HeLa TZM-bl cells were transfected with a plasmid vector expressing no protein, ACE2 with increasing amount of TMPRSS2 as indicated, or soluble ACE2. Fusion is represented as a ratio between the relative luminescence units (RLU) measured for each condition and the RLU measured in the co-culture between representative donor and acceptor cells. Data are presented as mean values f SD (n=3). Co-expression of ACE2 with various doses of TNPRSS2 in acceptor cells gradually promoted fusion of mS1/S2 expressing cells to similar level as WT S-induced fusion. However sACE2 had no effect on mS1/S2. So at high levels of TMPRSS in ACE2 acceptor cells fusion between cells is possible with or without cleavage at S1/S2 site.

FIG. 18 shows SARS-CoV-2 viral entry in HEK293 cells is primarily mediated via a pH-dependent pathway. Co-expression of ACE2 with various doses of TMPRSS2 in acceptor cells gradually promoted fusion of mS1/S2 expressing cells to similar level as WT S-induced fusion. However sACE2 had no effect on mS1/S2. So at high levels of TMPRSS in ACE2 acceptor cells fusion between cells is possible with or without cleavage at S1/S2 site. 293T-ACE2 were pretreated with chloroquine (CLQ; 100 μM) for 2 h and subsequently inoculated with luciferase-expressing HIV particles pseudotyped with SARS-CoV-2 wild-type (WT) or mutated (μS1/S2) spike (S). HIV particles made in the absence of S (no S) served as a negative control. Efficiency of viral entry was determined by luciferase activity. Each dot represents an independent experiment (median luciferase of biological triplicates). Error bars show SD.

FIG. 19 shows novel furin-like inhibitors block viral entry in human epithelial cells but not in model HEK293 cells stably expressing ACE2. Pseudoviruses were produced in 293T17 in the presence or absence of Compound 219 and Compound 192. 293T-ACE2 cells and Calu-3 cells were inoculated with these viruses, and viral entry was determined based upon luciferase activity (FIG. 19A, 19B). Each dot represents a different experiment (median luciferase of biological triplicates). Error bars show the SD from the mean.

FIG. 20 shows Furin-like inhibitors strongly reduce SARS-CoV-2 infection in Calu-3 cells. (FIGS. 20A and 20B) The virus titers (PFU per milliliter) released in the supernatant (24 hr post infection) of infected Calu-3 cells treated with indicated concentrations of (A) Compound 219, and (B) Compound 192 were determined by plaque assay (mean f SD of triplicates, *p<0.05, *p<0.01, **p<0.001). The selectivity index (SI) of (A) Compound 219, and (B) Compound 192 in Calu-3 cells as shown in the top right panel was determined by CC₅₀/IC₅₀. The left y axis indicates the inhibition of virus titer (percent) relative to that of the untreated control group (red). The right y axis indicates the cell viability (percent) relative to that of the untreated control group (green). Representative plaque images of infected Calu-3 cells treated with indicated doses of the compounds are shown in the bottom right panel.

FIG. 21 shows furin-like inhibitors modestly reduce virus production in SARS-CoV-2-infected Vero E6 cells in a concentration-dependent manner. Replication kinetics were studied at 12, 24 and 48 hr post infection by plaque assay to determine the plaque-forming units (PFUs) of SARS-CoV-2 virus in the supernatant of infected Vero E6 cells treated or not with 1 μM Compound 93, 219, and 192 (FIG. 21A). A line graph represents results of the triplicate plaque assay (mean f SD). Virus released in the supernatant (48 hr post infection) of infected Vero E6 cells treated with indicated concentrations of Compound 93 (FIG. 21B), Compound 219 (FIG. 21C), and Compound 192 (FIG. 21D) were determined by plaque assay (mean f SD of triplicates, *p<0.05, **p<0.01, ***p<0.001).

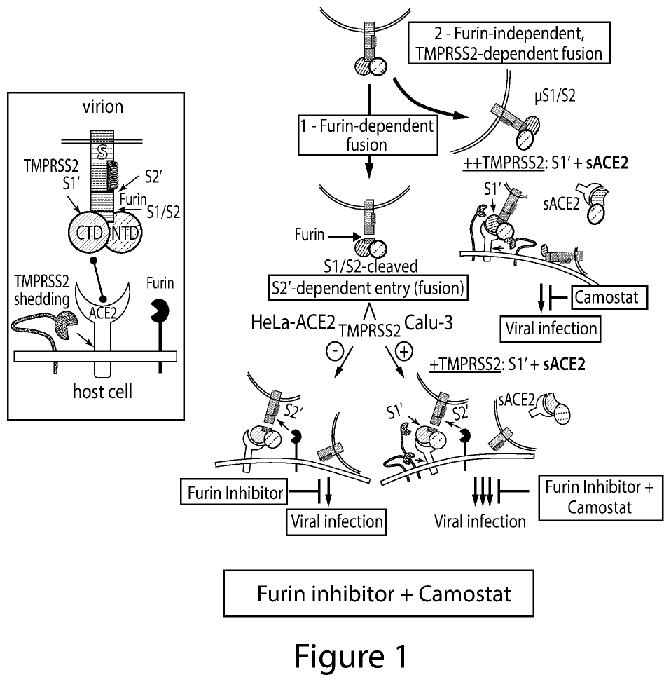

### DETAILED DESCRIPTION OF CERTAIN EMBODIMENTS OF THE INVENTION

The present disclosure provides methods, pharmaceutical compositions, and kits for the treatment and/or prevention of a viral infection caused by a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus), comprising administering to the subject a therapeutically effective amount of a compound of Formula (I), a pharmaceutically acceptable salt thereof, or a pharmaceutical composition comprising a compound of Formula (I) as described herein. Further provided herein are methods, pharmaceutical compositions, and kits for the treatment and/or prevention of a viral infection caused by a variant of a SARS-CoV-2 virus (e.g. B.1.351 (i.e., the South African COVID-19 variant), B.1.1.7 (i.e., the UK COVID-19 variant), P.1 (i.e., the Brazilian COVID-19 variant)).

Further provided herein are methods for inhibiting viral entry into a cell of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, comprising administering to the subject a therapeutically effective amount of a compound of Formula (I) or a pharmaceutically acceptable salt thereof, or a pharmaceutical composition comprising a compound of Formula (I) as described herein.

Further provided herein are methods for decreasing the pathogenicity of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, comprising administering to the subject a therapeutically effective amount of a compound of Formula (I) or a pharmaceutically acceptable salt thereof, or a pharmaceutical composition comprising a compound of Formula (I) as described herein.

Also provided herein are methods for inhibiting viral exit from a cell of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, comprising administering to the subject a therapeutically effective amount of a compound of Formula (I) or a pharmaceutically acceptable salt thereof, or a pharmaceutical composition comprising a compound of Formula (I) as described herein.

In certain embodiments, the present disclosure provides methods for the treatment and/or prevention of a viral infection caused by a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus), comprising administering to the subject a therapeutically effective amount of a compound of Formula (I) or pharmaceutically acceptable salt thereof, or a pharmaceutical composition comprising a compound of Formula (I) as described herein. In certain embodiments, the provided methods are for the treatment and/or prevention of viral infections caused by a coronaviridae family virus. In certain embodiments, the provided methods are for the treatment and/or prevention of viral infections caused by an alphacoronavirus. In certain embodiments, the provided methods are for the treatment and/or prevention of viral infections caused by HCoV-NL63 or HCoV-229E. In certain embodiments, the provided methods are for the treatment and/or prevention of viral infections caused by a betacoronavirus. In certain embodiments, the provided methods are for the treatment and/or prevention of viral infections caused by SARS-CoV, SARS-CoV-2, MERS-CoV, HCoV-OC43, or HCoV-HKU1. In certain embodiments, the provided methods are for the treatment and/or prevention of viral infections caused by SARS-CoV. In certain embodiments, the provided methods are for the treatment and/or prevention of viral infections caused by SARS-CoV-2. In certain embodiments, the provided methods are for the treatment and/or prevention of viral infections caused by MERS-CoV.

In another aspect, the present disclosure provides methods of treating a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, the method comprising administering to the subject in need thereof an effective amount (e.g., therapeutically effective amount) of a compound of Formula (I) or a pharmaceutical acceptable salt thereof, or a pharmaceutical composition comprising a compound of Formula (I) as described herein. In certain embodiments, provided herein are methods of treating viral infections resulting from an alphacoronavirus. In certain embodiments, provided herein are methods of treating viral infections resulting from HCoV-NL63 or HCoV-229E. In certain embodiments, provided herein are methods of treating viral infections resulting from a betacoronavirus. In certain embodiments, provided herein are methods of treating viral infections resulting from SARS-CoV, SARS-CoV-2, MERS-CoV, HCoV-OC43, or HCoV-HKU1. In certain embodiments, provided herein are methods of treating viral infections resulting from SARS-CoV. In certain embodiments, provided herein are methods of treating viral infections resulting from SARS-CoV-2. In certain embodiments, provided herein are methods of treating viral infections resulting from MERS-CoV. In certain embodiments, provided herein are methods of treating viral infections resulting from HCoV-OC43. In certain embodiments, provided herein are methods of treating viral infections resulting from HCoV-HKU1.

In another aspect, the present disclosure provides methods of preventing a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, the method comprising administering to the subject in need thereof an effective amount (e.g., a prophylactically effective amount) of a compound of Formula (I) or a pharmaceutically acceptable salt thereof, or a pharmaceutical composition comprising Formula (I) as described herein. In certain embodiments, the present disclosure provides methods of preventing a viral infection resulting from a betacoronavirus (e.g., SARS-CoV, SARS-CoV-2, MERS-CoV, HCoV-OC43, HCoV-HKU1), a deltacoronavirus, a gammacoronavirus) in a subject in need thereof, the method comprising administering to the subject in need thereof an effective amount (e.g., a prophylactically effective amount) of a compound of Formula (I) or a pharmaceutically acceptable salt thereof, or a pharmaceutical composition comprising Formula (I) as described herein. In certain embodiments, the present disclosure provides methods of preventing a viral infection resulting from SARS-CoV in a subject in need thereof, the method comprising administering to the subject in need thereof an effective amount (e.g., a prophylactically effective amount) of a compound of Formula (I), or a pharmaceutically acceptable salt thereof, or a pharmaceutical composition comprising Formula (I) as described herein.

In another aspect, the present disclosure provides methods of inhibiting the entry of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) into a cell, in a subject in need thereof, the method comprising administering to the subject in need thereof an effective amount of a compound of Formula (I) or pharmaceutically acceptable salt thereof, or a pharmaceutical composition comprising a compound of Formula (I) as described herein.

In certain embodiments, provided herein are methods of inhibiting the entry of a coronaviridae family virus into a cell, in a subject by at least 1%, at least 3%, at least 10%, at least 20%, at least 30%, at least 40%, at least 50%, at least 60%, at least 70%, at least 80%, or at least 90%. In certain embodiments, the entry of a coronaviridae family virus into a cell, in a subject is inhibited by at least 1%, at least 3%, at least 10%, at least 20%, at least 30%, at least 40%, at least 50%, at least 60%, at least 70%, at least 80%, or at least 90%. In certain embodiments, provided herein are methods of inhibiting the entry of a coronaviridae family virus into a cell, in a subject by at least 30%. In certain embodiments, provided herein are methods of inhibiting the entry of a coronaviridae family virus into a cell, in a subject by at least 50%. In certain embodiments, provided herein are methods of inhibiting the entry of a coronaviridae family virus into a cell, in a subject by at least 75%.

In another aspect, the present disclosure provides methods of inhibiting the replication of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof, the method comprising administering to the subject in need thereof an effective amount of a compound of Formula (I) or pharmaceutically acceptable salt thereof, or a pharmaceutical composition comprising a compound of Formula (I) as described herein.

In certain embodiments, provided herein are methods of inhibiting the replication of a coronaviridae family virus in a subject by at least 1%, at least 3%, at least 10%, at least 20%, at least 30%, at least 40%, at least 50%, at least 60%, at least 70%, at least 80%, or at least 90%. In certain embodiments, the replication of a coronaviridae family virus in a subject is inhibited by at least 1%, at least 3%, at least 10%, at least 20%, at least 30%, at least 40%, at least 50%, at least 60%, at least 70%, at least 80%, or at least 90%. In certain embodiments, provided herein are methods of inhibiting the replication of a coronaviridae family virus in a subject by at least 30%. In certain embodiments, provided herein are methods of inhibiting the replication of a coronaviridae family virus in a subject by at least 50%. In certain embodiments, provided herein are methods of inhibiting the replication of a coronaviridae family virus in a subject by at least 75%.

In another aspect, the present disclosure provides methods of decreasing viral infectivity of a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject, the method comprising administering to the subject an effective amount of a compound of Formula (I) or a pharmaceutically acceptable salt thereof, or a pharmaceutical composition comprising Formula (I) as described herein.

In another aspect, the present disclosure provides methods of inhibiting viral infectivity in a biological sample (e.g., an in vitro biological sample), the method comprising contacting the biological sample with an effective amount of a compound of Formula (I) or a pharmaceutical composition described herein. In another aspect, the present disclosure provides methods of inhibiting viral infectivity in a cell (e.g., an in vitro cell), the method comprising contacting the cell with an effective amount of a compound of Formula (I) or a pharmaceutical composition described herein.

In certain embodiments, the methods, uses, pharmaceutical compositions, kits, and compounds described herein further comprise administering one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a transcription factor in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds and the additional pharmaceutical agent, but not both. For example, the methods, uses, pharmaceutical compositions, kits, and compounds comprising a compound of Formula (I) and camostat may show a synergistic effect over compositions comprising Compound (I) or camostat in treating viral infections. The methods, uses, pharmaceutical compositions, kits, and compounds comprising a compound of Formula (I) and camostat may also show additive effects over compositions comprising Compound (I) or camostat in treating viral infections.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a viral infection (e.g., a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-inflammatory agents, immunosuppressants, antibacterial agents, antiviral agents, cardiovascular agents, anti-allergic agents, and pain-relieving agents. In certain embodiments, the additional pharmaceutical agent is an antiviral agent (e.g., Abacavir, Acyclovir, Amantadine, Atazanavir, Chloroquine, Darunavir, Elvitegravir, Fosamprenavir, Ganciclovir, Indinavir, Ledipasvir, Lopinavir, Nitazoxanide, Oseltamivir, Penciclovir, Peramivir, Raltegravir, Ribavirin, Rimantadine, Ritonavir, Saquinavir, Sofosbuvir, Tipranavir, Velpatasvir, Zanamivirfavipiravir, remdesivir, Oyal, galidesivir, umifenovir, hydroxychloroquine). In certain embodiments, the antiviral agent is chloroquine. In certain embodiments, the antiviral agent is hydroxychloroquine. In certain embodiments, the additional pharmaceutical agent is an antibacterial agent (e.g., azithromycin). In certain embodiments, the additional pharmaceutical agent is an anti-inflammatory (e.g., Gimsilumab, IL-6 antibodies, actemra, paracetamol, Nonsteroidal anti-inflammatory drugs (NSAIDs)). In certain embodiments, the anti-inflammatory may be a tumor necrosis factor (TNF) inhibitor (e.g., adalimumab, etanercept, infliximab, golimumab, certolizumab).

In certain embodiments, the additional pharmaceutical agent is an antifibrotic agent (e.g., Pirfenidone, Nintedanib). In certain embodiments, the additional pharmaceutical agent is Pirfenidone. In certain embodiments, the additional pharmaceutical agent is Nintedanib.

In certain embodiments, the additional pharmaceutical agent is in the form of an additional therapy (e.g., receiving antibodies from survivor patients' blood, DNA vaccines, RNA vaccines). In certain embodiments, the additional therapy is treatment with an antibody.

In certain embodiments, the additional therapy is treatment with a human antibody. In certain embodiments, the additional therapy is treatment with a human body from a survivor patients' blood. In certain embodiments, the additional therapy is treatment with a monoclonal antibody. In certain embodiments, the additional therapy is treatment with antibodies that bind the S-spike protein. In certain embodiments, the additional therapy is treatment with a monoclonal antibody that binds the S-spike protein.

In certain embodiments, the additional pharmaceutical agent is an N-methyl-D-aspartate (NDMA) receptor glutamate receptor antagonist (e.g., ifenprodil). In certain embodiments, the additional pharmaceutical agent is an ACE2 blocker (e.g., APNO1). In certain embodiments, the additional pharmaceutical agent is a CCR5 antagonist. In certain embodiments, the additional pharmaceutical agent is an antibody that bind S-spike protein (e.g., REGN3048-3051). In certain embodiments, the additional pharmaceutical agent is idebenone. In certain embodiments, the additional pharmaceutical agent is interferon beta. In certain embodiments, the additional pharmaceutical agent is an ADAM-17 inhibitor. In certain embodiments, the additional pharmaceutical agent is 4-methylumbelliferone.

Additional pharmaceutical agents may also include serine protease inhibitors (e.g., TMPRSS2 inhibitors (e.g., camostat, nafamostat)), ACE2 inhibitors (e.g., benazepril, captopril, enalapril, fosinopril, lisinopril, moexipril, perindopril, quinapril, ramipril, or trandolapril). In certain embodiments, the additional pharmaceutical agent is a TMPRSS2 inhibitor (e.g., camostat, nafamostat)). In certain embodiments, the additional pharmaceutical agent is camostat. In certain embodiments, the additional pharmaceutical agent is nafamostat. In certain embodiments, the additional pharmaceutical agent is benazepril, captopril, enalapril, fosinopril, lisinopril, moexipril, perindopril, quinapril, ramipril, or trandolapril. In certain embodiments, the additional pharmaceutical agent is benazepril. In certain embodiments, the additional pharmaceutical agent is captopril. In certain embodiments, the additional pharmaceutical agent is enalapril. In certain embodiments, the additional pharmaceutical agent is fosinopril. In certain embodiments, the additional pharmaceutical agent is lisinopril. In certain embodiments, the additional pharmaceutical agent is moexipril. In certain embodiments, the additional pharmaceutical agent is perindopril. In certain embodiments, the additional pharmaceutical agent is quinapril. In certain embodiments, the additional pharmaceutical agent is ramipril. In certain embodiments, the additional pharmaceutical agent is trandolapril.

In another aspect, the present disclosure provides compounds of Formula (I) or a pharmaceutically acceptable salt thereof, and pharmaceutical compositions described herein for use in treating and/or preventing a viral infection caused by a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus), comprising administering to the subject a therapeutically effective amount of a compound of Formula (I) or a pharmaceutically acceptable salt thereof, or a pharmaceutical composition comprising a compound of Formula (I) as described herein.

In another aspect, the present disclosure provides uses of compounds of Formula (I) or a pharmaceutically acceptable salt thereof, and pharmaceutical compositions as described herein in the manufacture of a medicament for treating a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In another aspect, the present disclosure provides uses of compounds of Formula (I) and pharmaceutical compositions described herein in the manufacture of a medicament for preventing a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) in a subject in need thereof.

In certain embodiments, the virus is a coronaviridae family virus. In certain embodiments, the coronaviridae family virus is an alphacoronavirus. In certain embodiments, the alphacoronavirus is HCoV-NL63. In certain embodiments, the alphacoronavirus is HCoV-229E. In certain embodiments, the coronaviridae family virus is a betacoronavirus. In certain embodiments, the betacoronavirus is SARS-CoV. In certain embodiments, the betacoronavirus is SARS-CoV-2. In certain embodiments, the betacoronavirus is MERS-CoV. In certain embodiments, the betacoronavirus is HCoV-OC43. In certain embodiments, the betacoronavirus is HCoV-HKU1. In certain embodiments, the coronaviridae family virus is a deltacoronavirus. In certain embodiments, the coronaviridae family virus is a gammacoronavirus.

In certain embodiments, the virus is a variant of a SARS-Cov-2 virus (e.g., B.1.351, B.1.1.7, P.1). In certain embodiments, the SARS-Cov-2 variant is the B.1.351 variant (i.e., the South African COVID-19 variant). In certain embodiments, the SARS-Cov-2 variant is the B.1.1.7 variant (i.e., the UK COVID-19 variant). In certain embodiments, the SARS-CoV-2 variant is the P.1 variant (i.e., the Brazilian COVID-19 variant).

Without wishing to be bound by any particular theory, in certain embodiments, the compounds of Formula (I) useful in the methods, compositions, and uses of this disclosure prevents or inhibits the furin-mediated processing Spike (S)-protein, which may be cleaved during virus egress.

Without wishing to be bound by any particular theory, in certain embodiments, the compounds of Formula (I) useful in the methods, compositions, and uses of this disclosure prevents or inhibits the furin-mediated processing Spike (S)-protein, which may be cleaved during virus entry into a cell.

Without wishing to be bound by any particular theory, in certain embodiments, the compounds of Formula (I) useful in the present disclosure inhibit viral fusion by cleaving the glycoproteins of a virus.

Without wishing to be bound by any particular theory, in certain embodiments, the compounds of Formula (I) useful in the present disclosure inhibit viral fusion (during viral entry or exit of the cell) by inhibiting the furin-mediated processing of the Spike (S)-protein.

Cleavage of the (S)-protein may be required to expose the fusion protein, which allows for viral entry and exit into the cell.

In certain embodiments, the compounds useful in the present disclosure are of Formula (I):

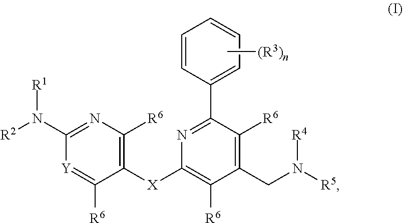

or a pharmaceutically acceptable salt thereof, wherein:

X is —O— or —N(R⁸)—;

Y is —N═ or —C(R⁶)═;

R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;

each R³ is independently halogen, —CN, —O(C₁-C₄)alkyl, or optionally substituted (C₁-C₄)alkyl;

R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;

each R⁶ is independently H, halogen, optionally substituted (C₁-C₄)alkyl, —OH, or optionally substituted (C₁-C₄)alkoxy;

each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);

each of R⁸ and R⁹ is independently H, optionally substituted (C₁-C₄)alkyl, or optionally substituted (C₃-C₆)cycloalkyl;

n is 1, 2, 3, or 4.

In certain embodiments, X is —O— or —NR⁸, wherein R⁸ is (C₁-C₄)alkyl. In another embodiment, X is —NR⁸, wherein R⁸ is (C₁-C₄)alkyl. In certain embodiments, X is —O—.

In certain embodiments, Y is —N═ or —C(R⁶)═, wherein R⁶ is independently H, halogen, optionally substituted (C₁-C₄)alkyl, —OH, or optionally substituted (C₁-C₄)alkoxy. In certain embodiments, Y is —N═. In certain embodiments, Y is —C(R⁶)═.

In certain embodiments, R³ is optionally substituted —O(C₁-C₄)alkyl. In certain embodiments, R³ is optionally substituted —OCF₃. In certain embodiments, R³ is optionally substituted (C₁-C₄)alkyl. In certain embodiments, R³ is -Me. In certain embodiments, R³ is —CF₃. In certain embodiments, R³ is —CHF₂. In certain embodiments, R³ is —CH₂F. In certain embodiments, R³ is halogen. In certain embodiments, R³ is —F. In certain embodiments, R³ is —Cl. In certain embodiments, R³ is —Br. In certain embodiments, R³ is —I. In certain embodiments, R³ is -Me. In certain embodiments, each R³ is independently halogen, methyl, or difluoromethyl. In another embodiment, each R³ is independently fluoro, chloro, bromo, methyl, or difluoromethyl. In one embodiment, each R³ is independently halogen. In another embodiment, each R³ is independently fluoro, chloro, or bromo. In another embodiment, each R³ is independently fluoro or chloro. In certain embodiments, each R³ is chloro. In certain embodiments, R³ is —CN.

In certain embodiments, R¹ and R² are each independently H, (C₁-C₄)alkyl, or (C₁-C₄)alkylNH₂. In certain embodiments, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In one embodiment, R¹ and R² are each independently H, (C₁-C₄)alkyl, or —(C₁-C₄)alkylNH₂. In another embodiment, R¹ and R² are each independently H or —(C₁-C₄)alkylNH₂. R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In certain embodiments, R¹ and R² taken together with the nitrogen atom to which they are attached form an optionally substituted pyrrolidine, pyrazolidine, imidazolidine, piperidine, piperazine, or morpholine ring.

In another embodiment, R¹ and R² taken together with the nitrogen atom to which they are attached represent a 6- or 7-membered monocyclic ring, optionally containing one or two additional heteroatoms independently selected from oxygen, nitrogen, and sulfur, wherein said ring is optionally substituted by one, two, or three substituents independently halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In another embodiment, R¹ and R² taken together with the nitrogen atom to which they are attached represent a 6- or 7-membered monocyclic ring, optionally containing one or two additional nitrogen heteroatoms, wherein said ring is optionally substituted by one, two, or three substituents independently selected from halogen, hydroxyl, oxo, R⁷, —OR⁷, —NHR⁸, —NR⁷R⁸, and —C(O)R⁷. In another embodiment, R¹ and R² taken together with the nitrogen atom to which they are attached represent a 6- or 7-membered monocyclic ring, optionally containing one additional nitrogen heteroatom, wherein said ring is optionally substituted by one substituent which is R⁷. In certain embodiments, R¹ and R² taken together with the nitrogen atom to which they are attached represent an optionally substituted piperazine ring.

In certain embodiments, R¹ and R² taken together with the nitrogen atom to which they are attached form an optionally substituted piperazine ring. In certain embodiments, R¹ and R² taken together with the nitrogen atom to which they are attached form a piperazine ring of the formula:

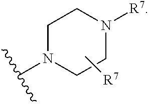

In certain embodiments, R¹ and R² taken together with the nitrogen atom to which they are attached form a piperazine ring of the formula:

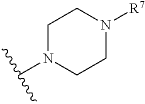

In certain embodiments, R¹ and R² taken together with the nitrogen atom to which they are attached form a piperazine ring of the formula:

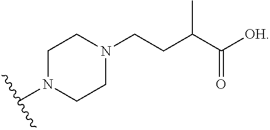 [SMILES: *N1CCN(CCC(C)C(=O)O)CC1]

In certain embodiments, R¹ and R² taken together with the nitrogen atom to which they are attached form a piperazine ring of the formula:

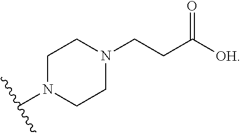 [SMILES: *N1CCN(CCC(=O)O)CC1]

In certain embodiments, R¹ and R² taken together with the nitrogen atom to which they are attached form a piperazine ring of the formula:

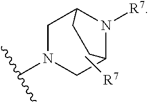

In certain embodiments, R¹ and R² taken together with the nitrogen atom to which they are attached form a piperazine ring of the formula:

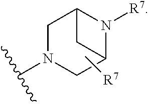

In certain embodiments, R¹ and R² taken together with the nitrogen atom to which they are attached form a piperazine ring of the formula:

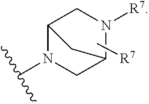

In certain embodiments, R¹ and R² taken together with the nitrogen atom to which they are attached form a piperazine ring of the formula:

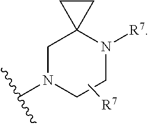

In certain embodiments, R¹ and R² taken together with the nitrogen atom to which they are attached form a ring of the formula:

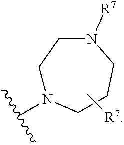

In certain embodiments, R¹ and R² taken together with the nitrogen atom to which they are attached form a ring of the formula:

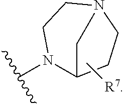

In certain embodiments, R¹ and R² taken together with the nitrogen atom to which they are attached form a piperidine ring of the formula:

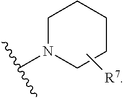

In certain embodiments, R¹ and R² taken together with the nitrogen atom to which they are attached form a piperidine ring of the formula:

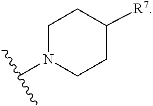

In certain embodiments, R¹ and R² taken together with the nitrogen atom to which they are attached form a ring of the formula:

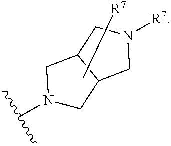

In certain embodiments, R¹ and R² taken together with the nitrogen atom to which they are attached form a ring of the formula:

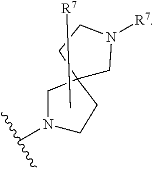

In certain embodiments, R¹ and R² taken together with the nitrogen atom to which they are attached form a pyrrolidine ring of the formula:

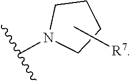

In certain embodiments, R⁴ and R⁵ are each independently H, or optionally substituted (C₁-C₄)alkyl. In certain embodiment, R⁴ and R⁵ are the same. In certain embodiments, R⁴ and R⁵ are different. In certain embodiments, R⁴ is H. In certain embodiments, R⁵ is H. In one embodiment, R⁴ and R⁵ are each independently H, (C₁-C₄)alkyl, or (C₂-C₄)alkyl(C₁-C₄)alkoxy. In certain embodiments, R⁴ is -Me. In certain embodiments, R4 is —C(O)R⁷. In certain embodiments, R⁴ is —C(O)Me. In another embodiment, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹. In another embodiment, R⁴ and R⁵ taken together with the nitrogen atom to which they are attached form a piperidine ring of the formula:

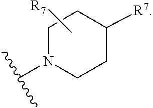

In another embodiment, R⁴ and R⁵ taken together with the nitrogen atom to which they are attached form a piperidine ring of the formula:

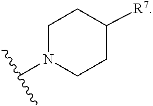

In another embodiment, R⁴ and R⁵ taken together with the nitrogen atom to which they are attached form a piperidine ring of the formula:

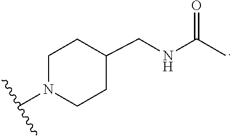 [SMILES: *N1CCC(CNC(C)=O)CC1]

In another embodiment, R⁴ and R⁵ taken together with the nitrogen atom to which they are attached form a ring of the formula:

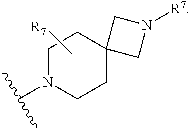

In another embodiment, R⁴ and R⁵ taken together with the nitrogen atom to which they are attached form a piperazine ring of the formula:

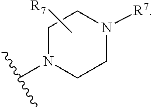

In another embodiment, R⁴ and R⁵ taken together with the nitrogen atom to which they are attached form ring of the formula:

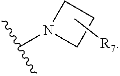

In another embodiment, R⁴ and R⁵ taken together with the nitrogen atom to which they are attached form a pyrrolidine ring of the formula:

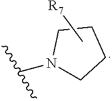

In another embodiment, R⁴ and R⁵ taken together with the nitrogen atom to which they are attached form a pyrrolidine ring of the formula:

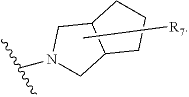

In another embodiment, R⁴ and R⁵ taken together with the nitrogen atom to which they are attached form a pyrrolidine ring of the formula:

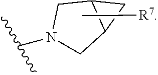

In another embodiment, R⁴ and R⁵ taken together with the nitrogen atom to which they are attached form a ring of the formula:

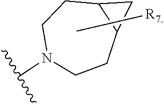

In another embodiment, R⁴ and R⁵ taken together with the nitrogen atom to which they are attached form a ring of the formula:

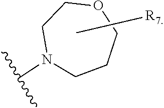

In one embodiment, each R⁶ is independently halogen or (C₁-C₄)alkyl. In another embodiment, each R⁶ is independently halogen. In another embodiment, each R⁶ is independently selected from the group consisting of fluoro, chloro, bromo, and methyl. In another embodiment, each R⁶ is independently selected from the group consisting of fluoro, chloro, and bromo. In another embodiment, each R⁶ is independently fluoro or chloro. In certain embodiments, each R⁶ is fluoro. In another embodiment, each R⁶ is chloro. In another embodiment, each R⁶ is independently (C₁-C₄)alkyl. In another embodiment, each R⁶ is methyl.

In one embodiment, each R⁷ is independently (C₁-C₆)alkyl, halo(C₁-C₆)alkyl, (C₃-C₆)cycloalkyl, or —(C₁-C₄)alkyl(C₃-C₆)cycloalkyl, each of which is optionally substituted by one or two of triazolyl, tetrazolyl, —CO₂R⁸, —CONR⁸R⁹, —CON(R⁸)CO₂(C₁-C₄)alkyl, —OH, (C₁-C₄)alkoxy, —OCONR⁸R⁹, —OCON(R⁸)C(O)R⁹, (C₁-C₄)alkyl, —(C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹). In another embodiment, each R⁷ is independently (C₁-C₄)alkyl, (C₂-C₄)alkenyl, halo(C₁-C₄)alkyl, (C₃-C₆)cycloalkyl, or —(C₁-C₂)alkyl(C₃-C₆)cycloalkyl, each of which is optionally substituted with —CO₂R⁸, —CONR⁸R⁹, —OH, oxo, —(C₁-C₄)alkoxy, —OCONR⁸R⁹, —(C₁-C₄)alkylOH, —NR⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)SO₂R⁹, —SO(C₁-C₄)alkyl, —SO₂(C₁-C₄)alkyl, —SO₃R⁸, —SO₂NR⁸R⁹, or —P(O)(OR⁸)(OR⁹). In another embodiment, each R⁷ is independently (C₁-C₄)alkyl, (C₂-C₄)alkenyl, halo(C₁-C₄)alkyl, (C₃-C₆)cycloalkyl, or —(C₁-C₂)alkyl(C₃-C₆)cycloalkyl, each of which is optionally substituted by one or two substituents —CO₂R⁸, —CONR⁸R⁹, —OH, (C₁-C₄)alkoxy, —OCONR⁸R⁹, —(C₁-C₄)alkylOH, —NR⁸R⁹, —N(R⁸)C(O)R⁹, —N(R⁸)CO₂(C₁-C₄)alkyl, —N(R⁸)CONR⁸R⁹, —N(R⁸)SO₂R⁹, —SO(C₁-C₄)alkyl, —SO₂(C₁-C₄)alkyl, —SO₃R⁸, —SO₂NR⁸R⁹, or —P(O)(OR⁸)(OR⁹). In another embodiment, each R⁷ is (C₁-C₆)alkyl which is optionally substituted by one substituent which is —CO₂H, —OH, —N(R⁸)C(O)R⁹, or —SO(C₁-C₄)alkyl. In another embodiment, each R⁷ is (C₁-C₄)alkyl which is optionally substituted by one substituent which is —CO₂H, —OH, —N(R⁸)C(O)R⁹, or —SO(C₁-C₄)alkyl.

In certain embodiments, each of R⁸ and R⁹ is independently H, optionally substituted (C₁-C₄)alkyl, or optionally substituted (C₃-C₆)cycloalkyl. In one embodiment, each R⁸ and R⁹ is independently H or (C₁-C₄)alkyl. In another embodiment, each R⁸ and R⁹ is independently (C₁-C₄)alkyl. In another embodiment, R⁸ and R⁹ are each methyl. In another embodiment, each R⁸ and R⁹ is H. In another embodiment, R⁸ is H; and R⁹ is (C₁-C₄)alkyl. In another embodiment, R⁸ is H; and R⁹ is -Me. In another embodiment, R⁸ is (C₁-C₄)alkyl. In another embodiment, R⁸ is -Me. In another embodiment, R⁸ is —H. In another embodiment, R⁹ is (C₁-C₄)alkyl. In another embodiment, R⁹ is -Me. In another embodiment, R⁹ is —H.

In one embodiment, n is 1, 2, or 3. In another embodiment, n is 2 or 3. In another embodiment, n is 2.

In certain embodiments, the compound of Formula (I) useful in the present disclosure is of the Formula (II):

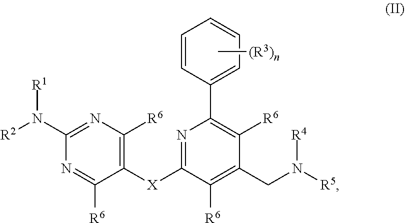

or a pharmaceutically acceptable salt thereof, wherein:

X is —O— or —N(R⁸)—;

R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR¹, —CONR⁷R⁸, or —SO₂R⁷;

each R³ is independently halogen, —CN, —O(C₁-C₄)alkyl, or optionally substituted (C₁-C₄)alkyl;

R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;

each R⁶ is independently H, halogen, optionally substituted (C₁-C₄)alkyl, —OH, or optionally substituted (C₁-C₄)alkoxy;

each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹); each of R⁸ and R⁹ is independently H, optionally substituted (C₁-C₄)alkyl, or optionally substituted (C₃-C₆)cycloalkyl;

n is 1, 2, 3, or 4.

In certain embodiments, the compound of Formula (II) useful in the present disclosure is of the formula:

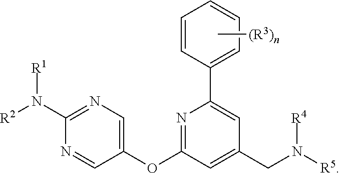

In certain embodiments, the compound of Formula (II) useful in the present disclosure is of the formula:

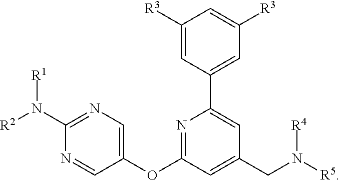

In certain embodiments, the compound of Formula (II) useful in the present disclosure is of the formula:

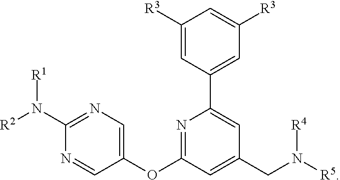

In certain embodiments, the compound of Formula (II) useful in the present disclosure is of the formula:

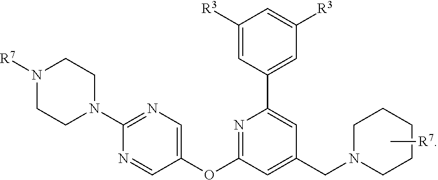

In certain embodiments, the compound of Formula (II) useful in the present disclosure is of the formula:

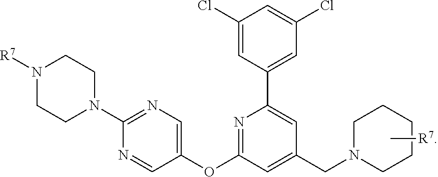

In certain embodiments, the compound of Formula (II) useful in the present disclosure is of the formula (Table 1, #192):

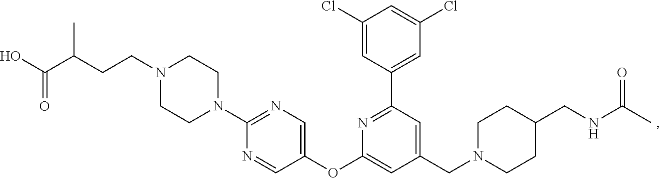 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]

or a pharmaceutically acceptable salt thereof.

In certain embodiments, a compound of Formula (I) or Formula (II) may be any one of the compounds found in Table 1 below. In certain embodiments, the disclosed compositions, methods, and uses comprise administering to the subject in need thereof a therapeutically effective amount of any one of the compounds found in Table 1 below.

TABLE 1
Compounds useful in the present disclosure

| # | Structure | Name |
|---|---|---|
| 1 | 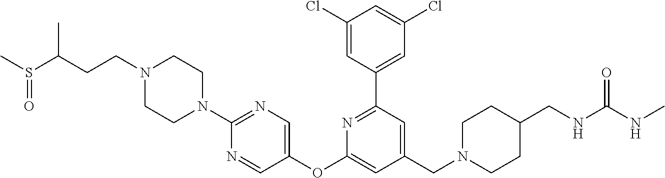 | 1-((1-((2-(3,5-dichloro- phenyl)-6-((2-(4-(3- (methylsulfinyl)butyl) piperazin-1-yl)pyrimidin- 5-yl)oxy)pyridin-4-yl) methyl)piperidin-4-yl) methyl)-3-methylurea |
| 2 | 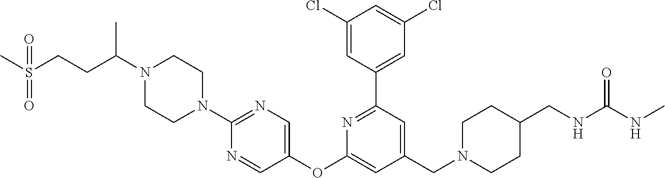 | 1-((1-((2-(3,5-dichloro- phenyl)-6-((2-(4-(4- (methylsulfonyl)butan- 2-yl)piperazin-1-yl) pyrimidin-5-yl)oxy) pyridin-4-yl)methyl) piperidin-4-yl)methyl)- 3-methylurea |
| 3 | 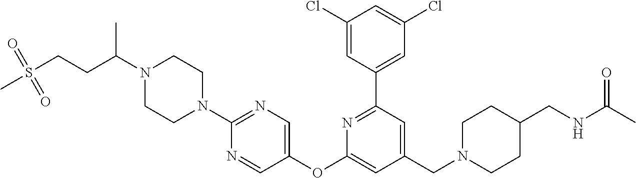 [SMILES: CC(=O)NCC1CCN(Cc2cc(Oc3cnc(N4CCN(C(C)CCS(C)(=O)=O)CC4)nc3)nc(-c3cc(Cl)cc(Cl)c3)c2)CC1] | N-((1-((2-(3,5-dichloro- phenyl)-6-((2-(4-(4- (methylsulfonyl)butan- 2-yl)piperazin-1-yl) pyrimidin-5-yl)oxy) pyridin-4-yl)methyl) piperidin-4-yl)methyl) acetamide |
| 4 | 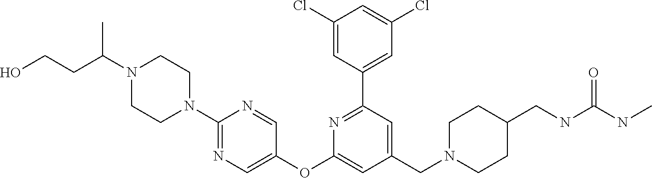 [SMILES: CNC(=O)NCC1CCN(Cc2cc(Oc3cnc(N4CCN(C(C)CCO)CC4)nc3)nc(-c3cc(Cl)cc(Cl)c3)c2)CC1] | 1-((1-((2-(3,5-dichloro- phenyl)-6-((2-(4-(4- hydroxybutan-2-yl) piperazin-1-yl)pyrimidin- 5-yl)oxy)pyridin-4-yl) methyl)piperidin-4-yl) methyl)-3-methylurea |
| 5 | 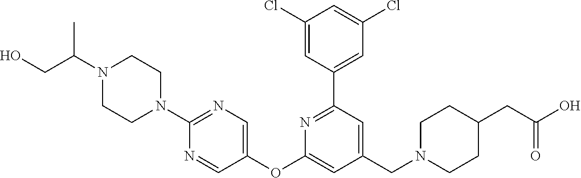 [SMILES: CC(CO)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(1- hydroxypropan-2-yl) piperazin-1-yl)pyrimidin- 5-yl)oxy)pyridin-4-yl) methyl)piperidin-4-yl) acetic acid |
| 6 | 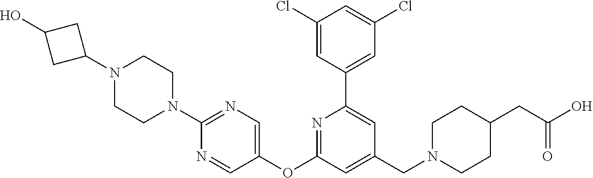 [SMILES: O=C(O)CC1CCN(Cc2cc(Oc3cnc(N4CCN(C5CC(O)C5)CC4)nc3)nc(-c3cc(Cl)cc(Cl)c3)c2)CC1] | 2-(1-((2-(3,5-dichloro- phenyl)-6-((2-(4-(3- hydroxycyclobutyl) piperazin-1-yl)pyrimidin- 5-yl)oxy)pyridin-4-yl) methyl)piperidin-4-yl) acetic acid |
| 7 | 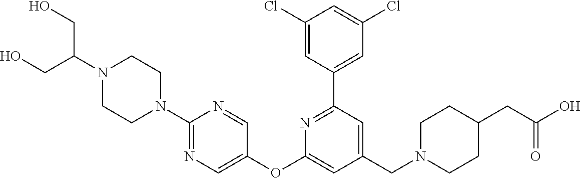 [SMILES: O=C(O)CC1CCN(Cc2cc(Oc3cnc(N4CCN(C(CO)CO)CC4)nc3)nc(-c3cc(Cl)cc(Cl)c3)c2)CC1] | 2-(1-((2-(3,5-dichloro- phenyl)-6-((2-(4-(1,3- dihydroxypropan-2-yl) piperazin-1-yl)pyrimidin- 5-yl)oxy)pyridin-4- yl)methyl)piperidin-4- yl)acetic acid |
| 8 | 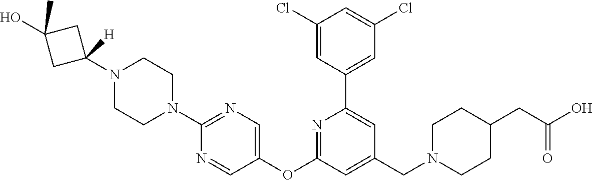 [SMILES: C[C@]1(O)C[C@@H](N2CCN(c3ncc(Oc4cc(CN5CCC(CC(=O)O)CC5)cc(-c5cc(Cl)cc(Cl)c5)n4)cn3)CC2)C1] | 2-(1-((2-(3,5-dichloro- phenyl)-6-((2-(4-((1s,3s)- 3-hydroxy-3-methyl- cyclobutyl)piperazin-1- yl)pyrimidin-5-yl)oxy) pyridin-4-yl)methyl) piperidin-4-yl)acetic acid |
| 9 | 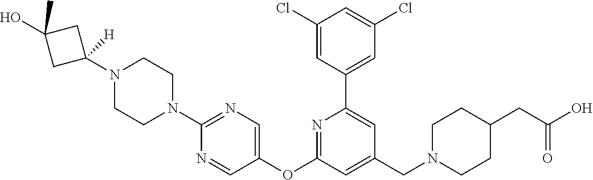 [SMILES: C[C@]1(O)C[C@H](N2CCN(c3ncc(Oc4cc(CN5CCC(CC(=O)O)CC5)cc(-c5cc(Cl)cc(Cl)c5)n4)cn3)CC2)C1] | 2-(1-((2-(3,5-dichloro- phenyl)-6-((2-(4-((1r,3r)- 3-hydroxy-3-methyl- cyclo-butyl)piperazin-1- yl)pyrimidin-5-yl)oxy) pyridin-4-yl)methyl) piperidin-4-yl)acetic acid |
| 10 | 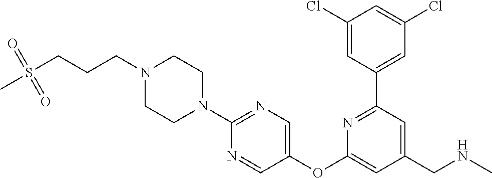 | 1-(2-(3,5-dichlorophenyl)- 6-((2-(4-(3-(methyl- sulfonyl)propyl)piperazin- 1-yl)pyrimidin-5-yl)oxy) pyridin-4-yl)-N-methyl- methanamine |
| 11 | 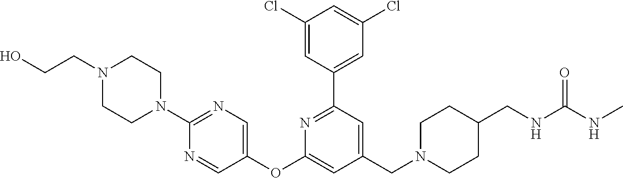 [SMILES: CNC(=O)NCC1CCN(Cc2cc(Oc3cnc(N4CCN(CCO)CC4)nc3)nc(-c3cc(Cl)cc(Cl)c3)c2)CC1] | 1-((1-((2-(3,5-dichloro- phenyl)-6-((2-(4-(2- hydroxyethyl)piperazin-1- yl)pyrimidin-5-yl)oxy) pyridin-4-yl)methyl) piperidin-4-yl)methyl)-3- methylurea |
| 12 | 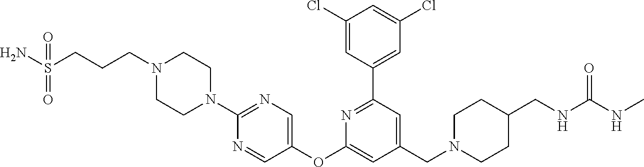 [SMILES: CNC(=O)NCC1CCN(Cc2cc(Oc3cnc(N4CCN(CCCS(N)(=O)=O)CC4)nc3)nc(-c3cc(Cl)cc(Cl)c3)c2)CC1] | 3-(4-(5-((6-(3,5-dichloro- phenyl)-4-((4-((3-methyl- ureido)methyl)piperidin- 1-yl)methyl)pyridin-2- yl)oxy)pyrimidin-2-yl) piperazin-1-yl)propane- 1-sulfonamide |
| 13 | 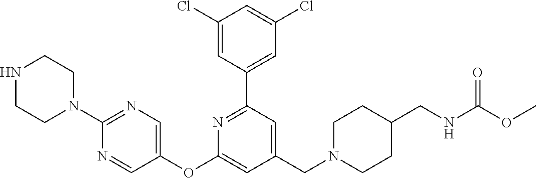 [SMILES: COC(=O)NCC1CCN(Cc2cc(Oc3cnc(N4CCNCC4)nc3)nc(-c3cc(Cl)cc(Cl)c3)c2)CC1] | methyl((1-((2-(3,5- dichloro-phenyl)-6-((2- (piperazin-1-yl)pyrimidin- 5-yl)oxy)pyridin-4-yl) methyl)piperidin-4- yl)methyl)carbamate |
| 14 | 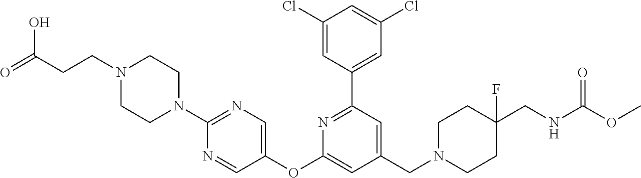 [SMILES: COC(=O)NCC1(F)CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-fluoro-4- (((methoxycarbonyl) amino)methyl)piperidin- 1-yl)methyl)pyridin-2- yl)oxy)pyrimidin-2- yl)piperazin-1- yl)propanoic acid |
| 15 | 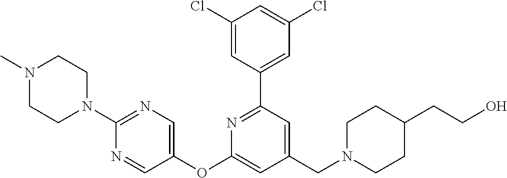 [SMILES: CN1CCN(c2ncc(Oc3cc(CN4CCC(CCO)CC4)cc(-c4cc(Cl)cc(Cl)c4)n3)cn2)CC1] | 2-(1-((2-(3,5-dichloro- phenyl)-6-((2-(4-methyl- piperazin-1-yl)pyrimidin- 5-yl)oxy)pyridin-4- yl)methyl)piperidin-4- yl)ethanol |
| 16 | 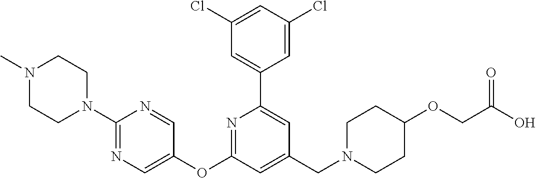 [SMILES: CN1CCN(c2ncc(Oc3cc(CN4CCC(OCC(=O)O)CC4)cc(-c4cc(Cl)cc(Cl)c4)n3)cn2)CC1] | 2-((1-((2-(3,5-dichloro- phenyl)-6-((2-(4-methyl- piperazin-1-yl)pyrimidin- 5-yl)oxy)pyridin-4-yl) methyl)piperidin-4- yl)oxy)acetic acid |
| 17 | 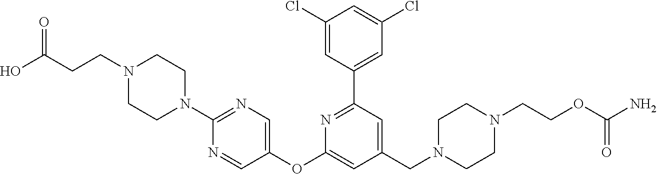 [SMILES: NC(=O)OCCN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(2- (carbamoyloxy)ethyl) piperazin-1-yl)methyl)-6- (3,5-dichlorophenyl) pyridin-2-yl)oxy) pyrimidin-2-yl)piperazin- 1-yl)propanoic acid |
| 18 | 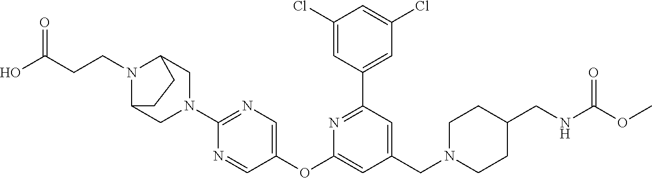 [SMILES: COC(=O)NCC1CCN(Cc2cc(Oc3cnc(N4CC5CCC(C4)N5CCC(=O)O)nc3)nc(-c3cc(Cl)cc(Cl)c3)c2)CC1] | 3-(3-(5-((6-(3,5-dichloro- phenyl)-4-((4-(((methoxy- carbonyl)amino)methyl) piperidin-1-yl)methyl) pyridin-2-yl)oxy) pyrimidin-2-yl)-3,8- diazabicyclo[3.2.1]octan- 8-yl)propanoic acid |
| 19 | 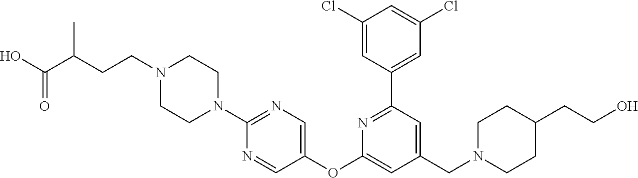 [SMILES: CC(CCN1CCN(c2ncc(Oc3cc(CN4CCC(CCO)CC4)cc(-c4cc(Cl)cc(Cl)c4)n3)cn2)CC1)C(=O)O] | 4-(4-(5-((6-(3,5-dichloro- phenyl)-4-((4-(2-hydroxy- ethyl)piperidin-1-yl) methyl)pyridin-2-yl)oxy) pyrimidin-2-yl)piperazin- 1-yl)-2-methylbutanoic acid |
| 20 | 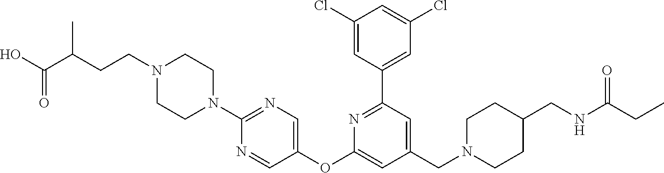 [SMILES: CCC(=O)NCC1CCN(Cc2cc(Oc3cnc(N4CCN(CCC(C)C(=O)O)CC4)nc3)nc(-c3cc(Cl)cc(Cl)c3)c2)CC1] | 4-(4-(5-((6-(3,5-dichloro- phenyl)-4-((4-(propion- amidomethyl)piperidin-1- yl)methyl)pyridin-2- yl)oxy)pyrimidin-2- yl)piperazin-1-yl)-2- methylbutanoic acid |
| 21 | 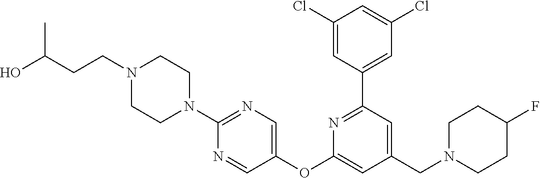 [SMILES: CC(O)CCN1CCN(c2ncc(Oc3cc(CN4CCC(F)CC4)cc(-c4cc(Cl)cc(Cl)c4)n3)cn2)CC1] | 4-(4-(5-((6-(3,5-dichloro- phenyl)-4-((4-fluoro- piperidin-1-yl)methyl) pyridin-2-yl)oxy) pyrimidin-2-yl)piperazin- 1-yl)butan-2-ol |
| 22 | 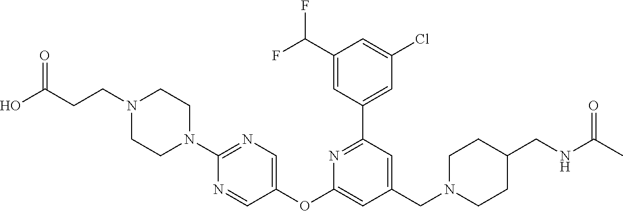 [SMILES: CC(=O)NCC1CCN(Cc2cc(Oc3cnc(N4CCN(CCC(=O)O)CC4)nc3)nc(-c3cc(Cl)cc(C(F)F)c3)c2)CC1] | 3-(4-(5-((4-((4- (acetamidomethyl) piperidin-1-yl)methyl)- 6-(3-chloro-5-(difluoro- methyl)phenyl)pyridin- 2-yl)oxy)pyrimidin-2- yl)piperazin-1- yl)propanoic acid |
| 23 | 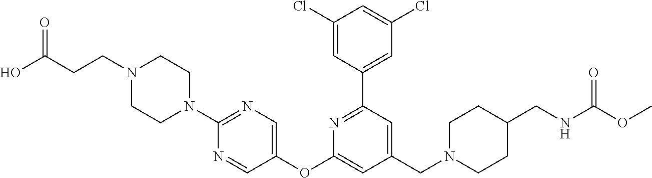 [SMILES: COC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-(((methoxy- carbonyl)amino)methyl) piperidin-1-yl)methyl) pyridin-2-yl)oxy) pyrimidin-2-yl)piperazin- 1-yl)propanoic acid |
| 24 | 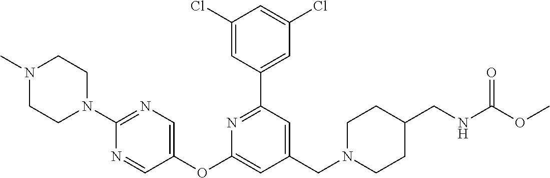 | methyl((1-((2-(3,5- dichlorophenyl)-6-((2- (4-methylpiperazin-1- yl)pyrimidin-5-yl)oxy) pyridin-4-yl)methyl) piperidin-4-yl)methyl) carbamate |
| 25 | 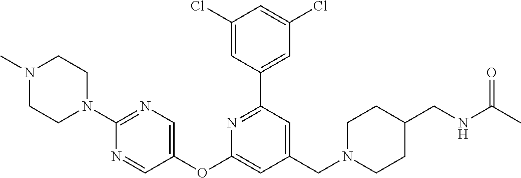 [SMILES: CC(=O)NCC1CCN(Cc2cc(Oc3cnc(N4CCN(C)CC4)nc3)nc(-c3cc(Cl)cc(Cl)c3)c2)CC1] | N-((1-((2-(3,5-dichloro- phenyl)-6-((2-(4-methyl- piperazin-1-yl)pyrimidin- 5-yl)oxy)pyridin-4-yl) methyl)piperidin-4- yl)methyl)acetamide |
| 26 | 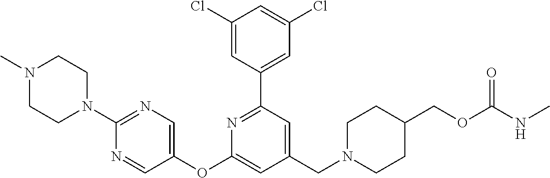 | (1-((2-(3,5-dichloro- phenyl)-6-((2-(4- methylpiperazin-1- yl)pyrimidin-5-yl)oxy) pyridin-4-yl)methyl) piperidin-4-yl)methyl- methylcarbamate |
| 27 | 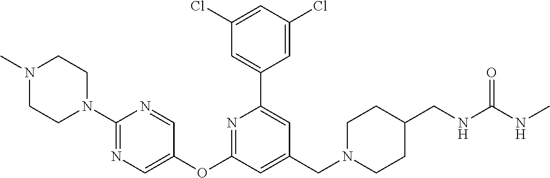 | 1-((1-((2-(3,5-dichloro- phenyl)-6-((2-(4- methylpiperazin-1- yl)pyrimidin-5-yl)oxy) pyridin-4-yl)methyl) piperidin-4-yl)methyl)- 3-methylurea |
| 28 | 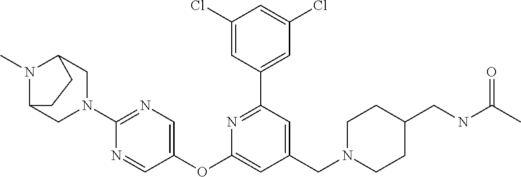 [SMILES: CC(=O)NCC1CCN(Cc2cc(Oc3cnc(N4CC5CCC(C4)N5C)nc3)nc(-c3cc(Cl)cc(Cl)c3)c2)CC1] | N-((1-((2-(3,5-dichloro- phenyl)-6-((2-(8-methyl- 3,8-diazabicyclo[3.2.1] octan-3-yl)pyrimidin-5- yl)oxy)pyridin-4- yl)methyl)piperidin-4- yl)methyl)acetamide |
| 29 | 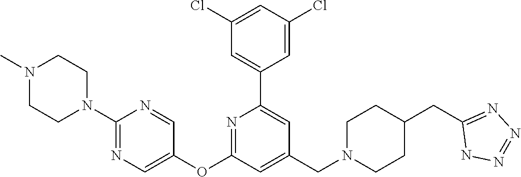 [SMILES: CN1CCN(c2ncc(Oc3cc(CN4CCC(Cc5nnn[nH]5)CC4)cc(-c4cc(Cl)cc(Cl)c4)n3)cn2)CC1] | 5-((4-((4-((1H-tetrazol-5- yl)methyl)piperidin-1- yl)methyl)-6-(3,5- dichlorophenyl)pyridin- 2-yl)oxy)-2-(4-methyl- piperazin-1-yl)pyrimidine |
| 30 | 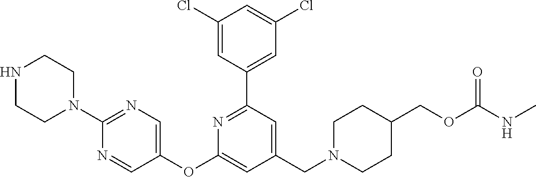 [SMILES: CNC(=O)OCC1CCN(Cc2cc(Oc3cnc(N4CCNCC4)nc3)nc(-c3cc(Cl)cc(Cl)c3)c2)CC1] | (1-((2-(3,5-dichloro- phenyl)-6-((2-(piperazin- 1-yl)pyrimidin-5-yl)oxy) pyridin-4-yl)methyl) piperidin-4-yl)methyl- methyl carbamate |
| 31 | 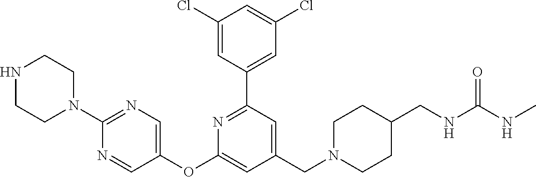 [SMILES: CNC(=O)NCC1CCN(Cc2cc(Oc3cnc(N4CCNCC4)nc3)nc(-c3cc(Cl)cc(Cl)c3)c2)CC1] | 1-((1-((2-(3,5-dichloro- phenyl)-6-((2-(piperazin- 1-yl)pyrimidin-5-yl)oxy) pyridin-4-yl)methyl) piperidin-4-yl)methyl)- 3-methylurea |
| 32 | 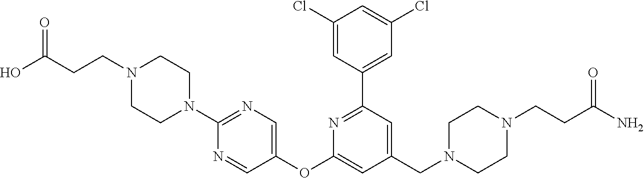 [SMILES: NC(=O)CCN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(3-amino- 3-oxopropyl)piperazin-1- yl)methyl)-6-(3,5- dichlorophenyl)pyridin- 2-yl)oxy)pyrimidin-2-yl) piperazin-1-yl)propanoic acid |
| 33 | 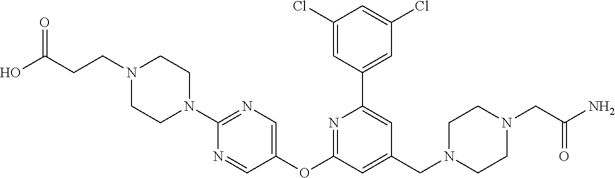 [SMILES: NC(=O)CN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(2-amino- 2-oxoethyl)piperazin-1- yl)methyl)-6-(3,5- dichlorophenyl)pyridin- 2-yl)oxy)pyrimidin-2-yl) piperazin-1-yl)propanoic acid |
| 34 | 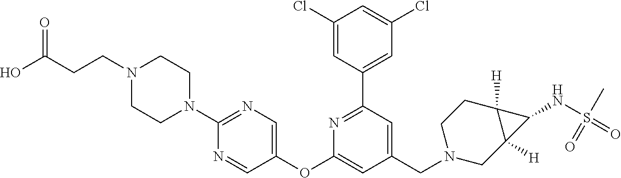 [SMILES: CS(=O)(=O)N[C@H]1[C@@H]2CCN(Cc3cc(Oc4cnc(N5CCN(CCC(=O)O)CC5)nc4)nc(-c4cc(Cl)cc(Cl)c4)c3)C[C@@H]21] | 3-(4-(5-((6-(3,5-dichloro- phenyl)-4-(((1R,7S,8r)- 8-(methyl-sulfonamido)- 4-azabicyclo[5.1.0]octan- 4-yl)methyl)pyridin-2- yl)oxy)pyrimidin-2-yl) piperazin-1-yl)propanoic acid |
| 35 | 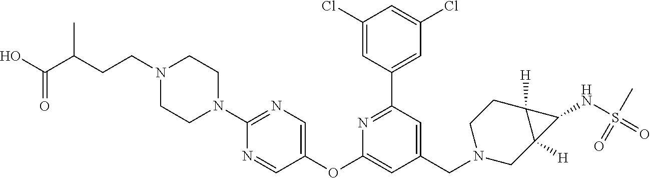 [SMILES: CC(CCN1CCN(c2ncc(Oc3cc(CN4CC[C@@H]5[C@H](C4)[C@H]5NS(C)(=O)=O)cc(-c4cc(Cl)cc(Cl)c4)n3)cn2)CC1)C(=O)O] | 4-(4-(5-((6-(3,5-dichloro- phenyl)-4-(((1R,7S,8r)-8- (methyl-sulfonamido)-4- azabicyclo[5.1.0]octan- 4-yl)methyl)pyridin-2- yl)oxy)pyrimidin-2- yl)piperazin-1-yl)-2- methylbutanoic acid |
| 36 | 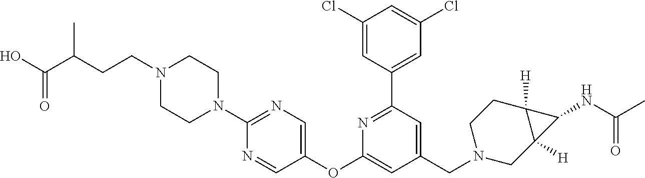 [SMILES: CC(=O)N[C@H]1[C@@H]2CCN(Cc3cc(Oc4cnc(N5CCN(CCC(C)C(=O)O)CC5)nc4)nc(-c4cc(Cl)cc(Cl)c4)c3)C[C@@H]21] | 4-(4-(5-((4-(((1R,7S,8r)- 8-acetamido-4- azabicyclo[5.1.0]octan- 4-yl)methyl)-6-(3,5- dichlorophenyl)pyridin- 2-yl)oxy)pyrimidin-2- yl)piperazin-1-yl)-2- methylbutanoic acid |
| 37 | 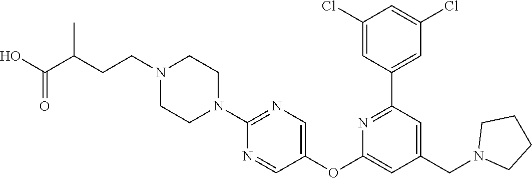 [SMILES: CC(CCN1CCN(c2ncc(Oc3cc(CN4CCCC4)cc(-c4cc(Cl)cc(Cl)c4)n3)cn2)CC1)C(=O)O] | 4-(4-(5-((6-(3,5-dichloro- phenyl)-4-(pyrrolidin-1- ylmethyl)pyridin-2- yl)oxy)pyrimidin-2-yl) piperazin-1-yl)-2-methyl- butanoic acid |
| 38 | 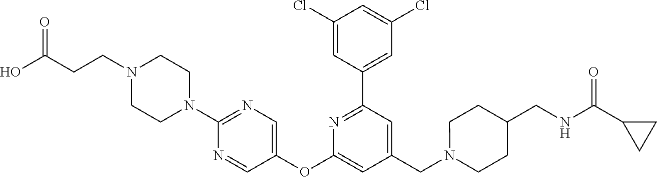 [SMILES: O=C(O)CCN1CCN(c2ncc(Oc3cc(CN4CCC(CNC(=O)C5CC5)CC4)cc(-c4cc(Cl)cc(Cl)c4)n3)cn2)CC1] | 3-(4-(5-((4-((4-(cyclo- propanecarboxamido- methyl)piperidin-1-yl) methyl)-6-(3,5- dichlorophenyl)pyridin- 2-yl)oxy)pyrimidin-2- yl)piperazin-1-yl) propanoic acid |
| 39 | 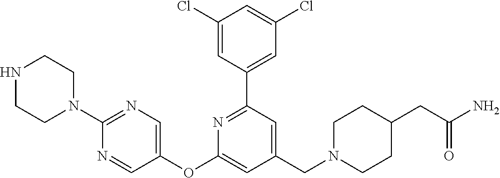 [SMILES: NC(=O)CC1CCN(Cc2cc(Oc3cnc(N4CCNCC4)nc3)nc(-c3cc(Cl)cc(Cl)c3)c2)CC1] | 2-(1-((2-(3,5-dichloro- phenyl)-6-((2-(piperazin- 1-yl)pyrimidin-5-yl)oxy) pyridin-4-yl)methyl) piperidin-4-yl)acetamide |
| 40 | 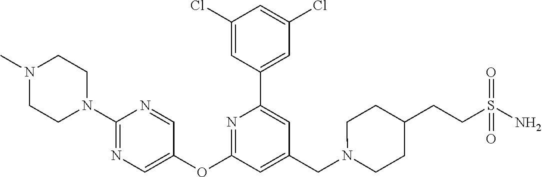 [SMILES: CN1CCN(c2ncc(Oc3cc(CN4CCC(CCS(N)(=O)=O)CC4)cc(-c4cc(Cl)cc(Cl)c4)n3)cn2)CC1] | 2-(1-((2-(3,5-dichloro- phenyl)-6-((2-(4-methyl- piperazin-1-yl)pyrimidin- 5-yl)oxy)pyridin-4- yl)methyl)piperidin-4- yl)ethanesulfonamide |
| 41 | 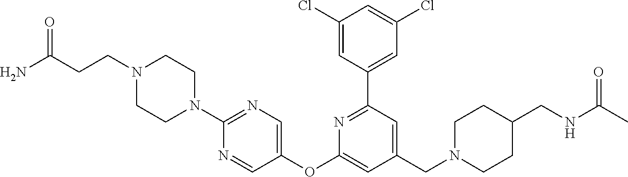 [SMILES: CC(=O)NCC1CCN(Cc2cc(Oc3cnc(N4CCN(CCC(N)=O)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichloro-phenyl) pyridin-2-yl)oxy) pyrimidin-2-yl)piperazin- 1-yl)propanamide |
| 42 | 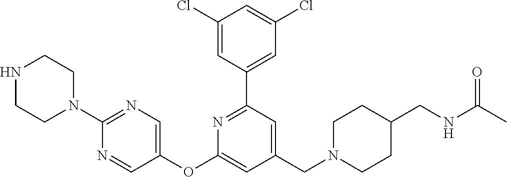 [SMILES: CC(=O)NCC1CCN(Cc2cc(Oc3cnc(N4CCNCC4)nc3)nc(-c3cc(Cl)cc(Cl)c3)c2)CC1] | N-((1-((2-(3,5-dichloro- phenyl)-6-((2-(piperazin- 1-yl)pyrimidin-5-yl)oxy) pyridin-4-yl)methyl) piperidin-4-yl)methyl) acetamide |
| 43 | 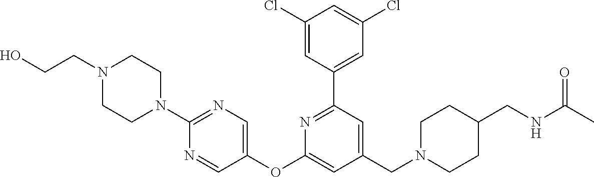 [SMILES: CC(=O)NCC1CCN(Cc2cc(Oc3cnc(N4CCN(CCO)CC4)nc3)nc(-c3cc(Cl)cc(Cl)c3)c2)CC1] | N-((1-((2-(3,5-dichloro- phenyl)-6-((2-(4-(2- hydroxyethyl)piperazin- 1-yl)pyrimidin-5-yl) oxy)pyridin-4-yl) methyl)piperidin-4- yl)methyl)acetamide |
| 44 | 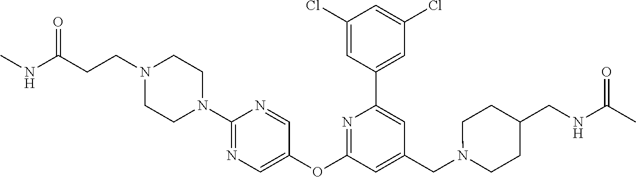 [SMILES: CNC(=O)CCN1CCN(c2ncc(Oc3cc(CN4CCC(CNC(C)=O)CC4)cc(-c4cc(Cl)cc(Cl)c4)n3)cn2)CC1] | 3-(4-(5-((4-((4- (acetamidomethyl) piperidin-1-yl)methyl)-6- (3,5-dichlorophenyl) pyridin-2-yl)oxy) pyrimidin-2-yl)piperazin- 1-yl)-N-methyl- propanamide |
| 45 | 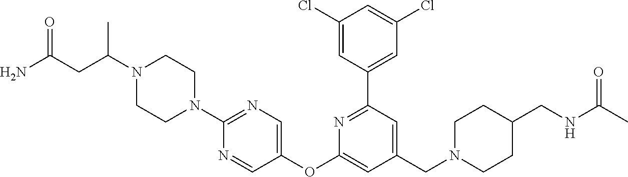 [SMILES: CC(=O)NCC1CCN(Cc2cc(Oc3cnc(N4CCN(C(C)CC(N)=O)CC4)nc3)nc(-c3cc(Cl)cc(Cl)c3)c2)CC1] | 3-(4-(5-((4-((4- (acetamidomethyl) piperidin-1-yl)methyl)-6- (3,5-dichlorophenyl) pyridin-2-yl)oxy) pyrimidin-2-yl)piperazin- 1-yl)butanamide |
| 46 | 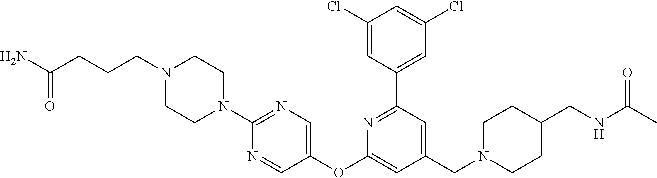 [SMILES: CC(=O)NCC1CCN(Cc2cc(Oc3cnc(N4CCN(CCCC(N)=O)CC4)nc3)nc(-c3cc(Cl)cc(Cl)c3)c2)CC1] | 4-(4-(5-((4-((4- (acetamidomethyl) piperidin-1-yl)methyl)-6- (3,5-dichlorophenyl) pyridin-2-yl)oxy) pyrimidin-2-yl)piperazin- 1-yl)butanamide |
| 47 | 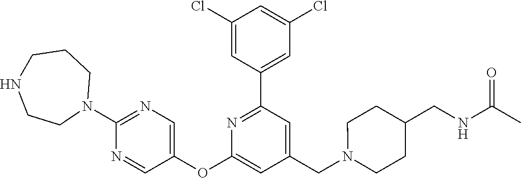 [SMILES: CC(=O)NCC1CCN(Cc2cc(Oc3cnc(N4CCCNCC4)nc3)nc(-c3cc(Cl)cc(Cl)c3)c2)CC1] | N-((1-((2-((2-(1,4- diazepan-1-yl)pyrimidin- 5-yl)oxy)-6-(3,5-dichloro- phenyl)pyridin-4-yl) methyl)piperidin-4- yl)methyl)acetamide |
| 48 | 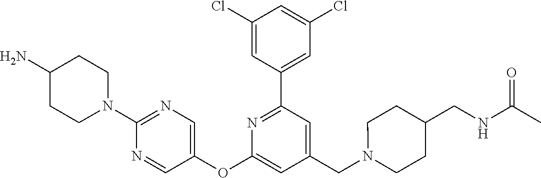 [SMILES: CC(=O)NCC1CCN(Cc2cc(Oc3cnc(N4CCC(N)CC4)nc3)nc(-c3cc(Cl)cc(Cl)c3)c2)CC1] | N-((1-((2-((2-(4-amino- piperidin-1-yl)pyrimidin- 5-yl)oxy)-6-(3,5-dichloro- phenyl)pyridin-4- yl)methyl)piperidin-4- yl)methyl)acetamide |
| 49 | 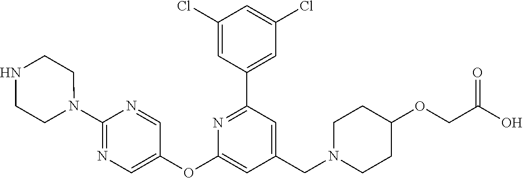 [SMILES: O=C(O)COC1CCN(Cc2cc(Oc3cnc(N4CCNCC4)nc3)nc(-c3cc(Cl)cc(Cl)c3)c2)CC1] | 2-((1-((2-(3,5-dichloro- phenyl)-6-((2-(piperazin- 1-yl)pyrimidin-5-yl)oxy) pyridin-4-yl)methyl) piperidin-4-yl)oxy)acetic acid |
| 50 | 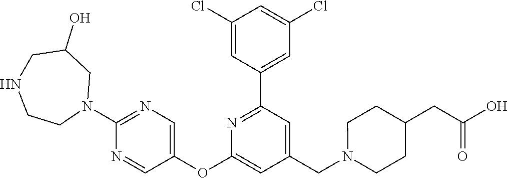 [SMILES: O=C(O)CC1CCN(Cc2cc(Oc3cnc(N4CCNCC(O)C4)nc3)nc(-c3cc(Cl)cc(Cl)c3)c2)CC1] | 2-(1-((2-(3,5-dichloro- phenyl)-6-((2-(6-hydroxy- 1,4-diazepan-1-yl) pyrimidin-5-yl)oxy) pyridin-4-yl)methyl) piperidin-4-yl)acetic acid |
| 51 | 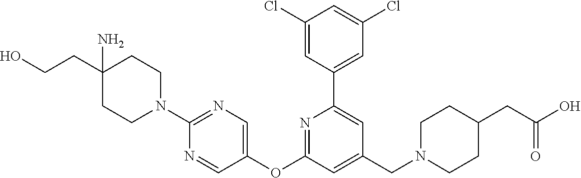 [SMILES: NC1(CCO)CCN(c2ncc(Oc3cc(CN4CCC(CC(=O)O)CC4)cc(-c4cc(Cl)cc(Cl)c4)n3)cn2)CC1] | 2-(1-((2-((2-(4-amino-4- (2-hydroxyethyl)piperidin- 1-yl)pyrimidin-5-yl)oxy)- 6-(3,5-dichlorophenyl) pyridin-4-yl)methyl) piperidin-4-yl)acetic acid |
| 52 | 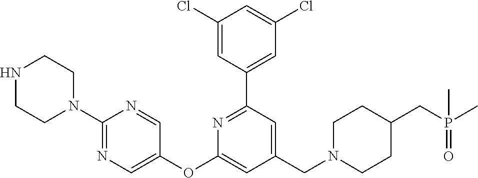 [SMILES: CP(C)(=O)CC1CCN(Cc2cc(Oc3cnc(N4CCNCC4)nc3)nc(-c3cc(Cl)cc(Cl)c3)c2)CC1] | ((1-((2-(3,5-dichloro- phenyl)-6-((2-(piperazin- 1-yl)pyrimidin-5-yl)oxy) pyridin-4-yl)methyl) piperidin-4-yl)methyl) dimethylphosphineoxide |
| 53 | 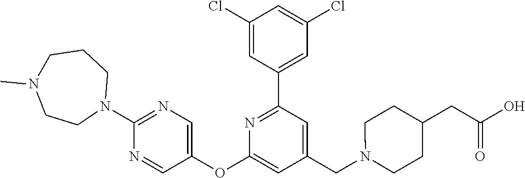 [SMILES: CN1CCCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-methyl- 1,4-diazepan-1-yl) pyrimidin-5-yl)oxy) pyridin-4-yl)methyl) piperidin-4-yl)acetic acid |
| 54 | 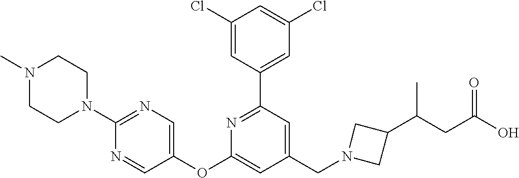 [SMILES: CC(CC(=O)O)C1CN(Cc2cc(Oc3cnc(N4CCN(C)CC4)nc3)nc(-c3cc(Cl)cc(Cl)c3)c2)C1] | 3-(1-((2-(3,5-dichloro- phenyl)-6-((2-(4-methyl- piperazin-1-yl) pyrimidin-5-yl)oxy) pyridin-4-yl)methyl) azetidin-3-yl)butanoic acid |
| 55 | 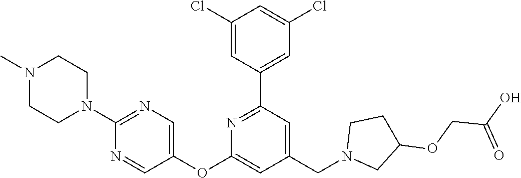 [SMILES: CN1CCN(c2ncc(Oc3cc(CN4CCC(OCC(=O)O)C4)cc(-c4cc(Cl)cc(Cl)c4)n3)cn2)CC1] | 2-((1-((2-(3,5-dichloro- phenyl)-6-((2-(4-methyl- piperazin-1-yl) pyrimidin-5-yl)oxy) pyridin-4-yl)methyl) pyrrolidin-3-yl)oxy)acetic acid |
| 56 | 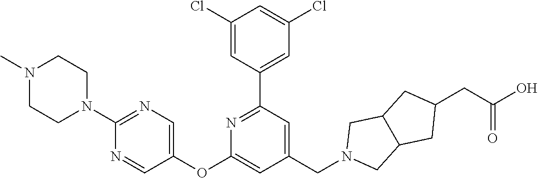 [SMILES: CN1CCN(c2ncc(Oc3cc(CN4CC5CC(CC(=O)O)CC5C4)cc(-c4cc(Cl)cc(Cl)c4)n3)cn2)CC1] | 2-(2-((2-(3,5-dichloro- phenyl)-6-((2-(4-methyl- piperazin-1-yl) pyrimidin-5-yl)oxy) pyridin-4-yl)methyl) octahydrocyclopenta[c] pyrrol-5-yl)acetic acid |
| 57 | 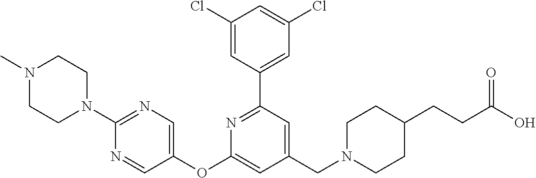 [SMILES: CN1CCN(c2ncc(Oc3cc(CN4CCC(CCC(=O)O)CC4)cc(-c4cc(Cl)cc(Cl)c4)n3)cn2)CC1] | 3-(1-((2-(3,5-dichloro- phenyl)-6-((2-(4-methyl- piperazin-1-yl) pyrimidin-5-yl)oxy) pyridin-4-yl)methyl) piperidin-4-yl)propanoic acid |
| 58 | 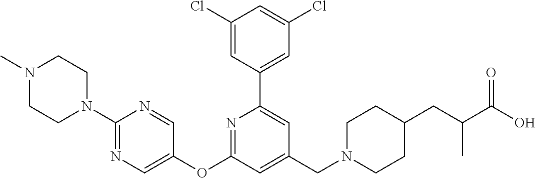 [SMILES: CC(CC1CCN(Cc2cc(Oc3cnc(N4CCN(C)CC4)nc3)nc(-c3cc(Cl)cc(Cl)c3)c2)CC1)C(=O)O] | 3-(1-((2-(3,5-dichloro- phenyl)-6-((2-(4-methyl- piperazin-1-yl)pyrimidin- 5-yl)oxy)pyridin-4-yl) methyl)piperidin-4-yl)- 2-methylpropanoic acid |
| 59 | 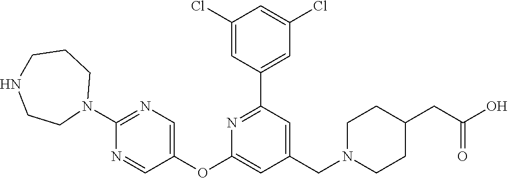 [SMILES: O=C(O)CC1CCN(Cc2cc(Oc3cnc(N4CCCNCC4)nc3)nc(-c3cc(Cl)cc(Cl)c3)c2)CC1] | 2-(1-((2-((2-(1,4-diazepan- 1-yl)pyrimidin-5-yl)oxy)- 6-(3,5-dichlorophenyl) pyridin-4-yl)methyl) piperidin-4-yl)acetic acid |
| 60 | 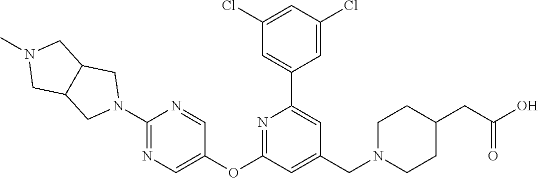 [SMILES: CN1CC2CN(c3ncc(Oc4cc(CN5CCC(CC(=O)O)CC5)cc(-c5cc(Cl)cc(Cl)c5)n4)cn3)CC2C1] | 2-(1-((2-(3,5-dichloro- phenyl)-6-((2-(5-methyl- hexahydropyrrolo[3,4- c]pyrrol-2(1H)-yl) pyrimidin-5-yl)oxy) pyridin-4-yl)methyl) piperidin-4-yl)acetic acid |
| 61 | 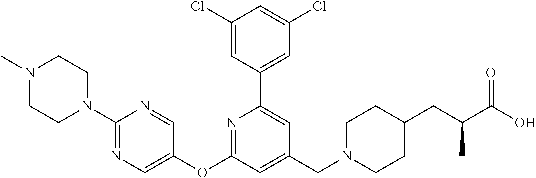 [SMILES: C[C@@H](CC1CCN(Cc2cc(Oc3cnc(N4CCN(C)CC4)nc3)nc(-c3cc(Cl)cc(Cl)c3)c2)CC1)C(=O)O] | (S)-3-(1-((2-(3,5- dichlorophenyl)-6-((2-(4- methylpiperazin-1-yl) pyrimidin-5-yl)oxy) pyridin-4-yl)methyl) piperidin-4-yl)-2-methyl- propanoic acid |
| 62 | 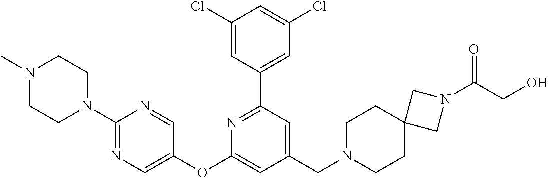 [SMILES: CN1CCN(c2ncc(Oc3cc(CN4CCC5(CC4)CN(C(=O)CO)C5)cc(-c4cc(Cl)cc(Cl)c4)n3)cn2)CC1] | 1-(7-((2-(3,5-dichloro- phenyl)-6-((2-(4-methyl- piperazin-1-yl)pyrimidin- 5-yl)oxy)pyridin-4-yl) methyl)-2,7-diazaspiro [3.5]nonan-2-yl)-2- hydroxyethanone |
| 63 | 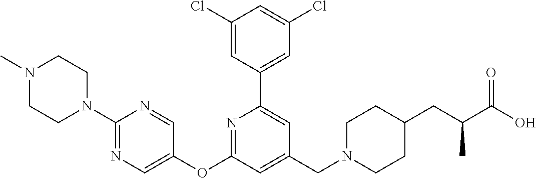 [SMILES: C[C@@H](CC1CCN(Cc2cc(Oc3cnc(N4CCN(C)CC4)nc3)nc(-c3cc(Cl)cc(Cl)c3)c2)CC1)C(=O)O] | (R)-3-(1-((2-(3,5-dichloro- phenyl)-6-((2-(4-methyl- piperazin-1-yl)pyrimidin- 5-yl)oxy)pyridin-4-yl) methyl)piperidin-4-yl)-2- methylpropanoic acid |
| 64 | 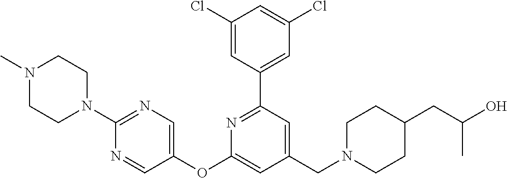 [SMILES: CC(O)CC1CCN(Cc2cc(Oc3cnc(N4CCN(C)CC4)nc3)nc(-c3cc(Cl)cc(Cl)c3)c2)CC1] | 1-(1-((2-(3,5-dichloro- phenyl)-6-((2-(4-methyl- piperazin-1-yl)pyrimidin- 5-yl)oxy)pyridin-4-yl) methyl)piperidin-4-yl) propan-2-ol |
| 65 | 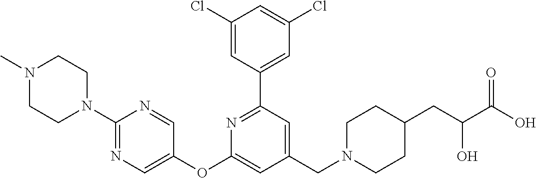 [SMILES: CN1CCN(c2ncc(Oc3cc(CN4CCC(CC(O)C(=O)O)CC4)cc(-c4cc(Cl)cc(Cl)c4)n3)cn2)CC1] | 3-(1-((2-(3,5-dichloro- phenyl)-6-((2-(4-methyl- piperazin-1-yl)pyrimidin- 5-yl)oxy)pyridin-4-yl) methyl)piperidin-4-yl)- 2-hydroxypropanoic acid |
| 66 | 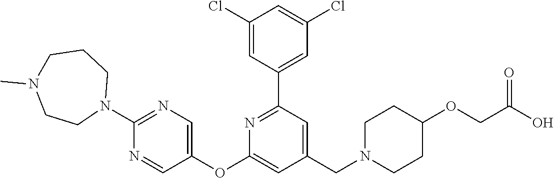 [SMILES: CN1CCCN(c2ncc(Oc3cc(CN4CCC(OCC(=O)O)CC4)cc(-c4cc(Cl)cc(Cl)c4)n3)cn2)CC1] | 2-((1-((2-(3,5-dichloro- phenyl)-6-((2-(4-methyl- 1,4-diazepan-1-yl) pyrimidin-5-yl)oxy) pyridin-4-yl)methyl) piperidin-4-yl)oxy)acetic acid |
| 67 | 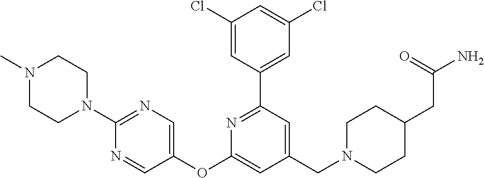 [SMILES: CN1CCN(c2ncc(Oc3cc(CN4CCC(CC(N)=O)CC4)cc(-c4cc(Cl)cc(Cl)c4)n3)cn2)CC1] | 2-(1-((2-(3,5-dichloro- phenyl)-6-((2-(4-methyl- piperazin-1-yl)pyrimidin- 5-yl)oxy)pyridin-4-yl) methyl)piperidin-4- yl)acetamide |
| 68 | 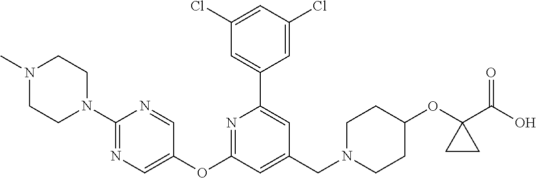 [SMILES: CN1CCN(c2ncc(Oc3cc(CN4CCC(OC5(C(=O)O)CC5)CC4)cc(-c4cc(Cl)cc(Cl)c4)n3)cn2)CC1] | 1-((1-((2-(3,5-dichloro- phenyl)-6-((2-(4-methyl- piperazin-1-yl)pyrimidin- 5-yl)oxy)pyridin-4-yl) methyl)piperidin-4-yl) oxy)cyclopropane- carboxylic acid |
| 69 | 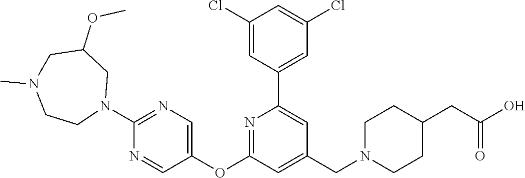 [SMILES: COC1CN(C)CCN(c2ncc(Oc3cc(CN4CCC(CC(=O)O)CC4)cc(-c4cc(Cl)cc(Cl)c4)n3)cn2)C1] | 2-(1-((2-(3,5-dichloro- phenyl)-6-((2-(6-methoxy- 4-methyl-1,4-diazepan-1- yl)pyrimidin-5-yl)oxy) pyridin-4-yl)methyl) piperidin-4-yl)acetic acid |
| 70 | 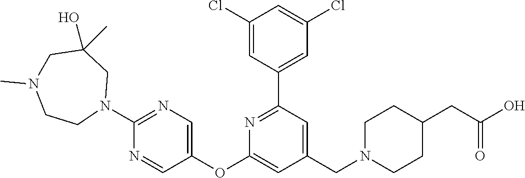 [SMILES: CN1CCN(c2ncc(Oc3cc(CN4CCC(CC(=O)O)CC4)cc(-c4cc(Cl)cc(Cl)c4)n3)cn2)CC(C)(O)C1] | 2-(1-((2-(3,5-dichloro- phenyl)-6-((2-(6-hydroxy- 4,6-dimethyl-1,4- diazepan-1-yl)pyrimidin- 5-yl)oxy)pyridin-4- yl)methyl)piperidin-4- yl)acetic acid |
| 71 | 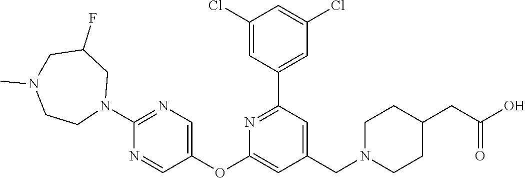 [SMILES: CN1CCN(c2ncc(Oc3cc(CN4CCC(CC(=O)O)CC4)cc(-c4cc(Cl)cc(Cl)c4)n3)cn2)CC(F)C1] | 2-(1-((2-(3,5-dichloro- phenyl)-6-((2-(6-fluoro- 4-methyl-1,4-diazepan-1- yl)pyrimidin-5-yl)oxy) pyridin-4-yl)methyl) piperidin-4-yl)acetic acid |
| 72 | 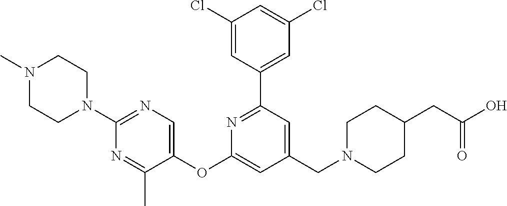 [SMILES: Cc1nc(N2CCN(C)CC2)ncc1Oc1cc(CN2CCC(CC(=O)O)CC2)cc(-c2cc(Cl)cc(Cl)c2)n1] | 2-(1-((2-(3,5-dichloro- phenyl)-6-((4-methyl-2- (4-methylpiperazin-1- yl)pyrimidin-5-yl)oxy) pyridin-4-yl)methyl) piperidin-4-yl)acetic acid |
| 73 | 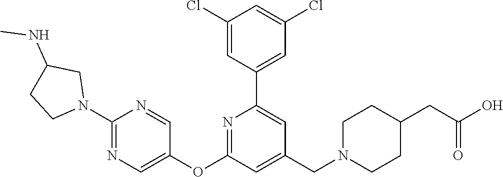 [SMILES: CNC1CCN(c2ncc(Oc3cc(CN4CCC(CC(=O)O)CC4)cc(-c4cc(Cl)cc(Cl)c4)n3)cn2)C1] | 2-(1-((2-(3,5-dichloro- phenyl)-6-((2-(3-(methyl- amino)pyrrolidin-1-yl) pyrimidin-5-yl)oxy) pyridin-4-yl)methyl) piperidin-4-yl)acetic acid |
| 74 | 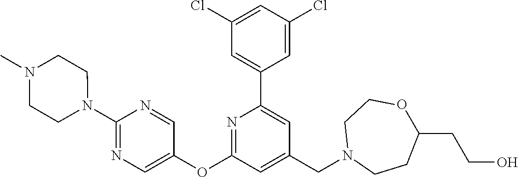 [SMILES: CN1CCN(c2ncc(Oc3cc(CN4CCOC(CCO)CC4)cc(-c4cc(Cl)cc(Cl)c4)n3)cn2)CC1] | (S)-2-(4-((2-(3,5-dichloro- phenyl)-6-((2-(4-methyl- piperazin-1-yl)pyrimidin- 5-yl)oxy)pyridin-4-yl) methyl)-1,4-oxazepan-7- yl)ethanol |
| 75 | 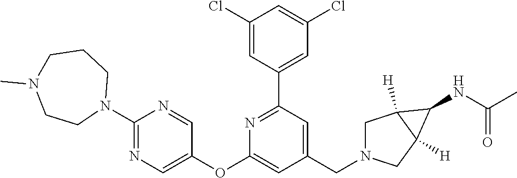 [SMILES: CC(=O)N[C@@H]1[C@@H]2CN(Cc3cc(Oc4cnc(N5CCCN(C)CC5)nc4)nc(-c4cc(Cl)cc(Cl)c4)c3)C[C@@H]21] | N-((1R,5S,6r)-3-((2-(3,5- dichlorophenyl)-6-((2-(4- methyl-1,4-diazepan-1- yl)pyrimidin-5-yl)oxy) pyridin-4-yl)methyl)-3- azabicyclo[3.1.0]hexan-6- yl)acetamide |
| 76 | 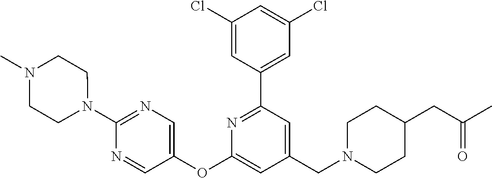 [SMILES: CC(=O)CC1CCN(Cc2cc(Oc3cnc(N4CCN(C)CC4)nc3)nc(-c3cc(Cl)cc(Cl)c3)c2)CC1] | 1-(1-((2-(3,5-dichloro- phenyl)-6-((2-(4-methyl- piperazin-1-yl)pyrimidin- 5-yl)oxy)pyridin-4-yl) methyl)piperidin-4- yl)propan-2-one |
| 77 | 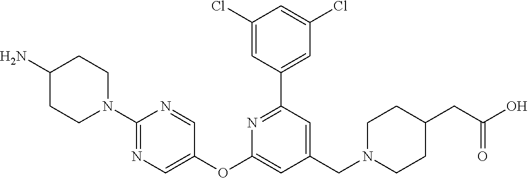 [SMILES: NC1CCN(c2ncc(Oc3cc(CN4CCC(CC(=O)O)CC4)cc(-c4cc(Cl)cc(Cl)c4)n3)cn2)CC1] | 2-(1-((2-((2-(4-amino- piperidin-1-yl)pyrimidin- 5-yl)oxy)-6-(3,5-dichloro- phenyl)pyridin-4- yl)methyl)piperidin-4- yl)acetic acid |
| 78 | 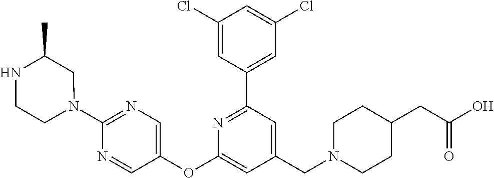 [SMILES: C[C@H]1CN(c2ncc(Oc3cc(CN4CCC(CC(=O)O)CC4)cc(-c4cc(Cl)cc(Cl)c4)n3)cn2)CCN1] | (S)-2-(1-((2-(3,5-dichloro- phenyl)-6-((2-(3-methyl- piperazin-1-yl)pyrimidin- 5-yl)oxy)pyridin-4-yl) methyl)piperidin-4- yl)acetic acid |
| 79 | 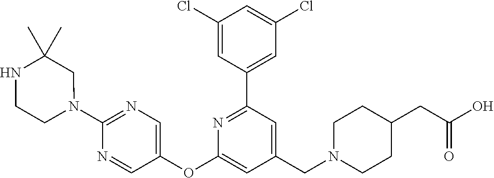 [SMILES: CC1(C)CN(c2ncc(Oc3cc(CN4CCC(CC(=O)O)CC4)cc(-c4cc(Cl)cc(Cl)c4)n3)cn2)CCN1] | 2-(1-((2-(3,5-dichloro- phenyl)-6-((2-(3,3- dimethylpiperazin-1- yl)pyrimidin-5-yl)oxy) pyridin-4-yl)methyl) piperidin-4-yl)acetic acid |
| 80 | 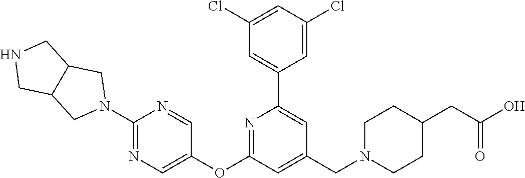 [SMILES: O=C(O)CC1CCN(Cc2cc(Oc3cnc(N4CC5CNCC5C4)nc3)nc(-c3cc(Cl)cc(Cl)c3)c2)CC1] | 2-(1-((2-(3,5-dichloro- phenyl)-6-((2-(hexahydro- pyrrolo[3,4-c]pyrrol- 2(1H)-yl)pyrimidin-5-yl) oxy)pyridin-4- yl)methyl)piperidin-4- yl)acetic acid |
| 81 | 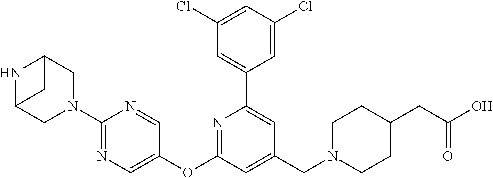 [SMILES: O=C(O)CC1CCN(Cc2cc(Oc3cnc(N4CC5CC(C4)N5)nc3)nc(-c3cc(Cl)cc(Cl)c3)c2)CC1] | 2-(1-((2-((2-(3,6-diaza- bicyclo[3.1.1]heptan-3- yl)pyrimidin-5-yl)oxy)- 6-(3,5-dichlorophenyl) pyridin-4-yl)methyl) piperidin-4-yl)acetic acid |
| 82 | 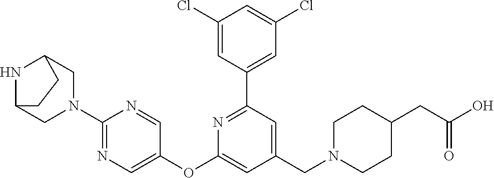 [SMILES: O=C(O)CC1CCN(Cc2cc(Oc3cnc(N4CC5CCC(C4)N5)nc3)nc(-c3cc(Cl)cc(Cl)c3)c2)CC1] | 2-(1-((2-((2-(3,8-diaza- bicyclo[3.2.1]octan-3- yl)pyrimidin-5-yl)oxy)- 6-(3,5-dichloro- phenyl)pyridin-4-yl) methyl)piperidin-4- yl)acetic acid |
| 83 | 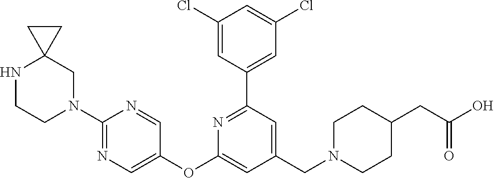 [SMILES: O=C(O)CC1CCN(Cc2cc(Oc3cnc(N4CCNC5(CC5)C4)nc3)nc(-c3cc(Cl)cc(Cl)c3)c2)CC1] | 2-(1-((2-((2-(4,7-diaza- spiro[2.5]octan-7- yl)pyrimidin-5-yl)oxy)- 6-(3,5-dichlorophenyl) pyridin-4-yl)methyl) piperidin-4-yl)acetic acid |
| 84 | 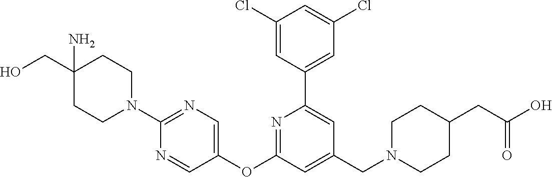 [SMILES: NC1(CO)CCN(c2ncc(Oc3cc(CN4CCC(CC(=O)O)CC4)cc(-c4cc(Cl)cc(Cl)c4)n3)cn2)CC1] | 2-(1-((2-((2-(4-amino-4- (hydroxymethyl)piperidin- 1-yl)pyrimidin-5-yl)oxy)- 6-(3,5-dichlorophenyl) pyridin-4-yl)methyl) piperidin-4-yl)acetic acid |
| 85 | 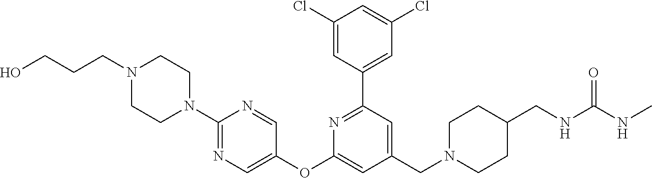 [SMILES: CNC(=O)NCC1CCN(Cc2cc(Oc3cnc(N4CCN(CCCO)CC4)nc3)nc(-c3cc(Cl)cc(Cl)c3)c2)CC1] | 1-((1-((2-(3,5-dichloro- phenyl)-6-((2-(4-(3- hydroxypropyl)piperazin- 1-yl)pyrimidin-5-yl)oxy) pyridin-4-yl)methyl) piperidin-4-yl)methyl)-3- methylurea |
| 86 | 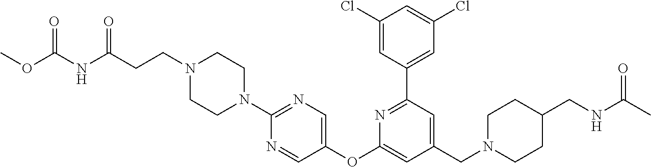 [SMILES: COC(=O)NC(=O)CCN1CCN(c2ncc(Oc3cc(CN4CCC(CNC(C)=O)CC4)cc(-c4cc(Cl)cc(Cl)c4)n3)cn2)CC1] | methyl (3-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)propanoyl)carbamate |
| 87 | 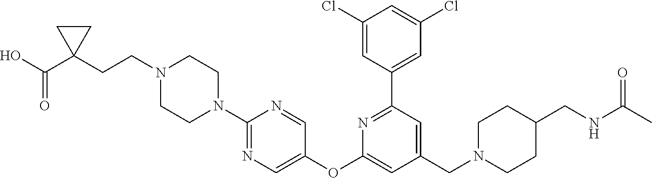 [SMILES: CC(=O)NCC1CCN(Cc2cc(Oc3cnc(N4CCN(CCC5(C(=O)O)CC5)CC4)nc3)nc(-c3cc(Cl)cc(Cl)c3)c2)CC1] | 1-(2-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)ethyl)cyclopropane- carboxylic acid |
| 88 | 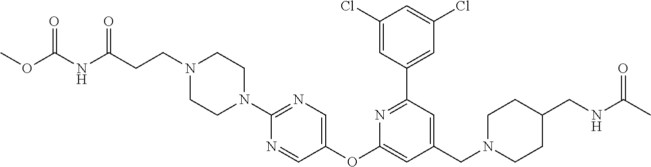 [SMILES: COC(=O)NC(=O)CCN1CCN(c2ncc(Oc3cc(CN4CCC(CNC(C)=O)CC4)cc(-c4cc(Cl)cc(Cl)c4)n3)cn2)CC1] | methyl (3-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)propanoyl)carbamate |
| 89 | 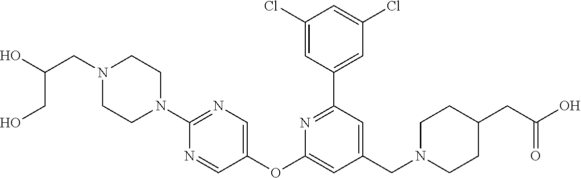 [SMILES: O=C(O)CC1CCN(Cc2cc(Oc3cnc(N4CCN(CC(O)CO)CC4)nc3)nc(-c3cc(Cl)cc(Cl)c3)c2)CC1] | 2-(1-((2-(3,5-dichloro- phenyl)-6-((2-(4-(2,3- dihydroxypropyl) piperazin-1-yl)pyrimidin- 5-yl)oxy)pyridin-4-yl) methyl)piperidin-4-yl) acetic acid |
| 90 | 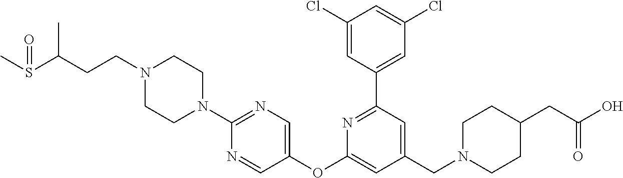 [SMILES: CC(CCN1CCN(c2ncc(Oc3cc(CN4CCC(CC(=O)O)CC4)cc(-c4cc(Cl)cc(Cl)c4)n3)cn2)CC1)S(C)=O] | 2-(1-((2-(3,5-dichloro- phenyl)-6-((2-(4-(3 - (methylsulfinyl)butyl) piperazin-1-yl)pyrimidin- 5-yl)oxy)pyridin-4- yl)methyl)piperidin-4- yl)acetic acid |
| 91 | 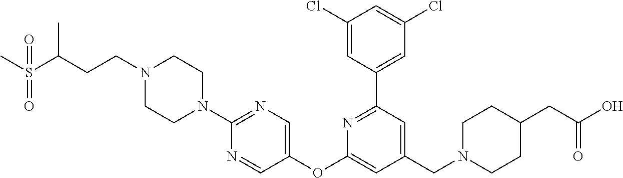 [SMILES: CC(CCN1CCN(c2ncc(Oc3cc(CN4CCC(CC(=O)O)CC4)cc(-c4cc(Cl)cc(Cl)c4)n3)cn2)CC1)S(C)(=O)=O] | 2-(1-((2-(3,5-dichloro- phenyl)-6-((2-(4-(3- (methylsulfonyl)butyl) piperazin-1-yl)pyrimidin- 5-yl)oxy)pyridin-4- yl)methyl)piperidin-4- yl)acetic acid |
| 92 | 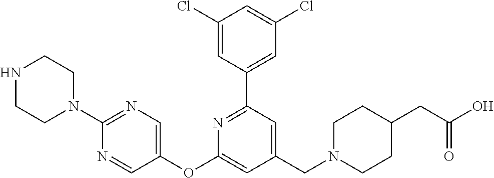 [SMILES: O=C(O)CC1CCN(Cc2cc(Oc3cnc(N4CCNCC4)nc3)nc(-c3cc(Cl)cc(Cl)c3)c2)CC1] | 2-(1-((2-(3,5-dichloro- phenyl)-6-((2-(piperazin- 1-yl)pyrimidin-5-yl)oxy) pyridin-4-yl)methyl) piperidin-4-yl)acetic acid |
| 93 | 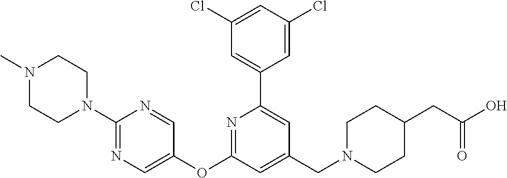 [SMILES: CN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-methyl- piperazin-1-yl)pyrimidin- 5-yl)oxy)pyridin-4-yl) methyl)piperidin-4- yl)acetic acid |
| 94 | 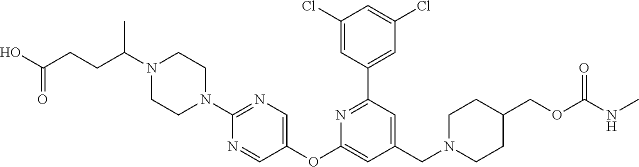 [SMILES: CNC(=O)OCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((6-(3,5-dichloro- phenyl)-4-((4-(((methyl- carbamoyl)oxy)methyl) piperidin-1-yl)methyl) pyridin-2-yl)oxy) pyrimidin-2-yl)piperazin- 1-yl)pentanoic acid |
| 95 | 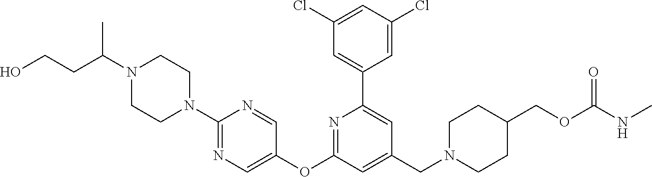 [SMILES: CNC(=O)OCC1CCN(Cc2cc(Oc3cnc(N4CCN(C(C)CCO)CC4)nc3)nc(-c3cc(Cl)cc(Cl)c3)c2)CC1] | (1-((2-(3,5-dichloro- phenyl)-6-((2-(4-(4- hydroxybutan-2-yl) piperazin-1-yl)pyrimidin- 5-yl)oxy)pyridin-4- yl)methyl)piperidin-4- yl)methylmethyl- carbamate |
| 96 | 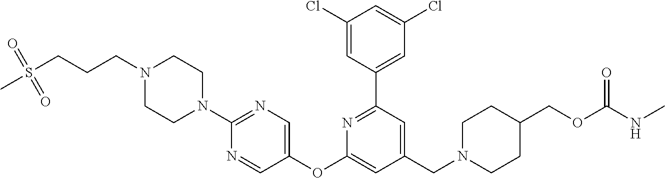 | (1-((2-(3,5-dichloro- phenyl)-6-((2-(4-(3- (methylsulfonyl)propyl) piperazin-1-yl)pyrimidin- 5-yl)oxy)pyridin-4- yl)methyl)piperidin-4- yl)methylmethyl- carbamate |
| 97 | 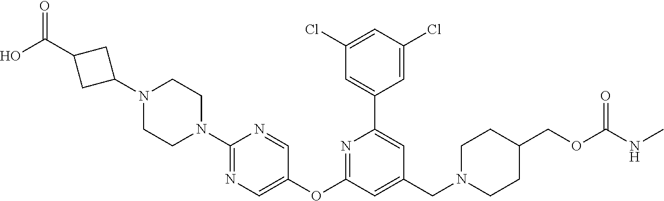 [SMILES: CNC(=O)OCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((6-(3,5-dichloro- phenyl)-4-((4-(((methyl- carbamoyl)oxy)methyl) piperidin-1-yl)methyl) pyridin-2-yl)oxy) pyrimidin-2-yl)piperazin- 1-yl)cyclobutane- carboxylic acid |
| 98 | 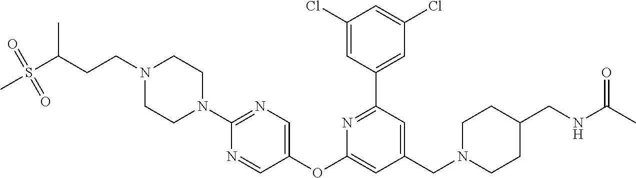 [SMILES: CC(=O)NCC1CCN(Cc2cc(Oc3cnc(N4CCN(CCC(C)S(C)(=O)=O)CC4)nc3)nc(-c3cc(Cl)cc(Cl)c3)c2)CC1] | N-((1-((2-(3,5-dichloro- phenyl)-6-((2-(4-(3- (methylsulfonyl)butyl) piperazin-1-yl)pyrimidin- 5-yl)oxy)pyridin-4- yl)methyl)piperidin-4- yl)methyl)acetamide |
| 99 | 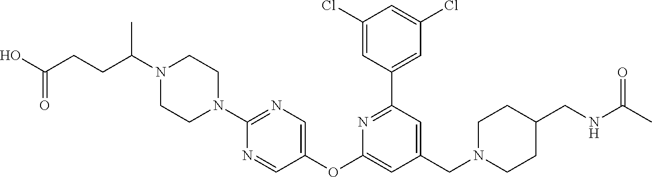 [SMILES: CC(=O)NCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)pentanoic acid |
| 100 | 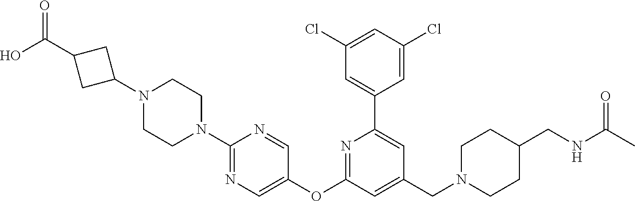 [SMILES: CC(=O)NCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)cyclobutane- carboxylic acid |
| 101 | 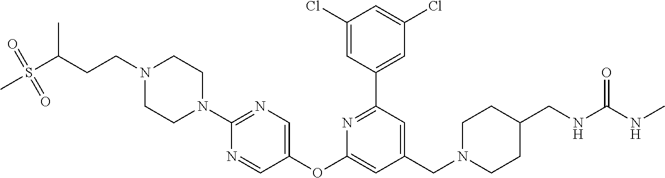 | 1-((1-((2-(3,5-dichloro- phenyl)-6-((2-(4-(3- (methylsulfonyl)butyl) piperazin-1-yl)pyrimidin- 5-yl)oxy)pyridin-4- yl)methyl)piperidin-4- yl)methyl)-3-methylurea |
| 102 | 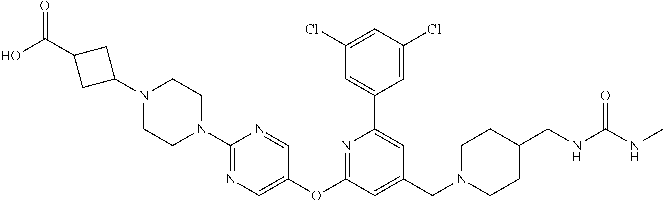 [SMILES: CNC(=O)NCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((6-(3,5-dichloro- phenyl)-4-((4-((3-methyl- ureido)methyl)piperidin- 1-yl)methyl)pyridin-2- yl)oxy)pyrimidin-2- yl)piperazin-1-yl)cyclo- butanecarboxylic acid |
| 103 | 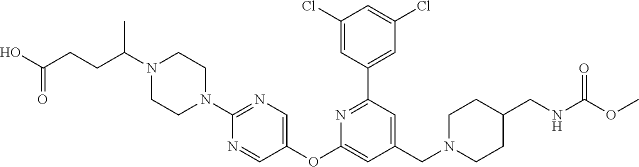 [SMILES: COC(=O)NCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((6-(3,5-dichloro- phenyl)-4-((4-(((methoxy- carbonyl)amino)methyl) piperidin-1-yl)methyl) pyridin-2-yl)oxy) pyrimidin-2-yl)piperazin- 1-yl)pentanoic acid |
| 104 | 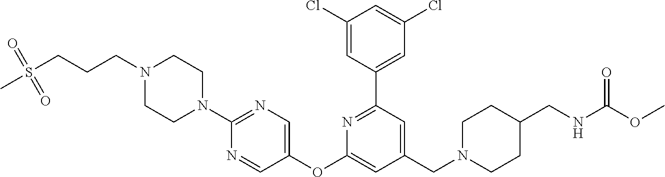 | methyl((1-((2-(3,5- dichlorophenyl)-6-((2- (4-(3-(methylsulfonyl) propyl)piperazin-1- yl)pyrimidin-5-yl)oxy) pyridin-4-yl)methyl) piperidin-4-yl)methyl) carbamate |
| 105 | 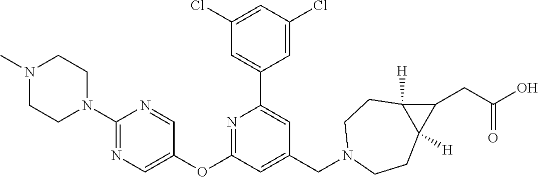 [SMILES: CN1CCN(c2ncc(Oc3cc(CN4CC[C@@H]5C(CC(=O)O)[C@@H]5CC4)cc(-c4cc(Cl)cc(Cl)c4)n3)cn2)CC1] | 2-((1R,7S,8r)-4-((2-(3,5- dichlorophenyl)-6-((2-(4- methylpiperazin-1-yl) pyrimidin-5-yl)oxy) pyridin-4-yl)methyl)-4- azabicyclo[5.1.0]octan- 8-yl)acetic acid |
| 106 | 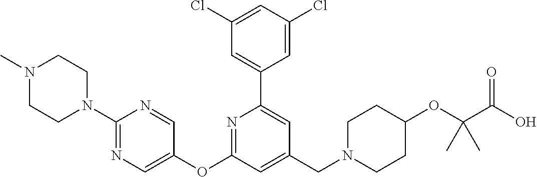 [SMILES: CN1CCN(c2ncc(Oc3cc(CN4CCC(OC(C)(C)C(=O)O)CC4)cc(-c4cc(Cl)cc(Cl)c4)n3)cn2)CC1] | 2-((1-((2-(3,5-dichloro- phenyl)-6-((2-(4-methyl- piperazin-1-yl)pyrimidin- 5-yl)oxy)pyridin-4-yl) methyl)piperidin-4- yl)oxy)-2-methyl- propanoic acid |
| 107 | 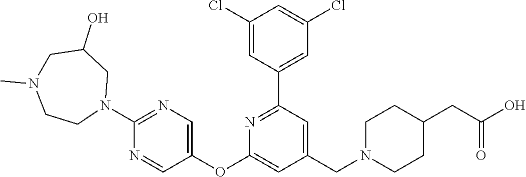 [SMILES: CN1CCN(c2ncc(Oc3cc(CN4CCC(CC(=O)O)CC4)cc(-c4cc(Cl)cc(Cl)c4)n3)cn2)CC(O)C1] | 2-(1-((2-(3,5-dichloro- phenyl)-6-((2-(6-hydroxy- 4-methyl-1,4-diazepan-1- yl)pyrimidin-5-yl)oxy) pyridin-4-yl)methyl) piperidin-4-yl)acetic acid |
| 108 | 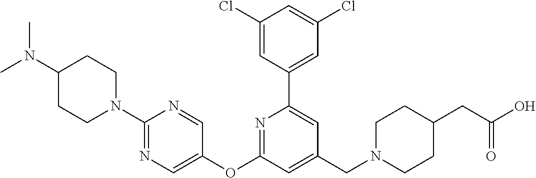 [SMILES: CN(C)C1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4- (dimethylamino) piperidin-1-yl)pyrimidin- 5-yl)oxy)pyridin-4-yl) methyl)piperidin-4- yl)acetic acid |
| 109 | 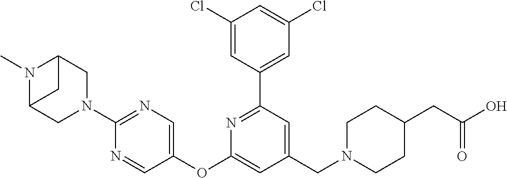 [SMILES: CN1C2CC1CN(c1ncc(Oc3cc(CN4CCC(CC(=O)O)CC4)cc(-c4cc(Cl)cc(Cl)c4)n3)cn1)C2] | 2-(1-((2-(3,5-dichloro- phenyl)-6-((2-(6-methyl- 3,6-diazabicyclo[3.1.1 heptan-3-yl)pyrimidin- 5-yl)oxy)pyridin-4-yl) methyl)piperidin-4- yl)acetic acid |
| 110 | 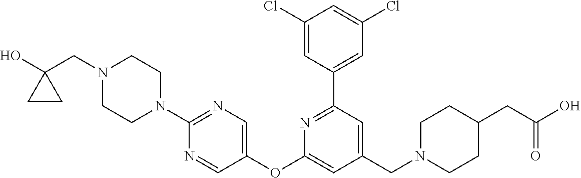 [SMILES: O=C(O)CC1CCN(Cc2cc(Oc3cnc(N4CCN(CC5(O)CC5)CC4)nc3)nc(-c3cc(Cl)cc(Cl)c3)c2)CC1] | 2-(1-((2-(3,5-dichloro- phenyl)-6-((2-(4-((1- hydroxycyclopropyl) methyl)piperazin-1- yl)pyrimidin-5-yl)oxy) pyridin-4-yl)methyl) piperidin-4-yl)acetic acid |
| 111 | 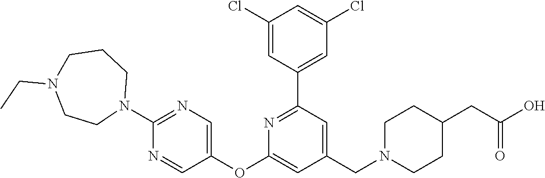 [SMILES: CCN1CCCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-ethyl- 1,4-diazepan-1-yl) pyrimidin-5-yl)oxy) pyridin-4-yl)methyl) piperidin-4-yl)acetic acid |
| 112 | 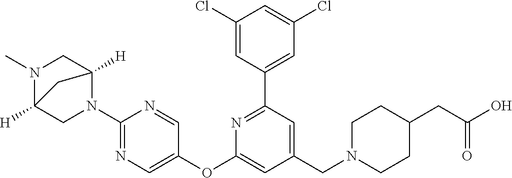 [SMILES: CN1C[C@@H]2C[C@H]1CN2c1ncc(Oc2cc(CN3CCC(CC(=O)O)CC3)cc(-c3cc(Cl)cc(Cl)c3)n2)cn1] | 2-(1-((2-(3,5-dichloro- phenyl)-6-((2-((1S,4S)-5- methyl-2,5-diazabicyclo [2.2.1]heptan-2-yl) pyrimidin-5-yl)oxy) pyridin-4-yl)methyl) piperidin-4-yl)acetic acid |
| 113 | 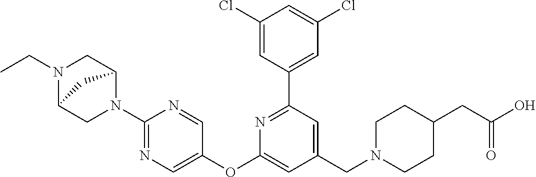 [SMILES: CCN1C[C@@H]2C[C@H]1CN2c1ncc(Oc2cc(CN3CCC(CC(=O)O)CC3)cc(-c3cc(Cl)cc(Cl)c3)n2)cn1] | 2-(1-((2-(3,5-dichloro- phenyl)-6-((2-((1S,4S)-5- ethyl-2,5-diazabicyclo [2.2.1]heptan-2-yl) pyrimidin-5-yl)oxy) pyridin-4-yl)methyl) piperidin-4-yl)acetic acid |
| 114 | 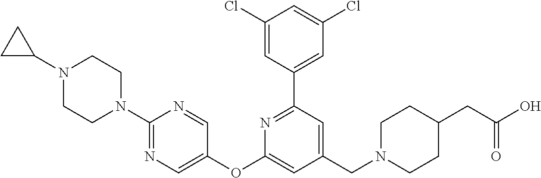 [SMILES: O=C(O)CC1CCN(Cc2cc(Oc3cnc(N4CCN(C5CC5)CC4)nc3)nc(-c3cc(Cl)cc(Cl)c3)c2)CC1] | 2-(1-((2-((2-(4-cyclo- propylpiperazin-1- yl)pyrimidin-5-yl)oxy)-6- (3,5-dichlorophenyl) pyridin-4-yl)methyl) piperidin-4-yl)acetic acid |
| 115 | 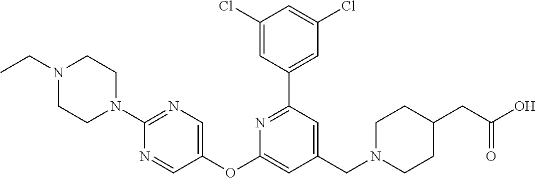 [SMILES: CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-ethyl- piperazin-1-yl)pyrimidin- 5-yl)oxy)pyridin-4-yl) methyl)piperidin-4- yl)acetic acid |
| 116 | 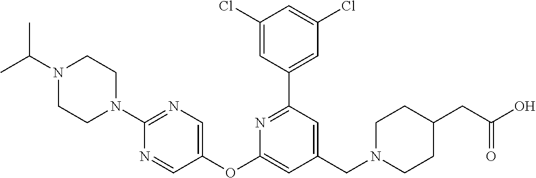 [SMILES: CC(C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4- isopropylpiperazin-1- yl)pyrimidin-5- yl)oxy)pyridin-4-yl) methyl)piperidin-4- yl)acetic acid |
| 117 | 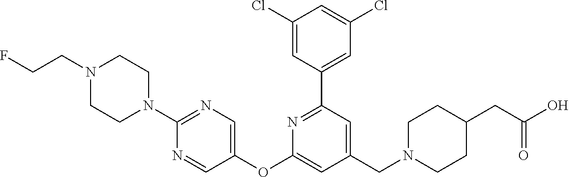 [SMILES: O=C(O)CC1CCN(Cc2cc(Oc3cnc(N4CCN(CCF)CC4)nc3)nc(-c3cc(Cl)cc(Cl)c3)c2)CC1] | 2-(1-((2-(3,5-dichloro- phenyl)-6-((2-(4-(2- fluoroethyl)piperazin- 1-yl)pyrimidin-5- yl)oxy)pyridin-4- yl)methyl)piperidin-4- yl)acetic acid |
| 118 | 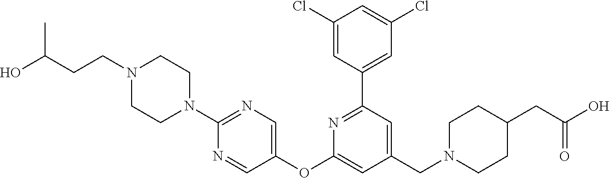 [SMILES: CC(O)CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(3- hydroxybutyl)piperazin- 1-yl)pyrimidin-5- yl)oxy)pyridin-4- yl)methyl)piperidin-4- yl)acetic acid |
| 119 | 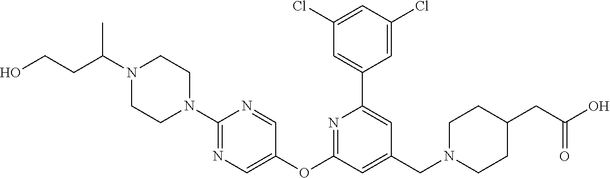 [SMILES: CC(CCO)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(4- hydroxybutan-2-yl) piperazin-1-yl)pyrimidin- 5-yl)oxy)pyridin-4- yl)methyl)piperidin-4- yl)acetic acid |
| 120 | 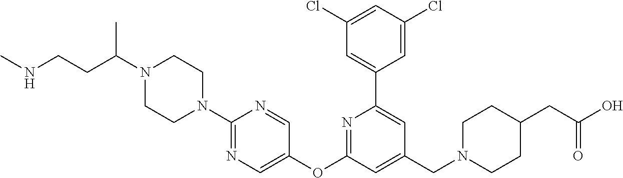 [SMILES: CNCCC(C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(4- (methylamino)butan-2- yl)piperazin-1-yl) pyrimidin-5-yl)oxy) pyridin-4-yl)methyl) piperidin-4-yl)acetic acid |
| 121 | 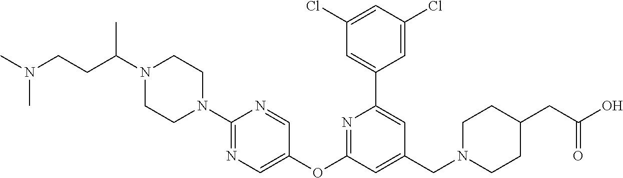 [SMILES: CC(CCN(C)C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(4- (dimethylamino)butan- 2-yl)piperazin-1- yl)pyrimidin-5-yl) oxy)pyridin-4- yl)methyl)piperidin-4- yl)acetic acid |
| 122 | 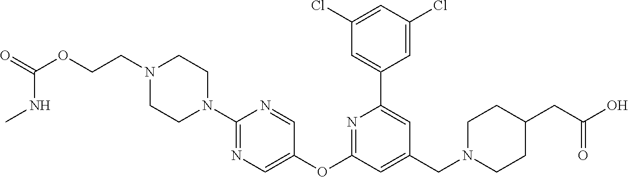 [SMILES: CNC(=O)OCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(2- ((methylcarbamoyl) oxy)ethyl)piperazin-1- yl)pyrimidin-5-yl)oxy) pyridin-4-yl)methyl) piperidin-4-yl)acetic acid |
| 123 | 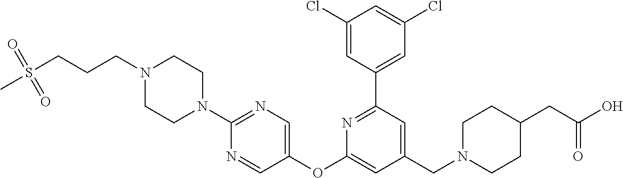 [SMILES: CS(=O)(=O)CCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(3- (methylsulfonyl)propyl) piperazin-1-yl)pyrimidin- 5-yl)oxy)pyridin-4- yl)methyl)piperidin-4- yl)acetic acid |
| 124 | 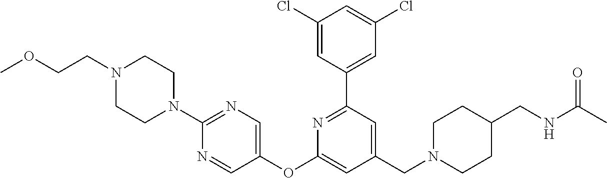 [SMILES: COCCN1CCN(c2ncc(Oc3cc(CN4CCC(CNC(C)=O)CC4)cc(-c4cc(Cl)cc(Cl)c4)n3)cn2)CC1] | N-((1-((2-(3,5-dichloro- phenyl)-6-((2-(4-(2- methoxyethyl)piperazin- 1-yl)pyrimidin-5-yl)oxy) pyridin-4-yl)methyl) piperidin-4-yl)methyl) acetamide |
| 125 | 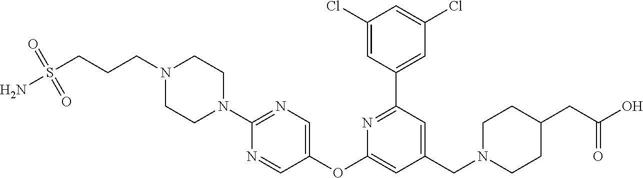 [SMILES: NS(=O)(=O)CCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(3- sulfamoylpropyl) piperazin-1-yl)pyrimidin- 5-yl)oxy)pyridin-4-yl) methyl)piperidin-4-yl) acetic acid |
| 126 | 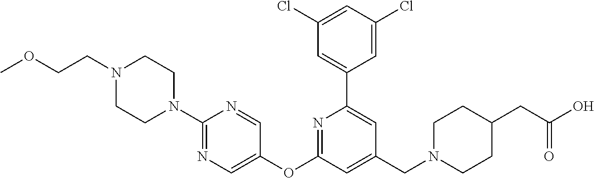 [SMILES: COCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(2- methoxyethyl)piperazin- 1-yl)pyrimidin-5-yl)oxy) pyridin-4-yl)methyl) piperidin-4-yl)acetic acid |
| 127 | 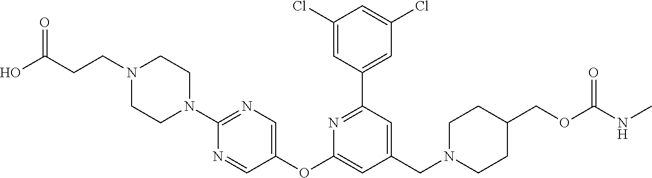 [SMILES: CNC(=O)OCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-(((methyl- carbamoyl)oxy)methyl) piperidin-1-yl)methyl) pyridin-2-yl)oxy) pyrimidin-2-yl)piperazin- 1-yl)propanoic acid |
| 128 | 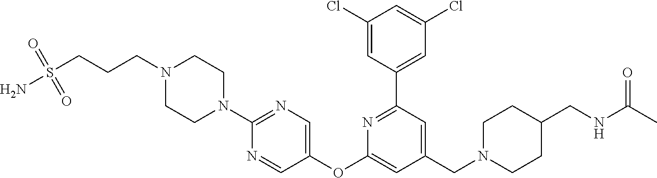 [SMILES: CC(=O)NCC1CCN(Cc2cc(Oc3cnc(N4CCN(CCCS(N)(=O)=O)CC4)nc3)nc(-c3cc(Cl)cc(Cl)c3)c2)CC1] | N-((1-((2-(3,5-dichloro- phenyl)-6-((2-(4-(3- sulfamoylpropyl) piperazin-1-yl)pyrimidin- 5-yl)oxy)pyridin-4- yl)methyl)piperidin-4- yl)methyl)acetamide |
| 129 | 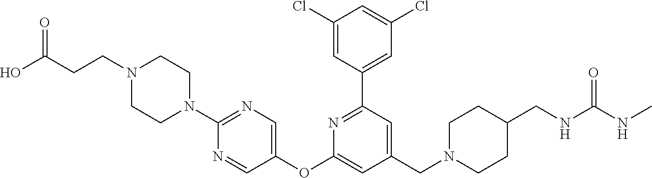 [SMILES: CNC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-((3-methyl- ureido)methyl)piperidin- 1-yl)methyl)pyridin-2-yl) oxy)pyrimidin-2-yl) piperazin-1-yl)propanoic acid |
| 130 | 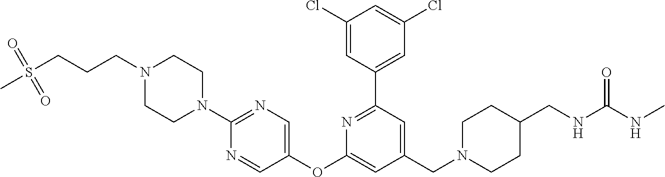 | 1-((1-((2-(3,5-dichloro- phenyl)-6-((2-(4-(3- (methylsulfonyl)propyl) piperazin-1-yl)pyrimidin- 5-yl)oxy)pyridin-4-yl) methyl)piperidin-4-yl) methyl)-3-methylurea |
| 131 | 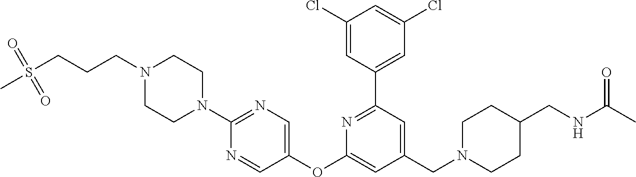 [SMILES: CC(=O)NCC1CCN(Cc2cc(Oc3cnc(N4CCN(CCCS(C)(=O)=O)CC4)nc3)nc(-c3cc(Cl)cc(Cl)c3)c2)CC1] | N-((1-((2-(3,5-dichloro- phenyl)-6-((2-(4-(3- (methylsulfonyl)propyl) piperazin-1-yl)pyrimidin- 5-yl)oxy)pyridin-4- yl)methyl)piperidin-4- yl)methyl)acetamide |
| 132 | 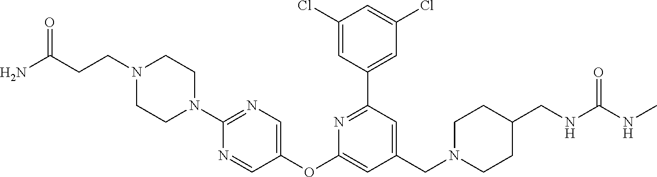 [SMILES: CNC(=O)NCC1CCN(Cc2cc(Oc3cnc(N4CCN(CCC(N)=O)CC4)nc3)nc(-c3cc(Cl)cc(Cl)c3)c2)CC1] | 3-(4-(5-((6-(3,5-dichloro- phenyl)-4-((4-((3-methyl- ureido)methyl)piperidin- 1-yl)methyl)pyridin-2- yl)oxy)pyrimidin-2- yl)piperazin-1- yl)propanamide |
| 133 | 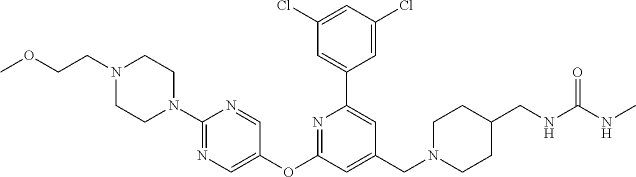 | 1-((1-((2-(3,5-dichloro- phenyl)-6-((2-(4-(2- methoxyethyl)piperazin- 1-yl)pyrimidin-5-yl)oxy) pyridin-4-yl)methyl) piperidin-4-yl)methyl)-3- methylurea |
| 134 | 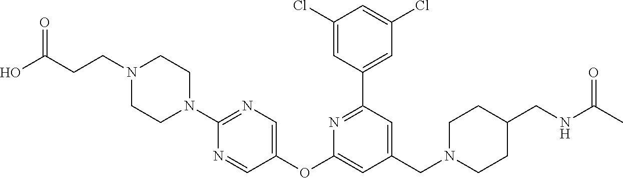 [SMILES: CC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichlorophenyl) pyridin-2-yl)oxy) pyrimidin-2-yl) piperazin-1-yl)propanoic acid |
| 135 | 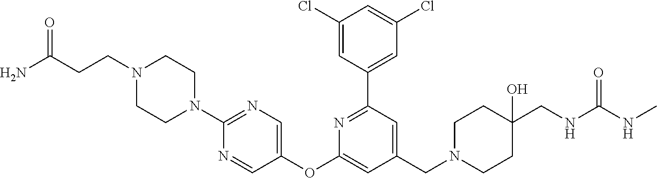 [SMILES: CNC(=O)NCC1(O)CCN(Cc2cc(Oc3cnc(N4CCN(CCC(N)=O)CC4)nc3)nc(-c3cc(Cl)cc(Cl)c3)c2)CC1] | 3-(4-(5-((6-(3,5-dichloro- phenyl)-4-((4-hydroxy-4- ((3-methylureido)methyl) piperidin-1-yl)methyl) pyridin-2-yl)oxy) pyrimidin-2-yl)piperazin- 1-yl)propanamide |
| 136 | 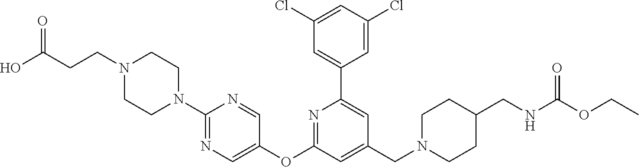 [SMILES: CCOC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-(((ethoxy- carbonyl)amino)methyl) piperidin-1-yl)methyl) pyridin-2-yl)oxy) pyrimidin-2-yl)piperazin- 1-yl)propanoic acid |
| 137 | 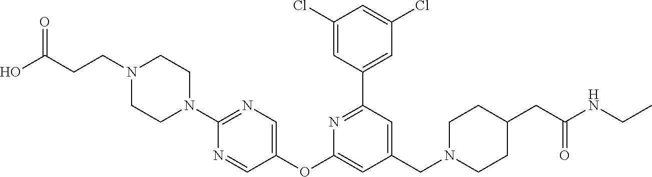 [SMILES: CCNC(=O)CC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichloro- phenyl)-4-((4-(2-(ethyl- amino)-2-oxoethyl) piperidin-1-yl)methyl) pyridin-2-yl)oxy) pyrimidin-2-yl)piperazin- 1-yl)propanoic acid |
| 138 | 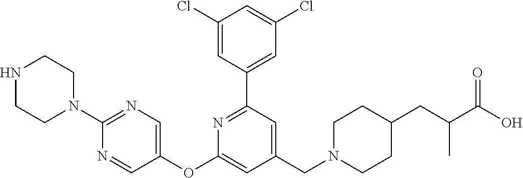 [SMILES: CC(CC1CCN(Cc2cc(Oc3cnc(N4CCNCC4)nc3)nc(-c3cc(Cl)cc(Cl)c3)c2)CC1)C(=O)O] | 3-(1-((2-(3,5-dichloro- phenyl)-6-((2-(piperazin- 1-yl)pyrimidin-5-yl)oxy) pyridin-4-yl)methyl) piperidin-4-yl)-2-methyl- propanoic acid |
| 139 | 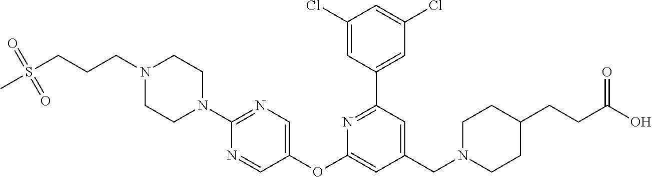 [SMILES: CS(=O)(=O)CCCN1CCN(c2ncc(Oc3cc(CN4CCC(CCC(=O)O)CC4)cc(-c4cc(Cl)cc(Cl)c4)n3)cn2)CC1] | 3-(1-((2-(3,5-dichloro- phenyl)-6-((2-(4-(3- (methylsulfonyl)propyl) piperazin-1-yl)pyrimidin- 5-yl)oxy)pyridin-4-yl) methyl)piperidin-4-yl) propanoic acid |
| 140 | 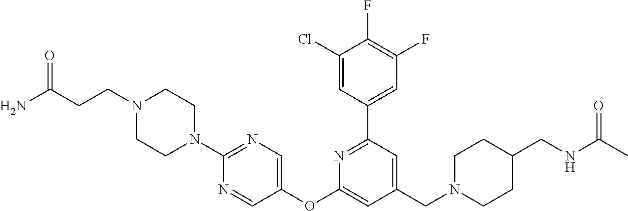 [SMILES: CC(=O)NCC1CCN(Cc2cc(Oc3cnc(N4CCN(CCC(N)=O)CC4)nc3)nc(-c3cc(F)c(F)c(Cl)c3)c2)CC1] | 3-(4-(5-((4-((4- (acetamidomethyl) piperidin-1-yl)methyl)- 6-(3-chloro-4,5-difluoro- phenyl)pyridin-2-yl) oxy)pyrimidin-2-yl) piperazin-1- yl)propanamide |
| 141 | 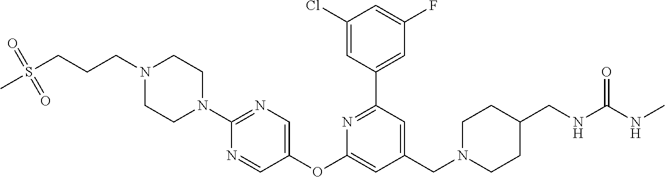 | 1-((1-((2-(3-chloro-5- fluorophenyl)-6-((2-(4-(3- (methylsulfonyl)propyl) piperazin-1-yl)pyrimidin- 5-yl)oxy)pyridin-4-yl) methyl)piperidin-4-yl) methyl)-3-methylurea |
| 142 | 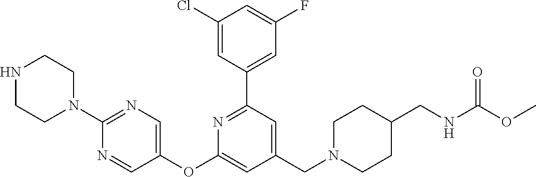 [SMILES: COC(=O)NCC1CCN(Cc2cc(Oc3cnc(N4CCNCC4)nc3)nc(-c3cc(F)cc(Cl)c3)c2)CC1] | methyl ((1-((2-(3-chloro- 5-fluorophenyl)-6-((2- (piperazin-1-yl)pyrimidin- 5-yl)oxy)pyridin-4-yl) methyl)piperidin-4- yl)methyl)carbamate |
| 143 | 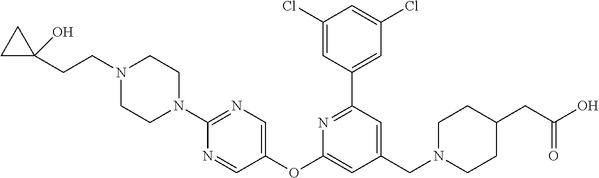 [SMILES: O=C(O)CC1CCN(Cc2cc(Oc3cnc(N4CCN(CCC5(O)CC5)CC4)nc3)nc(-c3cc(Cl)cc(Cl)c3)c2)CC1] | 2-(1-((2-(3,5-dichloro- phenyl)-6-((2-(4-(2-(1- hydroxycyclopropyl) ethyl)piperazin-1-yl) pyrimidin-5-yl)oxy) pyridin-4-yl)methyl) piperidin-4-yl)acetic acid |
| 144 | 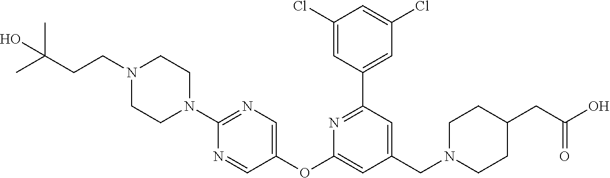 [SMILES: CC(C)(O)CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(3- hydroxy-3-methylbutyl) piperazin-1-yl)pyrimidin- 5-yl)oxy)pyridin-4- yl)methyl)piperidin-4- yl)acetic acid |
| 145 | 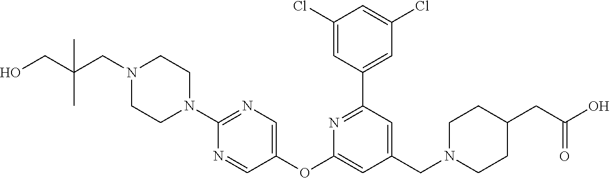 [SMILES: CC(C)(CO)CN1CCN(c2ncc(Oc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-(3- hydroxy-2,2-dimethyl- propyl)piperazin-1- yl)pyrimidin-5-yl)oxy) pyridin-4-yl)methyl) piperidin-4-yl)acetic acid |
| 146 | 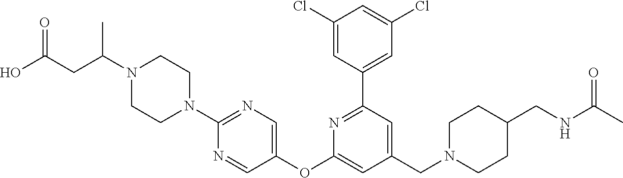 [SMILES: CC(=O)NCC1CCN(Cc2cc(Oc3cnc(N4CCN(C(C)CC(=O)O)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichlorophenyl) pyridin-2-yl)oxy) pyrimidin-2-yl) piperazin-1-yl)butanoic acid |
| 147 | 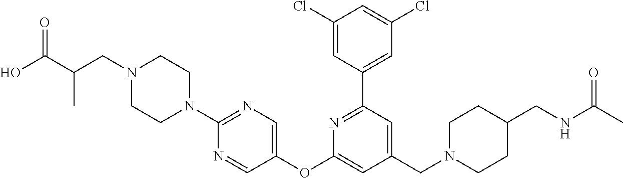 [SMILES: CC(=O)NCC1CCN(Cc2cc(Oc3cnc(N4CCN(CC(C)C(=O)O)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichlorophenyl) pyridin-2-yl)oxy) pyrimidin-2-yl) piperazin-1-yl)-2- methylpropanoic acid |
| 148 | 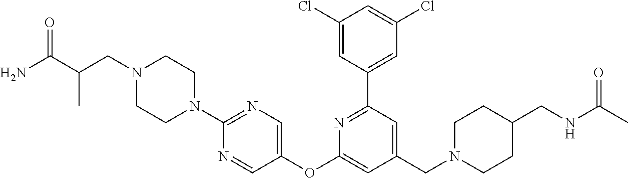 [SMILES: CC(=O)NCC1CCN(Cc2cc(Oc3cnc(N4CCN(CC(C)C(N)=O)CC4)nc3)nc(-c3cc(Cl)cc(Cl)c3)c2)CC1] | 3-(4-(5-((4-((4- (acetamidomethyl) piperidin-1-yl)methyl)- 6-(3,5-dichlorophenyl) pyridin-2-yl)oxy) pyrimidin-2-yl) piperazin-1-yl)-2- methylpropanamide |
| 149 | 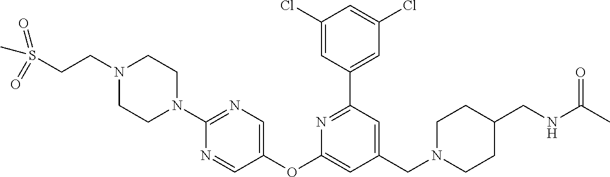 [SMILES: CC(=O)NCC1CCN(Cc2cc(Oc3cnc(N4CCN(CCS(C)(=O)=O)CC4)nc3)nc(-c3cc(Cl)cc(Cl)c3)c2)CC1] | N-((1-((2-(3,5-dichloro- phenyl)-6-((2-(4-(2- (methylsulfonyl)ethyl) piperazin-1-yl)pyrimidin- 5-yl)oxy)pyridin-4- yl)methyl)piperidin-4- yl)methyl)acetamide |
| 150 | 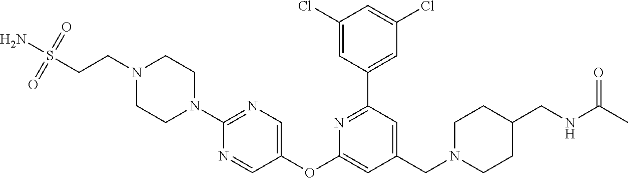 [SMILES: CC(=O)NCC1CCN(Cc2cc(Oc3cnc(N4CCN(CCS(N)(=O)=O)CC4)nc3)nc(-c3cc(Cl)cc(Cl)c3)c2)CC1] | N-((1-((2-(3,5-dichloro- phenyl)-6-((2-(4-(2- sulfamoylethyl)piperazin- 1-yl)pyrimidin-5-yl)oxy) pyridin-4-yl)methyl) piperidin-4-yl)methyl) acetamide |
| 151 | 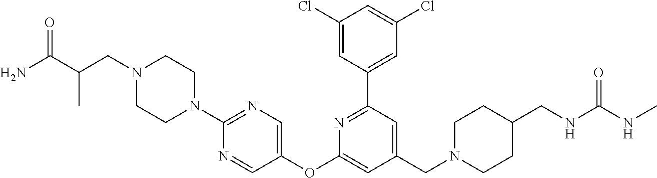 [SMILES: CNC(=O)NCC1CCN(Cc2cc(Oc3cnc(N4CCN(CC(C)C(N)=O)CC4)nc3)nc(-c3cc(Cl)cc(Cl)c3)c2)CC1] | 3-(4-(5-((6-(3,5-dichloro- phenyl)-4-((4-((3-methyl- ureido)methyl)piperidin- 1-yl)methyl)pyridin-2- yl)oxy)pyrimidin-2- yl)piperazin-1-yl)-2- methylpropanamide |
| 152 | 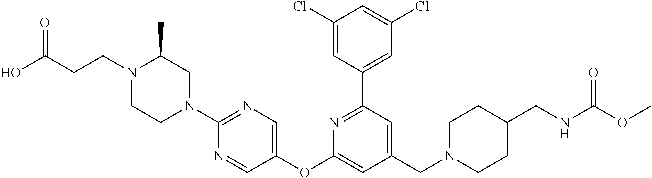 [SMILES: COC(=O)NCC1CCN(Cc2cc(Oc3cnc(N4CCN(CCC(=O)O)[C@@H](C)C4)nc3)nc(-c3cc(Cl)cc(Cl)c3)c2)CC1] | (S)-3-(4-(5-((6-(3,5- dichlorophenyl)-4-((4- (((methoxycarbonyl) amino)methyl)piperidin- 1-yl)methyl)pyridin-2- yl)oxy)pyrimidin-2-yl)- 2-methylpiperazin-1- yl)propanoic acid |
| 153 | 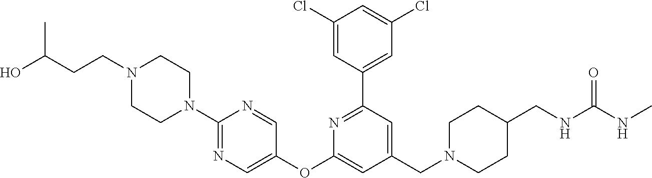 [SMILES: CNC(=O)NCC1CCN(Cc2cc(Oc3cnc(N4CCN(CCC(C)O)CC4)nc3)nc(-c3cc(Cl)cc(Cl)c3)c2)CC1] | 1-((1-((2-(3,5-dichloro- phenyl)-6-((2-(4-(3- hydroxybutyl)piperazin- 1-yl)pyrimidin-5-yl)oxy) pyridin-4-yl)methyl) piperidin-4-yl)methyl)- 3-methylurea |
| 154 | 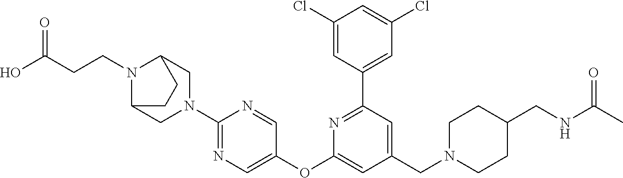 [SMILES: CC(=O)NCC1CCN(Cc2cc(Oc3cnc(N4CC5CCC(C4)N5CCC(=O)O)nc3)nc(-c3cc(Cl)cc(Cl)c3)c2)CC1] | 3-(3-(5-((4-((4- (acetamidomethyl) piperidin-1-yl)methyl)- 6-(3,5-dichlorophenyl) pyridin-2-yl)oxy) pyrimidin-2-yl)-3,8- diazabicyclo[3.2.1] octan-8-yl)propanoic acid |
| 155 | 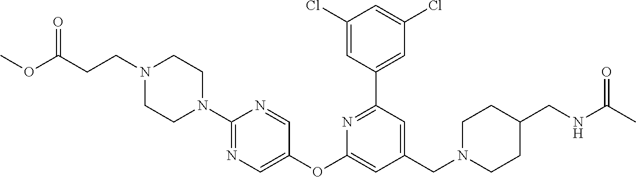 [SMILES: COC(=O)CCN1CCN(c2ncc(Oc3cc(CN4CCC(CNC(C)=O)CC4)cc(-c4cc(Cl)cc(Cl)c4)n3)cn2)CC1] | methyl3-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)propanoate |
| 156 | 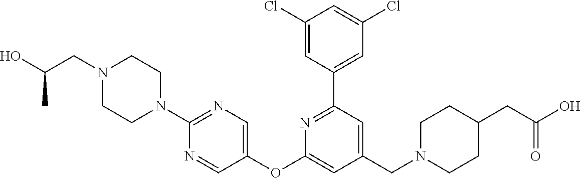 [SMILES: C[C@@H](O)CN1CCN(c2ncc(Oc3cc(CN4CCC(CC(=O)O)CC4)cc(-c4cc(Cl)cc(Cl)c4)n3)cn2)CC1] | (R)-2-(1-((2-(3,5- dichlorophenyl)-6-((2-(4- (2-hydroxypropyl) piperazin-1-yl)pyrimidin- 5-yl)oxy)pyridin-4- yl)methyl)piperidin-4- yl)acetic acid |
| 157 | 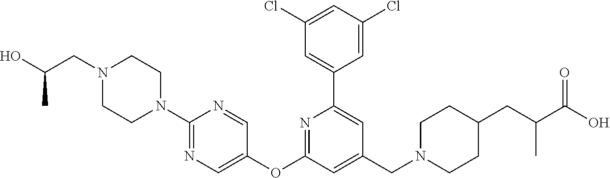 [SMILES: CC(CC1CCN(Cc2cc(Oc3cnc(N4CCN(C[C@@H](C)O)CC4)nc3)nc(-c3cc(Cl)cc(Cl)c3)c2)CC1)C(=O)O] | 3-(1-((2-(3,5-dichloro- phenyl)-6-((2-(4-((R)-2- hydroxypropyl)piperazin- 1-yl)pyrimidin-5-yl)oxy) pyridin-4-yl)methyl) piperidin-4-yl)-2- methylpropanoic acid |
| 158 | 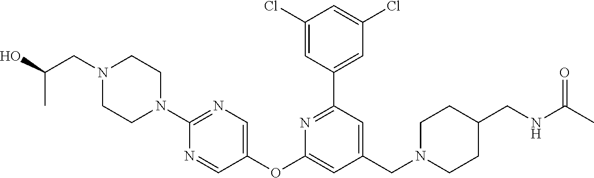 [SMILES: CC(=O)NCC1CCN(Cc2cc(Oc3cnc(N4CCN(C[C@@H](C)O)CC4)nc3)nc(-c3cc(Cl)cc(Cl)c3)c2)CC1] | (R)-N-((1-((2-(3,5- dichlorophenyl)-6-((2- (4-(2-hydroxypropyl) piperazin-1-yl)pyrimidin- 5-yl)oxy)pyridin-4- yl)methyl)piperidin-4- yl)methyl)acetamide |
| 159 | 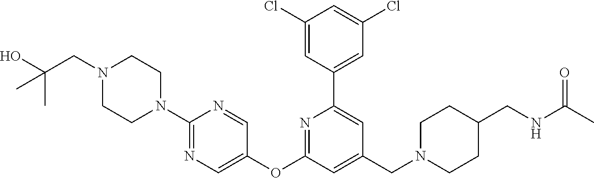 [SMILES: CC(=O)NCC1CCN(Cc2cc(Oc3cnc(N4CCN(CC(C)(C)O)CC4)nc3)nc(-c3cc(Cl)cc(Cl)c3)c2)CC1] | N-((1-((2-(3,5-dichloro- phenyl)-6-((2-(4-(2- hydroxy-2-methyl- propyl)piperazin-1- yl)pyrimidin-5-yl)oxy) pyridin-4-yl)methyl) piperidin-4-yl)methyl) acetamide |
| 160 | 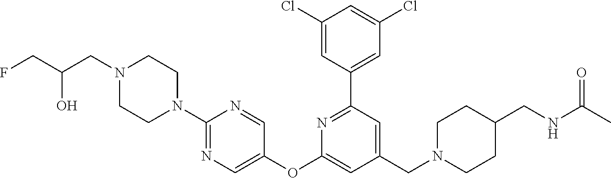 [SMILES: CC(=O)NCC1CCN(Cc2cc(Oc3cnc(N4CCN(CC(O)CF)CC4)nc3)nc(-c3cc(Cl)cc(Cl)c3)c2)CC1] | N-((1-((2-(3,5-dichloro- phenyl)-6-((2-(4-(3- fluoro-2-hydroxy- propyl)piperazin-1- yl)pyrimidin-5-yl)oxy) pyridin-4-yl)methyl) piperidin-4-yl)methyl) acetamide |
| 161 | 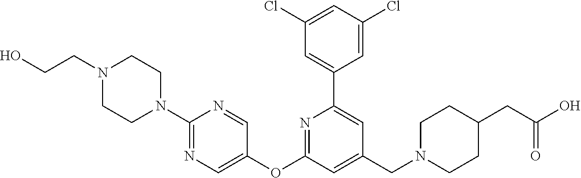 [SMILES: O=C(O)CC1CCN(Cc2cc(Oc3cnc(N4CCN(CCO)CC4)nc3)nc(-c3cc(Cl)cc(Cl)c3)c2)CC1] | 2-(1-((2-(3,5-dichloro- phenyl)-6-((2-(4-(2- hydroxyethyl)piperazin- 1-yl)pyrimidin-5-yl)oxy) pyridin-4-yl)methyl) piperidin-4-yl)acetic acid |
| 162 | 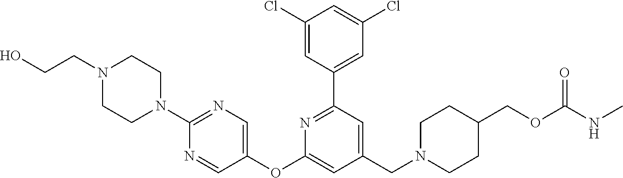 [SMILES: CNC(=O)OCC1CCN(Cc2cc(Oc3cnc(N4CCN(CCO)CC4)nc3)nc(-c3cc(Cl)cc(Cl)c3)c2)CC1] | (1-((2-(3,5-dichloro- phenyl)-6-((2-(4-(2- hydroxyethyl)piperazin- 1-yl)pyrimidin-5-yl)oxy) pyridin-4-yl)methyl) piperidin-4-yl)methyl- methylcarbamate |
| 163 | 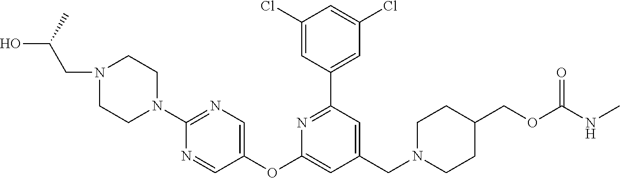 [SMILES: CNC(=O)OCC1CCN(Cc2cc(Oc3cnc(N4CCN(C[C@@H](C)O)CC4)nc3)nc(-c3cc(Cl)cc(Cl)c3)c2)CC1] | (R)-(1-((2-(3,5-dichloro- phenyl)-6-((2-(4-(2- hydroxypropyl)piperazin- 1-yl)pyrimidin-5-yl)oxy) pyridin-4-yl)methyl) piperidin-4-yl)methyl methylcarbamate |
| 164 | 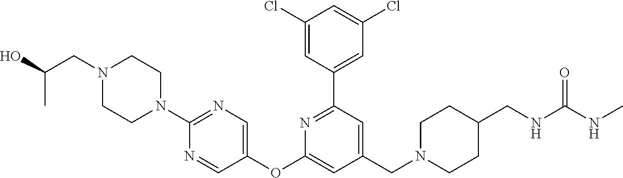 [SMILES: CNC(=O)NCC1CCN(Cc2cc(Oc3cnc(N4CCN(C[C@@H](C)O)CC4)nc3)nc(-c3cc(Cl)cc(Cl)c3)c2)CC1] | (R)-1-((1-((2-(3,5- dichlorophenyl)-6-((2-(4- (2-hydroxypropyl) piperazin-1-yl)pyrimidin- 5-yl)oxy)pyridin-4- yl)methyl)piperidin-4- yl)methyl)-3-methylurea |
| 165 | 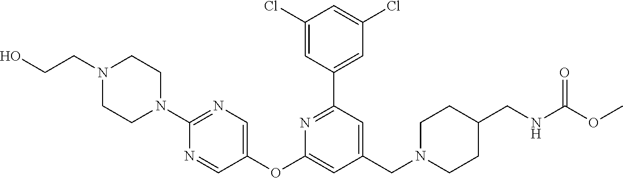 [SMILES: COC(=O)NCC1CCN(Cc2cc(Oc3cnc(N4CCN(CCO)CC4)nc3)nc(-c3cc(Cl)cc(Cl)c3)c2)CC1] | methyl((1-((2-(3,5- dichlorophenyl)-6-((2-(4- (2-hydroxyethyl) piperazin-1-yl)pyrimidin- 5-yl)oxy)pyridin-4-yl) methyl)piperidin-4- yl)methyl)carbamate |
| 166 | 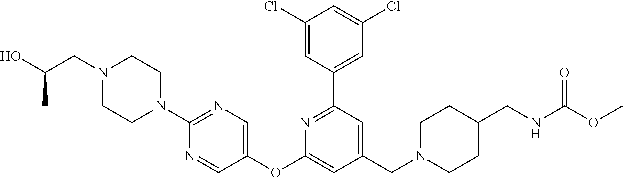 [SMILES: COC(=O)NCC1CCN(Cc2cc(Oc3cnc(N4CCN(C[C@@H](C)O)CC4)nc3)nc(-c3cc(Cl)cc(Cl)c3)c2)CC1] | (R)-methyl((1-((2-(3,5- dichlorophenyl)-6-((2-(4- (2-hydroxypropyl) piperazin-1-yl)pyrimidin- 5-yl)oxy)pyridin-4- yl)methyl)piperidin-4- yl)methyl)carbamate |
| 167 | 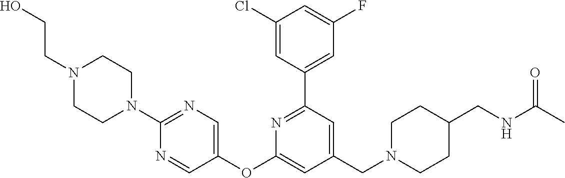 [SMILES: CC(=O)NCC1CCN(Cc2cc(Oc3cnc(N4CCN(CCO)CC4)nc3)nc(-c3cc(F)cc(Cl)c3)c2)CC1] | N-((1-((2-(3-chloro-5- fluorophenyl)-6-((2-(4-(2- hydroxyethyl)piperazin-1- yl)pyrimidin-5-yl)oxy) pyridin-4-yl)methyl) piperidin-4-yl)methyl) acetamide |
| 168 | 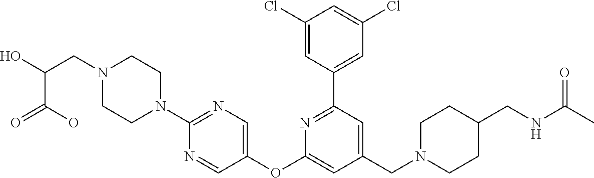 [SMILES: CC(=O)NCC1CCN(Cc2cc(Oc3cnc(N4CCN(CC(O)C(=O)O)CC4)nc3)nc(-c3cc(Cl)cc(Cl)c3)c2)CC1] | 3-(4-(5-((4-((4- (acetamidomethyl) piperidin-1-yl) methyl)-6-(3,5-dichloro- phenyl)pyridin-2-yl)oxy) pyrimidin-2-yl)piperazin- 1-yl)-2-hydroxy- propanoicacid |
| 169 | 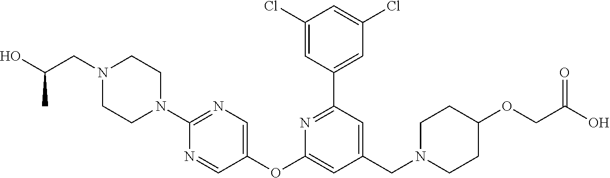 [SMILES: C[C@@H](O)CN1CCN(c2ncc(Oc3cc(CN4CCC(OCC(=O)O)CC4)cc(-c4cc(Cl)cc(Cl)c4)n3)cn2)CC1] | (R)-2-((1-((2-(3,5- dichlorophenyl)-6-((2-(4- (2-hydroxypropyl) piperazin-1-yl)pyrimidin- 5-yl)oxy)pyridin-4-yl) methyl)piperidin-4-yl) oxy)acetic acid |
| 170 | 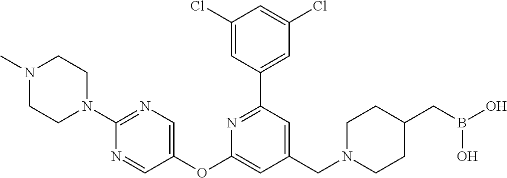 [SMILES: CN1CCN(c2ncc(Oc3cc(CN4CCC(CB(O)O)CC4)cc(-c4cc(Cl)cc(Cl)c4)n3)cn2)CC1] | ((1-((2-(3,5-dichloro- phenyl)-6-((2-(4-methyl- piperazin-1-yl)pyrimidin- 5-yl)oxy)pyridin-4-yl) methyl)piperidin-4- yl)methyl)boronic acid |
| 171 | 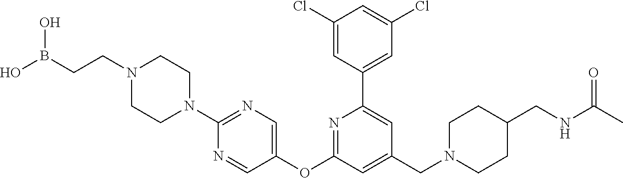 [SMILES: CC(=O)NCC1CCN(Cc2cc(Oc3cnc(N4CCN(CCB(O)O)CC4)nc3)nc(-c3cc(Cl)cc(Cl)c3)c2)CC1] | (2-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)ethyl)boronic acid |
| 172 | 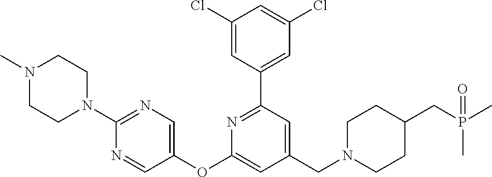 | ((1-((2-(3,5-dichloro- phenyl)-6-((2-(4-methyl- piperazin-1-yl)pyrimidin- 5-yl)oxy)pyridin-4-yl) methyl)piperidin-4- yl)methyl)dimethyl- phosphineoxide |
| 173 | 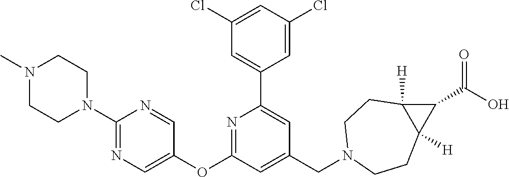 [SMILES: CN1CCN(c2ncc(Oc3cc(CN4CC[C@@H]5[C@H](CC4)[C@H]5C(=O)O)cc(-c4cc(Cl)cc(Cl)c4)n3)cn2)CC1] | (1R,7S,8r)-4-((2-(3,5- dichlorophenyl)-6-((2- (4-methylpiperazin-1- yl)pyrimidin-5-yl)oxy) pyridin-4-yl)methyl)-4- azabicyclo[5.1.0]octane- 8-carboxylic acid |
| 174 | 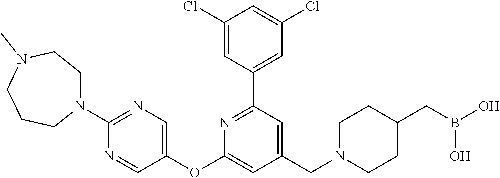 [SMILES: CN1CCCN(c2ncc(Oc3cc(CN4CCC(CB(O)O)CC4)cc(-c4cc(Cl)cc(Cl)c4)n3)cn2)CC1] | ((1-((2-(3,5-dichloro- phenyl)-6-((2-(4-methyl- 1,4-diazepan-1-yl) pyrimidin-5-yl)oxy) pyridin-4-yl)methyl) piperidin-4-yl)methyl) boronic acid |
| 175 | 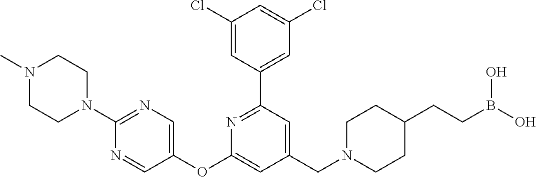 [SMILES: CN1CCN(c2ncc(Oc3cc(CN4CCC(CCB(O)O)CC4)cc(-c4cc(Cl)cc(Cl)c4)n3)cn2)CC1] | (2-(1-((2-(3,5-dichloro- phenyl)-6-((2-(4-methyl- piperazin-1-yl)pyrimidin- 5-yl)oxy)pyridin-4-yl) methyl)piperidin-4-yl) ethyl)boronic acid |
| 176 | 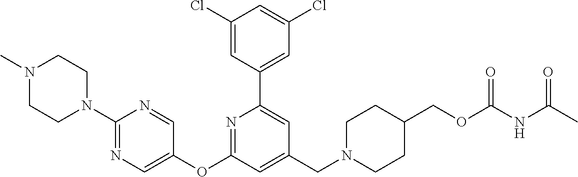 [SMILES: CC(=O)NC(=O)OCC1CCN(Cc2cc(Oc3cnc(N4CCN(C)CC4)nc3)nc(-c3cc(Cl)cc(Cl)c3)c2)CC1] | (1-((2-(3,5-dichloro- phenyl)-6-((6-(4-methyl- piperazin-1-yl)pyridin-3- yl)oxy)pyridin-4-yl) methyl)piperidin-4-yl) methylacetylcarbamate |
| 177 | 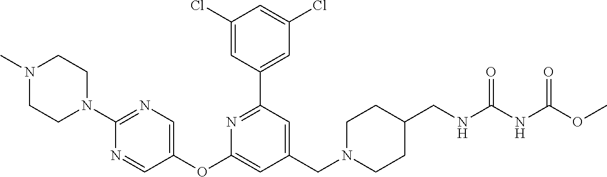 | N-1-((1-((2-(3,5- dichlorophenyl)-6-((2-(4- methylpiperazin-1-yl) pyrimidin-5-yl)oxy) pyridin-4-yl)methyl) piperidin-4-yl)methyl)- N′-methoxylcarbonylurea |
| 178 | 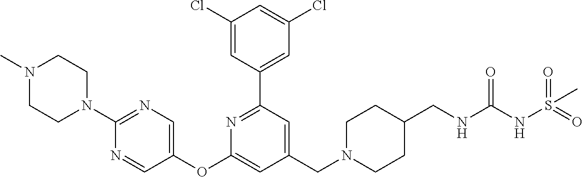 | N-(((1-((2-(3,5-dichloro- phenyl)-6-((2-(4-methyl- piperazin-1-yl)pyrimidin- 5-yl)oxy)pyridin-4-yl) methyl)piperidin-4-yl) methyl)carbamoyl) methanesulfonamide |
| 179 | 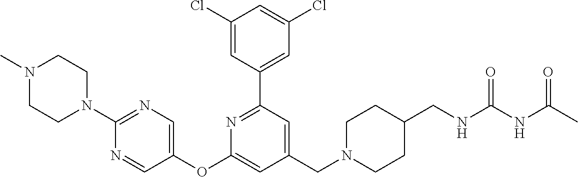 [SMILES: CC(=O)NC(=O)NCC1CCN(Cc2cc(Oc3cnc(N4CCN(C)CC4)nc3)nc(-c3cc(Cl)cc(Cl)c3)c2)CC1] | N-(((1-((2-(3,5-dichloro- phenyl)-6-((2-(4-methyl- piperazin-1-yl)pyrimidin- 5-yl)oxy)pyridin-4-yl) methyl)piperidin-4- yl)methyl)carbamoyl) acetamide |
| 180 | 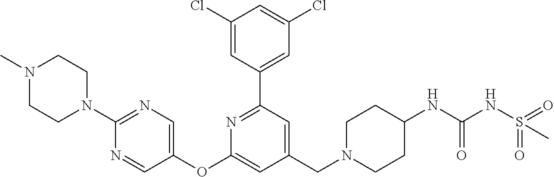 | N-((1-((2-(3,5-dichloro- phenyl)-6-((2-(4-methyl- piperazin-1-yl)pyrimidin- 5-yl)oxy)pyridin-4-yl) methyl)piperidin-4- yl)carbamoyl)methane- sulfonamide |
| 181 | 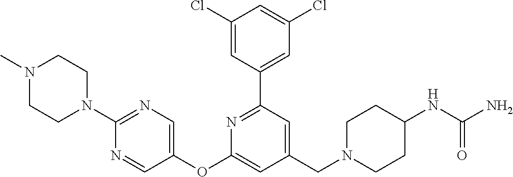 [SMILES: CN1CCN(c2ncc(Oc3cc(CN4CCC(NC(N)=O)CC4)cc(-c4cc(Cl)cc(Cl)c4)n3)cn2)CC1] | 1-(1-((2-(3,5-dichloro- phenyl)-6-((2-(4-methyl- piperazin-1-yl)pyrimidin- 5-yl)oxy)pyridin-4-yl) methyl)piperidin-4-yl)urea |
| 182 | 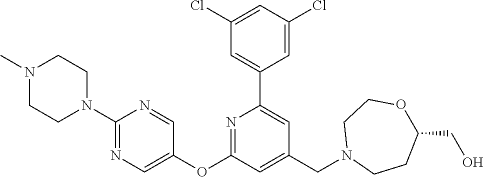 [SMILES: CN1CCN(c2ncc(Oc3cc(CN4CCO[C@H](CO)CC4)cc(-c4cc(Cl)cc(Cl)c4)n3)cn2)CC1] | (S)-(4-((2-(3,5-dichloro- phenyl)-6-((2-(4-methyl- piperazin-1-yl)pyrimidin- 5-yl)oxy)pyridin-4-yl) methyl)-1,4-oxazepan- 7-yl)methanol |
| 183 | 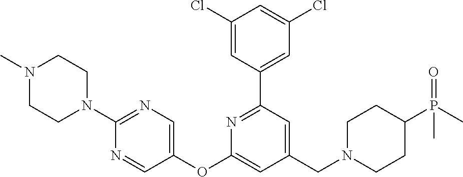 | (1-((2-(3,5-dichloro- phenyl)-6-((2-(4-methyl- piperazin-1-yl)pyrimidin- 5-yl)oxy)pyridin-4-yl) methyl)piperidin-4-yl) dimethylphosphineoxide |
| 184 | 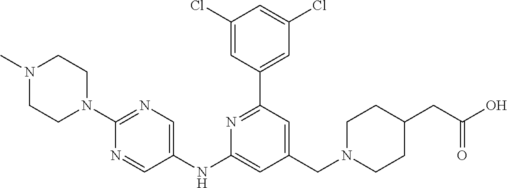 [SMILES: CN1CCN(c2ncc(Nc3cc(CN4CCC(CC(=O)O)CC4)cc(-c4cc(Cl)cc(Cl)c4)n3)cn2)CC1] | 2-(1-((2-(3,5-dichloro- phenyl)-6-((2-(4-methyl- piperazin-1-yl)pyrimidin- 5-yl)amino)pyridin-4- yl)methyl)piperidin-4- yl)acetic acid |
| 185 | 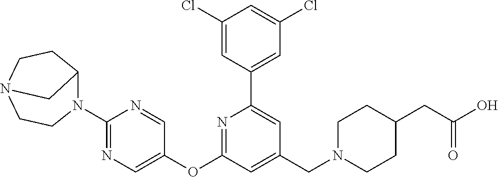 [SMILES: O=C(O)CC1CCN(Cc2cc(Oc3cnc(N4CCN5CCC4C5)nc3)nc(-c3cc(Cl)cc(Cl)c3)c2)CC1] | 2-(1-((2-((2-(1,4-diaza- bicyclo[3.2.1]octan-4- yl)pyrimidin-5-yl)oxy)-6- (3,5-dichlorophenyl) pyridin-4-yl)methyl) piperidin-4-yl)acetic acid |
| 186 | 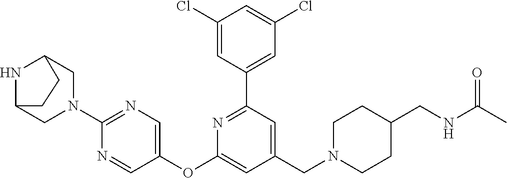 [SMILES: CC(=O)NCC1CCN(Cc2cc(Oc3cnc(N4CC5CCC(C4)N5)nc3)nc(-c3cc(Cl)cc(Cl)c3)c2)CC1] | N-((1-((2-((2-(3,8-diaza- bicyclo[3.2.1]octan-3- yl)pyrimidin-5-yl)oxy)-6- (3,5-dichlorophenyl) pyridin-4-yl)methyl) piperidin-4-yl)methyl) acetamide |
| 187 | 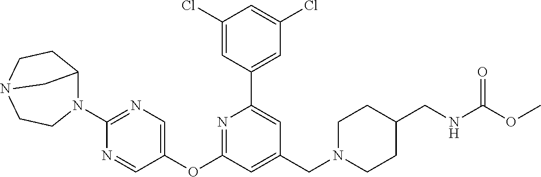 [SMILES: COC(=O)NCC1CCN(Cc2cc(Oc3cnc(N4CCN5CCC4C5)nc3)nc(-c3cc(Cl)cc(Cl)c3)c2)CC1] | methyl((1-((2-((2-(1,4- diazabicyclo[3.2.1]octan- 4-yl)pyrimidin-5-yl)oxy)- 6-(3,5-dichlorophenyl) pyridin-4-yl)methyl) piperidin-4-yl)methyl) carbamate |
| 188 | 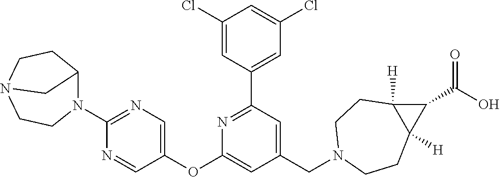 [SMILES: O=C(O)[C@H]1[C@@H]2CCN(Cc3cc(Oc4cnc(N5CCN6CCC5C6)nc4)nc(-c4cc(Cl)cc(Cl)c4)c3)CC[C@@H]21] | 4-((2-((2-(1,4-diaza- bicyclo[3.2.1]octan-4- yl)pyrimidin-5-yl)oxy)-6- (3,5-dichlorophenyl) pyridin-4-yl)methyl)-4- azabicyclo[5.1.0]octane- 8-carboxylic acid |
| 189 | 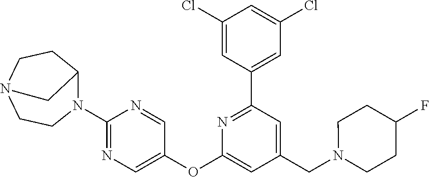 [SMILES: FC1CCN(Cc2cc(Oc3cnc(N4CCN5CCC4C5)nc3)nc(-c3cc(Cl)cc(Cl)c3)c2)CC1] | 4-(5-((6-(3,5-dichloro- phenyl)-4-((4-fluoro- piperidin-1-yl)methyl) pyridin-2-yl)oxy) pyrimidin-2-yl)-1,4- diazabicyclo[3.2.1]octane |
| 190 | 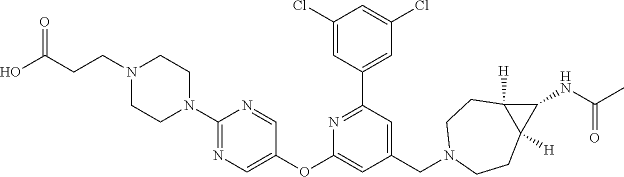 [SMILES: CC(=O)N[C@H]1[C@@H]2CCN(Cc3cc(Oc4cnc(N5CCN(CCC(=O)O)CC5)nc4)nc(-c4cc(Cl)cc(Cl)c4)c3)CC[C@@H]21] | 3-(4-(5-((4-(((1R,7S,8r)- 8-acetamido-4-azabicyclo [5.1.0]octan-4-yl)methyl)- 6-(3,5-dichlorophenyl) pyridin-2-yl)oxy) pyrimidin-2-yl)piperazin- 1-yl)propanoic acid |
| 191 | 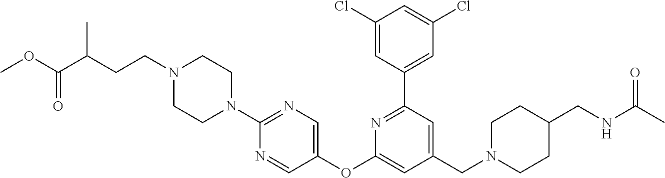 [SMILES: COC(=O)C(C)CCN1CCN(c2ncc(Oc3cc(CN4CCC(CNC(C)=O)CC4)cc(-c4cc(Cl)cc(Cl)c4)n3)cn2)CC1] | methyl4-(4-(5-((4-((4- (acetamidomethyl) piperidin-1-yl)methyl)- 6-(3,5-dichlorophenyl) pyridin-2-yl)oxy) pyrimidin-2-yl)piperazin- 1-yl)-2-methylbutanoate |
| 192 | 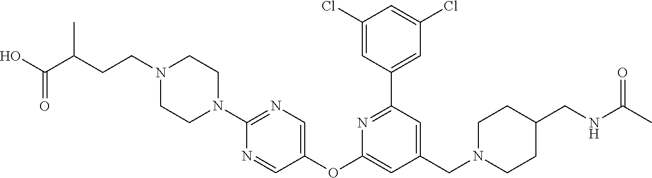 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1] | 4-(4-(5-((4-((4- (acetamidomethyl) piperidin-1-yl) methyl)-6-(3,5-dichloro- phenyl)pyridin-2-yl)oxy) pyrimidin-2-yl)piperazin- 1-yl)-2-methylbutanoic acid |
| 193 | 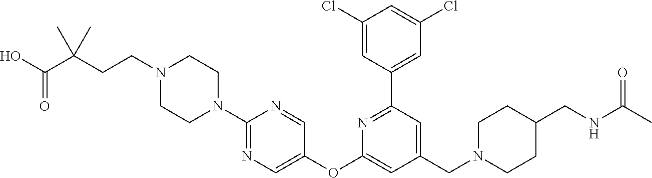 [SMILES: CC(=O)NCC1CCN(Cc2cc(Oc3cnc(N4CCN(CCC(C)(C)C(=O)O)CC4)nc3)nc(-c3cc(Cl)cc(Cl)c3)c2)CC1] | 4-(4-(5-((4-((4- (acetamidomethyl) piperidin-1-yl) methyl)-6-(3,5-dichloro- phenyl)pyridin-2-yl)oxy) pyrimidin-2-yl)piperazin- 1-yl)-2,2-dimethyl- butanoic acid |
| 194 | 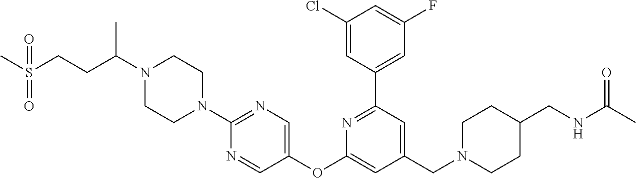 [SMILES: CC(=O)NCC1CCN(Cc2cc(Oc3cnc(N4CCN(C(C)CCS(C)(=O)=O)CC4)nc3)nc(-c3cc(F)cc(Cl)c3)c2)CC1] | N-((1-((2-(3-chloro-5- fluorophenyl)-6-((2-(4-(4- (methylsulfonyl)butan-2- yl)piperazin-1-yl) pyrimidin-5-yl)oxy) pyridin-4-yl)methyl) piperidin-4-yl)methyl) acetamide |
| 195 | 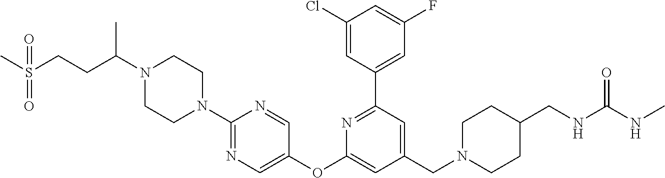 | 1-((1-((2-(3-chloro-5- fluorophenyl)-6-((2-(4-(4- (methylsulfonyl)butan-2- yl)piperazin-1-yl) pyrimidin-5-yl)oxy) pyridin-4-yl)methyl) piperidin-4-yl)methyl)- 3-methylurea |
| 196 | 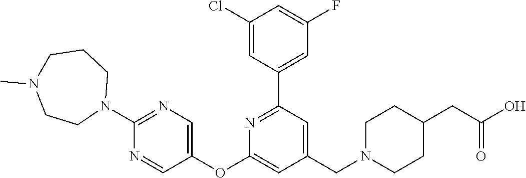 [SMILES: CN1CCCN(c2ncc(Oc3cc(CN4CCC(CC(=O)O)CC4)cc(-c4cc(F)cc(Cl)c4)n3)cn2)CC1] | 2-(1-((2-(3-chloro-5- fluorophenyl)-6-((2-(4- methyl-1,4-diazepan-1- yl)pyrimidin-5-yl)oxy) pyridin-4-yl)methyl) piperidin-4-yl)acetic acid |
| 197 | 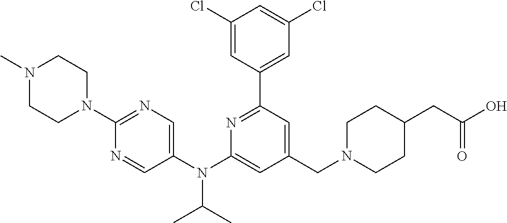 [SMILES: CC(C)N(c1cnc(N2CCN(C)CC2)nc1)c1cc(CN2CCC(CC(=O)O)CC2)cc(-c2cc(Cl)cc(Cl)c2)n1] | 2-(1-((2-(3,5-dichloro- phenyl)-6-(isopropyl(2- (4-methylpiperazin-1- yl)pyrimidin-5-yl)amino) pyridin-4-yl)methyl) piperidin-4-yl)acetic acid |
| 198 | 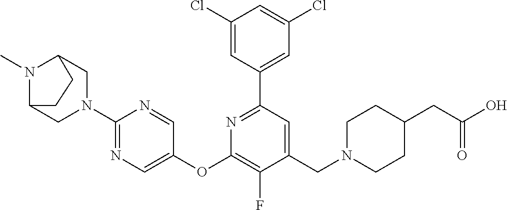 [SMILES: CN1C2CCC1CN(c1ncc(Oc3nc(-c4cc(Cl)cc(Cl)c4)cc(CN4CCC(CC(=O)O)CC4)c3F)cn1)C2] | 2-(1-((6-(3,5-dichloro- phenyl)-3-fluoro-2-((2- (8-methyl-3,8-diaza- bicyclo[3.2.1]octan-3-yl) pyrimidin-5-yl)oxy) pyridin-4-yl)methyl) piperidin-4-yl)acetic acid |
| 199 | 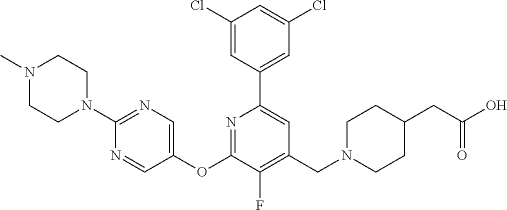 [SMILES: CN1CCN(c2ncc(Oc3nc(-c4cc(Cl)cc(Cl)c4)cc(CN4CCC(CC(=O)O)CC4)c3F)cn2)CC1] | 2-(1-((6-(3,5-dichloro- phenyl)-3-fluoro-2-((2- (4-methylpiperazin-1-yl) pyrimidin-5-yl)oxy) pyridin-4-yl)methyl) piperidin-4-yl)acetic acid |
| 200 | 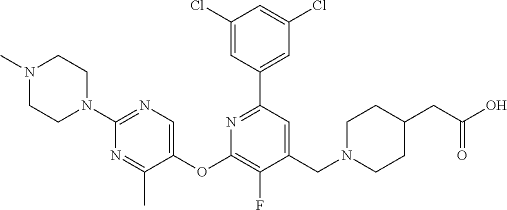 [SMILES: Cc1nc(N2CCN(C)CC2)ncc1Oc1nc(-c2cc(Cl)cc(Cl)c2)cc(CN2CCC(CC(=O)O)CC2)c1F] | 2-(1-((6-(3,5-dichloro- phenyl)-3-fluoro-2-((4- methyl-2-(4-methyl- piperazin-1-yl)pyrimidin- 5-yl)oxy)pyridin-4- yl)methyl)piperidin-4- yl)acetic acid |
| 201 | 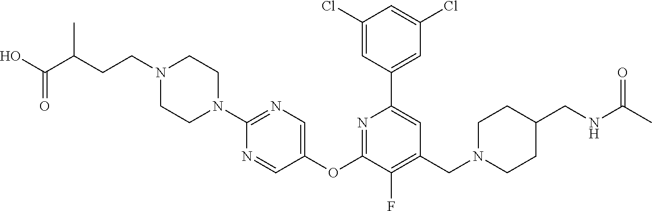 [SMILES: CC(=O)NCC1CCN(Cc2cc(-c3cc(Cl)cc(Cl)c3)nc(Oc3cnc(N4CCN(CCC(C)C(=O)O)CC4)nc3)c2F)CC1] | 4-(4-(5-((4-((4- (acetamido-methyl) piperidin-1-yl)methyl)- 6-(3,5-dichloro-phenyl)- 3-fluoropyridin-2-yl) oxy)pyrimidin-2-yl) piperazin-1-yl)-2- methyl-butanoic acid |
| 202 | 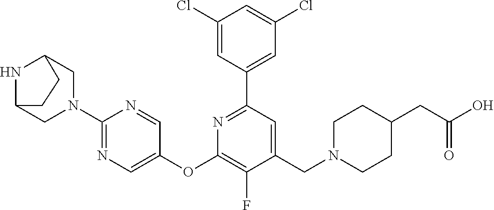 [SMILES: O=C(O)CC1CCN(Cc2cc(-c3cc(Cl)cc(Cl)c3)nc(Oc3cnc(N4CC5CCC(C4)N5)nc3)c2F)CC1] | 2-(1-((2-((2-(3,8-diaza- bicyclo[3.2.1]octan-3-yl) pyrimidin-5-yl)oxy)-6- (3,5-dichlorophenyl)-3- fluoropyridin-4-yl)methyl) piperidin-4-yl)acetic acid |
| 203 | 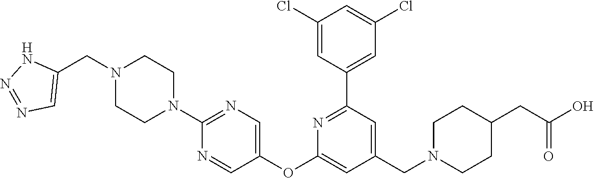 [SMILES: O=C(O)CC1CCN(Cc2cc(Oc3cnc(N4CCN(Cc5cnn[nH]5)CC4)nc3)nc(-c3cc(Cl)cc(Cl)c3)c2)CC1] | 2-(1-((2-((2-(4-((1H-1,2,3- triazol-5-yl)methyl) piperazin-1-yl)pyrimidin- 5-yl)oxy)-6-(3,5-dichloro- phenyl)pyridin-4-yl) methyl)piperidin-4-yl) acetic acid |
| 204 | 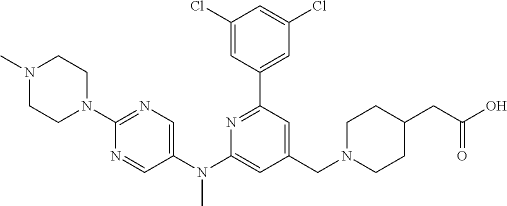 [SMILES: CN1CCN(c2ncc(N(C)c3cc(CN4CCC(CC(=O)O)CC4)cc(-c4cc(Cl)cc(Cl)c4)n3)cn2)CC1] | 2-(1-((2-(3,5-dichloro- phenyl)-6-(methyl(2-(4- methylpiperazin-1-yl) pyrimidin-5-yl)amino) pyridin-4-yl)methyl) piperidin-4-yl)acetic acid |
| 205 | 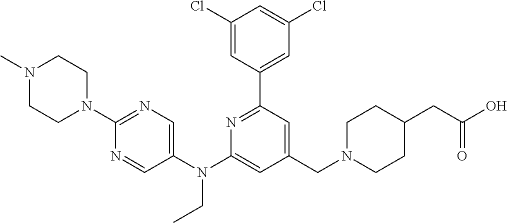 [SMILES: CCN(c1cnc(N2CCN(C)CC2)nc1)c1cc(CN2CCC(CC(=O)O)CC2)cc(-c2cc(Cl)cc(Cl)c2)n1] | 2-(1-((2-(3,5-dichloro- phenyl)-6-(ethyl(2-(4- methylpiperazin-1-yl) pyrimidin-5-yl)amino) pyridin-4-yl)methyl) piperidin-4-yl)acetic acid |
| 206 | 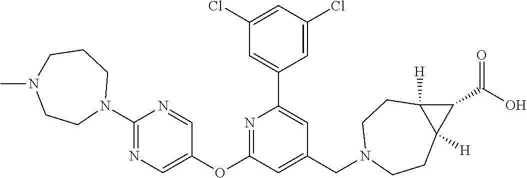 [SMILES: CN1CCCN(c2ncc(Oc3cc(CN4CC[C@@H]5[C@H](CC4)[C@H]5C(=O)O)cc(-c4cc(Cl)cc(Cl)c4)n3)cn2)CC1] | (1R,7S,8r)-4-((2-(3,5- dichlorophenyl)-6-((2-(4- methyl-1,4-diazepan-1- yl)pyrimidin-5-yl)oxy) pyridin-4-yl)methyl)-4- azabicyclo[5.1.0]octane- 8-carboxylic acid |
| 207 | 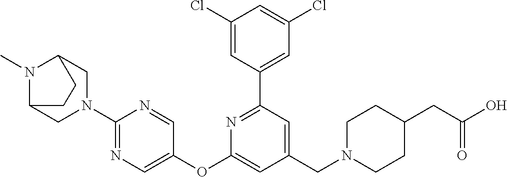 [SMILES: CN1C2CCC1CN(c1ncc(Oc3cc(CN4CCC(CC(=O)O)CC4)cc(-c4cc(Cl)cc(Cl)c4)n3)cn1)C2] | 2-(1-((2-(3,5-dichloro- phenyl)-6-((2-(8-methyl- 3,8-diazabicyclo[3.2.1] octan-3-yl)pyrimidin-5- yl)oxy)pyridin-4-yl) methyl)piperidin-4-yl) acetic acid |
| 208 | 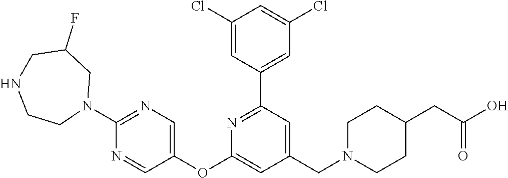 [SMILES: O=C(O)CC1CCN(Cc2cc(Oc3cnc(N4CCNCC(F)C4)nc3)nc(-c3cc(Cl)cc(Cl)c3)c2)CC1] | 2-(1-((2-(3,5-dichloro- phenyl)-6-((2-(6-fluoro- 1,4-diazepan-1-yl) pyrimidin-5-yl)oxy) pyridin-4-yl)methyl) piperidin-4-yl)acetic acid |

In certain embodiments, the compound of Formula (I) useful in the present disclosure is of the Formula (III):

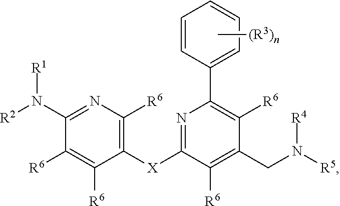

or a pharmaceutically acceptable salt thereof, wherein:

X is —O— or —N(R⁸)—;

R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁹, —C(O)CO₂R⁸, —R⁷, —OR⁷, —NHR⁸, —NR⁷R⁸, —C(O)R⁷, —CONHR⁸, CONR⁷R⁸, or —SO₂R⁷;

each R³ is independently halogen, —CN, —O(C₁-C₄)alkyl, or optionally substituted (C₁-C₄)alkyl;

R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;

each R⁶ is independently H, halogen, optionally substituted (C₁-C₄)alkyl, —OH, or optionally substituted (C₁-C₄)alkoxy;

each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);

each of R⁸ and R⁹ is independently H, optionally substituted (C₁-C₄)alkyl, or optionally substituted (C₃-C₆)cycloalkyl; and

n is 1, 2, 3, or 4.

In certain embodiments, the compound of Formula (III), or a pharmaceutically acceptable salt thereof, useful in the present disclosure is of the formula:

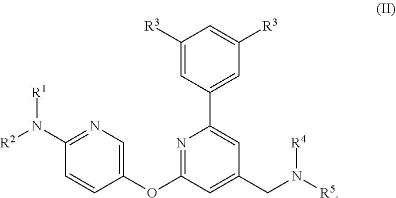

In certain embodiments, the compound of Formula (III), or a pharmaceutically acceptable salt thereof, useful in the present disclosure is of the formula:

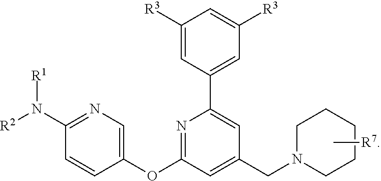

In certain embodiments, the compound of Formula (III), or a pharmaceutically acceptable salt thereof, useful in the present disclosure is of the formula:

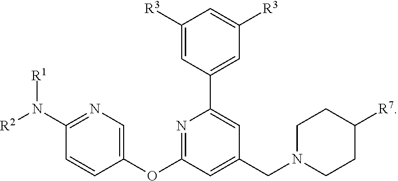

In certain embodiments, the compound of Formula (III), or a pharmaceutically acceptable salt thereof, useful in the present disclosure is of the formula:

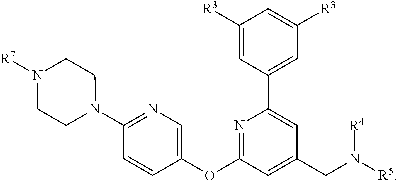

In certain embodiments, the compound of Formula (III), or a pharmaceutically acceptable salt thereof, useful in the present disclosure is of the formula:

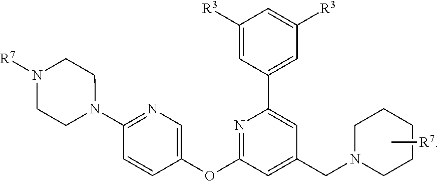

In certain embodiments, the compound of Formula (III) useful in the present disclosure is of the formula thereof, useful in the present disclosure is of the formula:

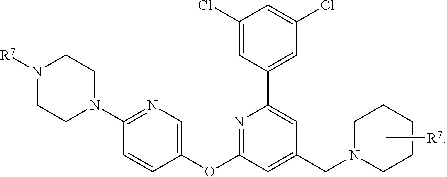

In certain embodiments, the compound of Formula (III) useful in the present disclosure is of the formula (Table 2, #219):

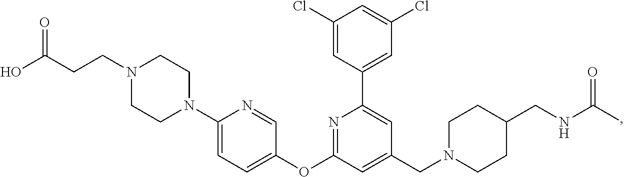

or a pharmaceutically acceptable salt thereof.

In certain embodiments, a compound of Formula (I) or Formula (III) may be any one of the compounds found in Table 2 below. In certain embodiments, the disclosed compositions, methods, and uses comprise administering to the subject in need thereof a therapeutically effective amount of any one of the compounds found in Table 2 below.

TABLE 2
Compounds useful in the present disclosure

| # | Structure | Name |
|---|---|---|
| 209 | 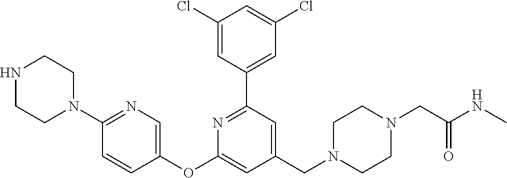 [SMILES: CNC(=O)CN1CCN(Cc2cc(Oc3ccc(N4CCNCC4)nc3)nc(-c3cc(Cl)cc(Cl)c3)c2)CC1] | 2-(4-((2-(3,5-dichlorophenyl)- 6-((6-(piperazin-1-yl)pyridin- 3-yl)oxy)pyridin-4-yl)methyl) piperazin-1-yl)-N- methylacetamide |
| 210 | 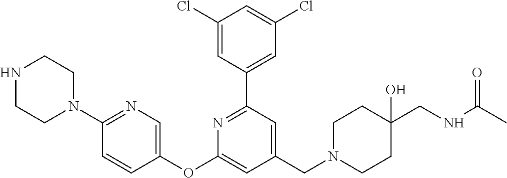 [SMILES: CC(=O)NCC1(O)CCN(Cc2cc(Oc3ccc(N4CCNCC4)nc3)nc(-c3cc(Cl)cc(Cl)c3)c2)CC1] | N-((1-((2-(3,5-dichlorophenyl)- 6-((6-(piperazin-1-yl)pyridin- 3-yl)oxy)pyridin-4-yl)methyl)- 4-hydroxypiperidin-4- yl)methyl)acetamide |
| 211 | 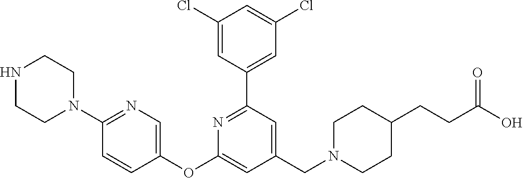 [SMILES: O=C(O)CCC1CCN(Cc2cc(Oc3ccc(N4CCNCC4)nc3)nc(-c3cc(Cl)cc(Cl)c3)c2)CC1] | 3-(1-((2-(3,5-dichlorophenyl)- 6-((6-(piperazin-1-yl)pyridin- 3-yl)oxy)pyridin-4-yl)methyl) piperidin-4-yl)propanoicacid |
| 212 | 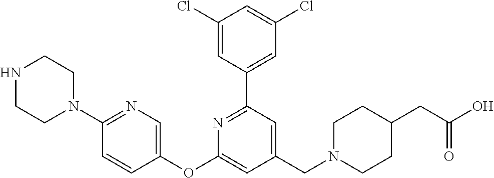 [SMILES: O=C(O)CC1CCN(Cc2cc(Oc3ccc(N4CCNCC4)nc3)nc(-c3cc(Cl)cc(Cl)c3)c2)CC1] | 2-(1-((2-(3,5-dichlorophenyl)- 6-((6-(piperazin-1-yl)pyridin- 3-yl)oxy)pyridin-4-yl)methyl) piperidin-4-yl)aceticacid |
| 213 | 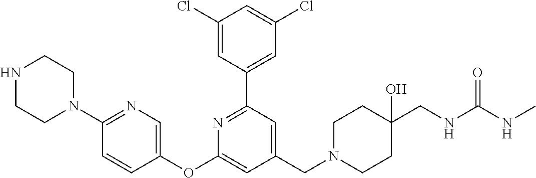 [SMILES: CNC(=O)NCC1(O)CCN(Cc2cc(Oc3ccc(N4CCNCC4)nc3)nc(-c3cc(Cl)cc(Cl)c3)c2)CC1] | 1-((1-((2-(3,5-dichlorophenyl)- 6-((6-(piperazin-1-yl)pyridin-3- yl)oxy)pyridin-4-yl)methyl)-4- hydroxypiperidin-4- yl)methyl)-3-methylurea |
| 214 | 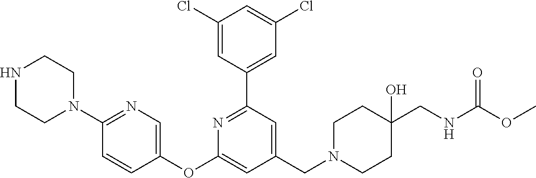 [SMILES: COC(=O)NCC1(O)CCN(Cc2cc(Oc3ccc(N4CCNCC4)nc3)nc(-c3cc(Cl)cc(Cl)c3)c2)CC1] | methyl((1-((2- (3,5-dichlorophenyl)-6-((6- (piperazin-1-yl)pyridin-3-yl) oxy)pyridin-4-yl)methyl)-4- hydroxypiperidin-4- yl)methyl)carbamate |
| 215 | 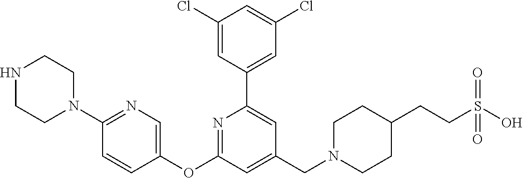 [SMILES: O=S(=O)(O)CCC1CCN(Cc2cc(Oc3ccc(N4CCNCC4)nc3)nc(-c3cc(Cl)cc(Cl)c3)c2)CC1] | 2-(1-((2-(3,5-dichlorophenyl)- 6-((6-(piperazin-1-yl)pyridin- 3-yl)oxy)pyridin-4-yl)methyl) piperidin-4-yl) ethanesulfonicacid |
| 216 | 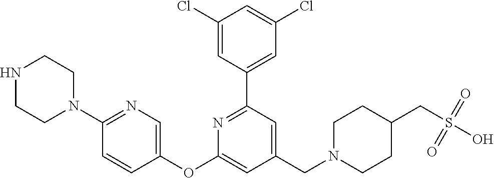 [SMILES: O=S(=O)(O)CC1CCN(Cc2cc(Oc3ccc(N4CCNCC4)nc3)nc(-c3cc(Cl)cc(Cl)c3)c2)CC1] | (1-((2-(3,5-dichlorophenyl)-6- ((6-(piperazin-1-yl)pyridin-3- yl)oxy)pyridin-4-yl)methyl) piperidin-4-yl) methanesulfonicacid |
| 217 | 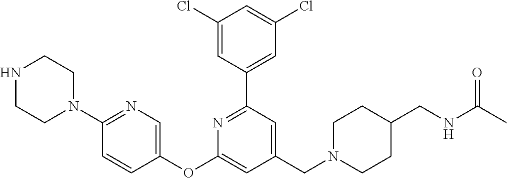 [SMILES: CC(=O)NCC1CCN(Cc2cc(Oc3ccc(N4CCNCC4)nc3)nc(-c3cc(Cl)cc(Cl)c3)c2)CC1] | N-((1-((2-(3,5-dichlorophenyl)- 6-((6-(piperazin-1-yl)pyridin-3- yl)oxy)pyridin-4-yl)methyl) piperidin-4-yl)methyl)acetamide |
| 218 | 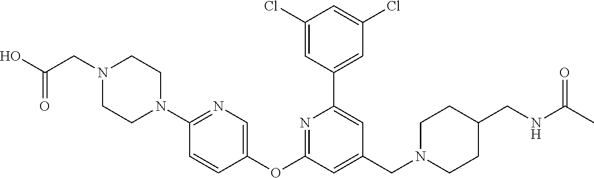 [SMILES: CC(=O)NCC1CCN(Cc2cc(Oc3ccc(N4CCN(CC(=O)O)CC4)nc3)nc(-c3cc(Cl)cc(Cl)c3)c2)CC1] | 2-(4-(5-((4-((4- (acetamidomethyl)piperidin- 1-yl)methyl)-6- (3,5-dichlorophenyl)pyridin- 2-yl)oxy)pyridin-2-yl) piperazin-1-yl)aceticacid |
| 219 | 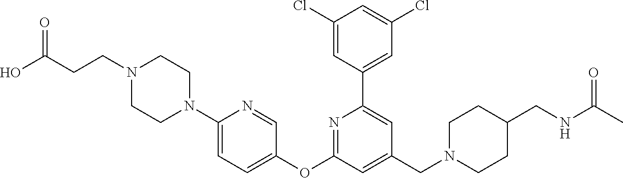 [SMILES: CC(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((4-((4- (acetamidomethyl)piperidin- 1-yl)methyl)-6- (3,5-dichlorophenyl)pyridin- 2-yl)oxy)pyridin-2-yl) piperazin-1-yl)propanoicacid |
| 220 | 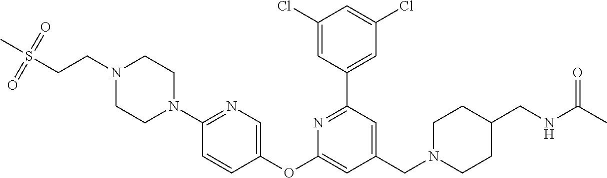 [SMILES: CC(=O)NCC1CCN(Cc2cc(Oc3ccc(N4CCN(CCS(C)(=O)=O)CC4)nc3)nc(-c3cc(Cl)cc(Cl)c3)c2)CC1] | N-((1-((2-(3,5-dichlorophenyl)- 6-((6-(4-(2-(methylsulfonyl) ethyl)piperazin-1-yl)pyridin- 3-yl)oxy)pyridin-4-yl)methyl) piperidin-4-yl)methyl)acetamide |
| 221 | 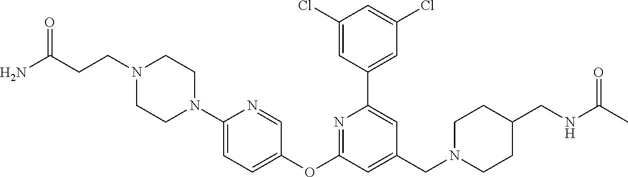 [SMILES: CC(=O)NCC1CCN(Cc2cc(Oc3ccc(N4CCN(CCC(N)=O)CC4)nc3)nc(-c3cc(Cl)cc(Cl)c3)c2)CC1] | 3-(4-(5-((4-((4- (acetamidomethyl)piperidin- 1-yl)methyl)-6- (3,5-dichlorophenyl)pyridin- 2-yl)oxy)pyridin-2-yl) piperazin-1-yl)propanamide |
| 222 | 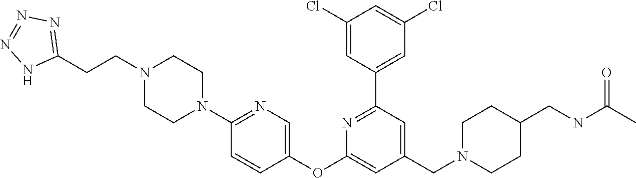 [SMILES: CC(=O)NCC1CCN(Cc2cc(Oc3ccc(N4CCN(CCc5nnn[nH]5)CC4)nc3)nc(-c3cc(Cl)cc(Cl)c3)c2)CC1] | N-((1-((2-((6-(4-(2-(1H- tetrazol-5-yl)ethyl)piperazin- 1-yl)pyridin-3-yl)oxy)-6- (3,5-dichlorophenyl)pyridin-4- yl)methyl)piperidin-4-yl) methyl)acetamide |
| 223 | 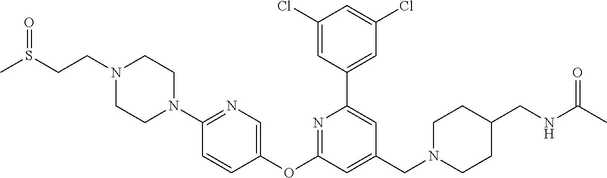 [SMILES: CC(=O)NCC1CCN(Cc2cc(Oc3ccc(N4CCN(CCS(C)=O)CC4)nc3)nc(-c3cc(Cl)cc(Cl)c3)c2)CC1] | N-((1-((2-(3,5-dichlorophenyl)- 6-((6-(4-(2-(methylsulfinyl) ethyl)piperazin-1-yl) pyridin-3-yl)oxy)pyridin-4- yl)methyl)piperidin-4-yl) methyl)acetamide |
| 224 | 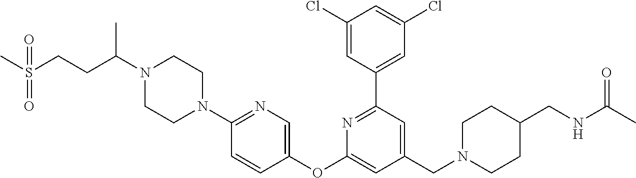 [SMILES: CC(=O)NCC1CCN(Cc2cc(Oc3ccc(N4CCN(C(C)CCS(C)(=O)=O)CC4)nc3)nc(-c3cc(Cl)cc(Cl)c3)c2)CC1] | N-((1-((2-(3,5-dichlorophenyl)- 6-((6-(4-(4-(methylsulfonyl) butan-2-yl)piperazin-1- yl)pyridin-3-yl)oxy) pyridin-4-yl)methyl) piperidin-4-yl)methyl) acetamide |
| 225 | 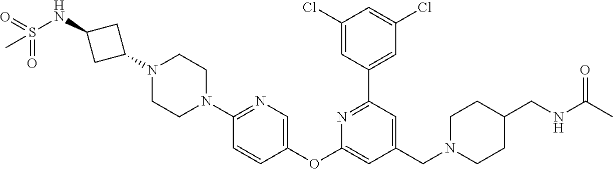 [SMILES: CC(=O)NCC1CCN(Cc2cc(Oc3ccc(N4CCN([C@H]5C[C@H](NS(C)(=O)=O)C5)CC4)nc3)nc(-c3cc(Cl)cc(Cl)c3)c2)CC1] | N-((1-((2-(3,5-dichlorophenyl)- 6-((6-(4-((trans)-3- (methylsulfonamido) cyclobutyl)piperazin-1-yl) pyridin-3-yl)oxy)pyridin-4-yl) methyl)piperidin-4-yl) methyl)acetamide |
| 226 | 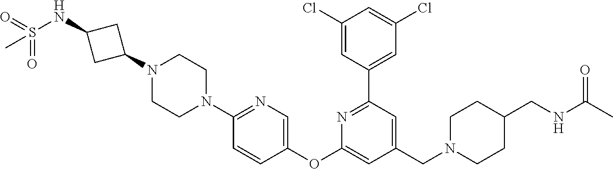 [SMILES: CC(=O)NCC1CCN(Cc2cc(Oc3ccc(N4CCN([C@H]5C[C@@H](NS(C)(=O)=O)C5)CC4)nc3)nc(-c3cc(Cl)cc(Cl)c3)c2)CC1] | N-((1-((2-(3,5-dichlorophenyl)- 6-((6-(4-((cis)-3- (methylsulfonamido)cyclobutyl) piperazin-1-yl)pyridin-3-yl) oxy)pyridin-4-yl)methyl) piperidin-4-yl)methyl) acetamide |
| 227 | 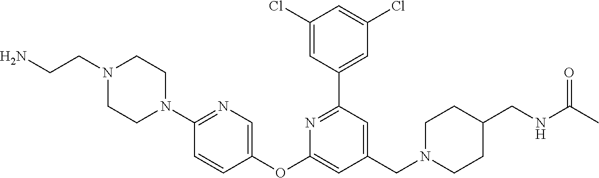 [SMILES: CC(=O)NCC1CCN(Cc2cc(Oc3ccc(N4CCN(CCN)CC4)nc3)nc(-c3cc(Cl)cc(Cl)c3)c2)CC1] | N-((1-((2-((6-(4-(2-aminoethyl) piperazin-1-yl)pyridin- 3-yl)oxy)-6-(3,5- dichlorophenyl)pyridin-4- yl)methyl)piperidin-4-yl) methyl)acetamide |
| 228 | 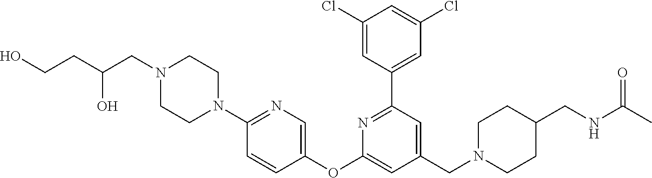 [SMILES: CC(=O)NCC1CCN(Cc2cc(Oc3ccc(N4CCN(CC(O)CCO)CC4)nc3)nc(-c3cc(Cl)cc(Cl)c3)c2)CC1] | N-((1-((2-(3,5-dichlorophenyl)- 6-((6-(4-(2,4-dihydroxybutyl) piperazin-1-yl)pyridin-3-yl) oxy)pyridin-4-yl)methyl) piperidin-4- yl)methyl)acetamide |
| 229 | 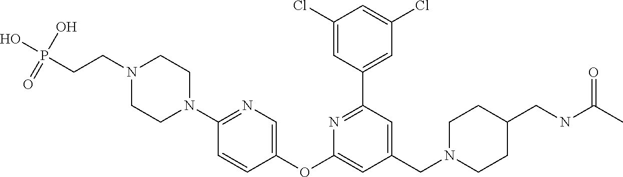 [SMILES: CC(=O)NCC1CCN(Cc2cc(Oc3ccc(N4CCN(CCP(=O)(O)O)CC4)nc3)nc(-c3cc(Cl)cc(Cl)c3)c2)CC1] | (2-(4-(5-((4-((4- (acetamidomethyl)piperidin- 1-yl)methyl)-6- (3,5-dichlorophenyl)pyridin- 2-yl)oxy)pyridin-2-yl) piperazin-1-yl)ethyl) phosphonicacid |
| 230 | 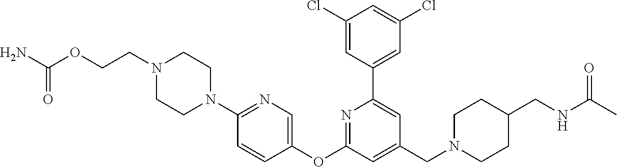 [SMILES: CC(=O)NCC1CCN(Cc2cc(Oc3ccc(N4CCN(CCOC(N)=O)CC4)nc3)nc(-c3cc(Cl)cc(Cl)c3)c2)CC1] | 2-(4-(5-((4-((4- (acetamidomethyl)piperidin- 1-yl)methyl)-6- (3,5-dichlorophenyl)pyridin- 2-yl)oxy)pyridin-2-yl) piperazin-1- yl)ethylcarbamate |
| 231 | 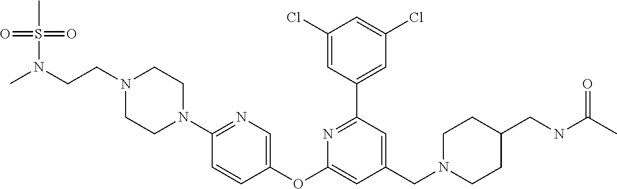 [SMILES: CC(=O)NCC1CCN(Cc2cc(Oc3ccc(N4CCN(CCN(C)S(C)(=O)=O)CC4)nc3)nc(-c3cc(Cl)cc(Cl)c3)c2)CC1] | N-((1-((2-(3,5-dichlorophenyl)- 6-((6-(4-(2-(N- methylmethylsulfonamido) ethyl)piperazin-1-yl)pyridin-3- yl)oxy)pyridin-4-yl)methyl) piperidin-4-yl)methyl) acetamide |
| 232 | 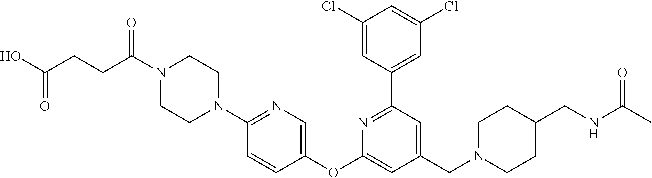 [SMILES: CC(=O)NCC1CCN(Cc2cc(Oc3ccc(N4CCN(C(=O)CCC(=O)O)CC4)nc3)nc(-c3cc(Cl)cc(Cl)c3)c2)CC1] | 4-(4-(5-((4-((4- (acetamidomethyl)piperidin- 1-yl)methyl)-6- (3,5-dichlorophenyl)pyridin- 2-yl)oxy)pyridin-2-yl) piperazin-1-yl)-4- oxobutanoicacid |
| 233 | 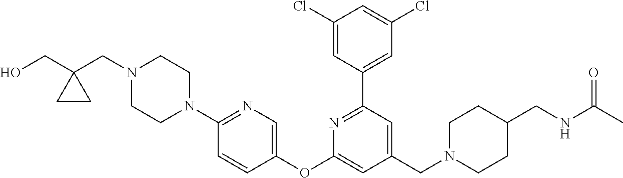 [SMILES: CC(=O)NCC1CCN(Cc2cc(Oc3ccc(N4CCN(CC5(CO)CC5)CC4)nc3)nc(-c3cc(Cl)cc(Cl)c3)c2)CC1] | N-((1-((2-(3,5-dichlorophenyl)- 6-((6-(4-((1-(hydroxymethyl) cyclopropyl)methyl)piperazin- 1-yl)pyridin-3-yl)oxy)pyridin- 4-yl)methyl)piperidin-4- yl)methyl)acetamide |
| 234 | 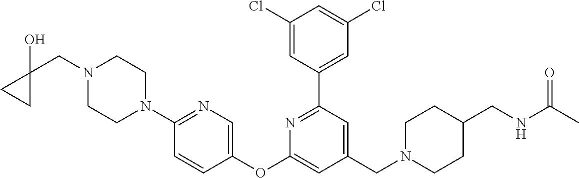 [SMILES: CC(=O)NCC1CCN(Cc2cc(Oc3ccc(N4CCN(CC5(O)CC5)CC4)nc3)nc(-c3cc(Cl)cc(Cl)c3)c2)CC1] | N-((1-((2-(3,5-dichlorophenyl)- 6-((6-(4-((1- hydroxycyclopropyl) methyl)piperazin-1- yl)pyridin-3-yl)oxy)pyridin-4- yl)methyl)piperidin-4-yl) methyl)acetamide |
| 235 | 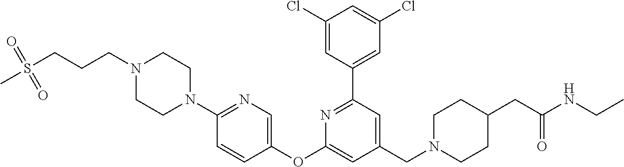 [SMILES: CCNC(=O)CC1CCN(Cc2cc(Oc3ccc(N4CCN(CCCS(C)(=O)=O)CC4)nc3)nc(-c3cc(Cl)cc(Cl)c3)c2)CC1] | 2-(1-((2-(3,5-dichlorophenyl)- 6-((6-(4-(3-(methylsulfonyl) propyl)piperazin-1-yl)pyridin-3- yl)oxy)pyridin-4- yl)methyl)piperidin-4-yl)- N-ethylacetamide |
| 236 | 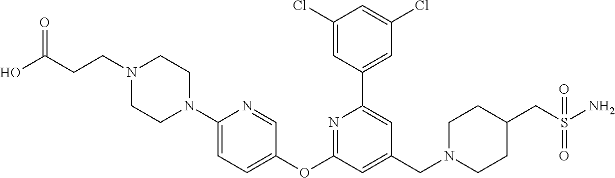 [SMILES: NS(=O)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)- 4-((4-(sulfamoylmethyl)piperidin- 1-yl)methyl)pyridin-2-yl)oxy) pyridin-2-yl)piperazin-1-yl) propanoicacid |
| 237 | 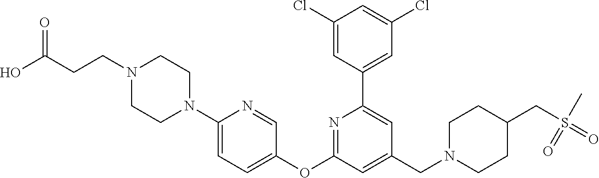 [SMILES: CS(=O)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)- 4-((4-((methylsulfonyl) methyl)piperidin-1- yl)methyl)pyridin-2-yl)oxy) pyridin-2-yl)piperazin-1-yl) propanoicacid |
| 238 | 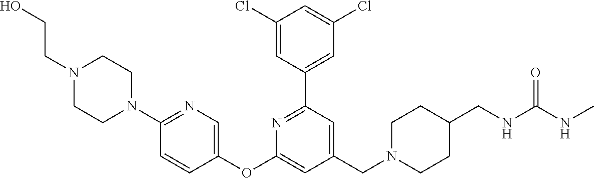 [SMILES: CNC(=O)NCC1CCN(Cc2cc(Oc3ccc(N4CCN(CCO)CC4)nc3)nc(-c3cc(Cl)cc(Cl)c3)c2)CC1] | 1-((1-((2-(3,5-dichlorophenyl)- 6-((6-(4-(2-hydroxyethyl) piperazin-1-yl)pyridin-3- yl)oxy)pyridin-4-yl)methyl) piperidin-4- yl)methyl)-3-methylurea |
| 239 | 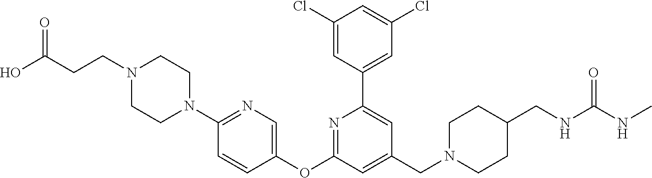 [SMILES: CNC(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)- 4-((4-((3-methylureido)methyl) piperidin-1-yl)methyl)pyridin- 2-yl)oxy)pyridin-2-yl) piperazin-1-yl)propanoicacid |
| 240 | 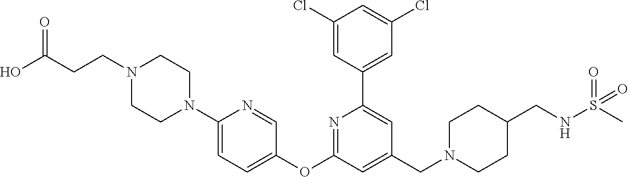 [SMILES: CS(=O)(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)- 4-((4-(methylsulfonamidomethyl) piperidin-1-yl)methyl)pyridin- 2-yl)oxy)pyridin-2- yl)piperazin-1-yl)propanoicacid |
| 241 | 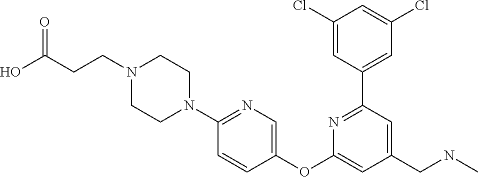 [SMILES: CNCc1cc(Oc2ccc(N3CCN(CCC(=O)O)CC3)nc2)nc(-c2cc(Cl)cc(Cl)c2)c1] | 3-(4-(5-((6-(3,5-dichlorophenyl)- 4-((methylamino)methyl)pyridin- 2-yl)oxy)pyridin-2-yl)piperazin- 1-yl)propanoicacid |
| 242 | 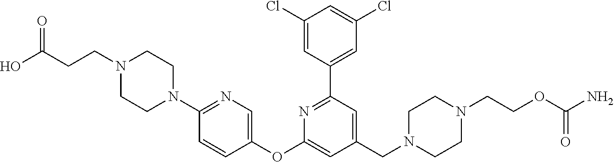 [SMILES: NC(=O)OCCN1CCN(Cc2cc(Oc3ccc(N4CCN(CCC(=O)O)CC4)nc3)nc(-c3cc(Cl)cc(Cl)c3)c2)CC1] | 3-(4-(5-((4-((4-(2- (carbamoyloxy)ethyl)piperazin- 1-yl)methyl)-6- (3,5-dichlorophenyl)pyridin-2- yl)oxy)pyridin-2-yl)piperazin- 1-yl)propanoicacid |
| 243 | 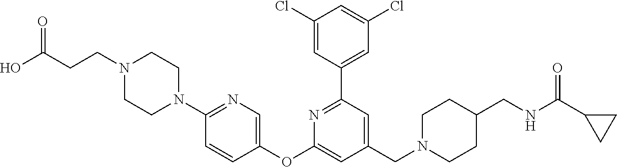 [SMILES: O=C(O)CCN1CCN(c2ccc(Oc3cc(CN4CCC(CNC(=O)C5CC5)CC4)cc(-c4cc(Cl)cc(Cl)c4)n3)cn2)CC1] | 3-(4-(5-((4-((4- (cyclopropane- carboxamidomethyl) piperidin-1-yl)methyl)-6-(3,5- dichlorophenyl)pyridin- 2-yl)oxy)pyridin-2-yl) piperazin-1- yl)propanoicacid |
| 244 | 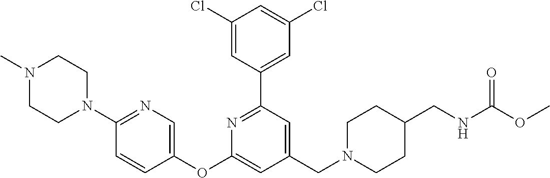 | methyl((1-((2-(3,5- dichlorophenyl)-6-((6-(4- methylpiperazin-1-yl)pyridin-3- yl)oxy)pyridin-4-yl)methyl) piperidin-4- yl)methyl)carbamate |
| 245 | 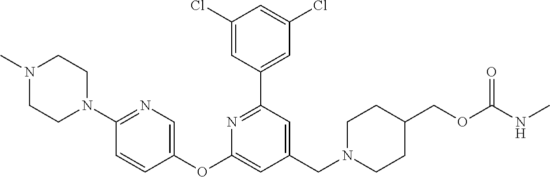 | (1-((2-(3,5-dichlorophenyl)-6- ((6-(4-methylpiperazin-1-yl) pyridin-3-yl)oxy)pyridin-4- yl)methyl)piperidin-4- yl)methylmethylcarbamate |
| 246 | 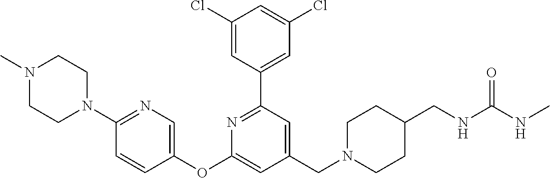 | 1-((1-((2-(3,5-dichlorophenyl)- 6-((6-(4-methylpiperazin- 1-yl)pyridin-3-yl)oxy)pyridin- 4-yl)methyl)piperidin-4- yl)methyl)-3-methylurea |
| 247 | 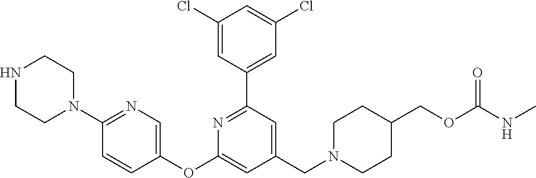 [SMILES: CNC(=O)OCC1CCN(Cc2cc(Oc3ccc(N4CCNCC4)nc3)nc(-c3cc(Cl)cc(Cl)c3)c2)CC1] | (1-((2-(3,5-dichlorophenyl)-6- ((6-(piperazin-1-yl)pyridin-3- yl)oxy)pyridin-4-yl)methyl) piperidin-4-yl) methylmethylcarbamate |
| 248 | 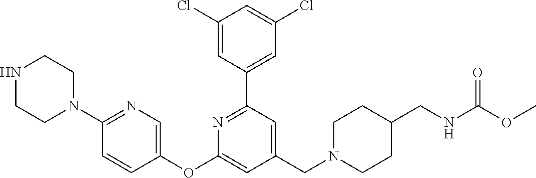 [SMILES: COC(=O)NCC1CCN(Cc2cc(Oc3ccc(N4CCNCC4)nc3)nc(-c3cc(Cl)cc(Cl)c3)c2)CC1] | methyl((1-((2- (3,5-dichlorophenyl)-6-((6- (piperazin-1-yl)pyridin-3-yl) oxy)pyridin-4-yl)methyl) piperidin-4-yl)methyl) carbamate |
| 249 | 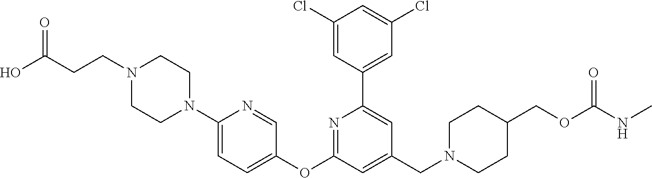 [SMILES: CNC(=O)OCC1CCN(Cc2cc(Oc3ccc(N4CCN(CCC(=O)O)CC4)nc3)nc(-c3cc(Cl)cc(Cl)c3)c2)CC1] | 3-(4-(5-((6- (3,5-dichlorophenyl)-4- ((4-(((methylcarbamoyl)oxy) methyl)piperidin-1-yl)methyl) pyridin-2-yl)oxy)pyridin-2- yl)piperazin-1-yl) propanoicacid |
| 250 | 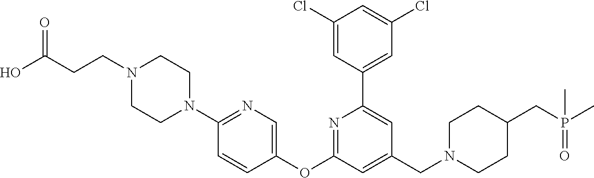 [SMILES: CP(C)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6- (3,5-dichlorophenyl)-4-((4- ((dimethylphosphoryl)methyl) piperidin-1-yl)methyl)pyridin- 2-yl)oxy)pyridin-2-yl) piperazin-1-yl)propanoicacid |
| 251 | 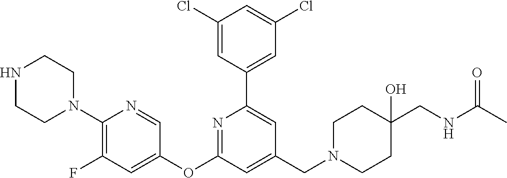 [SMILES: CC(=O)NCC1(O)CCN(Cc2cc(Oc3cnc(N4CCNCC4)c(F)c3)nc(-c3cc(Cl)cc(Cl)c3)c2)CC1] | N-((1-((2-(3,5-dichlorophenyl)- 6-((5-fluoro-6-(piperazin-1-yl) pyridin-3-yl)oxy)pyridin-4-yl) methyl)-4-hydroxypiperidin-4- yl)methyl)acetamide |
| 252 | 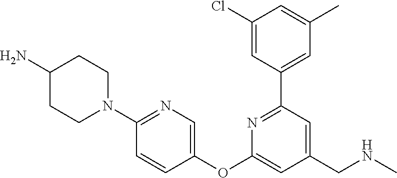 [SMILES: CNCc1cc(Oc2ccc(N3CCC(N)CC3)nc2)nc(-c2cc(C)cc(Cl)c2)c1] | 1-(5-((6-(3-chloro-5- methylphenyl)-4-((methylamino) methyl)pyridin-2-yl)oxy)pyridin- 2-yl)piperidin-4-amine |
| 253 | 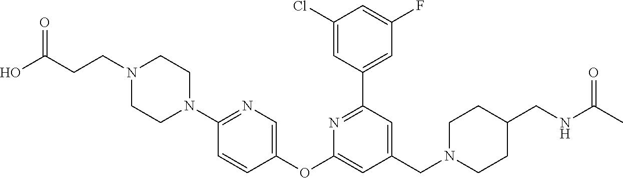 [SMILES: CC(=O)NCC1CCN(Cc2cc(Oc3ccc(N4CCN(CCC(=O)O)CC4)nc3)nc(-c3cc(F)cc(Cl)c3)c2)CC1] | 3-(4-(5-((4-((4- (acetamidomethyl)piperidin- 1-yl)methyl)-6-(3-chloro-5- fluorophenyl)pyridin-2-yl) oxy)pyridin-2- yl)piperazin-1-yl) propanoicacid |
| 254 | 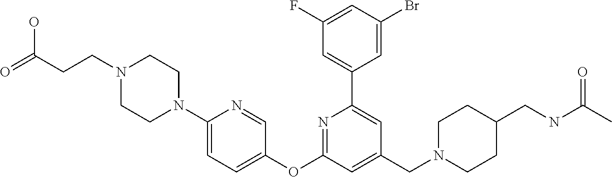 [SMILES: CC(=O)NCC1CCN(Cc2cc(Oc3ccc(N4CCN(CCC(=O)O)CC4)nc3)nc(-c3cc(F)cc(Br)c3)c2)CC1] | 3-(4-(5-((4-((4- (acetamidomethyl)piperidin- 1-yl)methyl)-6-(3-bromo-5- fluorophenyl)pyridin-2-yl) oxy)pyridin-2-yl) piperazin-1-yl) propanoicacid |
| 255 | 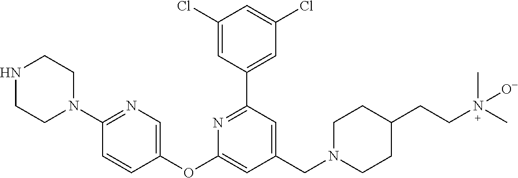 | 2-(1-((2-(3,5-dichlorophenyl)- 6-((6-(piperazin-1-yl)pyridin- 3-yl)oxy)pyridin-4- yl)methyl)piperidin-4-yl)-N,N- dimethylethanamineoxide |
| 256 | 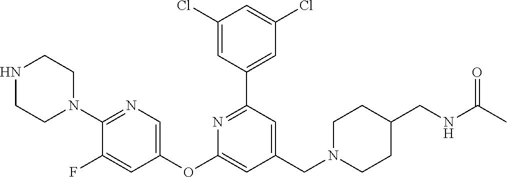 [SMILES: CC(=O)NCC1CCN(Cc2cc(Oc3cnc(N4CCNCC4)c(F)c3)nc(-c3cc(Cl)cc(Cl)c3)c2)CC1] | N-((1-((2-(3,5-dichlorophenyl)- 6-((5-fluoro-6-(piperazin-1-yl) pyridin-3-yl)oxy)pyridin- 4-yl)methyl)piperidin-4- yl)methyl)acetamide |
| 257 | 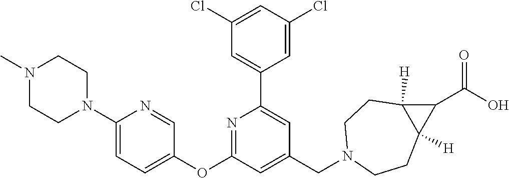 [SMILES: CN1CCN(c2ccc(Oc3cc(CN4CC[C@@H]5C(C(=O)O)[C@@H]5CC4)cc(-c4cc(Cl)cc(Cl)c4)n3)cn2)CC1] | (1R,7S,8r)-4-((2-(3,5- dichlorophenyl)-6-((6- (4-methylpiperazin-1-yl) pyridin-3-yl)oxy)pyridin-4-yl) methyl)-4-azabicyclo[5.1.0] octane-8-carboxylicacid |
| 258 | 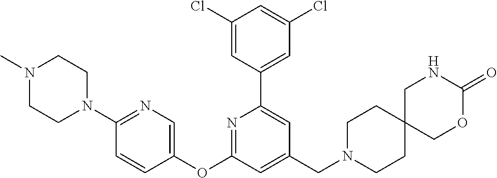 [SMILES: CN1CCN(c2ccc(Oc3cc(CN4CCC5(CC4)CNC(=O)OC5)cc(-c4cc(Cl)cc(Cl)c4)n3)cn2)CC1] | 9-((2-(3,5-dichlorophenyl)- 6-((6-(4-methylpiperazin-1- yl)pyridin-3-yl)oxy)pyridin-4- yl)methyl)-2-oxa-4,9- diazaspiro[5.5]undecan-3-one |
| 259 | 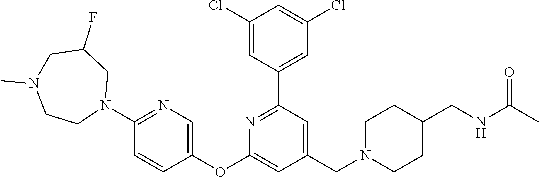 [SMILES: CC(=O)NCC1CCN(Cc2cc(Oc3ccc(N4CCN(C)CC(F)C4)nc3)nc(-c3cc(Cl)cc(Cl)c3)c2)CC1] | N-((1-((2-(3,5-dichlorophenyl)- 6-((6-(6-fluoro-4-methyl-1,4- diazepan-1-yl)pyridin- 3-yl)oxy)pyridin-4-yl)methyl) piperidin-4- yl)methyl)acetamide |
| 260 | 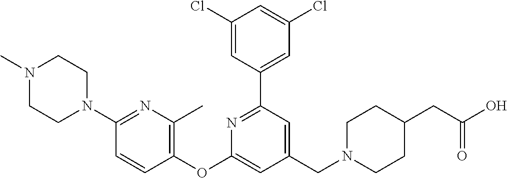 [SMILES: Cc1nc(N2CCN(C)CC2)ccc1Oc1cc(CN2CCC(CC(=O)O)CC2)cc(-c2cc(Cl)cc(Cl)c2)n1] | 2-(1-((2-(3,5-dichlorophenyl)- 6-((2-methyl-6- (4-methylpiperazin-1-yl) pyridin-3-yl)oxy)pyridin-4-yl) methyl)piperidin-4-yl) aceticacid |
| 261 | 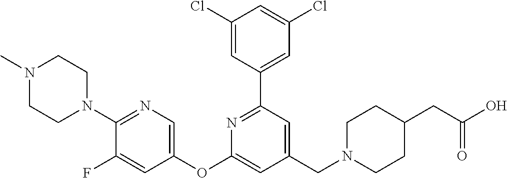 [SMILES: CN1CCN(c2ncc(Oc3cc(CN4CCC(CC(=O)O)CC4)cc(-c4cc(Cl)cc(Cl)c4)n3)cc2F)CC1] | 2-(1-((2-(3,5-dichlorophenyl)- 6-((5-fluoro-6-(4- methylpiperazin-1-yl)pyridin-3- yl)oxy)pyridin-4-yl)methyl) piperidin-4-yl)aceticacid |
| 262 | 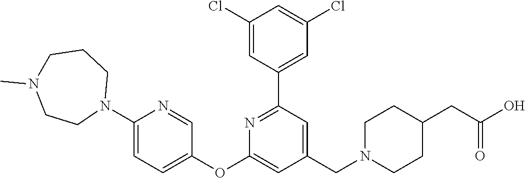 [SMILES: CN1CCCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)- 6-((6-(4-methyl-1,4-diazepan- 1-yl)pyridin-3-yl)oxy)pyridin- 4-yl)methyl)piperidin-4- yl)aceticacid |
| 263 | 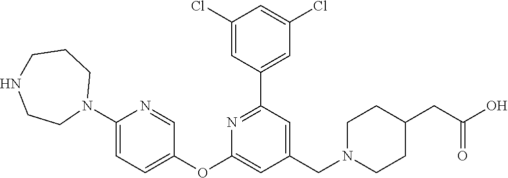 [SMILES: O=C(O)CC1CCN(Cc2cc(Oc3ccc(N4CCCNCC4)nc3)nc(-c3cc(Cl)cc(Cl)c3)c2)CC1] | 2-(1-((2-((6-(1,4-diazepan-1-yl) pyridin-3-yl)oxy)-6-(3,5- dichlorophenyl)pyridin-4- yl)methyl)piperidin-4-yl) aceticacid |
| 264 | 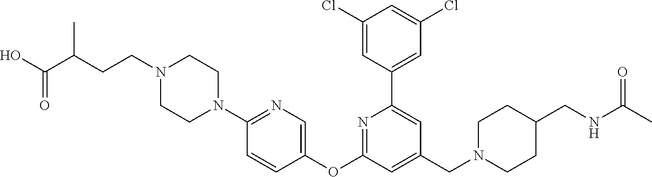 [SMILES: CC(=O)NCC1CCN(Cc2cc(Oc3ccc(N4CCN(CCC(C)C(=O)O)CC4)nc3)nc(-c3cc(Cl)cc(Cl)c3)c2)CC1] | 4-(4-(5-((4-((4- (acetamidomethyl)piperidin- 1-yl)methyl)-6-(3,5- dichlorophenyl)pyridin- 2-yl)oxy)pyridin-2-yl) piperazin-1-yl)-2- methylbutanoicacid |
| 265 | 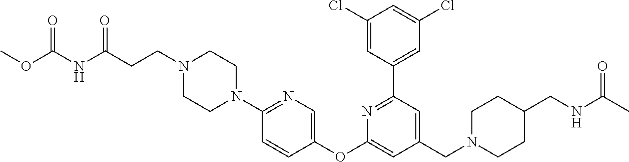 [SMILES: COC(=O)NC(=O)CCN1CCN(c2ccc(Oc3cc(CN4CCC(CNC(C)=O)CC4)cc(-c4cc(Cl)cc(Cl)c4)n3)cn2)CC1] | methyl(3-(4-(5-((4-((4- (acetamidomethyl)piperidin- 1-yl)methyl)-6- (3,5-dichlorophenyl)pyridin-2- yl)oxy)pyridin-2-yl)piperazin- 1-yl)propanoyl)carbamate |
| 266 | 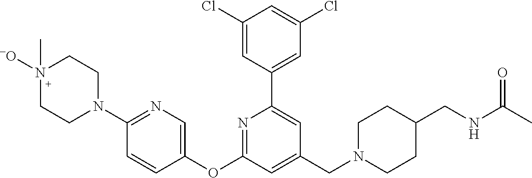 | 4-(5-((4-((4-(acetamidomethyl) piperidin-1-yl)methyl)-6-(3,5- dichlorophenyl)pyridin-2- yl)oxy)pyridin-2-yl)-1- methylpiperazine1- oxide |
| 267 | 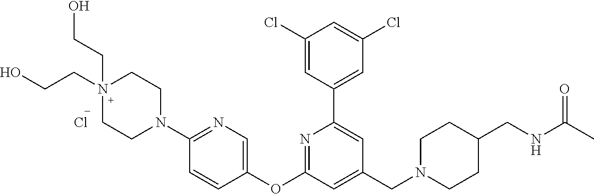 | 4-(5-((4-((4-(acetamidomethyl) piperidin-1-yl)methyl)-6-(3,5- dichlorophenyl)pyridin-2- yl)oxy)pyridin-2-yl)-1,1-bis (2-hydroxyethyl)piperazin- 1-ium |
| 268 | 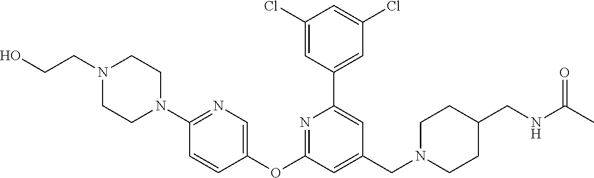 [SMILES: CC(=O)NCC1CCN(Cc2cc(Oc3ccc(N4CCN(CCO)CC4)nc3)nc(-c3cc(Cl)cc(Cl)c3)c2)CC1] | N-((1-((2-(3,5-dichlorophenyl)- 6-((6-(4-(2-hydroxyethyl) piperazin-1-yl)pyridin-3- yl)oxy)pyridin-4-yl)methyl) piperidin-4- yl)methyl)acetamide |
| 269 | 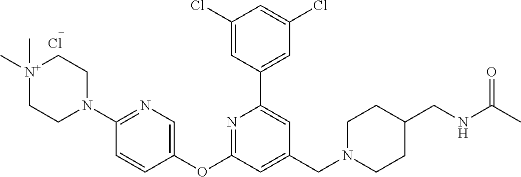 [SMILES: CC(=O)NCC1CCN(Cc2cc(Oc3ccc(N4CC[N+](C)(C)CC4)nc3)nc(-c3cc(Cl)cc(Cl)c3)c2)CC1 ; Cl] | 4-(5-((4-((4-(acetamidomethyl) piperidin-1-yl)methyl)-6-(3,5- dichlorophenyl)pyridin-2- yl)oxy)pyridin-2-yl)-1,1- dimethylpiperazin-1-ium |
| 270 | 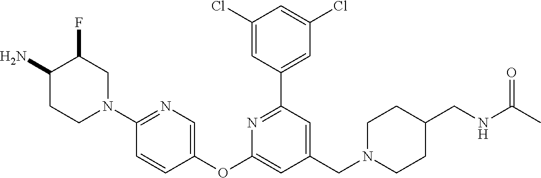 [SMILES: CC(=O)NCC1CCN(Cc2cc(Oc3ccc(N4CC[C@@H](N)[C@@H](F)C4)nc3)nc(-c3cc(Cl)cc(Cl)c3)c2)CC1] | N-((1-((2-((6-(4-amino-3- fluoropiperidin-1-yl)pyridin-3- yl)oxy)-6-(3,5-dichlorophenyl) pyridin-4-yl)methyl)piperidin- 4-yl)methyl)acetamide |
| 271 | 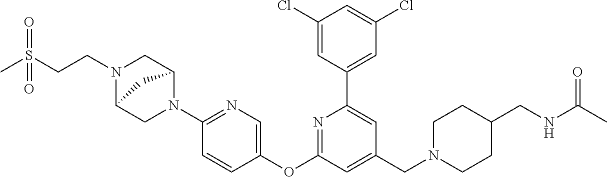 [SMILES: CC(=O)NCC1CCN(Cc2cc(Oc3ccc(N4C[C@@H]5C[C@H]4CN5CCS(C)(=O)=O)nc3)nc(-c3cc(Cl)cc(Cl)c3)c2)CC1] | N-((1-((2-(3,5-dichlorophenyl)- 6-((6-((1S,4S)-5-(2- (methylsulfonyl)ethyl)-2,5- diazabicyclo[2.2.1]heptan-2-yl) pyridin-3-yl)oxy)pyridin-4-yl) methyl)piperidin-4- yl)methyl)acetamide |
| 272 | 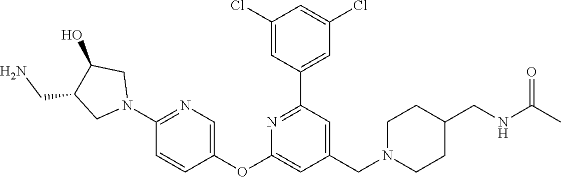 [SMILES: CC(=O)NCC1CCN(Cc2cc(Oc3ccc(N4C[C@H](CN)[C@@H](O)C4)nc3)nc(-c3cc(Cl)cc(Cl)c3)c2)CC1] | N-((1-((2-((6-((3S,4R)-3- (aminomethyl)-4- hydroxypyrrolidin-1-yl) pyridin-3-yl)oxy)-6- (3,5-dichlorophenyl)pyridin-4- yl)methyl)piperidin-4-yl) methyl)acetamide |
| 273 | 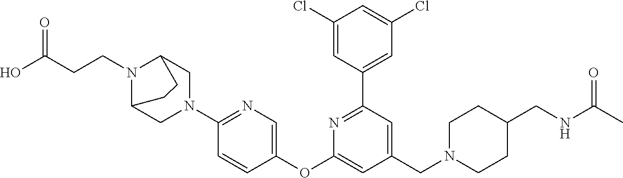 [SMILES: CC(=O)NCC1CCN(Cc2cc(Oc3ccc(N4CC5CCC(C4)N5CCC(=O)O)nc3)nc(-c3cc(Cl)cc(Cl)c3)c2)CC1] | 3-((1R,5S)-3-(5-((4-((4- (acetamidomethyl)piperidin- 1-yl)methyl)-6- (3,5-dichlorophenyl)pyridin-2- yl)oxy)pyridin-2-yl)-3,8- diazabicyclo[3.2.1]octan-8-yl) propanoicacid |
| 274 | 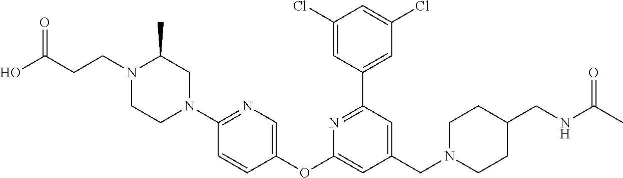 [SMILES: CC(=O)NCC1CCN(Cc2cc(Oc3ccc(N4CCN(CCC(=O)O)[C@@H](C)C4)nc3)nc(-c3cc(Cl)cc(Cl)c3)c2)CC1] | (S)-3-(4-(5-((4-((4- (acetamidomethyl)piperidin- 1-yl)methyl)-6- (3,5-dichlorophenyl)pyridin- 2-yl)oxy)pyridin-2-yl)-2- methylpiperazin-1- yl)propanoicacid |
| 275 | 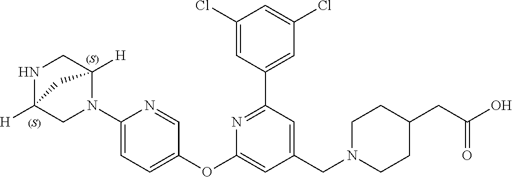 | 2-(1-((2-((6-((1S,4S)-2,5- diazabicyclo[2.2.1]heptan-2- yl)pyridin-3-yl)oxy)-6-(3,5- dichlorophenyl)pyridin-4- yl)methyl)piperidin-4-yl) aceticacid |
| 276 | 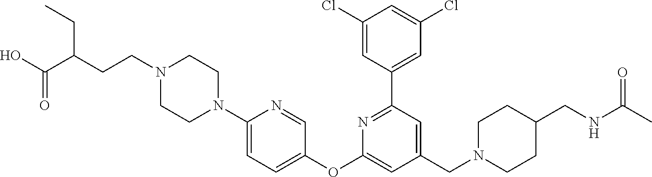 [SMILES: CCC(CCN1CCN(c2ccc(Oc3cc(CN4CCC(CNC(C)=O)CC4)cc(-c4cc(Cl)cc(Cl)c4)n3)cn2)CC1)C(=O)O] | 4-(4-(5-((4-((4- (acetamidomethyl)piperidin- 1-yl)methyl)-6-(3,5- dichlorophenyl)pyridin- 2-yl)oxy)pyridin-2-yl) piperazin-1-yl)-2- ethylbutanoicacid |
| 277 | 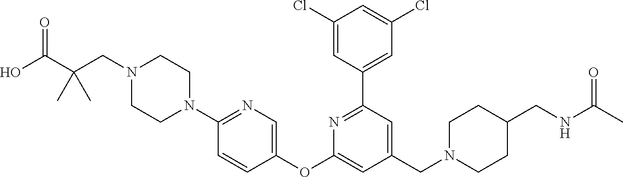 [SMILES: CC(=O)NCC1CCN(Cc2cc(Oc3ccc(N4CCN(CC(C)(C)C(=O)O)CC4)nc3)nc(-c3cc(Cl)cc(Cl)c3)c2)CC1] | 3-(4-(5-((4-((4- (acetamidomethyl)piperidin- 1-yl)methyl)-6-(3,5- dichlorophenyl)pyridin- 2-yl)oxy)pyridin-2-yl) piperazin-1-yl)-2,2- dimethylpropanoicacid |
| 278 | 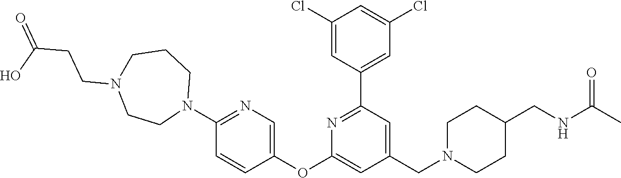 [SMILES: CC(=O)NCC1CCN(Cc2cc(Oc3ccc(N4CCCN(CCC(=O)O)CC4)nc3)nc(-c3cc(Cl)cc(Cl)c3)c2)CC1] | 3-(4-(5-((4-((4- (acetamidomethyl)piperidin- 1-yl)methyl)-6-(3,5- dichlorophenyl)pyridin- 2-yl)oxy)pyridin-2-yl)- 1,4-diazepan-1- yl)propanoicacid |
| 279 | 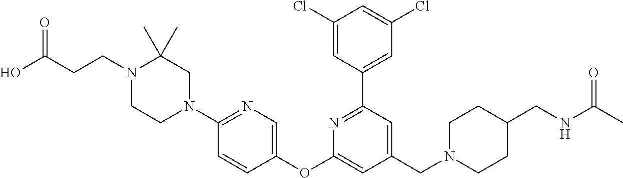 [SMILES: CC(=O)NCC1CCN(Cc2cc(Oc3ccc(N4CCN(CCC(=O)O)C(C)(C)C4)nc3)nc(-c3cc(Cl)cc(Cl)c3)c2)CC1] | 3-(4-(5-((4-((4- (acetamidomethyl)piperidin- 1-yl)methyl)-6-(3,5- dichlorophenyl)pyridin- 2-yl)oxy)pyridin-2-yl)-2,2- dimethylpiperazin-1-yl) propanoicacid |
| 280 | 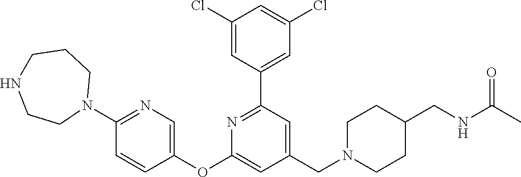 [SMILES: CC(=O)NCC1CCN(Cc2cc(Oc3ccc(N4CCCNCC4)nc3)nc(-c3cc(Cl)cc(Cl)c3)c2)CC1] | N-((1-((2-((6-(1,4-diazepan- 1-yl)pyridin-3-yl)oxy)-6- (3,5-dichlorophenyl)pyridin- 4-yl)methyl)piperidin-4-yl) methyl)acetamide |
| 281 | 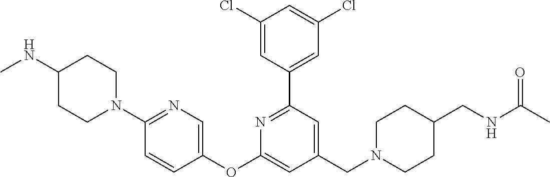 [SMILES: CNC1CCN(c2ccc(Oc3cc(CN4CCC(CNC(C)=O)CC4)cc(-c4cc(Cl)cc(Cl)c4)n3)cn2)CC1] | N-((1-((2-(3,5-dichlorophenyl)- 6-((6-(4-(methylamino) piperidin-1-yl)pyridin-3- yl)oxy)pyridin-4-yl) methyl)piperidin-4- yl)methyl)acetamide |
| 282 | 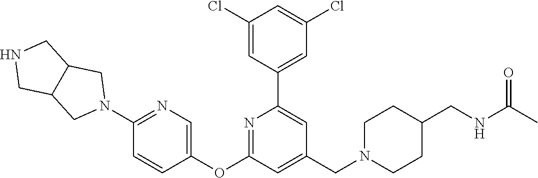 [SMILES: CC(=O)NCC1CCN(Cc2cc(Oc3ccc(N4CC5CNCC5C4)nc3)nc(-c3cc(Cl)cc(Cl)c3)c2)CC1] | N-((1-((2-(3,5-dichlorophenyl)- 6-((6-(hexahydropyrrolo[3,4-c] pyrrol-2(1H)-yl)pyridin-3-yl) oxy)pyridin-4-yl)methyl) piperidin-4-yl)methyl) acetamide |
| 283 | 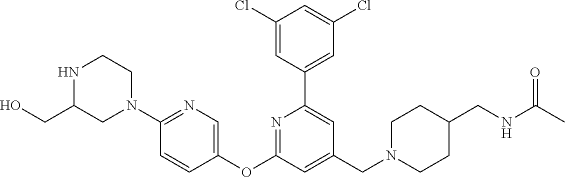 [SMILES: CC(=O)NCC1CCN(Cc2cc(Oc3ccc(N4CCNC(CO)C4)nc3)nc(-c3cc(Cl)cc(Cl)c3)c2)CC1] | N-((1-((2-(3,5-dichlorophenyl)- 6-((6-(3-(hydroxymethyl) piperazin-1-yl)pyridin-3- yl)oxy)pyridin-4-yl) methyl)piperidin-4- yl)methyl)acetamide |
| 284 | 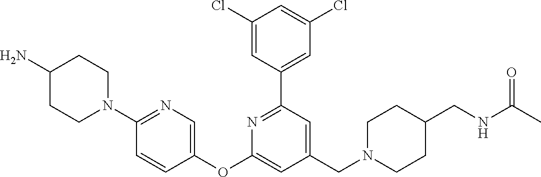 [SMILES: CC(=O)NCC1CCN(Cc2cc(Oc3ccc(N4CCC(N)CC4)nc3)nc(-c3cc(Cl)cc(Cl)c3)c2)CC1] | N-((1-((2-((6-(4- aminopiperidin-1-yl)pyridin- 3-yl)oxy)-6-(3,5- dichlorophenyl)pyridin-4- yl)methyl)piperidin-4-yl) methyl)acetamide |
| 285 | 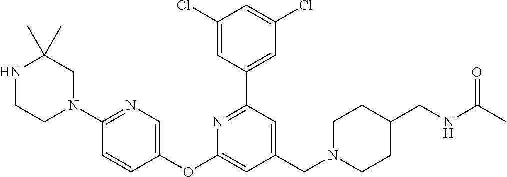 [SMILES: CC(=O)NCC1CCN(Cc2cc(Oc3ccc(N4CCNC(C)(C)C4)nc3)nc(-c3cc(Cl)cc(Cl)c3)c2)CC1] | N-((1-((2-(3,5-dichlorophenyl)- 6-((6-(3,3-dimethylpiperazin-1- yl)pyridin-3-yl)oxy)pyridin-4- yl)methyl)piperidin-4- yl)methyl)acetamide |
| 286 | 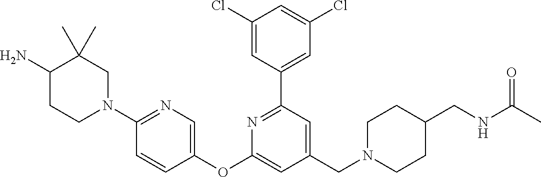 [SMILES: CC(=O)NCC1CCN(Cc2cc(Oc3ccc(N4CCC(N)C(C)(C)C4)nc3)nc(-c3cc(Cl)cc(Cl)c3)c2)CC1] | N-((1-((2-((6-(4-amino-3,3- dimethylpiperidin-1-yl) pyridin-3-yl)oxy)-6-(3,5- dichlorophenyl)pyridin-4- yl)methyl)piperidin-4-yl) methyl)acetamide |
| 287 | 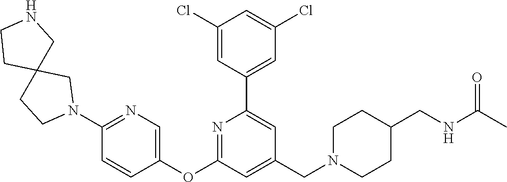 [SMILES: CC(=O)NCC1CCN(Cc2cc(Oc3ccc(N4CCC5(CCNC5)C4)nc3)nc(-c3cc(Cl)cc(Cl)c3)c2)CC1] | N-((1-((2-((6-(2,7-diazaspiro [4.4]nonan-2-yl)pyridin-3-yl) oxy)-6-(3,5- dichlorophenyl)pyridin-4- yl)methyl)piperidin-4-yl) methyl)acetamide |
| 288 | 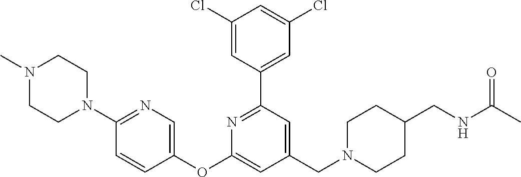 [SMILES: CC(=O)NCC1CCN(Cc2cc(Oc3ccc(N4CCN(C)CC4)nc3)nc(-c3cc(Cl)cc(Cl)c3)c2)CC1] | N-((1-((2-(3,5-dichlorophenyl)- 6-((6-(4-methylpiperazin-1-yl) pyridin-3-yl)oxy)pyridin-4-yl) methyl)piperidin-4- yl)methyl)acetamide |
| 289 | 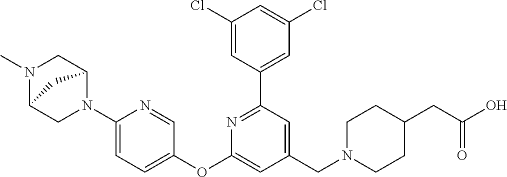 [SMILES: CN1C[C@@H]2C[C@H]1CN2c1ccc(Oc2cc(CN3CCC(CC(=O)O)CC3)cc(-c3cc(Cl)cc(Cl)c3)n2)cn1] | 2-(1-((2-(3,5-dichlorophenyl)- 6-((6-((1S,4S)-5-methyl-2,5- diazabicyclo[2.2.1]heptan-2-yl) pyridin-3-yl)oxy)pyridin-4-yl) methyl)piperidin-4- yl)aceticacid |
| 290 | 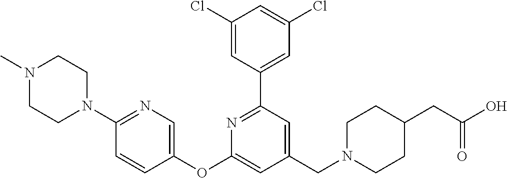 [SMILES: CN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)- 6-((6-(4-methylpiperazin-1-yl) pyridin-3-yl)oxy)pyridin-4-yl) methyl)piperidin-4- yl)aceticacid |
| 291 | 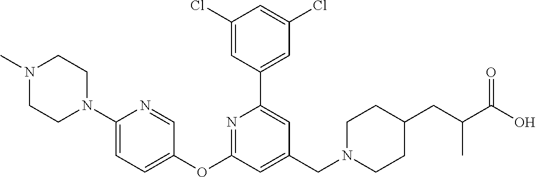 [SMILES: CC(CC1CCN(Cc2cc(Oc3ccc(N4CCN(C)CC4)nc3)nc(-c3cc(Cl)cc(Cl)c3)c2)CC1)C(=O)O] | 3-(1-((2-(3,5-dichlorophenyl)- 6-((6-(4-methylpiperazin-1-yl) pyridin-3-yl)oxy)pyridin-4-yl) methyl)piperidin-4-yl)- 2-methylpropanoicacid |
| 292 | 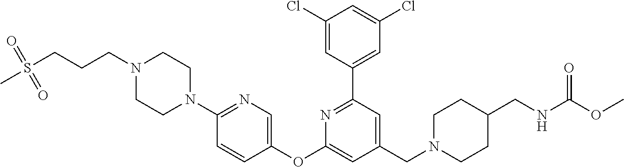 | methyl((1-((2-(3,5- dichlorophenyl)-6-((6-(4- (3-(methylsulfonyl) propyl)piperazin-1-yl) pyridin-3-yl)oxy)pyridin-4- yl)methyl)piperidin-4-yl) methyl)carbamate |
| 293 | 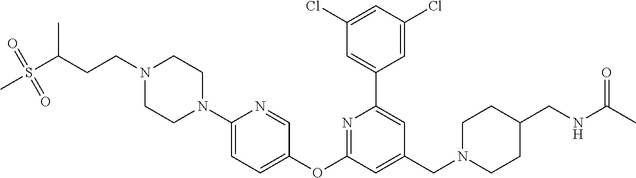 [SMILES: CC(=O)NCC1CCN(Cc2cc(Oc3ccc(N4CCN(CCC(C)S(C)(=O)=O)CC4)nc3)nc(-c3cc(Cl)cc(Cl)c3)c2)CC1] | N-((1-((2-(3,5-dichlorophenyl)- 6-((6-(4-(3-(methylsulfonyl) butyl)piperazin-1-yl)pyridin-3- yl)oxy)pyridin-4-yl)methyl) piperidin-4-yl) methyl)acetamide |
| 294 | 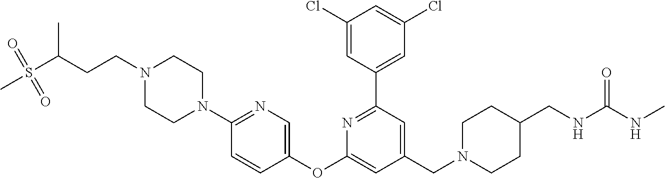 | 1-((1-((2-(3,5-dichlorophenyl)- 6-((6-(4-(3-(methylsulfonyl) butyl)piperazin-1-yl) pyridin-3-yl)oxy)pyridin-4- yl)methyl)piperidin-4-yl) methyl)-3-methylurea |
| 295 | 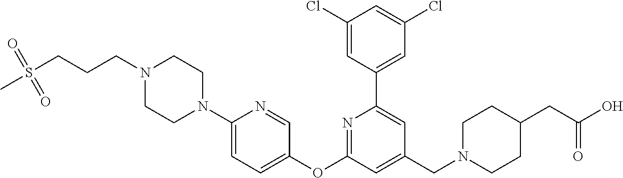 [SMILES: CS(=O)(=O)CCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)- 6-((6-(4-(3-(methylsulfonyl) propyl)piperazin-1-yl)pyridin- 3-yl)oxy)pyridin-4- yl)methyl)piperidin-4-yl) aceticacid |
| 296 | 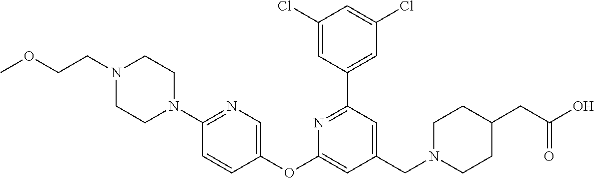 [SMILES: COCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)- 6-((6-(4-(2-methoxyethyl) piperazin-1-yl)pyridin-3-yl) oxy)pyridin-4-yl)methyl) piperidin-4-yl)aceticacid |
| 297 | 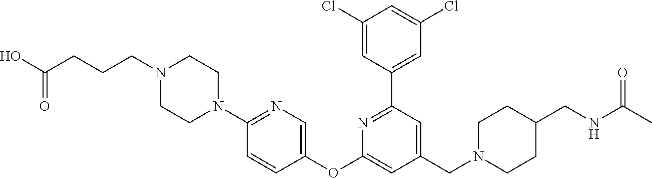 [SMILES: CC(=O)NCC1CCN(Cc2cc(Oc3ccc(N4CCN(CCCC(=O)O)CC4)nc3)nc(-c3cc(Cl)cc(Cl)c3)c2)CC1] | 4-(4-(5-((4-((4- (acetamidomethyl)piperidin- 1-yl)methyl)-6-(3,5- dichlorophenyl)pyridin- 2-yl)oxy)pyridin-2-yl) piperazin-1- yl)butanoicacid |
| 298 | 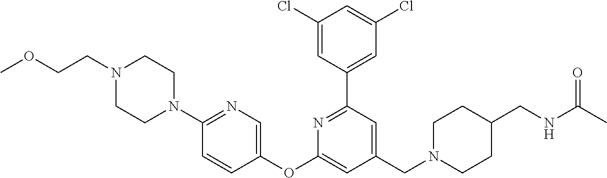 [SMILES: COCCN1CCN(c2ccc(Oc3cc(CN4CCC(CNC(C)=O)CC4)cc(-c4cc(Cl)cc(Cl)c4)n3)cn2)CC1] | N-((1-((2-(3,5-dichlorophenyl)- 6-((6-(4-(2-methoxyethyl) piperazin-1-yl)pyridin-3-yl) oxy)pyridin-4-yl)methyl) piperidin-4-yl)methyl) acetamide |
| 299 | 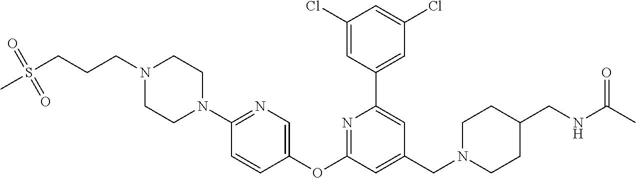 [SMILES: CC(=O)NCC1CCN(Cc2cc(Oc3ccc(N4CCN(CCCS(C)(=O)=O)CC4)nc3)nc(-c3cc(Cl)cc(Cl)c3)c2)CC1] | N-((1-((2-(3,5-dichlorophenyl)- 6-((6-(4-(3-(methylsulfonyl) propyl)piperazin-1-yl) pyridin-3-yl)oxy)pyridin- 4-yl)methyl)piperidin-4-yl) methyl)acetamide |
| 300 | 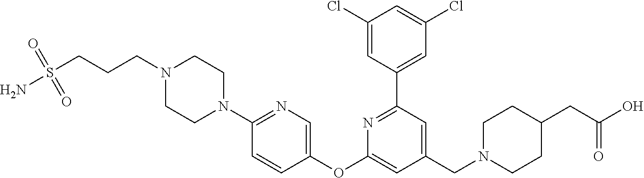 [SMILES: NS(=O)(=O)CCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)- 6-((6-(4-(3-sulfamoylpropyl) piperazin-1-yl)pyridin-3-yl) oxy)pyridin-4-yl)methyl) piperidin-4-yl)aceticacid |
| 301 | 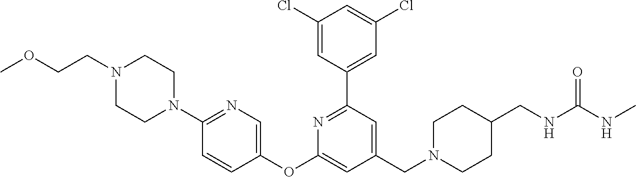 | 1-((1-((2-(3,5-dichlorophenyl)- 6-((6-(4-(2-methoxyethyl) piperazin-1-yl)pyridin-3-yl) oxy)pyridin-4-yl)methyl) piperidin-4-yl)methyl)-3- methylurea |
| 302 | 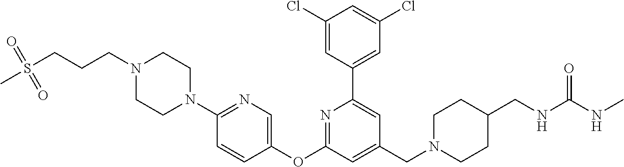 | 1-((1-((2-(3,5-dichlorophenyl)- 6-((6-(4-(3-(methylsulfonyl) propyl)piperazin-1-yl)pyridin- 3-yl)oxy)pyridin-4-yl)methyl) piperidin-4-yl)methyl)-3- methylurea |
| 303 | 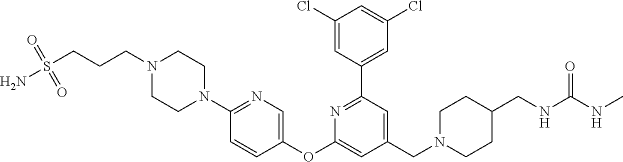 [SMILES: CNC(=O)NCC1CCN(Cc2cc(Oc3ccc(N4CCN(CCCS(N)(=O)=O)CC4)nc3)nc(-c3cc(Cl)cc(Cl)c3)c2)CC1] | 3-(4-(5-((6- (3,5-dichlorophenyl)-4-((4-((3- methylureido)methyl)piperidin- 1-yl)methyl)pyridin-2-yl)oxy) pyridin-2-yl)piperazin-1-yl) propane-1-sulfonamide |
| 304 | 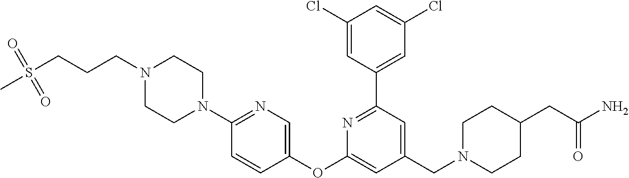 [SMILES: CS(=O)(=O)CCCN1CCN(c2ccc(Oc3cc(CN4CCC(CC(N)=O)CC4)cc(-c4cc(Cl)cc(Cl)c4)n3)cn2)CC1] | 2-(1-((2-(3,5-dichlorophenyl)- 6-((6-(4-(3-(methylsulfonyl) propyl)piperazin-1-yl)pyridin- 3-yl)oxy)pyridin-4- yl)methyl)piperidin-4-yl) acetamide |
| 305 | 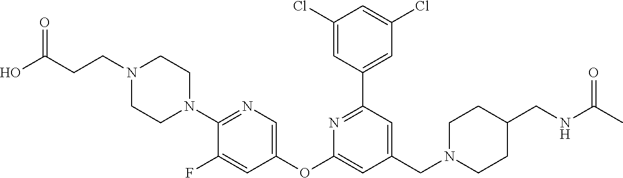 [SMILES: CC(=O)NCC1CCN(Cc2cc(Oc3cnc(N4CCN(CCC(=O)O)CC4)c(F)c3)nc(-c3cc(Cl)cc(Cl)c3)c2)CC1] | 3-(4-(5-((4-((4- (acetamidomethyl)piperidin- 1-yl)methyl)-6-(3,5- dichlorophenyl)pyridin- 2-yl)oxy)-3-fluoropyridin- 2-yl)piperazin-1- yl)propanoicacid |
| 306 | 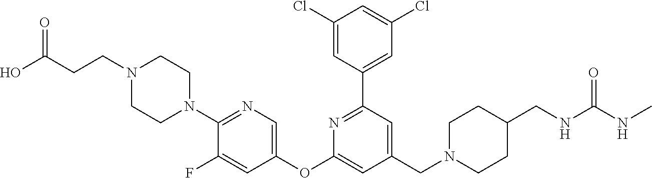 [SMILES: CNC(=O)NCC1CCN(Cc2cc(Oc3cnc(N4CCN(CCC(=O)O)CC4)c(F)c3)nc(-c3cc(Cl)cc(Cl)c3)c2)CC1] | 3-(4-(5-((6-(3,5- dichlorophenyl)-4-((4- ((3-methylureido)methyl) piperidin-1-yl)methyl)pyridin- 2-yl)oxy)-3-fluoropyridin- 2-yl)piperazin-1- yl)propanoicacid |
| 307 | 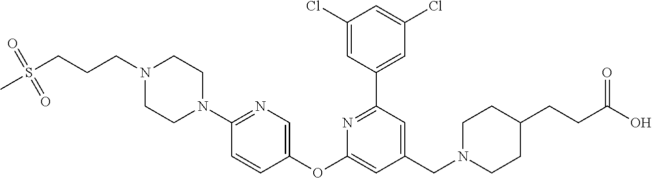 [SMILES: CS(=O)(=O)CCCN1CCN(c2ccc(Oc3cc(CN4CCC(CCC(=O)O)CC4)cc(-c4cc(Cl)cc(Cl)c4)n3)cn2)CC1] | 3-(1-((2-(3,5-dichlorophenyl)- 6-((6-(4-(3-(methylsulfonyl) propyl)piperazin-1-yl)pyridin- 3-yl)oxy)pyridin-4- yl)methyl)piperidin-4-yl) propanoicacid |
| 308 | 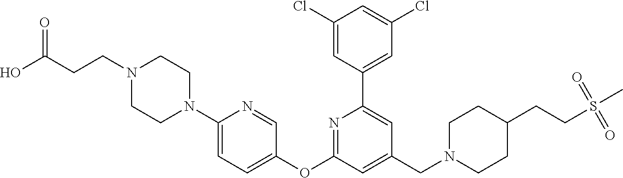 [SMILES: CS(=O)(=O)CCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5- dichlorophenyl)-4-((4-(2- (methylsulfonyl)ethyl) piperidin-1-yl)methyl)pyridin- 2-yl)oxy)pyridin-2- yl)piperazin-1-yl) propanoicacid |
| 309 | 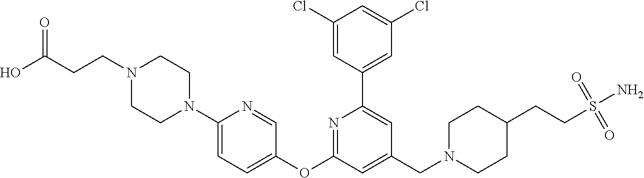 [SMILES: NS(=O)(=O)CCC1CCN(Cc2cc(Oc3ccc(N4CCN(CCC(=O)O)CC4)nc3)nc(-c3cc(Cl)cc(Cl)c3)c2)CC1] | 3-(4-(5-((6- (3,5-dichlorophenyl)-4-((4-(2- sulfamoylethyl)piperidin-1- yl)methyl)pyridin-2-yl)oxy) pyridin-2-yl)piperazin-1-yl) propanoicacid |
| 310 | 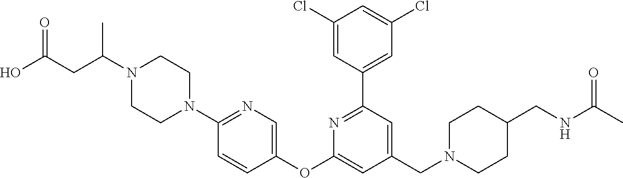 [SMILES: CC(=O)NCC1CCN(Cc2cc(Oc3ccc(N4CCN(C(C)CC(=O)O)CC4)nc3)nc(-c3cc(Cl)cc(Cl)c3)c2)CC1] | 3-(4-(5-((4-((4- (acetamidomethyl)piperidin- 1-yl)methyl)-6-(3,5- dichlorophenyl)pyridin- 2-yl)oxy)pyridin-2-yl) piperazin-1-yl) butanoicacid |
| 311 | 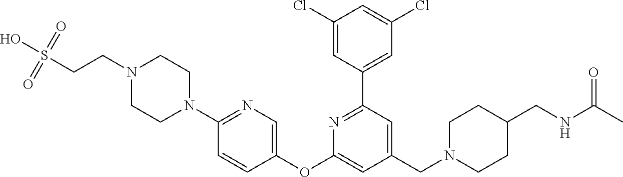 [SMILES: CC(=O)NCC1CCN(Cc2cc(Oc3ccc(N4CCN(CCS(=O)(=O)O)CC4)nc3)nc(-c3cc(Cl)cc(Cl)c3)c2)CC1] | 2-(4-(5-((4-((4- (acetamidomethyl)piperidin- 1-yl)methyl)-6-(3,5- dichlorophenyl)pyridin- 2-yl)oxy)pyridin-2-yl) piperazin-1-yl) ethanesulfonicacid |
| 312 | 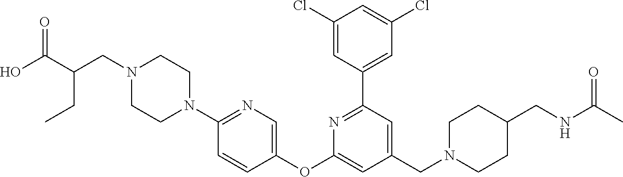 [SMILES: CCC(CN1CCN(c2ccc(Oc3cc(CN4CCC(CNC(C)=O)CC4)cc(-c4cc(Cl)cc(Cl)c4)n3)cn2)CC1)C(=O)O] | 2-((4-(5-((4-((4- (acetamidomethyl)piperidin- 1-yl)methyl)-6-(3,5- dichlorophenyl)pyridin- 2-yl)oxy)pyridin-2-yl) piperazin-1-yl)methyl) butanoicacid |
| 313 | 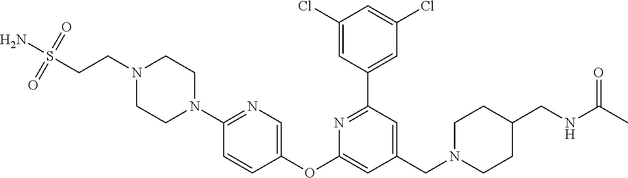 [SMILES: CC(=O)NCC1CCN(Cc2cc(Oc3ccc(N4CCN(CCS(N)(=O)=O)CC4)nc3)nc(-c3cc(Cl)cc(Cl)c3)c2)CC1] | N-((1-((2-(3,5-dichlorophenyl)- 6-((6-(4-(2-sulfamoylethyl) piperazin-1-yl)pyridin-3- yl)oxy)pyridin-4-yl)methyl) piperidin-4-yl)methyl)acetamide |
| 314 | 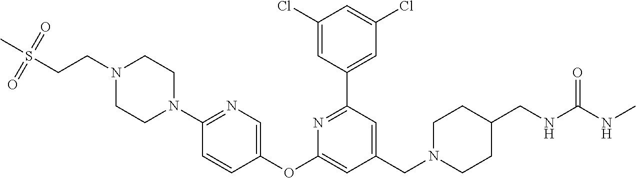 | 1-((1-((2-(3,5-dichlorophenyl)- 6-((6-(4-(2-(methylsulfonyl) ethyl)piperazin-1-yl)pyridin- 3-yl)oxy)pyridin-4-yl)methyl) piperidin-4-yl)methyl)-3- methylurea |
| 315 | 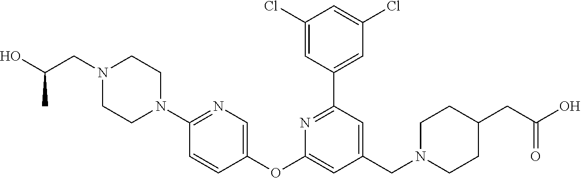 [SMILES: C[C@@H](O)CN1CCN(c2ccc(Oc3cc(CN4CCC(CC(=O)O)CC4)cc(-c4cc(Cl)cc(Cl)c4)n3)cn2)CC1] | (R)-2-(1-((2-(3,5- dichlorophenyl)-6-((6-(4- (2-hydroxypropyl)piperazin- 1-yl)pyridin-3-yl)oxy)pyridin- 4-yl)methyl)piperidin-4- yl)aceticacid |
| 316 | 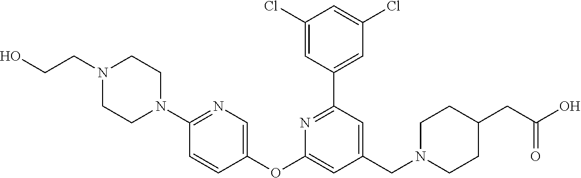 [SMILES: O=C(O)CC1CCN(Cc2cc(Oc3ccc(N4CCN(CCO)CC4)nc3)nc(-c3cc(Cl)cc(Cl)c3)c2)CC1] | 2-(1-((2-(3,5-dichlorophenyl)- 6-((6-(4-(2-hydroxyethyl) piperazin-1-yl)pyridin-3-yl) oxy)pyridin-4-yl)methyl) piperidin-4-yl)aceticacid |
| 317 | 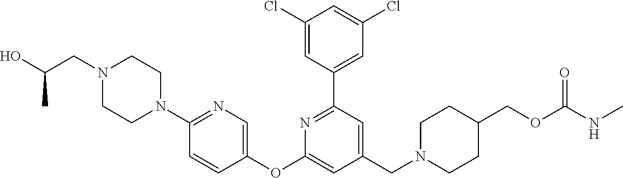 [SMILES: CNC(=O)OCC1CCN(Cc2cc(Oc3ccc(N4CCN(C[C@@H](C)O)CC4)nc3)nc(-c3cc(Cl)cc(Cl)c3)c2)CC1] | (R)-(1-((2-(3,5- dichlorophenyl)-6-((6-(4-(2- hydroxypropyl)piperazin-1- yl)pyridin-3-yl)oxy)pyridin- 4-yl)methyl)piperidin-4- yl)methylmethylcarbamate |
| 318 | 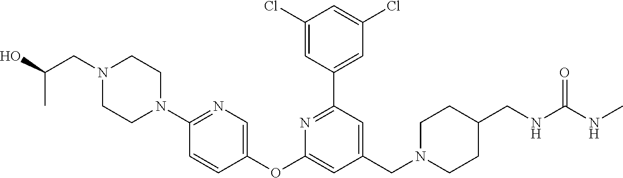 [SMILES: CNC(=O)NCC1CCN(Cc2cc(Oc3ccc(N4CCN(C[C@@H](C)O)CC4)nc3)nc(-c3cc(Cl)cc(Cl)c3)c2)CC1] | (R)-1-((1-((2-(3,5- dichlorophenyl)-6-((6-(4- (2-hydroxypropyl)piperazin-1- yl)pyridin-3-yl)oxy)pyridin-4- yl)methyl)piperidin-4- yl)methyl)-3-methylurea |
| 319 | 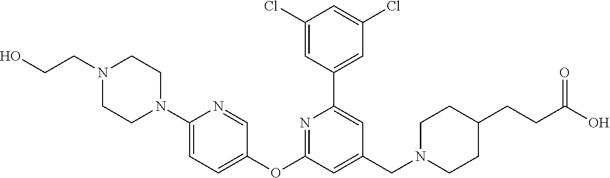 [SMILES: O=C(O)CCC1CCN(Cc2cc(Oc3ccc(N4CCN(CCO)CC4)nc3)nc(-c3cc(Cl)cc(Cl)c3)c2)CC1] | 3-(1-((2-(3,5-dichlorophenyl)- 6-((6-(4-(2-hydroxyethyl) piperazin-1-yl)pyridin-3- yl)oxy)pyridin-4-yl) methyl)piperidin-4- yl)propanoicacid |
| 320 | 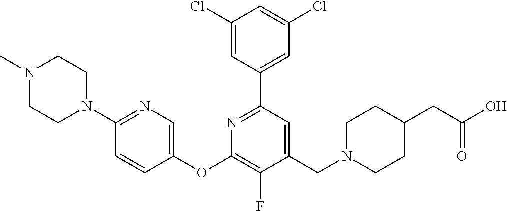 [SMILES: CN1CCN(c2ccc(Oc3nc(-c4cc(Cl)cc(Cl)c4)cc(CN4CCC(CC(=O)O)CC4)c3F)cn2)CC1] | 2-(1-((6-(3,5-dichlorophenyl)- 3-fluoro-2-((6-(4- methylpiperazin-1-yl) pyridin-3-yl)oxy)pyridin-4-yl) methyl)piperidin-4-yl) aceticacid |
| 321 | 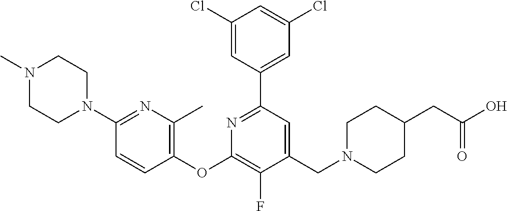 [SMILES: Cc1nc(N2CCN(C)CC2)ccc1Oc1nc(-c2cc(Cl)cc(Cl)c2)cc(CN2CCC(CC(=O)O)CC2)c1F] | 2-(1-((6-(3,5-dichlorophenyl)- 3-fluoro-2-((2-methyl-6-(4- methylpiperazin-1-yl)pyridin- 3-yl)oxy)pyridin-4-yl)methyl) piperidin-4-yl)aceticacid |
| 322 | 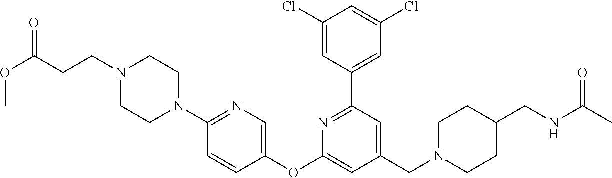 [SMILES: COC(=O)CCN1CCN(c2ccc(Oc3cc(CN4CCC(CNC(C)=O)CC4)cc(-c4cc(Cl)cc(Cl)c4)n3)cn2)CC1] | methyl3-(4-(5-((4-((4- (acetamidomethyl)piperidin- 1-yl)methyl)-6-(3,5- dichlorophenyl)pyridin-2- yl)oxy)pyridin-2-yl)piperazin- 1-yl)propanoate |
| 323 | 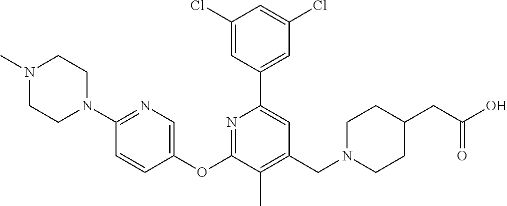 [SMILES: Cc1c(CN2CCC(CC(=O)O)CC2)cc(-c2cc(Cl)cc(Cl)c2)nc1Oc1ccc(N2CCN(C)CC2)nc1] | 2-(1-((6-(3,5-dichlorophenyl)- 3-methyl-2-((6-(4- methylpiperazin-1-yl)pyridin- 3-yl)oxy)pyridin-4-yl)methyl) piperidin-4-yl)aceticacid |

The synthesis and characterization of the compounds in Table 1 and Table 2 can be found in international PCT application no.: PCT/EP2019/062098, filed May 10, 2019, published on Nov. 14, 2019 with publication No. WO 2019/215341, which is incorporated herein by reference.

Typically, but not absolutely, the salts of the present disclosure are pharmaceutically acceptable salts. Salts of the disclosed compounds containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such asp-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates, and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts, and ammonium salts, as well as salts made from physiologically acceptable organic bases, such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other salts, which are not pharmaceutically acceptable, may be useful in the preparation of compounds of this disclosure and these should be considered to form a further aspect of this disclosure. These salts, such as oxalic or trifluoroacetate, while not in themselves pharmaceutically acceptable, may be useful in the preparation of salts useful as intermediates in obtaining the compounds of this disclosure and their pharmaceutically acceptable salts.

This disclosure further provides a pharmaceutical composition useful in the present disclosure (also referred to as pharmaceutical formulation) comprising a compound of Formula (I), or pharmaceutically acceptable salt thereof, and one or more excipients (also referred to as carriers and/or diluents in the pharmaceutical arts). The excipients are acceptable in the sense of being compatible with the other ingredients of the formulation and not deleterious to the recipient thereof (i.e., the patient).

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of this disclosure once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses).

In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A therapeutically effective amount of a compound of the present disclosure will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of Formula (I) for the treatment of a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus) will generally be in the range of 0.001 to 100 mg/kg body weight of recipient per day, suitably in the range of 0.01 to 10 mg/kg body weight per day. In certain embodiments, the effective amount of a compound of Formula (I) for the treatment of a viral infection resulting from SARS-CoV-2 is in the range of 0.001 to 100 mg/kg body weight of recipient per day. For example, a 70 kg adult mammal, the actual amount per day would suitably be from 7 to 700 mg and this amount may be given in a single dose per day or in a number (such as two, three, four, five or six) of sub-doses per day such that the total daily dose is the same. Inhaled daily dosages range from 10 μg -10 mg/day, with preferred 10 μg-2 mg/day, and more preferred 50 μg -500 μg/day. An effective amount of a salt or solvate, etc., may be determined as a proportion of the effective amount of the compound of Formula (I)per se. It is envisaged that similar dosages would be appropriate for treatment of the other conditions referred to above.

Also encompassed by the present disclosure are kits (e.g., pharmaceutical packs). In certain embodiments, the kit comprises a compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, each of the first or second containers are independently a vial, ampule, bottle, syringe, dispenser package, tube, or inhaler.

In certain embodiments, a kit described herein includes a first container comprising a compound of Formula (I), or a pharmaceutical composition, as described herein. In certain embodiments, a kit described herein is useful in treating and/or preventing a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus).

In certain embodiments, the kit comprises a compound of Formula (I), or a pharmaceutical composition thereof, and instructions for using the compound or pharmaceutical composition.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a viral infection resulting from a coronaviridae family virus (e.g., an alphacoronavirus (e.g., HCoV-NL63, HCoV-229E), a betacoronavirus (e.g., SARS-CoV, SARS-CoV-2, MERS-CoV, HCoV-OC43, HCoV-HKU1), a deltacoronavirus, a gammacoronavirus).

In certain embodiments, the instructions are for administering the compound or pharmaceutical composition to a subject (e.g., a subject in need of treatment or prevention of a disease described herein). In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

### EXAMPLES

In order that the disclosure described herein may be more fully understood, the following examples are set forth. The examples described in this application are offered to illustrate the compounds, pharmaceutical compositions, methods, and uses provided herein and are not meant to be limiting in any way.

### Example 1. In-Vitro Inhibition of Furin-Like Enzymes in Different Cell Lines

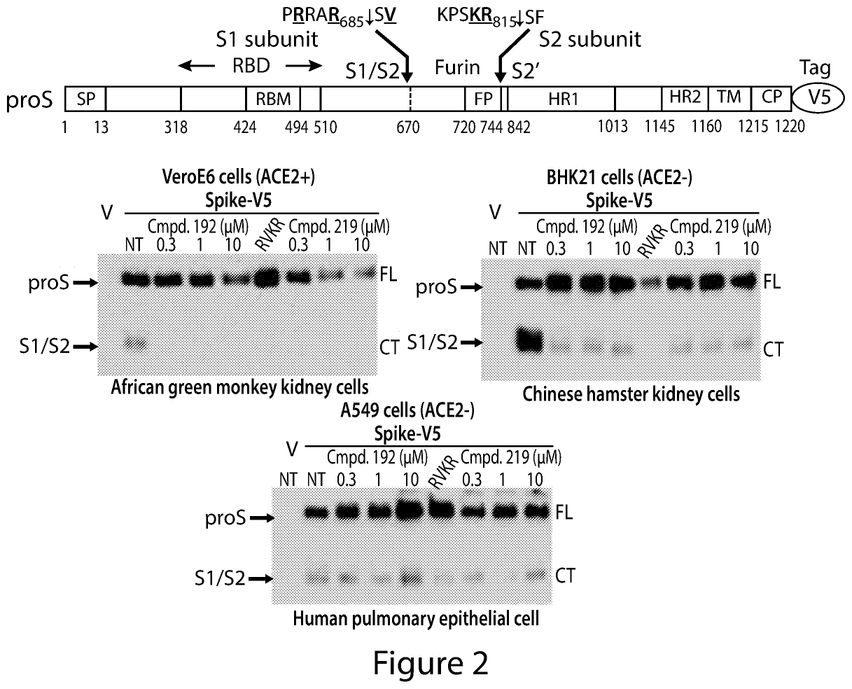

To test the effect of the compounds on the processing of the pro-(S) protein to active (S)-protein by endogenous furin-like enzymes, three cell lines (VeroE6 (African green monkey kidney), BHK21 (Chinese hamster kidney), and A549 (human pulmonary epithelial)) were transfected to transiently express the proS Sars-Cov-2 protein. Each cell line was incubated for 5 h with either Compound 192, Compound 219, or a cell/permeable pan-PCSK inhibitor, decanoyl-RVKR-chloromethylketone (RVKR). The cells were washed and transfected (Lipofectamine™) with 1 microgram of a cDNA coding for codon optimized (S)-protein (obtained from Sino Biologicals) inserted in a pIRES expression vector with a V5 tagged at the C-terminus of the S-protein. All three cell lines were then incubated for 24 h with Compound 192 or Compound 219 at 0.3 μM, 1 μM, or 10 μM, or decanoyl-RVKR-CMK (RVKR) at 50 μM (FIG. 2). Next, cell lysates were obtained. Separation by SDS-PAGE (8%) and Western blot analyses were done with a V5 mAb. Inhibition was observed in BHK21 cells and somewhat in VeroE6 cells. However, there was less inhibition in the A549 cells. One explanation could be that A549 cells do not process the pro-(S) protein by endogenous furin-like enzymes.

### Example 2. ProS Processing by Furin-Like Convertases and TMPRSS2

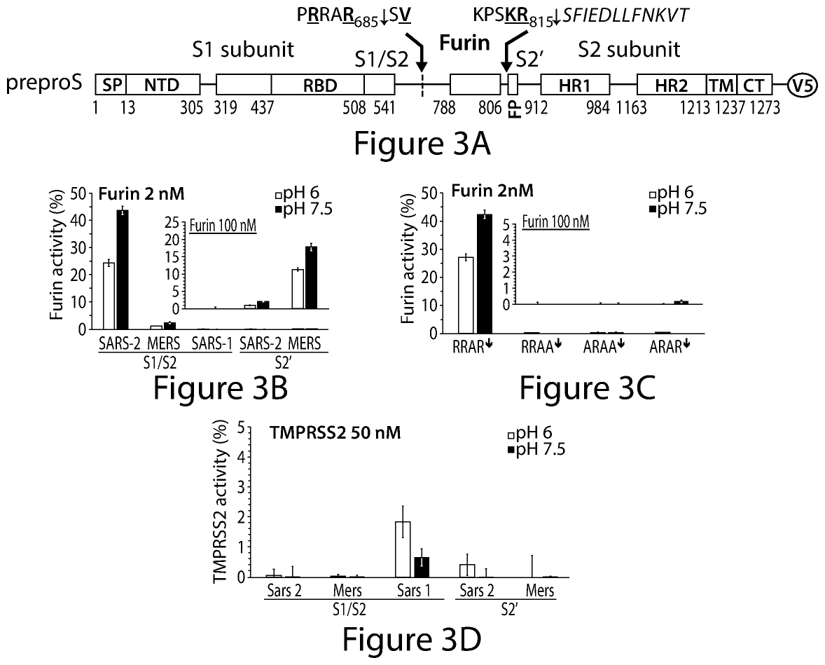

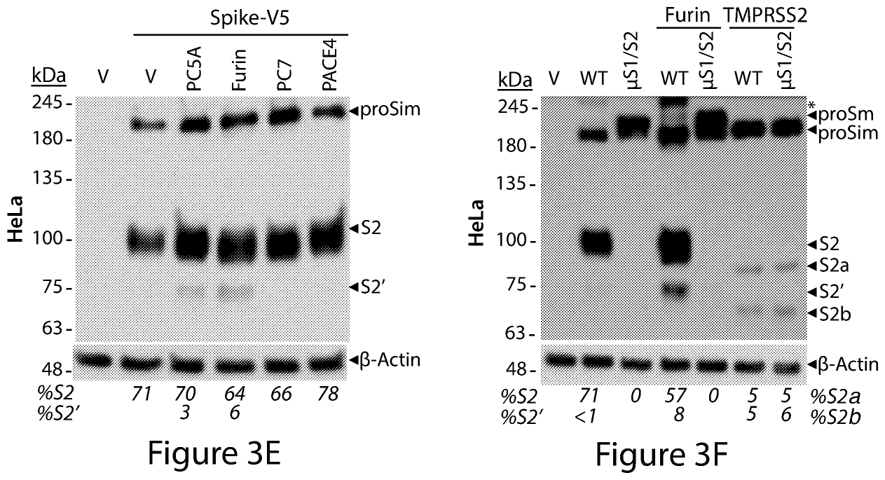

The susceptibility to furin-cleavages of SARS-CoV-2'S-glycoprotein was first assessed in vitro. Incubation of quenched fluorogenic peptides encompassing S1/S2 and S2′ sites (Table 3), demonstrated that the S1/S2 cleavage of SARS-CoV-2 is efficiently hydrolysed by furin at pH 7.5 and less at pH 6, whereas the SARS-CoV-1 S1/S2 and MERS-CoV are poorly cleaved (FIG. 3B).

TABLE 3
Sequence of different peptides mimicking
the Covid spike cleavage sites
that has been tested in the enzymatic
assay (The arrow indicates the expected
cleavage site).

| | S1/S2 Site | S2′ Site |
|---|---|---|
| Sars-CoV 1 | YHTVSLLR↓ISTSQ | |
| Mers CoV | TLTPRSVR↓SVPG | GSRSAR↓SAIE |
| Sars-CoV 2 | TNSPRRAR↓SVAS | SKPSKR↓SFIE |
| Sars-CoV 2 | TNSPRRAA SVAS | |
| Mutant 1 | | |
| Sars-CoV 1 | TNSPARAA SVAS | |
| Mutant 2 | | |
| Sars-CoV 2 | TNSPARAR SVAS | |
| Mutant 3 | | |

Furin less efficiently cleaved the SARS-CoV-2 and MERS-CoV at S2′, requiring 50-fold higher enzyme concentrations to detect cleavage (inset FIG. 3B). The high specificity of the SARS-CoV-2 to cleavage at furin-like motifs was next confirmed by demonstrating that the substitution of basic residues at the S1/S2 cleavage site (PRRAA₆₈₄←S, PARAR₆₈₅←S, PARAA₆₈₅←S) dramatically impaired the S1/S2 cleavage (FIG. 3C). Altogether these data show that furin best cleaves at S1/S2 and less efficiently at S2′. Based on camostat inhibition, TMPRSS2 was also proposed to participate in SARS-CoV-2 entry in some cells. Accordingly, it was then determined whether TMPRSS2 can cleave at S1/S2 or S2′ in vitro. However, TMPRSS2 that cleaves a peptide mimicking SARS-CoV-1 at S1/S2, was unable to process SARS-CoV-2 at S1/S2 or S2′ (FIG. 3D).

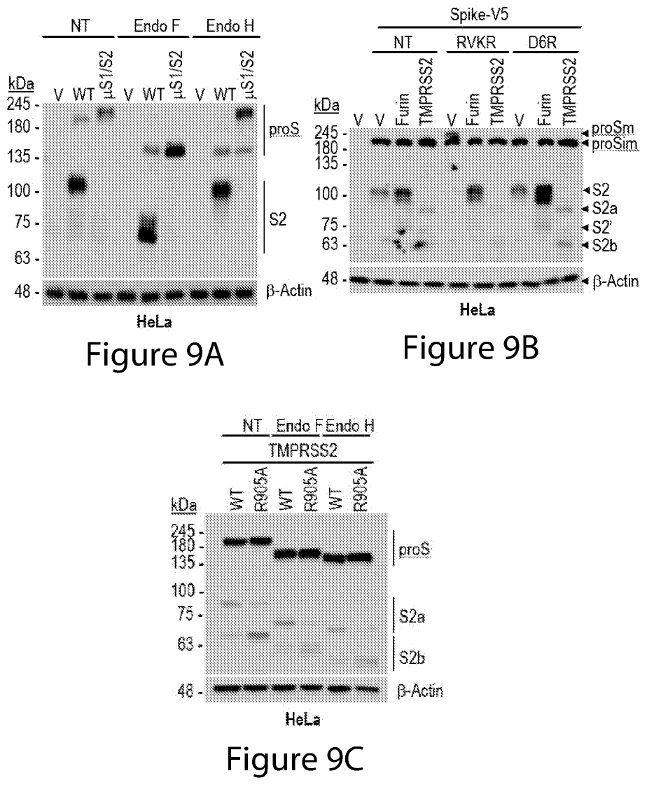

To further decipher the cellular role of furin-like enzymes, HeLa cells were co-transfected with a plasmid containing a codon optimized cDNA coding for V5-tagged proS (FIG. 3A) with cDNAs encoding furin, PC5A, PACE4 and PC7. Cell lysates were analyzed by Western blot (WB) after SDS-PAGE separation and probed with a V5-mAb. Endogenous proteases expressed in HeLa cells were found to process proS, likely at S1/S2, into a ˜100 kDa S2-like product (FIG. 3E). Furthermore, only overexpression of furin and PC5A enhanced the production of the less abundant ˜75 kDa S2′-like fragment (FIG. 3E). The remaining ˜200 kDa proSᵢₘ corresponds to an immature precursor form that has not exited the ER, as attested by its sensitivity to endoglycosidase-F and endoglycosidase-H (FIG. 9A), and insensitivity to furin-like convertases that are only active in the trans Golgi network (TGN) and/or cell surface/endosomes.

The double Ala-mutant [R685A+R682A] (denoted μS1/S2) of the S1/S2 site RRAR₆₈₅←S eliminated the P1 and P4 Arg critical for recognition by furin-like enzymes, and completely abrogated processing of proS at S1/S2 and putative S2′ by endogenous enzymes or by overexpressed furin (FIG. 3F). These data support a role of furin in the S1/S2 cleavage and revealed that the latter may be a prerequisite for the subsequent S2′ processing. The loss of furin-like cleavage at S1/S2 resulted in the accumulation of a higher molecular sized proSm (˜230 kDa), which likely represents a mature form of this precursor that exited the ER and became endoglycosidase-H-resistant but remained endoglycosidase-F-sensitive (FIG. 9A). The cell-permeable PC-inhibitor decanoyl-RVKR-cmk (RVKR) effectively prevented the endogenous formation of S2, but not the cell-impermeable D6R inhibitor, suggesting that proS cleavage by furin into S1 and S2 occurs intracellularly and not at the cell surface (FIG. 9B).

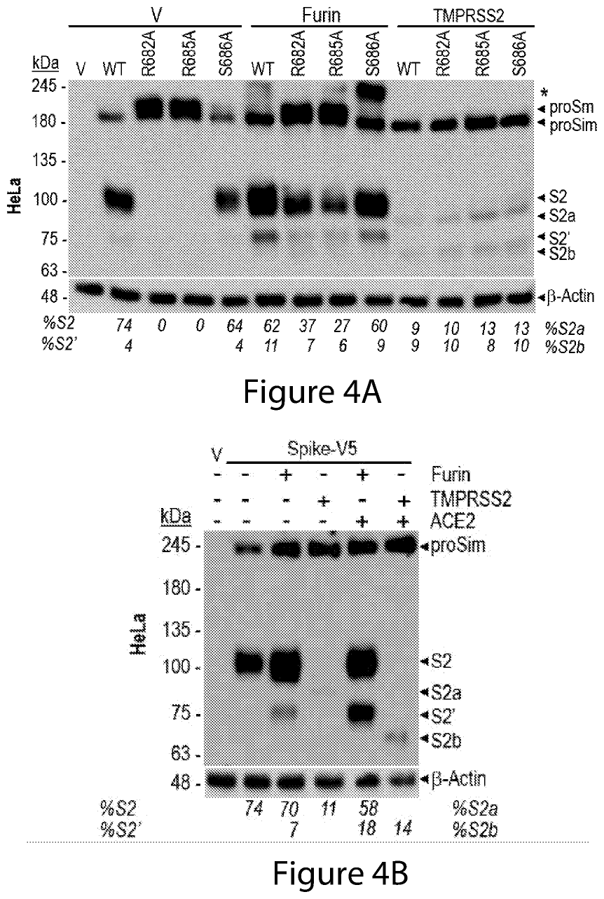

In order to better define the Arg-residues critical for processing at S1/S2, HeLa cells were expressed with the proS carrying single residue mutations: R682A, R685A and S686A in the absence or presence of furin (FIG. 4A). The latter was based on a prediction that Ser6 ms could be O-glycosylated, which may hamper processing at S1/S2. However, as for the WT, the S686A mutant was similarly processed by furin into S2 and S2′ (FIG. 4A). The data confirmed the critical importance of P4-Arg₆₈₂ or P1-Arg₆₈₅ for the generation of S2 by endogenous furin. However, in contrast to the μS1/S2 double Ala mutant (FIG. 3F), under conditions of excess furin these single mutants were partially cleaved (FIG. 4A). This reflects the multi-basic nature of the S1/S2 recognition sequence RRAR₆₈₅←S, whereby RRAA₆₈₅ and ARAR₆₈₅ may still be cleavable, but not ARAA₆₈₅, suggesting that the P3 site may also be important.

The processing of proS by TMPRSS2 in HeLa cells was also examined (FIG. 3F). In accordance with our in vitro data (FIG. 3D), overexpressed TMPRSS2 did not cleave proS at S1/S2 or S2′, but rather generated two minor distinct C-terminal products, herein called S2a (˜85 kDa) and S2b (˜70 kDa). These fragments were seen with both wild-type (WT)-S and its μS1/S2 mutant (FIG. 3F), revealing that they are S1/S2-independent. The S2 product generated by endogenous furin-like enzymes is absent when TMPRSS2 is co-expressed with WT proS (FIG. 3F), suggesting that TMPRSS2 generates S2a and S2b before proS encounters endogenous active furin, i.e., before it reaches the trans-golgi network. Indeed, like proSᵢₘ, both S2a and S2b are endoglycosidase-H-sensitive (FIG. 9C), indicating that they are generated in the ER and can no longer exit this compartment. Thus, high levels of TMPRSS2 would effectively inactivate S2 by preventing its ER-exit to reach the cell surface. As expected, single-Arg mutations in the S1/S2 site did not affect the ability of TMPRSS2 to generate S2a and S2b (FIG. 4A).

The implication of ACE2 in the processing of proS in HeLa cells, was next assessed by co-expression of proS with furin or TMPRSS2 in the absence or presence of ACE2. While not significantly affecting S1/S2 cleavage, the expression of ACE2 strongly enhanced the generation of smaller-sized S2′ by furin, and S2b by TMPRSS2 (FIG. 4B), likely reflecting a change in the proS conformation upon ACE2-binding.

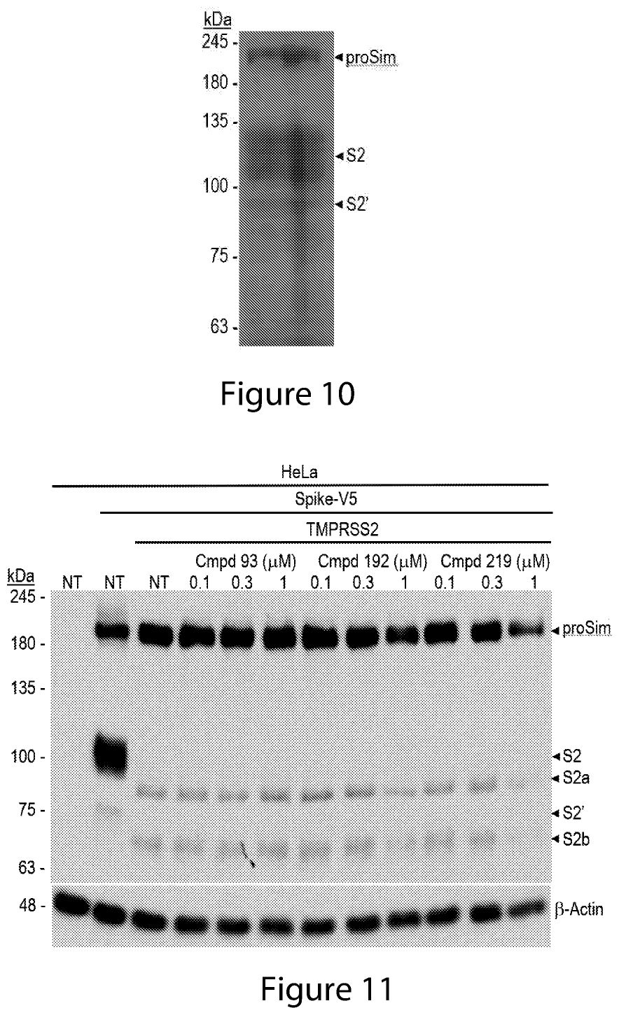

Proteomic analysis of the V5 labeled S2 product confirmed the assignment of the primary PRRAR₆₈₅← and secondary KR₈₁₅← furin cleavage sites (FIG. 10).

### Example 3. Furin-Inhibitors Block S/S2 Cleavage, without Affecting TMPRSS2 Processing

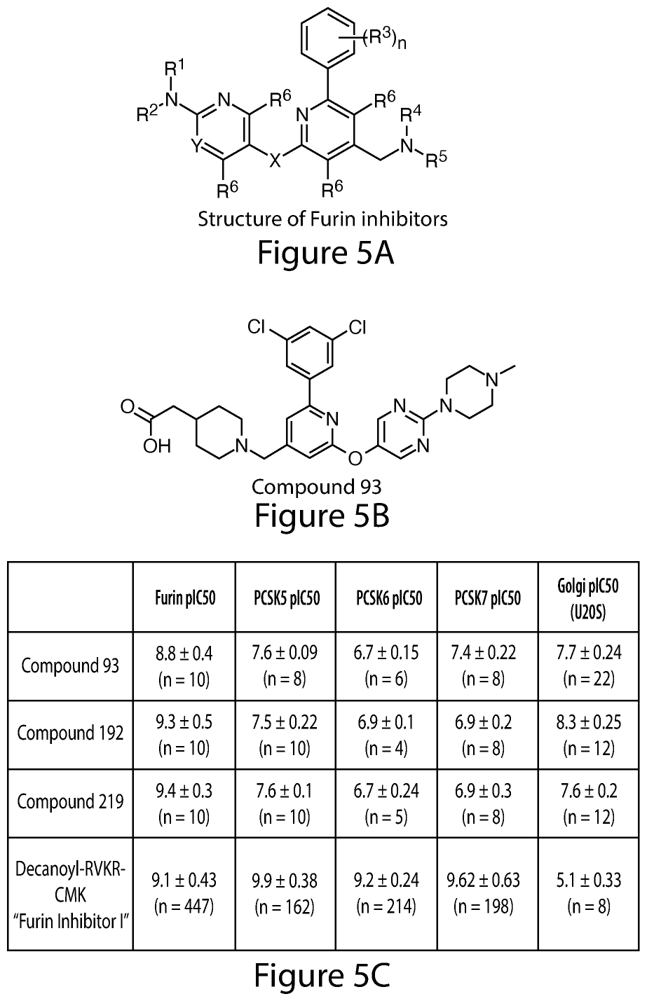

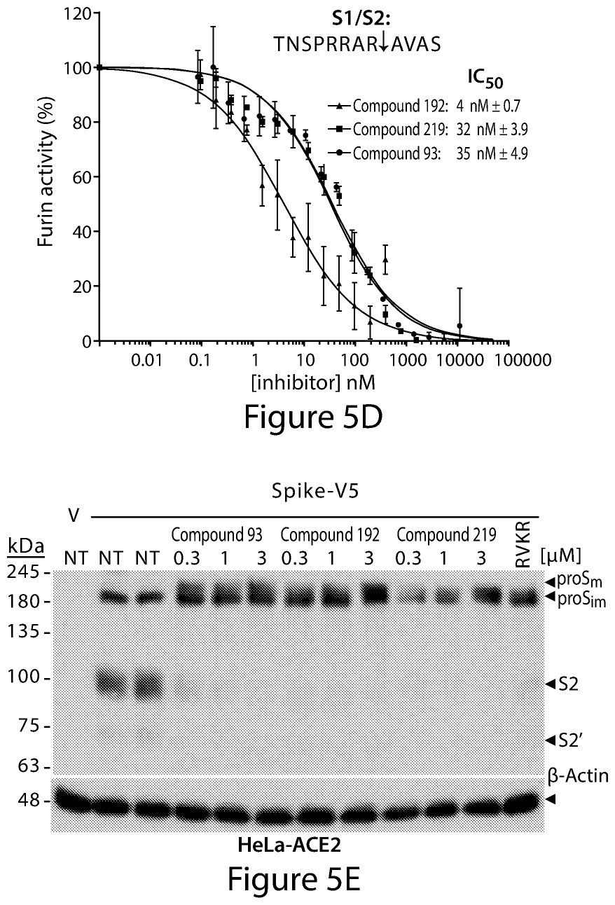

The efficacy and selectivity of representative Compounds 93, 192, and 219 was tested in vitro on purified soluble forms of furin, PC5A, PACE4, and PC7. The enzymatic activity was determined using a quenched fluorogenic substrate FAM-QRVRRAVGIDK-TAMRA, and compared to those obtained with the known PC-inhibitor RVKR-cmk. The data showed that all three inhibitors effectively blocked the processing of the above dibasic substrate by all convertases with an IC₅₀ of ˜7 nM compared to ˜9 nM for RVKR-cmk (FIG. 5C). The furin S1/S2 cleavage was also validated in vitro using a 12-residue quenched fluorogenic substrate DABSYL/Glu-TNSPRRAR←SVAS-EDANS. The inhibition deduced after hill-plot curve fitting (FIG. 5D) gave an estimated IC₅₀ of 4±0.7 nM (Compound 192), 32 f 4 nM (Compound 219), and 35±5 nM (Compound 93).

The inhibition of PC-activities by the compounds of this disclosure was next assessed intracellularly using a cell-based Golgi imaging assay of U2OS cells. The data demonstrated that the compounds inhibited endogenous furin processing of a BMP10-mimic with an IC₅₀ of ˜8 nM versus 5 nM for RVKR-cmk (FIG. 5C). The above enzymatic assays showed that all 3 inhibitors can inhibit furin, but may also inhibit other members of the PC-family such as PC5A, PACE4, and PC7.

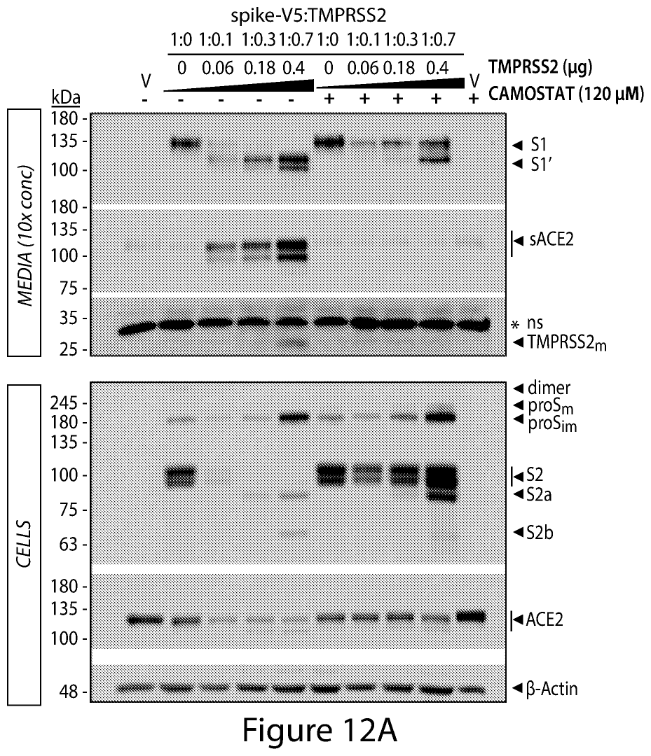

The effects of Compound 93, 192, and 219 on the processing of proS in HeLa cells stably expressing ACE2 was evaluated (HeLa-ACE2; FIG. 5E). In agreement with the in vitro data (FIG. 5D), all three compounds blocked the S1/S2 and S2′ processing by endogenous furin-like enzymes with Compound 192 showing almost complete inhibition at 300 nM (FIG. 5E), comparable to that obtained with a control 50 μM decanoyl-RVKR-cmk. In contrast, in HeLa cells none of the compounds affected the generation of S2a and S2b by TMPRSS2 (FIG. 11), in agreement with the distinct cleavage specificities of furin and TMPRSS2.

### Example 4. TMPRSS2 Sheds ACE2 and Cleaves SL

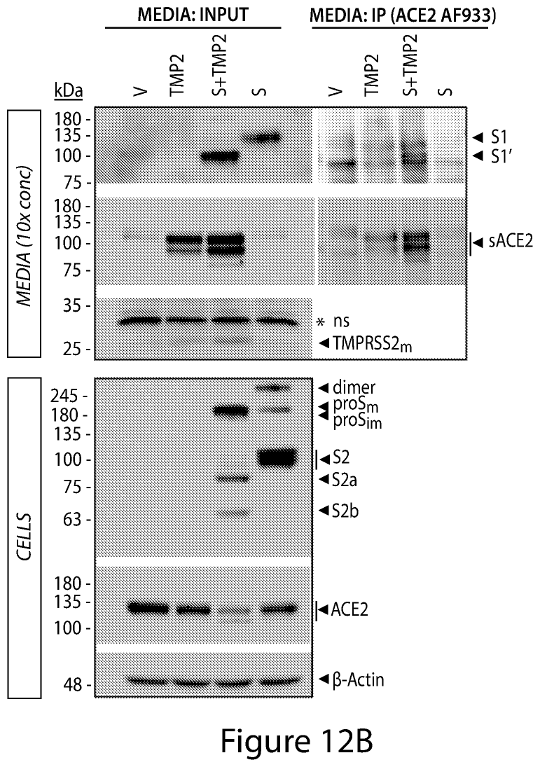

The data does not support the direct implication of TMPRSS2 in the generation of S2 or S2′, since it was found that overexpression of TMPRSS2 cleaves proS to generate ER-retained S2a and less so S2b (FIG. 3F, FIGS. 9B, 9C) and does not cleave proS at S2′. To confirm that S2a, and S2b are generated by TMPRSS2 activity in the ER, HeLa-ACE2 cells were incubated with 120 μM Camostat, known to inhibit TMPRSS2. The data showed that this inhibitor can reach the ER as it blocked the autocatalytic activation of TMPRSS2 at RQSR₂₅₅← (loss of ˜25 kDa form in the media), prevented the formation of both S2a and S2b with increasing concentrations of TMPRSS2, and gradually allowed the resumption of the furin-like cleavage at S1/S2 (FIG. 12A).

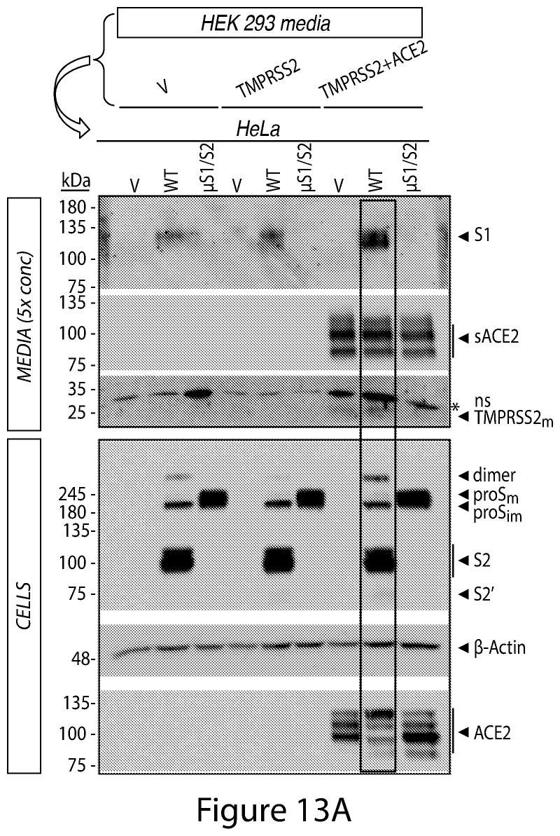

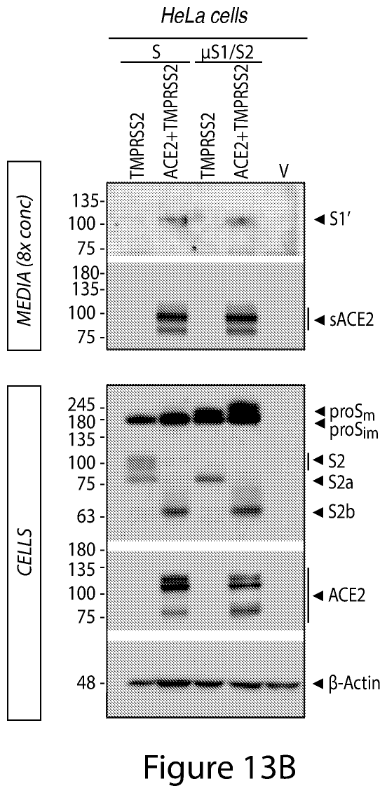

Accordingly, other functions that TMPRSS2 may exert were explored to explain its reported enhancement of viral entry. Hence, increasing amounts of TRMPSS2 were expressed in HeLa-ACE2 cells and followed the S1 and TRMPSS2 processing by WB-analysis using anti-S1 and anti-TRMPSS2 antibodies. First, it was found that TMPRSS2 cleaved the furin-generated S1-subunit (˜135 kDa) into a shorter S1′ fragment (˜115 kDa) secreted into the medium (FIG. 12A). This cleavage may enhance the efficacy of separation of the S1 and S2 domains when S1 is bound to ACE-2, but before membrane fusion by the S2-subunit. It was previously reported that TMPRSS2 sheds ACE2 into a soluble form (sACE2), and the latter activity may be associated with enhanced kinetics of cell-to-cell fusion (syncytia) and ACE2-receptor viral uptake. In agreement, overexpression of TMPRSS2 in HeLa-ACE2 cells enhanced the shedding of ACE2 into ˜120 and ˜110 kDa sACE2 forms. The generation of both sACE2 and in large part S1′ are inhibited by 120 μM Camostat (FIG. 12A). Note that the small background shedding of ACE2 is not sensitive to Camostat, suggesting that another endogenous protease, possibly ADAM17, is also implicated. Co-immunoprecipitation experiments showed that sACE2 and S1′ are found as a complex bound to each other in the media (FIG. 12B). Incubation of HeLa cells expressing S with media containing sACE2 and active mature ˜25 kDa TMPRSS2m generated by co-expression of full length ACE2 with TMPRSS2 in HEK293 cells revealed that sACE2 enhanced the levels of S2′ in cells and S1 in media (FIG. 13A). Finally, co-expression of TMPRSS2 with WT proS or its μS1/S2 mutant in HeLa cells in the absence or presence of ACE2 resulted in the similar generation of: (i) secreted S1′ only in the presence of ACE2 and (ii) secreted sACE2 (FIG. 13B). Furthermore, these data revealed that furin-processing at S1/S2 is not a prerequisite for these TMPRSS2-mediated cleavages.

### Example 5. Immunocytochemistry

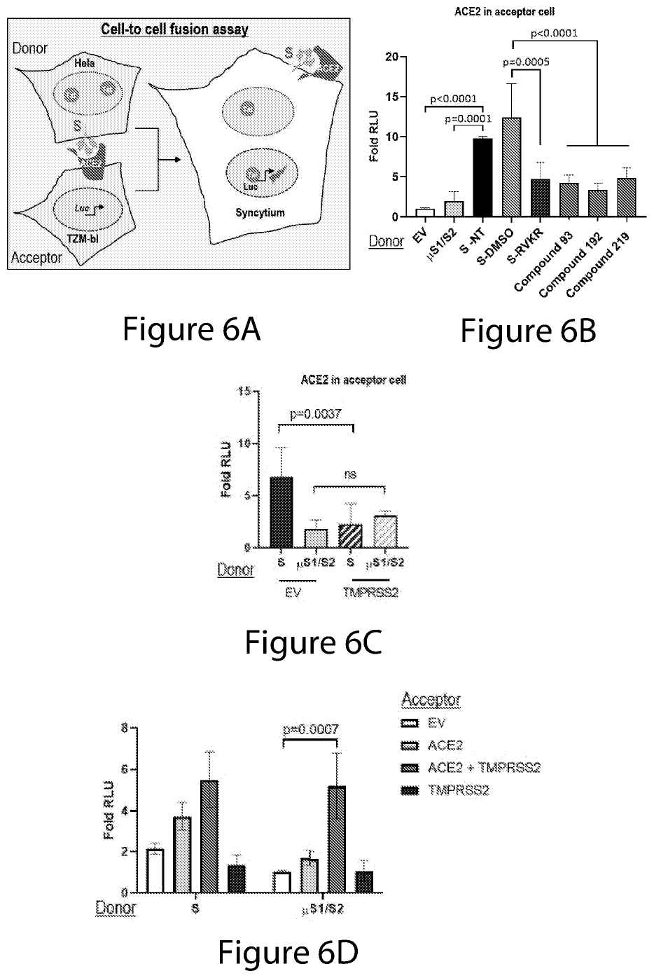

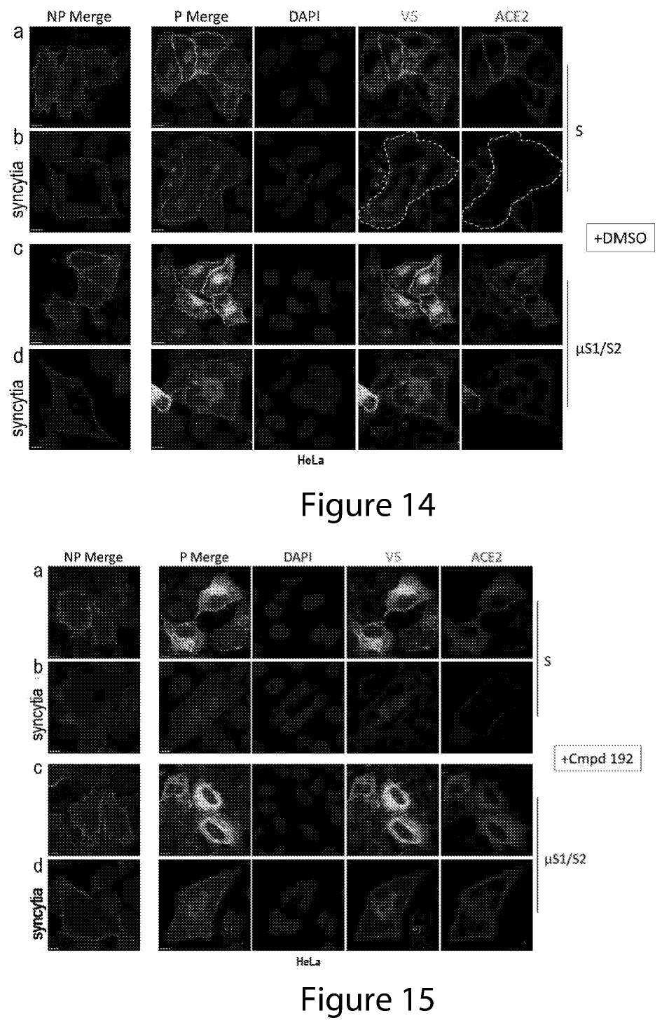

Immunocytochemical analyses of HeLa cells co-expressing the S-protein or μS1/S2 with ACE2 in the absence or presence of 1 μM Compound 192 was also investigated under cell-permeabilization (P; V5 and ACE2 antibodies) and non-permeabilization (NP; S2 and ACE2 antibodies) conditions (FIGS. 14, 15). In the absence of Compound 192, the S-protein and ACE2 co-localized abundantly at the cell surface (FIG. 14-a). HeLa cells expressing both S-protein and ACE2 formed many syncytia, associated with reduced cell surface expression of the S-protein, and an even greater reduction of ACE2 (FIG. 14-b). Cells expressing both μS1/S2 and ACE2 showed an accumulation of both proS and ACE2 inside the cells and at the cell surface (FIG. 14-c). However, they barely induced the formation of syncytia, and when they did, the cell surface expression of S-protein and to a lesser extent ACE2 were decreased (FIG. 14-d). In the presence of 1 μM Compound 192, S-expressing HeLa cells (FIGS. 15-a,b) phenocopy those expressing μS1/S2 (FIGS. 6B-c,d).

### Example 6. Cell-to-Cell Fusion

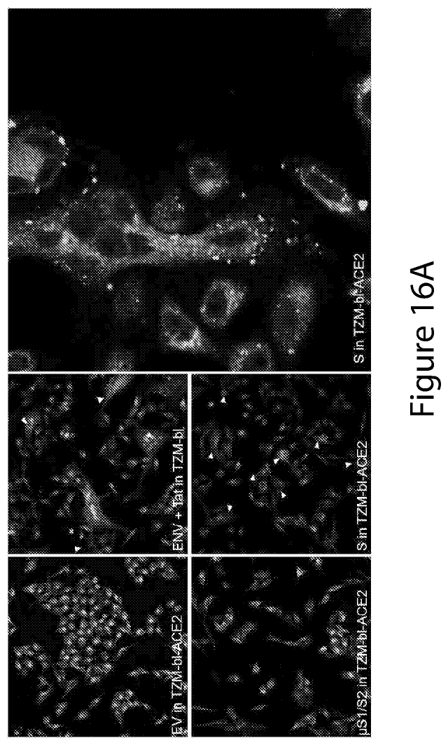

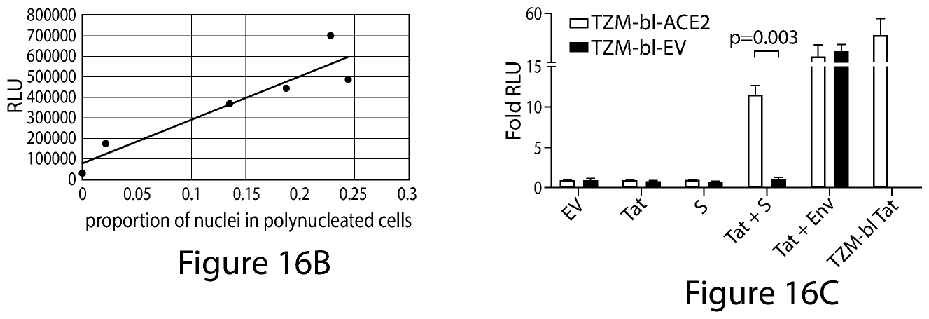

Having established that S-protein and ACE2 co-localize at the cell surface, the impact of furin-cleavage at S1/S2 on the ability of S-protein to induce cell-to-cell fusion was analyzed. Accordingly, a luminescence-based assay was developed using HeLa TZM-bl reporter cells stably transfected with an HIV-1-based vector expressing luciferase under the control of the HIV-1 long terminal repeat (LTR), which can be activated by HIV Tat protein. These cells endogenously express the HIV receptor CD4 and its co-receptors CCR5 and CXCR4. Without wishing to be bound by any particular theory, it is possible that fusion of donor WT HeLa cells (expressing Tat and the fusogenic S-protein) with acceptor TZM-bl cells expressing ACE2 would result in accrued luciferase activity (FIG. 6A). When donor cells expressing HIV gp160 and Tat fuse with TZM-bl acceptor cells, luciferase activity increases compared to that observed in TZM-bl control cells co-cultured with donor Hela cells expressing only Tat. (FIG. 16C). The expression of S-protein in HeLa cells did not induce fusion with TZM-bl control cells (FIGS. 16A, 16C). However, ACE2 expression in TZM-bl allowed fusion with HeLa-expressing S-protein in a dose-dependent manner (FIG. 16B). The linearity of our assay (correlation coefficient of 0.87) validated the use of luminescence as an indicator of cell-to-cell fusion. Conversely, expression of μS1/S2 in donor cells did not enhance fusion with TZM-bl expressing ACE2 and >60% fusion-inhibition was observed upon incubation of cells with 300 nM of Compound 93, 192, or 219 or 10 μM of the PC-inhibitor RVKR-cmk (FIG. 6B), indicating that S1/S2 cleavage promotes ACE2-dependent cell-to-cell fusion.

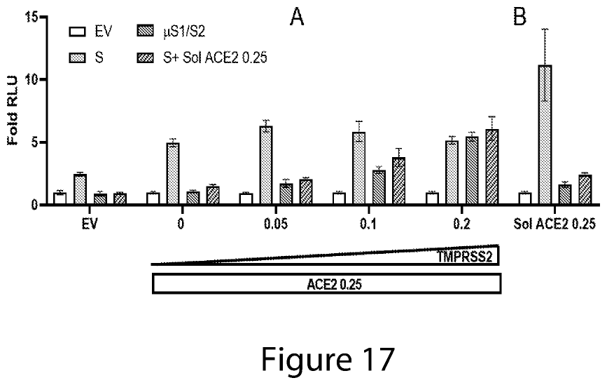

TMPRSS2 was co-expressed with S-protein or with μS1/S2 in donor cells to assess the role of TMPRSS2 in cell-to-cell fusion. In agreement with our cell-biology data (FIGS. 3F, 4), TMPRSS2 abolished the fusogenic activity of S, providing evidence that TMPRSS2-mediated retention of S-protein in the ER by the generation of S2a and S2b impaired the cell-to-cell fusion activity of S-protein at the plasma membrane (FIG. 6C). However, co-expression of TMPRSS2 and ACE2 in acceptor cells tended to enhance the fusion with donor S-containing cells, an effect much more evident with μS1/S2-containing donor cells, resulting in similar cell-to-cell fusion between donor cells expressing either WT-S or μS1/S2 and acceptor ACE2-TMPRSS2 cells (FIG. 6D). This phenotype suggests that in the absence of furin-cleavage (μS1/S2) the TMPRSS2-generated S1′ releases the N-terminal part of the S1 cap, thereby favoring furin-cleavage at S2′ and cell-cell-fusion. Indeed, co-expression of ACE2 with various doses of TMPRSS2 in acceptor cells gradually promoted the fusion of the μS1/S2 to similar levels as the WT-S-induced fusion (FIG. 17). However, sACE2 alone had no effect on μS1/S2 (panels A versus B), as the S2′ site would still be capped by the un-cleaved S1-subunit (FIG. 17). Thus, only high levels of TMPRSS2 in ACE2-acceptor cells allow similar fusion with donor cells expressing WT-S and μS1/S2 (FIG. 17A). Interestingly, overexpression of a soluble form of ACE2 (sACE2) in acceptor cells significantly enhanced fusion with donor cells containing WT-S. Here the sACE2-S1 complex may bind a receptor on acceptor cells, e.g., to integrins via their RGD motifs or S1-binding to neuropilin 1, 2, to promote cell-to-cell fusion.

### Example 7. Effects of Furin Inhibitors on Entry of Pseudoviruses

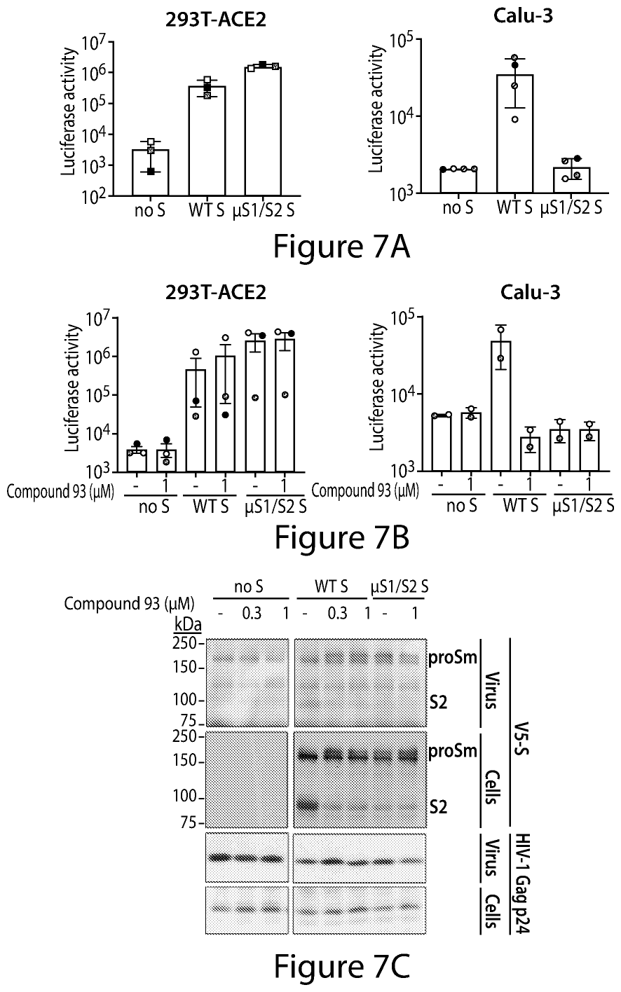

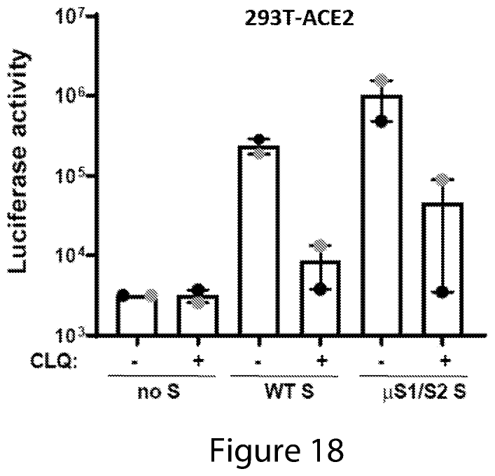

To asses the importance of spike processing at the S1/S2 site in SARS-CoV-2 entry, gp160-defective HIV with WT or μS1/S2 S-protein was pseudotyped and tested viral entry in different target cells. Using lung Calu-3 and kidney HEK293T-ACE2 as model cell targets, cell-entry of viruses expressing μS1/S2 were completely defective in Calu-3, but not in 293T-ACE2 that exhibited enhanced viral-entry (FIG. 7A), similar to Vero E6 cells. Since SARS-CoV-2 can enter target cells via “pH-independent” or “pH-dependent” pathways and the virus reportedly uses the latter to infect Vero E6 cells. The pH-raising chloroquine efficiently blocked entry of pseudotyped SARS-CoV-2 and its μS1/S2 mutant (FIG. 18), suggesting that in the 293T-ACE2 system the S-protein, which mediates viral entry, is activated by endosomal pH-dependent proteases. This aligns with previous findings that HEK293 cells allow endocytosis of pseudovirions carrying SARS-CoV-2 spike protein via clathrin-coated vesicles.

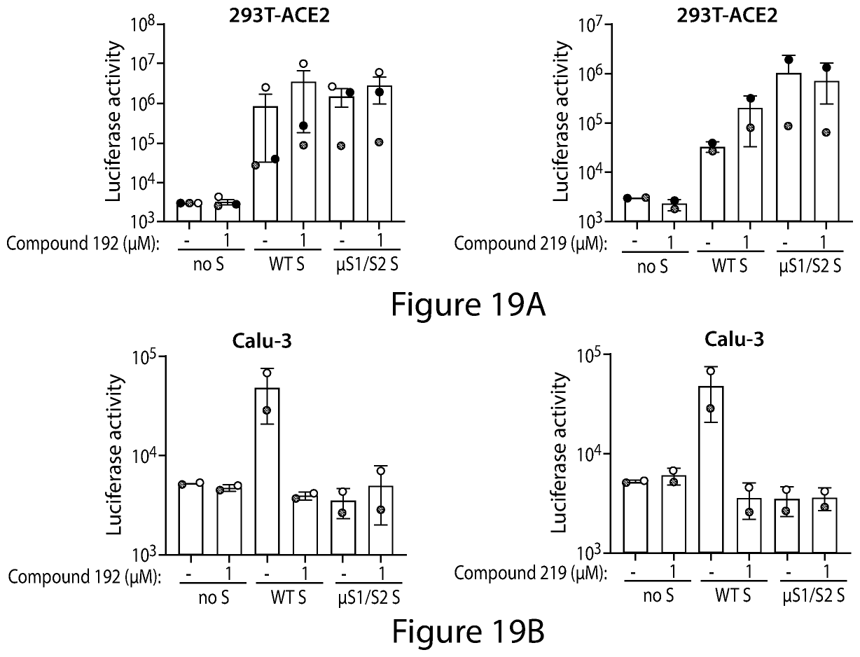

When 293T17 producing cells were treated with representative compounds of this disclosure during viral packaging, HIV particles expressing the WT proS-protein remained highly infectious in 293T-ACE2 but were completely defective in Calu-3 (FIG. 7B; FIG. 19). Thus, treatment of the cells with the compounds of this disclosure phenocopied the effect of the μS1/S2 in both target cells. Importantly, these phenotypes were not due to increased pseudoviral production/release since levels of HIV p24 were comparable in all cases (FIG. 7C). Similarly, in the presence of 1 μM Compound 93, processing of WT-S was clearly impaired, while the overall μS1/S2 expression profile was not affected (FIG. 7C). The data indicate that processing of S-protein by furin-like convertases is essential for the pH-independent viral entry in Calu-3 cells but not in HEK293 cells stably expressing ACE2 where the virus enters by the endocytic pathway.

### Example 8. Furin-Like Inhibitors Reduce Virus Production in SARS-CoV-2-Infected Cells

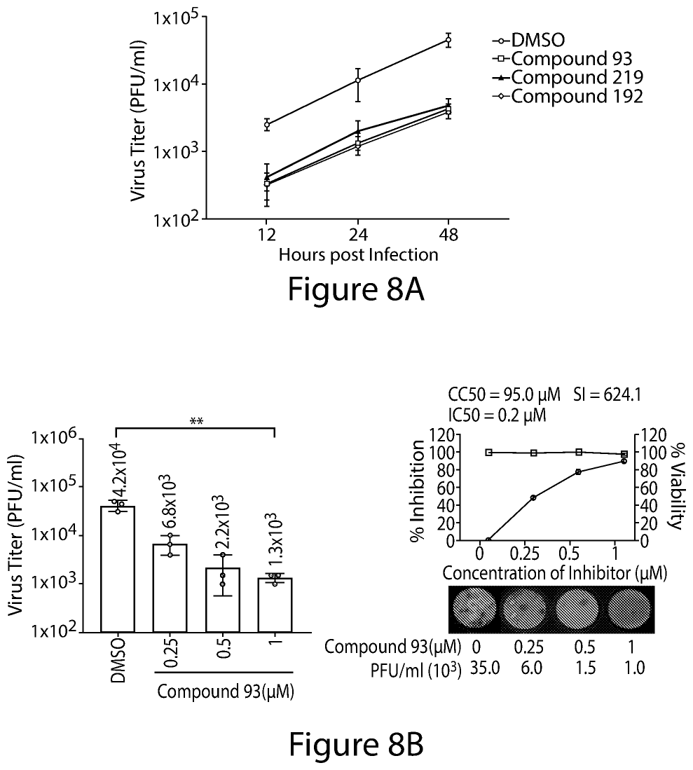

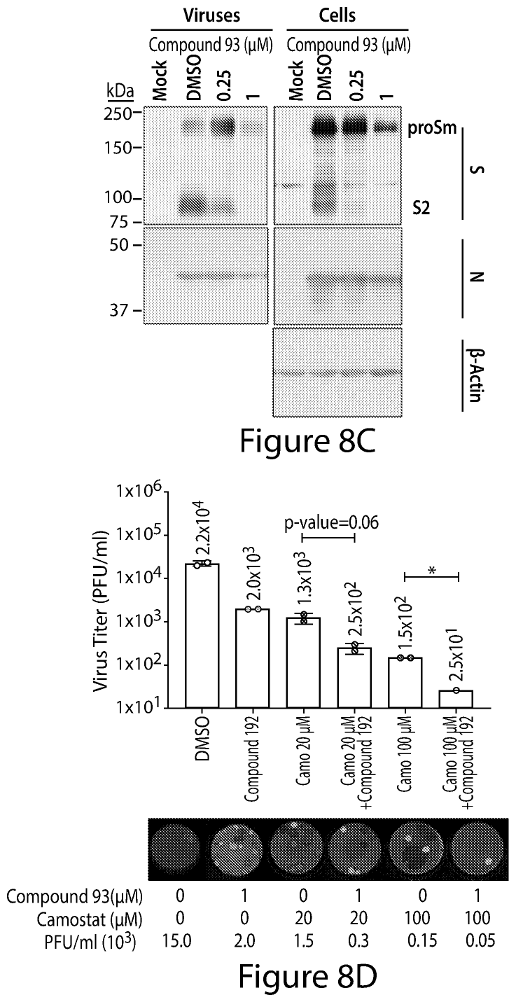

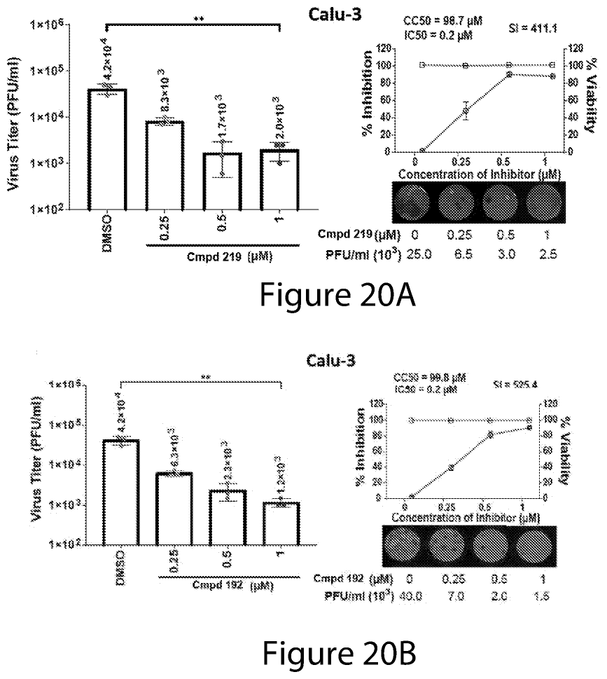

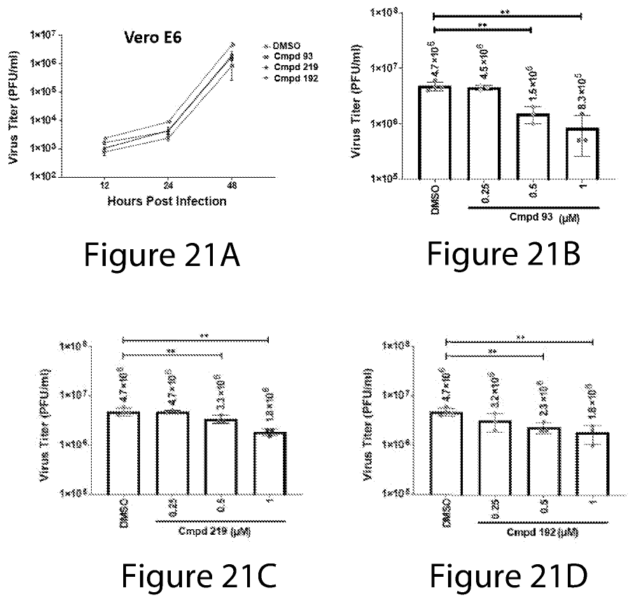

The possible antiviral effects of these furin-like inhibitors on SARS-CoV-2 replication was evaluated in Calu-3 cells pretreated with 1 μM Compound 93, 192, or 219 24h before infection with laboratory isolated SARS-CoV-2 virus (MOI: 0.01) and harvested at 12, 24 and 48h post infection for plaque assay analysis. Compound 93, 192, or 219 significantly decreased viral titers at 12, 24 and 48h post-infection (FIG. 8A). The inhibitory effect of various doses of these inhibitors on the yield of infectious virus produced 24h post-infection was investigated, and it was found that the titer of progeny viruses was reduced by more than 30-fold with 1 μM Compound 93 although the inhibitory effect could be observed starting at 0.25 μM (FIG. 8B; left panel). As well, the IC₅₀ and selectivity index (SI) of Compound 93 were found to be 0.2 μM and 624.1, respectively, underlining the inhibitor's bonafide efficacy (FIG. 8B; right panel). A similar analysis with Compound 219 and Compound 192 revealed comparable antiviral effects and selectivity index (FIGS. 20A, 20B). Importantly, the levels of viral spike (full length and cleaved S) and nucleocapsid proteins in Calu-3 cells treated with different doses of Compound 93 and the corresponding progeny virus levels were similarly decreased (FIG. 8C), underscoring the crucial role played by furin-like convertases in the production of infectious SARS-CoV-2 during infection of lung epithelial cells. In addition, the antiviral effect of these inhibitors for SARS-CoV-2 infection was also evaluated in Vero E6, a cell target that is reported to be primarily infected via the endocytic pathway. In this system, the best inhibitory effect with 1 μM Compound 93 demonstrated a ˜5.7-fold decrease in virus production sustained over a 12-48 h-infection period (FIG. 21), reflecting some furin-activity in endosomes.

Based on the SI of the representative compounds in Vero E6 and Calu-3 cells, Compound 192 was further used in combination with Camostat to explore a potential synergistic effect of these inhibitors on viral replication in Calu-3 cells. To this end, it was observed that the two inhibitors could individually and meaningfully reduce viral replication, but co-treatment with both (1 μM Compound 192+100 μM Camostat) inhibited >99% of progeny viruses (FIG. 8D). This highlights a synergistic effect of these drugs and the importance of endogenous furin-like proteases, and presumably TMPRSS2, in the efficient infection of Calu-3 cells by SARS-CoV-2.

### Example 9. Experimental Methods

### Enzymatic PC-Inhibition and Cellular Furin Inhibition of Furin-Inhibitors

Biochemical assay: The proprotein convertases furin (108-574-Tev-Flag-6His), PC5A (PCSK5; 115-63-Tev-Flag-6His), PACE4 (PCSK6; 150-693-Tev-Flag-6His), and PC7 (PCSK7; 142-634-Tev-Flag-6His) enzymes were purified from BacMam transduced CHO cells. Reactions were performed in black 384-well polystyrene low volume plates (Greiner) at a final volume of 10 μL. Small-molecule inhibitors (e.g., Compound 93, Compound 219, and Compound 192) were dissolved in DMSO (1 mM) and serially diluted 1 to 3 with DMSO through eleven dilutions to provide a final compound concentration range from 0.00017 to 10 μM. 0.05 μl of each concentration was transferred to the corresponding well of an assay plate, and then 5 μl of enzyme (furin, PCSK5, PCSK6, and PCSK7) in assay buffer (100 mM HEPES pH 7.5, 1 mM CaCl₂) and 0.005% Triton X-100) was added using a Multidrop Combi (Thermo) to the compound plates to give a final protein concentration of 0.02, 0.5, 2.5, and 1.0 nM respectively. The plates were mixed by inversion, and following a 30 min preincubation of enzyme with compound at room temperature (˜22° C.), the substrate FAM-QRVRRAVGIDK-TAMRA (AnaSpec #808143, 5 μl of a 1, 0.25, 0.20, and 0.5 μM solution in assay buffer for furin, PCSK5, PCSK6, and PCSK7 respectively) was added using a Multidrop Combi to the entire assay plate. The plates were centrifuged at 500×g for 1 minute and incubated at room temperature for two hours. Enzyme inhibition was then quantified using an Envision instrument (PerkinElmer). Data were normalized to maximal inhibition determined by 1 μM Decanoyl-Arg-Val-Lys-Arg-Chloromethylketone (Calbiochem #344930). Golgi imaging assay: This assay uses an image-based platform to evaluate the intracellular activity of furin inhibitors. Reactions were performed in black 384-well, tissue culture-treated, clear bottom plates (Greiner). Compounds under analysis were dissolved in DMSO (1.0 mM) and serially diluted 1 to 3 with DMSO through eleven dilutions. This creates a final compound concentration range from 0.00017 to 10 μM, and 0.1 μL of each concentration was transferred to the corresponding well of the assay plate.

Cellular assay: Analyses were initiated by the addition of U2OS cells simultaneously transduced with a BacMam-delivered construct containing a Golgi-targeting sequence followed by a 12-amino acid furin/PCSK cleavage site from Bone Morphogenic Protein 10 (BMP10) and then GFP at the C terminus. The dibasic furin cleavage site sequence was flanked by glycine rich linkers (GalNAc-T2-GGGGS-DSTARIRRNAKGGGGGS-GFP). Briefly, frozen cells are thawed in assay media (Dulbecco's Modified Eagles Medium Nutritional Mixture F-12 (Ham) without phenol red containing 5% FBS) and diluted to deliver 6000 cells/well (50 μl) to the plate using a Multidrop Combi (Thermo). After a 24-hour incubation period at 37° C., the cells are stained with Cell Mask Deep Red, fixed in paraformaldehyde and the nuclei stained using Ho33342. The Golgi-targeted GFP forms bright punctate clusters within the cell. In the absence of a furin/PCSK inhibitor, the endogenous protease cleaves GFP from its N-acetylgalactosaminyltransferase-2 Golgi tether, releasing GFP into the Golgi lumen where fluorescence is diluted below the threshold of assay sensitivity. In the presence of a cell permeable furin/PCSK inhibitor, GFP fluorescence increases as intra-Golgi protease activity is reduced. Cellular GFP intensity is determined by image-based acquisition (Incell 2200, Perkin Elmer) at 40× magnification with 4 fields measured per well. Multi-scale top hat segmentation is used to identify the GFP-tagged puncta and to quantitate the average fluorescence of all puncta on a per cell basis. Cellular toxicity is determined in parallel.

Furin and TRMPSS2 fluorogenic assays: Recombinant furin was purchased from BioLegend (#719406), TRMPSS2 from Cusabio and the DABCYLGlu-EDANS labelled peptides encompassing the different cleavage sites (Supplementary Table 1) were purchased from Genscript. Reactions were performed at room temperature in black 384-well polystyrene low volume plates (CELLSTAR-Greiner Bio-One #784476) at a final volume of 15 μL. The fluorescent peptides were used at 5 μM and the reactions were performed in 50 mM Tris buffer (pH 6.5 or 7.5), 0.2% Triton X-100, 1 mM CaCl₂) and furin was added at a final concentration of 2-100 nM. Small-molecule inhibitors (Compound 93, Compound 219, and Compound 192) were dissolved in DMSO (1 mM) and serially diluted 1 to 2 with DMSO to provide a final compound concentration range from 50 μM to 0.01 nM with 5% DMSO in the enzymatic assay. For TMPRSS2, the fluorescent peptides were used at 5 μM and the reactions were performed in 50 mM Tris buffer (pH 6.5 or 7.5), 0.2% Triton X-100, 50 mM NaCl and TMPRSS2 was added at final concentrations of 25-100 nM. Cleavage of the synthetic peptides was quantitated by determining the increase of EDANS (493 nM) fluorescence following release of the DABCYL quencher, which is excited at 335 nM using a Safire 2 Tecan fluorimeter. The fluorescence was followed during 90 min, and the enzymatic activity was deduced by measurement of the increase of fluorescence during the linear phase of the reaction. Each reaction was performed in triplicate and the standard deviation was calculated using Excel-ecart type function

$$\sqrt{\frac{\sum {( x - \overline{x} )}^{2}}{( n - 1 )}} ) .$$

### Plasmids

C-terminal V5 tagged Spike glycoprotein of SARS-CoV-2 (optimized sequence) and its mutants were cloned into the pIRES-neo3 vector. Site-directed mutagenesis was achieved using a Quick-Change kit (Stratagene, CA) according to the manufacturer's instructions. The plasmids pCI-NEO-hACE2 received from DW Lambert (University of Leeds) and pIRES-NEO3-hTMPRSS2 from P Jolicoeur (IRCM). The AEnv Vpr Luciferase Reporter Vector (pNL4-3.Luc.R-E-) was obtained from Dr. Nathaniel Landau through the NIH AIDS Reagent Program whereas the pHIV-1NL4-3 AEnv-NanoLuc construct was a kind gift from Dr. P Bieniasz. Plasmids encoding VSV-G, as HIV-1 Env and tat were previously described.

### Cell Culture and Transfection

Monolayers of HeLa, HEK293T, HEK293T17 and Vero E6 cells were cultured in 5% CO₂ at 37° C. in Dulbecco's modified Eagle's medium (DMEM; Invitrogen) supplemented with 10% (v/v) fetal bovine serum (FBS; Invitrogen). HEK293T-ACE2, a generous gift from Dr. Paul Bieniasz, were maintained in DMEM containing 10% FBS, 1% nonessential amino acids (NEAA) and 50 μg/ml blasticidin (Invivogen). Calu-3 were cultivated in F12K/DMEM containing 10% FBS. The cells were cultured in 5% CO₂ at 37° C. in Dulbecco's modified Eagle's medium (DMEM; Invitrogen) supplemented with 10% (v/v) fetal bovine serum (FBS; Invitrogen). The cells were transfected with JetPrime transfection reagent according to the manufacturer's instructions (Polyplus transfection, New York, USA). At 24h post transfection the culture media were changed to serum-free DMEM and incubated for an additional 24h. To establish the stable HeLa cells over-expressing human ACE2, the cells were maintained in media containing 500 μg/mL of neomycin (G418, Wisent) for two weeks.

To generate HIV particles pseudotyped with SARS-CoV-2 S, 293T17 cells (600,000 cells plated in a 6-well vessel) were transfected with 1 μg pNL4-3.Luc.R-E- (or pHIV-1NLΔEnv-NanoLuc) in the presence or absence of 0.3 μg pIR-2019-nCoV-S V5 plasmids using Lipofectamine-3000 (Life Technologies). In certain experiments, 293T17 cells were treated with small-molecule inhibitors (e.g., Compound 93, 219, or 192) at 6 h post transfection. Pseudovirions expressing the nano- or firefly-luciferase were collected at 24 h or 48 h post transfection, respectively. Viral supernatants were clarified by centrifugation at 300×g, passed through a 0.45-μm pore-size polyvinylidene fluoride (PVDF; Milipore) syringe filter (Millipore; SLGVR33RS), and aliquots frozen at −80° C. For WB analysis of purified pseudovirions, viral supernatants were concentrated by ultracentrifugation on a 20% sucrose cushion for 3h at 35,000 RPM; Beckman Couter OPTIMA XE; Ti70.1 rotor). HIV particles lacking the SARS-CoV-2 S glycoprotein served as a negative control in all experiments.

### Cell Viability Assay Using MTT

Cells, seeded in a 96-well plate, the day before, at 10,000 (HEK-293T and Vero E6) or 50,000 (Calu-3) cells, were treated with serial 10-fold dilutions of small-molecule inhibitors (e.g., Compound 93, 192, or 219) for up to 48h. Cells treated with vehicle alone were used as negative control. MTT was subsequently added to the medium (final concentration: 2.5 mg/ml) and cells were further incubated for 4h at 37° C. After removal of the culture media, DMSO was added and absorbance read at 595 nm using a microplate spectrophotometer. The data from two independent experiments done in triplicates was used to calculate the CC50 by nonlinear regression using GraphPad Prism V5.0 software.

### Western Blots

The cells were washed with PBS and then lysed using RIPA buffer (1% Triton X-100, 150 mM NaCl, 5 mM EDTA, and 50 mM Tris, pH 7.5) for 30 min at 4° C. The cell lysates were collected after centrifugation at 14,000×g for 10 min. The proteins were separated on 7% tris-glycine or 8% tricine gels by SDS-PAGE and transferred to a PVDF membrane (Perkin Elmer). When specified, media from cultured and transfected cells were collected and concentrated 10x using Amicon Ultra 2 ml devices with a 10 kDa cut-off (Millipore; UFC 201024), as specified by the manufacturer, and analyzed by SDS-PAGE followed by Western blotting. The proteins were revealed using a V5-monoclonal antibody (V5-mAb V2660; 1:5000; Invitrogen), ACE2 antibody (rabbit monoclonal ab108252; 1:3,000; Abcam), TMPRSS2 antibody (rabbit polyclonal; 14427-1-AP; 1:1,000; Proteintech), Actin antibody (rabbit polyclonal A2066; 1:5,000; Sigma), or SARS-CoV-2 spike antibody (rabbit polyclonal GenTex GTX135356; 1:2,000; GenTex). The antigen-antibody complexes were visualized using appropriate HRP conjugated secondary antibodies and enhanced chemiluminescence kit (ECL; Amersham or Bio-Rad) and normalization was reported to β-actin. The quantification of the bands was performed using Image Lab software (Bio-Rad).

For analysis of SARS-CoV-2 S virions or pseudovirions, protein extracts of purified viral particles and corresponding producing cells (Calu-3 or 293T17, respectively) were resolved on 10% tris-glycine gels and immunoblotted for spike, nucleocapsid, HIV-1 Gag p24 or actin using anti-V5 (for pseudovirion detection; V2660)/anti-S2 (for virion detection; Sino Biologicals; 40590-T62), anti-N(Sino Biologicals; 40143-MM05), anti-p24 (MBS Hybridoma line 31-90-25) or anti-actin (MP Biomedicals, SKU 08691001), respectively.

### Glycosidase Treatment

30 to 50 μg proteins were digested for 90 min at 37° C. with endoglycosidase-H (Endo-H; P0702L) or endoglycosidase-F (Endo-F; P0705S) as recommended by the manufacturer (New England Biolabs).

### Inhibitor Treatment

At 24h post transfection, cells were incubated for 6h with two pan-PC inhibitors: the cell permeable decanoyl-RVKR-chloromethylketone (cmk; 50 mM; 4026850.001; Bachem), or with the cell surface PC-inhibitor hexa-D-arginine (D6R; 20 μM; 344931; EMD). Culture media were then replaced with fresh ones containing the inhibitors for an additional 24h. For the selective cell-permeable furin-like inhibitors, the cells were treated with the inhibitors at the specified concentration starting at 5h pre-transfection and throughout the duration of the experiment.

### Cell-to-Cell Fusion Assay

HeLa or HeLa TZM-bl cells were plated at 200,000 cells in 12-well plates. HeLa cells were transiently transfected with different constructs of SARS-COV-2 Spike or NL4.3-HIV Env, or an empty vector and 0.2 μg of CMV-Tat plasmid. HeLa TZM-bl cells were transfected with human ACE2, TMPRSS2 or a combination of both. At 6h post-transfection, media were replaced with fresh ones containing furin-inhibitors, and 24h later the cells were detached with PBS-EDTA (1 μM). Different combinations of HeLa and HeLa-TZM-bl cells were placed in co-culture plate at a ratio of 1:1 for a total of 60,000 cells/well of a 96 well place. After 18-24h the media were removed and 50 μl of cell lysis reagent was added in each well. 20 μl of the cell lysate was used for luciferase reading using 50 μl of Renilla luciferase reagent (Promega, Madison, Wis., USA). The relative light units (RLU) were measured using a Promega GLOMAX plate reader (Promega, Madison, Wis., USA) and values were reported as fold increase over the RLU measured in co-culture of HeLa cells transfected EV with respective TZM-bl cells.

### Microscopy

To establish the luciferase assay, cell co-cultures were plated on glass coverslips.

After 18-24h, the cells were incubated with 488 CellMask™ to stain the membrane and then fixed with 4% PFA for 15 min at 4° C. The glass coverslips were mounted on glass slides using ProLong™ Gold Antifade containing DAPI (Invitrogen). The number of syncytia were counted over 10 fields.

### Immunofluorescence

Cell culture and transfection were performed on glass coverslips. Cells were washed twice with PBS and fixed with fresh 4% paraformaldehyde for 10 min at room temperature. Following washes, cells were either non-permeabilized or permeabilized with 0.2% Triton X-100 in PBS containing 2% BSA for 5 min, washed, and then blocking was performed with PBS containing 2% BSA for 1 h. Cells were incubated with primary antibodies overnight at 4° C. using an antibody against V5 (mouse monoclonal R960-25; 1:1000; Invitrogen), Spike (mouse monoclonal GTX632604; 1:500; GeneTex) and ACE2 (goat polyclonal AF933; 1:500; RnDsystems). Following wash, corresponding species-specific Alexa-Fluor (488 or 555)-tagged antibodies (Molecular Probes) were incubated for 1 h at room temperature. Coverslips were mounted on a glass slide using ProLong Gold Reagent with DAPI (P36935, Life Technologies). Samples were visualized using a confocal laser-scanning microscope (LSM710, Carl Zeiss) with Plan-Apochromat 63x/1.40 Oil DIC M27 objective on ZEN software.

### Pseudovirus Entry

293T-ACE2 or Calu-3 (10,000 cells/well plated in a 96-well dish the day before) were incubated with up to 200 μl filtered pseudovirions for overnight. Viral inoculum was removed, then fresh media were added and the cells cultured for up to 72h. Upon removal of spent media, 293T-ACE2 and Calu-3 cells were gently washed twice with PBS and analyzed for firefly- or nano-luciferase activity, respectively using Promega luciferase assay (Cat #E1501) or Nano-Glo luciferase system (Cat #N1110), respectively.

### Live Virus Assays

### Replication Competent SARS-CoV-2 Viruses

SARS-CoV-2, which served as the viral source, was originally isolated from a COVID-19 patient in Quebec, Canada and was designated as LSPQ1. The clinical isolate was amplified, tittered in Vero E6 using a plaque assay as detailed below, and the integrity of the S-protein multi-basic protein convertase site validated by sequencing. All experiments involving infectious SARS-CoV-2 virus were performed in the designated areas of the Biosafety level 3 laboratory (IRCM) previously approved for SARS-CoV-2 work.

### Plaque Assay in Vero E6

Vero E6 cells (1.2×10⁵ cells/well) were seeded in quadruplicate in 24-well tissue culture plates in DMEM supplemented with 10% FBS two days before infection. Cells were infected with up to six ten-fold serial dilutions (10⁻²-10⁻⁶) of viral supernatant containing SARS-CoV-2 for 1 h at 37° C. (200 μl infection volume). The plates were manually rocked every 15 min during the 1-hour period. Subsequently, virus was removed, cells were washed and overlaying media (containing 0.6% low melt agarose in DMEM with 10% FBS) was added, and incubated undisturbed for 60-65 h at 37° C. Post incubation, cells were fixed with 4% formaldehyde and stained with 0.25% crystal violet (prepared in 30% methanol). High quality plaque pictures were taken using a high resolution DLSR camera (Nikon model: D80, objective: “AF Micro-Nikkor 60 mm f/2.8D”). Plaques were counted manually and in parallel, imaged plaque plates were processed and plaques enumerated using an automated algorithm based Matlab software. Virus titer is expressed as plaque-forming units per ml (PFU/ml): (number of plaques×dilution factor of the virus)×1000/volume of virus dilution used for infection (in μl). Multiplicity of infection (MOI) expressed as: MOI=PFU of virus used for infection/number of cells.

Cell Infections with Fully Replicative SARS-CoV-2

Vero E.6 and Calu-3 cells were seeded in duplicates in 12-well plates (2.3×10⁵ cells/well) the day before. Cells were pre-treated with various concentrations (0.1-1 μM) of the small-molecule inhibitor (e.g., Compound 93, 192, or 219) and vehicle alone (DMSO) for up to 24h. In certain experiments, Calu-3 were also pre-treated with Camostat for 1 h. Thereafter, the cells were infected with SARS-CoV-2 virus at MOI of 0.001 for 1 h (Vero E6) or 0.01 for 3h (Calu-3 cells) in 350 μl of serum-free DMEM at 37° C. with occasional manual rocking of plates. Cells plus media only were used as a control. After incubation, virus was removed, and the cell monolayer was washed twice successively with PBS and serum-free DMEM. New media (total 1 ml) containing the aforementioned concentrations of the small molecule inhibitor was subsequently added to cells. Cell-free supernatant (250 μl) was removed at 12, 24 and 48h post infection. The drugs were replenished for 1 ml media at 24h post-infection. The virus supernatants were stored at −80° C. until further use. Viral production in the supernatant was quantified using a plaque assay on Vero E6.1 cells as described above. In certain experiments, viral supernatants were harvested at the end of infection and purified on a 20% sucrose cushion using ultracentrifugation as described above. The resulting concentrated virus and corresponding infected cells were analyzed by Western blotting as appropriate.

Quantification and statistical analysis: Virus titers quantified by plaque assay in triplicate were shown as mean f standard deviation. The results from experiments done in triplicates were used to calculate the IC₅₀ by nonlinear regression using GraphPad Prism V5.0 software. The difference between the control cells (virus with 0.001% DMSO) and the cells treated with the small-molecule inhibitors (e.g., Compound 93, 192, or 219) were evaluated by Student's t test. The P values of 0.05 or lower were considered statistically significant (*, p<0.05; **, p<0.01; ***, p<0.001).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a viral infection resulting from coronaviridae family virus in a subject in need thereof comprising administering to the subject a compound of Formula (I), wherein the compound of Formula (I) is of the formula: 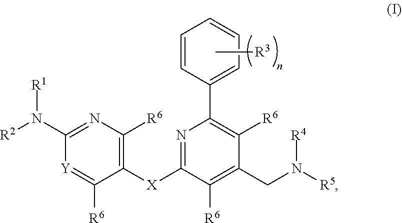
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₅(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)z, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

2. A method of inhibiting the entry of a coronaviridae family virus into a cell in a subject in need thereof, comprising administering to the subject an effective amount of a compound of Formula (I), wherein the compound of Formula (I) is of the formula: 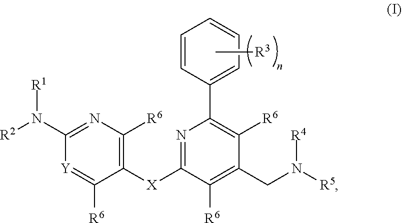
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

3. A method of inhibiting the maturation of a coronaviridae family virus in a subject in need thereof, comprising administering to the subject an effective amount of a compound of Formula (I), wherein the compound of Formula (I) is of the formula: 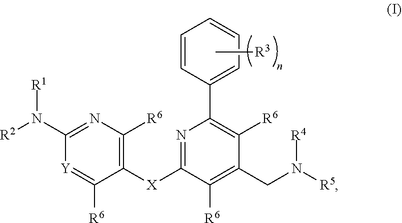
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁹, —C(O)CO₂R⁸, —R⁷, —OR⁷, —NHR⁸, —NR⁷R⁸, —C(O)R⁷, —CONHR⁸, CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

4. A compound of Formula (I), or a pharmaceutically acceptable salt thereof, for use in treating and/or preventing a viral infection resulting from a coronaviridae family virus in a subject in need thereof, wherein the compound of Formula (I) is of the formula: 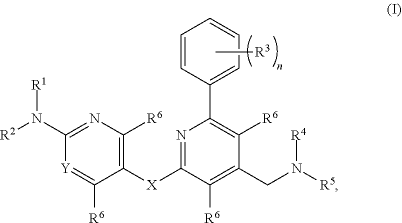
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

5. Use of a compound of Formula (I), or a pharmaceutically acceptable salt thereof, in the manufacture of a medicament for the treatment and/or prevention of a viral infection resulting from a coronaviridae family virus in a subject in need thereof, wherein the compound of Formula (I) is of the formula: 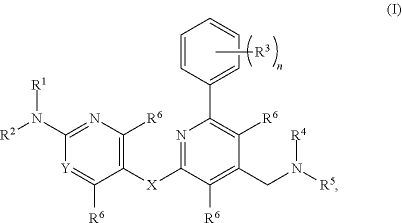
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl;
n is 1, 2, 3, or 4.

6. A pharmaceutical composition comprising a compound of Formula (I), for use in the methods of any one of the preceding claims, wherein the compound of Formula (I) is of the formula: 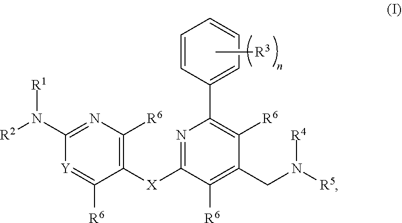
or a pharmaceutically acceptable salt thereof, wherein:
X is —O— or —N(R⁸)—;
Y is —N═ or —C(R⁶)═;
R¹ and R² are each independently H or optionally substituted (C₁-C₄)alkyl; optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl; optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

7. The method, compound, pharmaceutical composition, or use of any one of claims 1-6, further comprising administering an additional pharmaceutical agent.

8. The method, compound, pharmaceutical composition, or use of claim 7, wherein the additional pharmaceutical agent is an antiviral agent.

9. The method, compound, pharmaceutical composition, or use of claim 8, wherein the antiviral agent is Abacavir, Acyclovir, Amantadine, Atazanavir, Chloroquine, Darunavir, Elvitegravir, Fosamprenavir, Ganciclovir, Indinavir, Ledipasvir, Lopinavir, Nitazoxanide, Oseltamivir, Penciclovir, Peramivir, Raltegravir, Ribavirin, Rimantadine, Ritonavir, Saquinavir, Sofosbuvir, Tipranavir, Velpatasvir, Zanamivirfavipiravir, remdesivir, Oyal, galidesivir, umifenovir, or hydroxychloroquine.

10. The method, compound, pharmaceutical composition, or use of claim any one of claims 1-6, wherein the additional pharmaceutical agent is an antibacterial agent.

11. The method, compound, pharmaceutical composition, or use of claim 10, wherein the antibacterial agent is azithromycin.

12. The method, compound, pharmaceutical composition, or use of claim 7, wherein the additional pharmaceutical agent is an anti-inflammatory agent.

13. The method, compound, pharmaceutical composition, or use of claim 12, wherein the anti-inflammatory agent is Gimsilumab, IL-6 antibodies, actemra, paracetamol, or non-steroidal anti-inflammatory drugs (NSAIDs).

14. The method, compound, pharmaceutical composition, or use of claim 7, wherein the additional pharmaceutical agent is human antibodies to SARS-CoV-2.

15. The method, compound, pharmaceutical composition, or use of claim 7, wherein the additional pharmaceutical agent is antibodies that bind the S-spike protein of SARS-CoV-2.

16. The method, compound, pharmaceutical composition, or use of claim 7, wherein the additional pharmaceutical agent is a serine protease inhibitor.

17. The method, compound, pharmaceutical composition, or use of claim 16, wherein the serine protease inhibitor is a TMPRSS2 inhibitor.

18. The method, compound, pharmaceutical composition, or use of claim 16 or 17, wherein the serine protease inhibitor is camostat.

19. The method, compound, pharmaceutical composition, or use of claim 7, wherein the additional pharmaceutical agent is an ACE2 inhibitor.

20. The method, compound, pharmaceutical composition, or use of claim 19, wherein the ACE2 inhibitor is benazepril, captopril, enalapril, fosinopril, lisinopril, moexipril, perindopril, quinapril, ramipril, or trandolapril.

21. The method, compound, pharmaceutical composition, or use of any one of claims 1 to 20, wherein the coronaviridae family virus is an alphacoronavirus.

22. The method, compound, pharmaceutical composition, or use of claim 21, wherein the alphacoronavirus is HCoV-NL63.

23. The method, compound, pharmaceutical composition, or use of claim 21, wherein the alphacoronavirus is HCoV-229E.

24. The method, compound, pharmaceutical composition, or use of any one of claims 1 to 20, wherein the coronaviridae family virus is a betacoronavirus.

25. The method, compound, pharmaceutical composition, or use of claim 24, wherein the betacoronavirus is SARS-CoV.

26. The method, compound, pharmaceutical composition, or use of claim 24, wherein the betacoronavirus is SARS-CoV-2.

27. The method, compound, pharmaceutical composition, or use of claim 24, wherein the betacoronavirus is MERS-CoV.

28. The method, compound, pharmaceutical composition, or use of claim 24, wherein the betacoronavirus is HCoV-OC43.

29. The method, compound, pharmaceutical composition, or use of claim 24, wherein the betacoronavirus is HCoV-HKU1.

30. The method, compound, pharmaceutical composition, or use of any one of claims 1 to 29, wherein Y is —N═.

31. The method, compound, pharmaceutical composition, or use of any one of claims 1 to 30, wherein the compound of Formula (I) is of Formula (H): 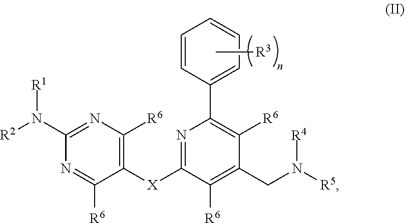
or a pharmaceutically acceptable salt thereof.

32. The method, compound, pharmaceutical composition, or use of any one of claims 1 to 30, wherein Y is —C(R⁶).

33. The method, compound, pharmaceutical composition, or use of any one of claims 1 to 30, or 31, wherein the compound of Formula (I) is of Formula (III): 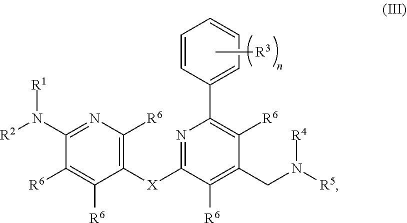
or a pharmaceutically acceptable salt thereof.

34. The method, compound, pharmaceutical composition, or use of any one of claims 1 to 33, wherein R³ is halogen.

35. The method, compound, pharmaceutical composition, or use of any one of claims 1 to 34, wherein R³ is —Cl.

36. The method, compound, pharmaceutical composition, or use of any one of claims 1 to 35, wherein n is 2.

37. The method, compound, pharmaceutical composition, or use of any one of claims 1 to 36, wherein X is —O—.

38. The method, compound, pharmaceutical composition, or use of any one of claims 1 to 37, wherein R⁴ and R⁵ taken together with the nitrogen atom to which they are attached form a 4-11 membered heterocyclic ring, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹.

39. The method, compound, pharmaceutical composition, or use of any one of claims 1 to 38, wherein R⁴ and R⁵ taken together with the nitrogen atom to which they are attached form an optionally substituted pyrrolidine, pyrazolidine, imidazolidine, piperidine, piperazine, or morpholine ring.

40. The method, compound, pharmaceutical composition, or use of any one of claims 1 to 39, wherein R⁴ and R⁵ taken together with the nitrogen atom to which they are attached form a heterocyclic ring of the formula:

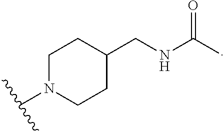

41. The method, compound, pharmaceutical composition, or use of any one of claims 1 to 40, wherein R¹ and R² taken together with the nitrogen atom to which they are attached form a 4-11 membered heterocyclic ring, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, CONR⁷R⁸, or —SO₂R⁷.

42. The method, compound, pharmaceutical composition, or use of any one of claims 1 to 37, wherein R¹ and R² taken together with the nitrogen atom to which they are attached form an optionally substituted an optionally substituted pyrrolidine, pyrazolidine, imidazolidine, piperidine, piperazine, or morpholine ring.

43. The method, compound, pharmaceutical composition, or use of any one of claims 1 to 31 or 34 to 42, wherein R¹ and R² taken together with the nitrogen atom to which they are attached form a piperazine ring of the formula:

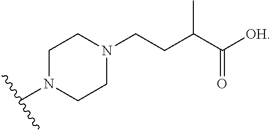 [SMILES: *N1CCN(CCC(C)C(=O)O)CC1]

44. The method, compound, pharmaceutical composition, or use of any claims 1 to 31, or 34 to 43, wherein the compound, or pharmaceutically acceptable salt thereof, is:
1-((1-((2-(3,5-dichlorophenyl)-6-((2-(4-(3-(methylsulfinyl)butyl)piperazin-1-yl)pyrimidin-5-yl)oxy)pyridin-4-yl)methyl)piperidin-4-yl)methyl)-3-methylurea;
1-((1-((2-(3,5-dichlorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)-3-methylurea;
N-((1-((2-(3,5-dichlorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)acetamide;
1-((1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)methyl)-3-methylurea;
2-(1-((2-(3,5-dichlorophenyl)-6-((2-(4-(1-hydroxypropan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxycyclobutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(1,3-dihydroxypropan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-((1s,3s)-3-hydroxy-3-methylcyclobutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-((1r,3r)-3-hydroxy-3-methylcyclobutyl)piperazin-1-yl)pyrimidin-5-yl)oxy)pyridin-4-yl)methyl)piperidin-4-yl)acetic acid;
1-(2-(3,5-dichlorophenyl)-6-((2-(4-(3-(methylsulfonyl)propyl)piperazin-1-yl)pyrimidin-5-yl)oxy)pyridin-4-yl)-N-methylmethanamine;
1-((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl) pyridin-2-yl)oxy)pyrimidin-2-yl)piperazin-1-yl)propane-1-sulfonamide;
methyl((1-((2-(3,5-dichlorophenyl)-6-((2-(piperazin-1-yl)pyrimidin-5-yl)oxy)pyridin-4-yl)methyl)piperidin-4-yl)methyl)carbamate;
3-(4-(5-((6-(3,5-dichlorophenyl)-4-((4-fluoro-4-(((methoxycarbonyl)amino)methyl) piperidin-1-yl)methyl)pyridin-2-yl)oxy)pyrimidin-2-yl)piperazin-1-yl)propanoic acid;
2-(1-((2-(3,5-dichlorophenyl)-6-((2-(4-methylpiperazin-1-yl)pyrimidin-5-yl)oxy)pyridin-4-yl)methyl)piperidin-4-yl)ethanol;
2-((1-((2-(3,5-dichlorophenyl)-6-((2-(4-methylpiperazin-1-yl)pyrimidin-5-yl)oxy)pyridin-4-yl)methyl)piperidin-4-yl)oxy)acetic acid;
3-(4-(5-((4-((4-(2-(carbamoyloxy)ethyl)piperazin-1-yl)methyl)-6-(3,5-dichlorophenyl) pyridin-2-yl)oxy)pyrimidin-2-yl)piperazin-1-yl)propanoic acid;
3-(3-(5-((6-(3,5-dichlorophenyl)-4-((4-(((methoxycarbonyl)amino)methyl)piperidin-1-yl)methyl)pyridin-2-yl)oxy)pyrimidin-2-yl)-3,8-diazabicyclo[3.2.1]octan-8-yl)propanoic acid;
4-(4-(5-((6-(3,5-dichlorophenyl)-4-((4-(2-hydroxyethyl)piperidin-1-yl)methyl)pyridin-2-yl)oxy)pyrimidin-2-yl)piperazin-1-yl)-2-methylbutanoic acid;
4-(4-(5-((6-(3,5-dichlorophenyl)-4-((4-(propionamidomethyl)piperidin-1-yl)methyl) pyridin-2-yl)oxy)pyrimidin-2-yl)piperazin-1-yl)-2-methylbutanoic acid;
4-(4-(5-((6-(3,5-dichlorophenyl)-4-((4-fluoropiperidin-1-yl)methyl)pyridin-2-yl)oxy)pyrimidin-2-yl)piperazin-1-yl)butan-2-ol;
3-(4-(5-((4-((4-(acetamidomethyl)piperidin-1-yl)methyl)-6-(3-chloro-5-(difluoromethyl)phenyl)pyridin-2-yl)oxy)pyrimidin-2-yl)piperazin-1-yl)propanoic acid;
3-(4-(5-((6-(3,5-dichlorophenyl)-4-((4-(((methoxycarbonyl)amino)methyl)piperidin-1-yl)methyl)pyridin-2-yl)oxy)pyrimidin-2-yl)piperazin-1-yl)propanoic acid;
methyl ((1-((2-(3,5-dichlorophenyl)-6-((2-(4-methylpiperazin-1-yl)pyrimidin-5-yl)oxy)pyridin-4-yl)methyl)piperidin-4-yl)methyl)carbamate;
N-((1-((2-(3,5-dichlorophenyl)-6-((2-(4-methylpiperazin-1-yl)pyrimidin-5-yl)oxy)pyridin-4-yl)methyl)piperidin-4-yl)methyl)acetamide;
(1-((2-(3,5-dichlorophenyl)-6-((2-(4-methylpiperazin-1-yl)pyrimidin-5-yl)oxy)pyridin-4-yl)methyl)piperidin-4-yl)methylmethylcarbamate;
1-((1-((2-(3,5-dichlorophenyl)-6-((2-(4-methylpiperazin-1-yl)pyrimidin-5-yl)oxy)pyridin-4-yl)methyl)piperidin-4-yl)methyl)-3-methylurea;
N-((1-((2-(3,5-dichlorophenyl)-6-((2-(8-methyl-3,8-diazabicyclo[3.2.1]octan-3-yl)pyrimidin-5-yl)oxy)pyridin-4-yl)methyl)piperidin-4-yl)methyl)acetamide;
5-((4-((4-((1H-tetrazol-5-yl)methyl)piperidin-1-yl)methyl)-6-(3,5-dichlorophenyl)pyridin-2-yl)oxy)-2-(4-methylpiperazin-1-yl)pyrimidine;
(1-((2-(3,5-dichlorophenyl)-6-((2-(piperazin-1-yl)pyrimidin-5-yl)oxy)pyridin-4-yl)methyl)piperidin-4-yl)methylmethylcarbamate;
1-((1-((2-(3,5-dichlorophenyl)-6-((2-(piperazin-1-yl)pyrimidin-5-yl)oxy)pyridin-4-yl)methyl)piperidin-4-yl)methyl)-3-methylurea;
3-(4-(5-((4-((4-(3-amino-3-oxopropyl)piperazin-1-yl)methyl)-6-(3,5-dichlorophenyl)pyridin-2-yl)oxy)pyrimidin-2-yl)piperazin-1-yl)propanoic acid;
3-(4-(5-((4-((4-(2-amino-2-oxoethyl)piperazin-1-yl)methyl)-6-(3,5-dichlorophenyl)pyridin-2-yl)oxy)pyrimidin-2-yl)piperazin-1-yl)propanoic acid;
3-(4-(5-((6-(3,5-dichlorophenyl)-4-(((1R,7S,8r)-8-(methylsulfonamido)-4-azabicyclo[5.1.0]octan-4-yl)methyl)pyridin-2-yl)oxy)pyrimidin-2-yl)piperazin-1-yl)propanoic acid;
4-(4-(5-((6-(3,5-dichlorophenyl)-4-(((1R,7S,8r)-8-(methylsulfonamido)-4-azabicyclo[5.1.0]octan-4-yl)methyl)pyridin-2-yl)oxy)pyrimidin-2-yl)piperazin-1-yl)-2-methylbutanoic acid;
4-(4-(5-((4-(((1R,7S,8r)-8-acetamido-4-azabicyclo[5.1.0]octan-4-yl)methyl)-6-(3,5-dichlorophenyl)pyridin-2-yl)oxy)pyrimidin-2-yl)piperazin-1-yl)-2-methylbutanoic acid;
4-(4-(5-((6-(3,5-dichlorophenyl)-4-(pyrrolidin-1-ylmethyl)pyridin-2-yl)oxy)pyrimidin-2-yl)piperazin-1-yl)-2-methylbutanoic acid;
3-(4-(5-((4-((4-(cyclopropanecarboxamidomethyl)piperidin-1-yl)methyl)-6-(3,5-dichlorophenyl)pyridin-2-yl)oxy)pyrimidin-2-yl)piperazin-1-yl)propanoic acid;
2-(1-((2-(3,5-dichlorophenyl)-6-((2-(piperazin-1-yl)pyrimidin-5-yl)oxy)pyridin-4-yl)methyl)piperidin-4-yl)acetamide;
2-(1-((2-(3,5-dichlorophenyl)-6-((2-(4-methylpiperazin-1-yl)pyrimidin-5-yl)oxy)pyridin-4-yl)methyl)piperidin-4-yl)ethanesulfonamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanamide;
N-((1-((2-(3,5-dichlorophenyl)-6-((2-(piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-N-methylpropanamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanamide;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanamide;
N-((1-((2-((2-(1,4-diazepan-1-yl)pyrimidin-5-yl)oxy)-6-(3,5-dichlorophenyl)pyridin-4-yl)methyl)piperidin-4-yl)methyl)acetamide;
N-((1-((2-((2-(4-aminopiperidin-1-yl)pyrimidin-5-yl)oxy)-6-(3,5-dichlorophenyl)pyridin-4-yl)methyl)piperidin-4-yl)methyl)acetamide;
2-((1-((2-(3,5-dichlorophenyl)-6-((2-(piperazin-1-yl)pyrimidin-5-yl)oxy)pyridin-4-yl)methyl)piperidin-4-yl)oxy)acetic acid;
2-(1-((2-(3,5-dichlorophenyl)-6-((2-(6-hydroxy-1,4-diazepan-1-yl)pyrimidin-5-yl)oxy)pyridin-4-yl)methyl)piperidin-4-yl)acetic acid;
2-(1-((2-((2-(4-amino-4-(2-hydroxyethyl)piperidin-1-yl)pyrimidin-5-yl)oxy)-6-(3,5-dichlorophenyl)pyridin-4-yl)methyl)piperidin-4-yl)acetic acid;
((1-((2-(3,5-dichlorophenyl)-6-((2-(piperazin-1-yl)pyrimidin-5-yl)oxy)pyridin-4-yl)methyl)piperidin-4-yl)methyl)dimethylphosphineoxide;
2-(1-((2-(3,5-dichlorophenyl)-6-((2-(4-methyl-1,4-diazepan-1-yl)pyrimidin-5-yl)oxy)pyridin-4-yl)methyl)piperidin-4-yl)acetic acid;
3-(1-((2-(3,5-dichlorophenyl)-6-((2-(4-methylpiperazin-1-yl)pyrimidin-5-yl)oxy)pyridin-4-yl)methyl)azetidin-3-yl)butanoic acid;
2-((1-((2-(3,5-dichlorophenyl)-6-((2-(4-methylpiperazin-1-yl)pyrimidin-5-yl)oxy)pyridin-4-yl)methyl)pyrrolidin-3-yl)oxy)acetic acid;
2-(2-((2-(3,5-dichlorophenyl)-6-((2-(4-methylpiperazin-1-yl)pyrimidin-5-yl)oxy)pyridin-4-yl)methyl)octahydrocyclopenta[c]pyrrol-5-yl)acetic acid;
3-(1-((2-(3,5-dichlorophenyl)-6-((2-(4-methylpiperazin-1-yl)pyrimidin-5-yl)oxy)pyridin-4-yl)methyl)piperidin-4-yl)propanoic acid;
3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
2-(1-((2-((2-(1,4-diazepan-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-((2-(5-methylhexahydropyrrolo[3,4-c]pyrrol-2(1H)-yl)pyrimidin-5-yl)oxy)pyridin-4-yl)methyl)piperidin-4-yl)acetic acid;
(S)-3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
1-(7-((2-(3,5-dichlorophenyl)-6-((2-(4-methylpiperazin-1-yl)pyrimidin-5-yl)oxy)pyridin-4-yl)methyl)-2,7-diazaspiro[3.5]nonan-2-yl)-2-hydroxyethanone;
(R)-3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
1-(1-((2-(3,5-dichlorophenyl)-6-((2-(4-methylpiperazin-1-yl)pyrimidin-5-yl)oxy)pyridin-4-yl)methyl)piperidin-4-yl)propan-2-ol;
3-(1-((2-(3,5-dichlorophenyl)-6-((2-(4-methylpiperazin-1-yl)pyrimidin-5-yl)oxy)pyridin-4-yl)methyl)piperidin-4-yl)-2-hydroxypropanoic acid;
2-((1-((2-(3,5-dichlorophenyl)-6-((2-(4-methyl-1,4-diazepan-1-yl)pyrimidin-5-yl)oxy)pyridin-4-yl)methyl)piperidin-4-yl)oxy)acetic acid;
2-(1-((2-(3,5-dichlorophenyl)-6-((2-(4-methylpiperazin-1-yl)pyrimidin-5-yl)oxy)pyridin-4-yl)methyl)piperidin-4-yl)acetamide;
1-((1-((2-(3,5-dichlorophenyl)-6-((2-(4-methylpiperazin-1-yl)pyrimidin-5-yl)oxy)pyridin-4-yl)methyl)piperidin-4-yl)oxy)cyclopropanecarboxylic acid;
2-(1-((2-(3,5-dichlorophenyl)-6-((2-(6-methoxy-4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-(6-hydroxy-4,6-dimethyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-(6-fluoro-4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((4-methyl-2-(4-methylpiperazin-1-yl)pyrimidin-5-yl)oxy)pyridin-4-yl)methyl)piperidin-4-yl)acetic acid;
2-(1-((2-(3,5-dichlorophenyl)-6-((2-(3-(methylamino)pyrrolidin-1-yl)pyrimidin-5-yl)oxy)pyridin-4-yl)methyl)piperidin-4-yl)acetic acid;
(S)-2-(4-((2-(3,5-dichlorophenyl)-6-((2-(4-methylpiperazin-1-yl)pyrimidin-5-yl)oxy)pyridin-4-yl)methyl)-1,4-oxazepan-7-yl)ethanol;
N-((1R,5S,6r)-3-((2-(3,5-dichlorophenyl)-6-((2-(4-methyl-1,4-diazepan-1-yl)pyrimidin-5-yl)oxy)pyridin-4-yl)methyl)-3-azabicyclo[3.1.0]hexan-6-yl)acetamide;
1-(1-((2-(3,5-dichlorophenyl)-6-((2-(4-methylpiperazin-1-yl)pyrimidin-5-yl)oxy)pyridin-4-yl)methyl)piperidin-4-yl)propan-2-one;
2-(1-((2-((2-(4-aminopiperidin-1-yl)pyrimidin-5-yl)oxy)-6-(3,5-dichlorophenyl)pyridin-4-yl)methyl)piperidin-4-yl)acetic acid;
(S)-2-(1-((2-(3,5-dichlorophenyl)-6-((2-(3-methylpiperazin-1-yl)pyrimidin-5-yl)oxy)pyridin-4-yl)methyl)piperidin-4-yl)acetic acid;
2-(1-((2-(3,5-dichlorophenyl)-6-((2-(3,3-dimethylpiperazin-1-yl)pyrimidin-5-yl)oxy)pyridin-4-yl)methyl)piperidin-4-yl)acetic acid;
2-(1-((2-(3,5-dichlorophenyl)-6-((2-(hexahydropyrrolo[3,4-c]pyrrol-2(1H)-yl)pyrimidin-5-yl)oxy)pyridin-4-yl)methyl)piperidin-4-yl)acetic acid;
2-(1-((2-((2-(3,6-diazabicyclo[3.1.1]heptan-3-yl)pyrimidin-5-yl)oxy)-6-(3,5-dichlorophenyl)pyridin-4-yl)methyl)piperidin-4-yl)acetic acid;
2-(1-((2-((2-(3,8-diazabicyclo[3.2.1]octan-3-yl)pyrimidin-5-yl)oxy)-6-(3,5-dichlorophenyl)pyridin-4-yl)methyl)piperidin-4-yl)acetic acid;
2-(1-((2-((2-(4,7-diazaspiro[2.5]octan-7-yl)pyrimidin-5-yl)oxy)-6-(3,5-dichlorophenyl)pyridin-4-yl)methyl)piperidin-4-yl)acetic acid;
2-(1-((2-((2-(4-amino-4-(hydroxymethyl)piperidin-1-yl)pyrimidin-5-yl)oxy)-6-(3,5-dichlorophenyl)pyridin-4-yl)methyl)piperidin-4-yl)acetic acid;
1-((1-((2-(3,5-dichlorophenyl)-6-((2-(4-(3-hydroxypropyl)piperazin-1-yl)pyrimidin-5-yl)oxy)pyridin-4-yl)methyl)piperidin-4-yl)methyl)-3-methylurea;
methyl (3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoyl)carbamate;
1-(2-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)ethyl)cyclopropanecarboxylic acid;
methyl (3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoyl)carbamate;
2-(1-((2-(3,5-dichlorophenyl)-6-((2-(4-(2,3-dihydroxypropyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-(methylsulfinyl)butyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)acetic acid;
2-(1-((2-(3,5-dichlorophenyl)-6-((2-(piperazin-1-yl)pyrimidin-5-yl)oxy)pyridin-4-yl)methyl)piperidin-4-yl)acetic acid;
2-(1-((2-(3,5-dichlorophenyl)-6-((2-(4-methylpiperazin-1-yl)pyrimidin-5-yl)oxy)pyridin-4-yl)methyl)piperidin-4-yl)acetic acid;
4-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)pentanoic acid;
(1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)methylmethylcarbamate;
(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methylmethylcarbamate;
3-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)cyclobutanecarboxylic acid;
N-((1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)methyl)acetamide;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)pentanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)cyclobutanecarboxylic acid;
1-((1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)cyclobutanecarboxylic acid;
4-(4-(5-((6-(3,5-dichlorophenyl)-4-((4-(((methoxycarbonyl)amino)methyl)piperidin-1-yl)methyl)pyridin-2-yl)oxy)pyrimidin-2-yl)piperazin-1-yl)pentanoic acid;
methyl ((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)carbamate;
2-((1R,7S,8r)-4-((2-(3,5-dichlorophenyl)-6-((2-(4-methylpiperazin-1-yl)pyrimidin-5-yl)oxy)pyridin-4-yl)methyl)-4-azabicyclo[5.1.0]octan-8-yl)acetic acid;
2-((1-((2-(3,5-dichlorophenyl)-6-((2-(4-methylpiperazin-1-yl)pyrimidin-5-yl)oxy)pyridin-4-yl)methyl)piperidin-4-yl)oxy)-2-methylpropanoic acid;
2-(1-((2-(3,5-dichlorophenyl)-6-((2-(6-hydroxy-4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-(4-(dimethylamino)piperidin-1-yl)pyrimidin-5-yl)oxy)pyridin-4-yl)methyl)piperidin-4-yl)acetic acid;
2-(1-((2-(3,5-dichlorophenyl)-6-((2-(6-methyl-3,6-diazabicyclo[3.1.1]heptan-3-yl)pyrimidin-5-yl)oxy)pyridin-4-yl)methyl)piperidin-4-yl)acetic acid;
2-(1-((2-(3,5-dichlorophenyl)-6-((2-(4-((1-hydroxycyclopropyl)methyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-ethyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-((1S,4S)-5-methyl-2,5-diazabicyclo[2.2.1]heptan-2-yl)pyrimidin-5-yl)oxy)pyridin-4-yl)methyl)piperidin-4-yl)acetic acid;
2-(1-((2-(3,5-dichlorophenyl)-6-((2-((1S,4S)-5-ethyl-2,5-diazabicyclo[2.2.1]heptan-2-yl)pyrimidin-5-yl)oxy)pyridin-4-yl)methyl)piperidin-4-yl)acetic acid;
2-(1-((2-((2-(4-cyclopropylpiperazin-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-((2-(4-ethylpiperazin-1-yl)pyrimidin-5-yl)oxy)pyridin-4-yl)methyl)piperidin-4-yl)acetic acid;
2-(1-((2-(3,5-dichlorophenyl)-6-((2-(4-isopropylpiperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-fluoroethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxybutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-(methylamino)butan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-(dimethylamino)butan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-((methylcarbamoyl)oxy)ethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)acetic acid;
N-((1-((2-(3,5-dichlorophenyl)-6-((2-(4-(2-methoxyethyl)piperazin-1-yl)pyrimidin-5-yl)oxy)pyridin-4-yl)methyl)piperidin-4-yl)methyl)acetamide;
2-(1-((2-(3,5-dichlorophenyl)-6-((2-(4-(3-sulfamoylpropyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-methoxyethyl)piperazin-1-yl)pyrimidin-5-yl)oxy)pyridin-4-yl)methyl)piperidin-4-yl)acetic acid;
3-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)propanoic acid;
N-((1-((2-(3,5-dichlorophenyl)-6-((2-(4-(3-sulfamoylpropyl)piperazin-1-yl)pyrimidin-5-yl)oxy)pyridin-4-yl)methyl)piperidin-4-yl)methyl)acetamide;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)propanoic acid;
1-((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)-3-methylurea;
N-((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)acetamide;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)propanamide;
1-((1-((2-(3,5-dichlorophenyl)-6-((2-(4-(2-methoxyethyl)piperazin-1-yl)pyrimidin-5-yl)oxy)pyridin-4-yl)methyl)piperidin-4-yl)methyl)-3-methylurea;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoic acid;
3-(4-(5-((6-(3,5-dichlorophenyl)-4-((4-hydroxy-4-((3-methylureido)methyl)piperidin-1-yl)methyl)pyridin-2-yl)oxy)pyrimidin-2-yl)piperazin-1-yl)propanamide;
3-(4-(5-((6-(3,5-dichlorophenyl)-4-((4-(((ethoxycarbonyl)amino)methyl)piperidin-1-yl)methyl)pyridin-2-yl)oxy)pyrimidin-2-yl)piperazin-1-yl)propanoic acid;
3-(4-(5-((6-(3,5-dichlorophenyl)-4-((4-(2-(ethylamino)-2-oxoethyl)piperidin-1-yl)methyl)pyridin-2-yl)oxy)pyrimidin-2-yl)piperazin-1-yl)propanoic acid;
3-(1-((2-(3,5-dichlorophenyl)-6-((2-(piperazin-1-yl)pyrimidin-5-yl)oxy)pyridin-4-yl)methyl)piperidin-4-yl)-2-methylpropanoic acid;
3-(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)propanoic acid;
3-(4-(5-((4-((4-(acetamidomethyl)piperidin-1-yl)methyl)-6-(3-chloro-4,5-difluorophenyl)pyridin-2-yl)oxy)pyrimidin-2-yl)piperazin-1-yl)propanamide;
1-((1-((2-(3-chloro-5-fluorophenyl)-6-((2-(4-(3-(methylsulfonyl)propyl)piperazin-1-yl)pyrimidin-5-yl)oxy)pyridin-4-yl)methyl)piperidin-4-yl)methyl)-3-methylurea;
methyl ((1-((2-(3-chloro-5-fluorophenyl)-6-((2-(piperazin-1-yl)pyrimidin-5-yl)oxy)pyridin-4-yl)methyl)piperidin-4-yl)methyl)carbamate;
2-(1-((2-(3,5-dichlorophenyl)-6-((2-(4-(2-(1-hydroxycyclopropyl)ethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxy-3-methylbutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxy-2,2-dimethylpropyl)piperazin-1-yl)pyrimidin-5-yl)oxy)pyridin-4-yl)methyl)piperidin-4-yl)acetic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylpropanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylpropanamide;
N-((1-((2-(3,5-dichlorophenyl)-6-((2-(4-(2-(methylsulfonyl)ethyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-sulfamoylethyl)piperazin-1-yl)pyrimidin-5-yl)oxy)pyridin-4-yl)methyl)piperidin-4-yl)methyl)acetamide;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)-2-methylpropanamide;
(S)-3-(4-(5-((6-(3,5-dichlorophenyl)-4-((4-(((methoxycarbonyl)amino)methyl)piperidin-1-yl)methyl)pyridin-2-yl)oxy)pyrimidin-2-yl)-2-methylpiperazin-1-yl)propanoic acid;
1-((1-((2-(3,5-dichlorophenyl)-6-((2-(4-(3-hydroxybutyl)piperazin-1-yl)pyrimidin-5-yl)oxy)pyridin-4-yl)methyl)piperidin-4-yl)methyl)-3-methylurea;
3-(3-(5-((4-((4-(acetamidomethyl)piperidin-1-yl)methyl)-6-(3,5-dichlorophenyl)pyridin-2-yl)oxy)pyrimidin-2-yl)-3,8-diazabicyclo[3.2.1]octan-8-yl)propanoic acid;
methyl3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoate;
(R)-2-(1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)acetic acid;
3-(1-((2-(3,5-dichlorophenyl)-6-((2-(4-((R)-2-hydroxypropyl)piperazin-1-yl)pyrimidin-5-yl)oxy)pyridin-4-yl)methyl)piperidin-4-yl)-2-methylpropanoic acid;
(R)-N-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-hydroxy-2-methylpropyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(3-fluoro-2-hydroxypropyl)piperazin-1-yl)pyrimidin-5-yl)oxy)pyridin-4-yl)methyl)piperidin-4-yl)methyl)acetamide;
2-(1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)acetic acid;
(1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methylmethylcarbamate;
(R)-(1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methylmethylcarbamate;
(R)-1-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)-3-methylurea;
methyl ((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)carbamate;
(R)-methyl ((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)carbamate;
N-((1-((2-(3-chloro-5-fluorophenyl)-6-((2-(4-(2-hydroxyethyl)piperazin-1-yl)pyrimidin-5-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-hydroxypropanoic acid;
(R)-2-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)oxy)acetic acid;
((1-((2-(3,5-dichlorophenyl)-6-((2-(4-methylpiperazin-1-yl)pyrimidin-5-yl)oxy)pyridin-4-yl)methyl)piperidin-4-yl)methyl)boronic acid;
(2-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)ethyl)boronic acid;
((1-((2-(3,5-dichlorophenyl)-6-((2-(4-methylpiperazin-1-yl)pyrimidin-5-yl)oxy)pyridin-4-yl)methyl)piperidin-4-yl)methyl)dimethylphosphineoxide;
(1R,7S,8r)-4-((2-(3,5-dichlorophenyl)-6-((2-(4-methylpiperazin-1-yl)pyrimidin-5-yl)oxy)pyridin-4-yl)methyl)-4-azabicyclo[5.1.0]octane-8-carboxylic acid;
((1-((2-(3,5-dichlorophenyl)-6-((2-(4-methyl-1,4-diazepan-1-yl)pyrimidin-5-yl)oxy)pyridin-4-yl)methyl)piperidin-4-yl)methyl)boronic acid;
(2-(1-((2-(3,5-dichlorophenyl)-6-((2-(4-methylpiperazin-1-yl)pyrimidin-5-yl)oxy)pyridin-4-yl)methyl)piperidin-4-yl)ethyl)boronic acid;
(1-((2-(3,5-dichlorophenyl)-6-((6-(4-methylpiperazin-1-yl)pyridin-3-yl)oxy)pyridin-4-yl)methyl)piperidin-4-yl)methylacetylcarbamate;
N-1-((1-((2-(3,5-dichlorophenyl)-6-((2-(4-methylpiperazin-1-yl)pyrimidin-5-yl)oxy)pyridin-4-yl)methyl)piperidin-4-yl)methyl)-N′-methoxylcarbonylurea;
N-(((1-((2-(3,5-dichlorophenyl)-6-((2-(4-methylpiperazin-1-yl)pyrimidin-5-yl)oxy)pyridin-4-yl)methyl)piperidin-4-yl)methyl)carbamoyl)methanesulfonamide;
N-(((1-((2-(3,5-dichlorophenyl)-6-((2-(4-methylpiperazin-1-yl)pyrimidin-5-yl)oxy)pyridin-4-yl)methyl)piperidin-4-yl)methyl)carbamoyl)acetamide;
N-((1-((2-(3,5-dichlorophenyl)-6-((2-(4-methylpiperazin-1-yl)pyrimidin-5-yl)oxy)pyridin-4-yl)methyl)piperidin-4-yl)carbamoyl)methanesulfonamide;
1-(1-((2-(3,5-dichlorophenyl)-6-((2-(4-methylpiperazin-1-yl)pyrimidin-5-yl)oxy)pyridin-4-yl)methyl)piperidin-4-yl)urea;
(S)-(4-((2-(3,5-dichlorophenyl)-6-((2-(4-methylpiperazin-1-yl)pyrimidin-5-yl)oxy)pyridin-4-yl)methyl)-1,4-oxazepan-7-yl)methanol;
(1-((2-(3,5-dichlorophenyl)-6-((2-(4-methylpiperazin-1-yl)pyrimidin-5-yl)oxy)pyridin-4-yl)methyl)piperidin-4-yl)dimethylphosphineoxide;
2-(1-((2-(3,5-dichlorophenyl)-6-((2-(4-methylpiperazin-1-yl)pyrimidin-5-yl)amino)pyridin-4-yl)methyl)piperidin-4-yl)acetic acid;
2-(1-((2-((2-(1,4-diazabicyclo[3.2.1]octan-4-yl)pyrimidin-5-yl)oxy)-6-(3,5-dichlorophenyl)pyridin-4-yl)methyl)piperidin-4-yl)acetic acid;
N-((1-((2-((2-(3,8-diazabicyclo[3.2.1]octan-3-yl)pyrimidin-5-yl)oxy)-6-(3,5-dichlorophenyl)pyridin-4-yl)methyl)piperidin-4-yl)methyl)acetamide;
methyl ((1-((2-((2-(1,4-diazabicyclo[3.2.1]octan-4-yl)pyrimidin-5-yl)oxy)-6-(3,5-dichlorophenyl)pyridin-4-yl)methyl)piperidin-4-yl)methyl)carbamate;
4-((2-((2-(1,4-diazabicyclo[3.2.1]octan-4-yl)pyrimidin-5-yl)oxy)-6-(3,5-dichlorophenyl)pyridin-4-yl)methyl)-4-azabicyclo[5.1.0]octane-8-carboxylic acid;
4-(5-((6-(3,5-dichlorophenyl)-4-((4-fluoropiperidin-1-yl)methyl)pyridin-2-yl)oxy)pyrimidin-2-yl)-1,4-diazabicyclo[3.2.1]octane;
3-(4-(5-((4-(((1R,7S,8r)-8-acetamido-4-azabicyclo[5.1.0]octan-4-yl)methyl)-6-(3,5-dichlorophenyl)pyridin-2-yl)oxy)pyrimidin-2-yl)piperazin-1-yl)propanoic acid;
methyl4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoate;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoic acid;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2,2-dimethylbutanoic acid;
N-((1-((2-(3-chloro-5-fluorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)acetamide;
1-((1-((2-(3-chloro-5-fluorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)-3-methylurea;
2-(1-((2-(3-chloro-5-fluorophenyl)-6-((2-(4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-(isopropyl(2-(4-methylpiperazin-1-yl)pyrimidin-5-yl)amino)pyridin-4-yl)methyl)piperidin-4-yl)acetic acid;
2-(1-((6-(3,5-dichlorophenyl)-3-fluoro-2-((2-(8-methyl-3,8-diazabicyclo[3.2.1]octan-3-yl)pyrimidin-5-yl)oxy)pyridin-4-yl)methyl)piperidin-4-yl)acetic acid;
2-(1-((6-(3,5-dichlorophenyl)-3-fluoro-2-((2-(4-methylpiperazin-1-yl)pyrimidin-5-yl)oxy)pyridin-4-yl)methyl)piperidin-4-yl)acetic acid;
2-(1-((6-(3,5-dichlorophenyl)-3-fluoro-2-((4-methyl-2-(4-methylpiperazin-1-yl)pyrimidin-5-yl)oxy)pyridin-4-yl)methyl)piperidin-4-yl)acetic acid;
4-(4-(5-((4-((4-(acetamidomethyl)piperidin-1-yl)methyl)-6-(3,5-dichlorophenyl)-3-fluoropyridin-2-yl)oxy)pyrimidin-2-yl)piperazin-1-yl)-2-methylbutanoic acid;
2-(1-((2-((2-(3,8-diazabicyclo[3.2.1]octan-3-yl)pyrimidin-5-yl)oxy)-6-(3,5-dichlorophenyl)-3-fluoropyridin-4-yl)methyl)piperidin-4-yl)acetic acid;
2-(1-((2-((2-(4-((1H-1,2,3-triazol-5-yl)methyl)piperazin-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-(methyl(2-(4-methylpiperazin-1-yl)pyrimidin-5-yl)amino)pyridin-4-yl)methyl)piperidin-4-yl)acetic acid;
2-(1-((2-(3,5-dichlorophenyl)-6-(ethyl(2-(4-methylpiperazin-1-yl)pyrimidin-5-yl)amino)pyridin-4-yl)methyl)piperidin-4-yl)acetic acid;
(1R,7S,8r)-4-((2-(3,5-dichlorophenyl)-6-((2-(4-methyl-1,4-diazepan-1-yl)pyrimidin-5-yl)oxy)pyridin-4-yl)methyl)-4-azabicyclo[5.1.0]octane-8-carboxylic acid;
2-(1-((2-(3,5-dichlorophenyl)-6-((2-(8-methyl-3,8-diazabicyclo[3.2.1]octan-3-yl)pyrimidin-5-yl)oxy)pyridin-4-yl)methyl)piperidin-4-yl)acetic acid; or
2-(1-((2-(3,5-dichlorophenyl)-6-((2-(6-fluoro-1,4-diazepan-1-yl)pyrimidin-5-yl)oxy)pyridin-4-yl)methyl)piperidin-4-yl)acetic acid;
or a pharmaceutically acceptable salt thereof.

45. The method, compound, pharmaceutical composition, or use of any one of claims 1 to 31 or 34 to 44, wherein the compound, or pharmaceutically acceptable salt thereof, is of the formula:

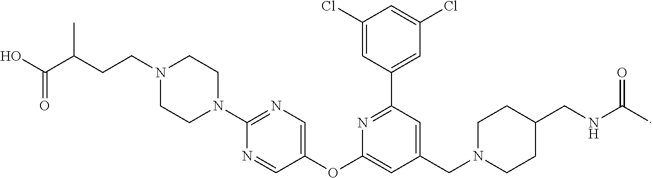

46. The method, compound, pharmaceutical composition, or use of any one of claims 1 to 29 or 32 to 37, wherein R¹ and R² taken together with the nitrogen atom to which they are attached form a piperazine ring of the formula:

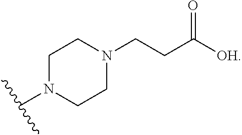 [SMILES: *N1CCN(CCC(=O)O)CC1]

47. The method, compound, pharmaceutical composition, or use of any one of claims 1 to 29, 32 to 42 or 46, wherein the compound, or pharmaceutically acceptable salt thereof, is:
2-(4-((2-(3,5-dichlorophenyl)-6-((6-(piperazin-1-yl)pyridin-3-yl)oxy)pyridin-4-yl)methyl)piperazin-1-yl)-N-methylacetamide;
N-((1-((2-(3,5-dichlorophenyl)-6-((6-(piperazin-1-yl)pyridin-3-yl)oxy)pyridin-4-yl)methyl)-4-hydroxypiperidin-4-yl)methyl)acetamide;
3-(1-((2-(3,5-dichlorophenyl)-6-((6-(piperazin-1-yl)pyridin-3-yl)oxy)pyridin-4-yl)methyl)piperidin-4-yl)propanoicacid;
2-(1-((2-(3,5-dichlorophenyl)-6-((6-(piperazin-1-yl)pyridin-3-yl)oxy)pyridin-4-yl)methyl)piperidin-4-yl)aceticacid;
1-((1-((2-(3,5-dichlorophenyl)-6-((6-(piperazin-1-yl)pyridin-3-yl)oxy)pyridin-4-yl)methyl)-4-hydroxypiperidin-4-yl)methyl)-3-methylurea;
methyl((1-((2-(3,5-dichlorophenyl)-6-((6-(piperazin-1-yl)pyridin-3-yl)oxy)pyridin-4-yl)methyl)-4-hydroxypiperidin-4-yl)methyl)carbamate;
2-(1-((2-(3,5-dichlorophenyl)-6-((6-(piperazin-1-yl)pyridin-3-yl)oxy)pyridin-4-yl)methyl)piperidin-4-yl)ethanesulfonicacid;
(1-((2-(3,5-dichlorophenyl)-6-((6-(piperazin-1-yl)pyridin-3-yl)oxy)pyridin-4-yl)methyl)piperidin-4-yl)methanesulfonicacid;
N-((1-((2-(3,5-dichlorophenyl)-6-((6-(piperazin-1-yl)pyridin-3-yl)oxy)pyridin-4-yl)methyl)piperidin-4-yl)methyl)acetamide;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)aceticacid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoicacid;
N-((1-((2-(3,5-dichlorophenyl)-6-((6-(4-(2-(methylsulfonyl)ethyl)piperazin-1-yl)pyridin-3-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanamide;
N-((1-((2-((6-(4-(2-(1H-tetrazol-5-yl)ethyl)piperazin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(2-(methylsulfinyl)ethyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-(4-(methylsulfonyl)butan-2-yl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((trans)-3-(methylsulfonamido)cyclobutyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((cis)-3-(methylsulfonamido)cyclobutyl)piperazin-1-yl)pyridin-3-yl)oxy)pyridin-4-yl)methyl)piperidin-4-yl)methyl)acetamide;
N-((1-((2-((6-(4-(2-aminoethyl)piperazin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(2,4-dihydroxybutyl)piperazin-1-yl)pyridin-3-yl)oxy)pyridin-4-yl)methyl)piperidin-4-yl)methyl)acetamide;
(2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethyl)phosphonicacid;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethylcarbamate;
N-((1-((2-(3,5-dichlorophenyl)-6-((6-(4-(2-(N-methylmethylsulfonamido)ethyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((1-(hydroxymethyl)cyclopropyl)methyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((1-hydroxycyclopropyl)methyl)piperazin-1-yl)pyridin-3-yl)oxy)pyridin-4-yl)methyl)piperidin-4-yl)methyl)acetamide;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)-N-ethylacetamide;
3-(4-(5-((6-(3,5-dichlorophenyl)-4-((4-(sulfamoylmethyl)piperidin-1-yl)methyl)pyridin-2-yl)oxy)pyridin-2-yl)piperazin-1-yl)propanoicacid;
3-(4-(5-((6-(3,5-dichlorophenyl)-4-((4-((methylsulfonyl)methyl)piperidin-1-yl)methyl)pyridin-2-yl)oxy)pyridin-2-yl)piperazin-1-yl)propanoicacid;
1-((1-((2-(3,5-dichlorophenyl)-6-((6-(4-(2-hydroxyethyl)piperazin-1-yl)pyridin-3-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyridin-2-yl)piperazin-1-yl)propanoicacid;
3-(4-(5-((6-(3,5-dichlorophenyl)-4-((4-(methylsulfonamidomethyl)piperidin-1-yl)methyl)pyridin-2-yl)oxy)pyridin-2-yl)piperazin-1-yl)propanoicacid;
3-(4-(5-((6-(3,5-dichlorophenyl)-4-((methylamino)methyl)pyridin-2-yl)oxy)pyridin-2-yl)piperazin-1-yl)propanoicacid;
3-(4-(5-((4-((4-(2-(carbamoyloxy)ethyl)piperazin-1-yl)methyl)-6-(3,5-dichlorophenyl)pyridin-2-yl)oxy)pyridin-2-yl)piperazin-1-yl)propanoicacid;
3-(4-(5-((4-((4-(cyclopropanecarboxamidomethyl)piperidin-1-yl)methyl)-6-(3,5-dichlorophenyl)pyridin-2-yl)oxy)pyridin-2-yl)piperazin-1-yl)propanoicacid;
methyl((1-((2-(3,5-dichlorophenyl)-6-((6-(4-methylpiperazin-1-yl)pyridin-3-yl)oxy)pyridin-4-yl)methyl)piperidin-4-yl)methyl)carbamate;
(1-((2-(3,5-dichlorophenyl)-6-((6-(4-methylpiperazin-1-yl)pyridin-3-yl)oxy)pyridin-4-yl)methyl)piperidin-4-yl)methylmethylcarbamate;
1-((1-((2-(3,5-dichlorophenyl)-6-((6-(4-methylpiperazin-1-yl)pyridin-3-yl)oxy)pyridin-4-yl)methyl)piperidin-4-yl)methyl)-3-methylurea;
(1-((2-(3,5-dichlorophenyl)-6-((6-(piperazin-1-yl)pyridin-3-yl)oxy)pyridin-4-yl)methyl)piperidin-4-yl)methylmethylcarbamate;
methyl((1-((2-(3,5-dichlorophenyl)-6-((6-(piperazin-1-yl)pyridin-3-yl)oxy)pyridin-4-yl)methyl)piperidin-4-yl)methyl)carbamate;
3-(4-(5-((6-(3,5-dichlorophenyl)-4-((4-(((methylcarbamoyl)oxy)methyl)piperidin-1-yl)methyl)pyridin-2-yl)oxy)pyridin-2-yl)piperazin-1-yl)propanoicacid;
3-(4-(5-((6-(3,5-dichlorophenyl)-4-((4-((dimethylphosphoryl)methyl)piperidin-1-yl)methyl)pyridin-2-yl)oxy)pyridin-2-yl)piperazin-1-yl)propanoicacid;
N-((1-((2-(3,5-dichlorophenyl)-6-((5-fluoro-6-(piperazin-1-yl)pyridin-3-yl)oxy)pyridin-4-yl)methyl)-4-hydroxypiperidin-4-yl)methyl)acetamide;
1-(5-((6-(3-chloro-5-methylphenyl)-4-((methylamino)methyl)pyridin-2-yl)oxy)pyridin-2-yl)piperidin-4-amine;
3-(4-(5-((4-((4-(acetamidomethyl)piperidin-1-yl)methyl)-6-(3-chloro-5-fluorophenyl)pyridin-2-yl)oxy)pyridin-2-yl)piperazin-1-yl)propanoicacid;
3-(4-(5-((4-((4-(acetamidomethyl)piperidin-1-yl)methyl)-6-(3-bromo-5-fluorophenyl)pyridin-2-yl)oxy)pyridin-2-yl)piperazin-1-yl)propanoicacid;
2-(1-((2-(3,5-dichlorophenyl)-6-((6-(piperazin-1-yl)pyridin-3-yl)oxy)pyridin-4-yl)methyl)piperidin-4-yl)-N,N-dimethylethanamineoxide;
N-((1-((2-(3,5-dichlorophenyl)-6-((5-fluoro-6-(piperazin-1-yl)pyridin-3-yl)oxy)pyridin-4-yl)methyl)piperidin-4-yl)methyl)acetamide;
(1R,7S,8r)-4-((2-(3,5-dichlorophenyl)-6-((6-(4-methylpiperazin-1-yl)pyridin-3-yl)oxy)pyridin-4-yl)methyl)-4-azabicyclo[5.1.0]octane-8-carboxylicacid;
9-((2-(3,5-dichlorophenyl)-6-((6-(4-methylpiperazin-1-yl)pyridin-3-yl)oxy)pyridin-4-yl)methyl)-2-oxa-4,9-diazaspiro[5.5]undecan-3-one;
N-((1-((2-(3,5-dichlorophenyl)-6-((6-(6-fluoro-4-methyl-1,4-diazepan-1-yl)pyridin-3-yl)oxy)pyridin-4-yl)methyl)piperidin-4-yl)methyl)acetamide;
2-(1-((2-(3,5-dichlorophenyl)-6-((2-methyl-6-(4-methylpiperazin-1-yl)pyridin-3-yl)oxy)pyridin-4-yl)methyl)piperidin-4-yl)aceticacid;
2-(1-((2-(3,5-dichlorophenyl)-6-((5-fluoro-6-(4-methylpiperazin-1-yl)pyridin-3-yl)oxy)pyridin-4-yl)methyl)piperidin-4-yl)aceticacid;
2-(1-((2-(3,5-dichlorophenyl)-6-((6-(4-methyl-1,4-diazepan-1-yl)pyridin-3-yl)oxy)pyridin-4-yl)methyl)piperidin-4-yl)aceticacid;
2-(1-((2-((6-(1,4-diazepan-1-yl)pyridin-3-yl)oxy)-6-(3,5-dichlorophenyl)pyridin-4-yl)methyl)piperidin-4-yl)aceticacid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2-methylbutanoicacid;
methyl(3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoyl)carbamate;
4-(5-((4-((4-(acetamidomethyl)piperidin-1-yl)methyl)-6-(3,5-dichlorophenyl)pyridin-2-yl)oxy)pyridin-2-yl)-1-methylpiperazine1-oxide;
4-(5-((4-((4-(acetamidomethyl)piperidin-1-yl)methyl)-6-(3,5-dichlorophenyl)pyridin-2-yl)oxy)pyridin-2-yl)-1,1-bis(2-hydroxyethyl)piperazin-1-ium;
N-((1-((2-(3,5-dichlorophenyl)-6-((6-(4-(2-hydroxyethyl)piperazin-1-yl)pyridin-3-yl)oxy)pyridin-4-yl)methyl)piperidin-4-yl)methyl)acetamide;
4-(5-((4-((4-(acetamidomethyl)piperidin-1-yl)methyl)-6-(3,5-dichlorophenyl)pyridin-2-yl)oxy)pyridin-2-yl)-1,1-dimethylpiperazin-1-ium;
N-((1-((2-((6-(4-amino-3-fluoropiperidin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-((1S,4S)-5-(2-(methylsulfonyl)ethyl)-2,5-diazabicyclo[2.2.1]heptan-2-yl)pyridin-3-yl)oxy)pyridin-4-yl)methyl)piperidin-4-yl)methyl)acetamide;
N-((1-((2-((6-((3S,4R)-3-(aminomethyl)-4-hydroxypyrrolidin-1-yl)pyridin-3-yl)oxy)-6-(3,5-dichlorophenyl)pyridin-4-yl)methyl)piperidin-4-yl)methyl)acetamide;
3-((1R,5S)-3-(5-((4-((4-(acetamidomethyl)piperidin-1-yl)methyl)-6-(3,5-dichlorophenyl)pyridin-2-yl)oxy)pyridin-2-yl)-3,8-diazabicyclo[3.2.1]octan-8-yl)propanoicacid;
(S)-3-(4-(5-((4-((4-(acetamidomethyl)piperidin-1-yl)methyl)-6-(3,5-dichlorophenyl)pyridin-2-yl)oxy)pyridin-2-yl)-2-methylpiperazin-1-yl)propanoicacid;
2-(1-((2-((6-((1S,4S)-2,5-diazabicyclo[2.2.1]heptan-2-yl)pyridin-3-yl)oxy)-6-(3,5-dichlorophenyl)pyridin-4-yl)methyl)piperidin-4-yl)aceticacid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2-ethylbutanoicacid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2,2-dimethylpropanoicacid;
3-(4-(5-((4-((4-(acetamidomethyl)piperidin-1-yl)methyl)-6-(3,5-dichlorophenyl)pyridin-2-yl)oxy)pyridin-2-yl)-1,4-diazepan-1-yl)propanoicacid;
3-(4-(5-((4-((4-(acetamidomethyl)piperidin-1-yl)methyl)-6-(3,5-dichlorophenyl)pyridin-2-yl)oxy)pyridin-2-yl)-2,2-dimethylpiperazin-1-yl)propanoicacid;
N-((1-((2-((6-(1,4-diazepan-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(methylamino)piperidin-1-yl)pyridin-3-yl)oxy)pyridin-4-yl)methyl)piperidin-4-yl)methyl)acetamide;
N-((1-((2-(3,5-dichlorophenyl)-6-((6-(hexahydropyrrolo[3,4-c]pyrrol-2(1H)-yl)pyridin-3-yl)oxy)pyridin-4-yl)methyl)piperidin-4-yl)methyl)acetamide;
N-((1-((2-(3,5-dichlorophenyl)-6-((6-(3-(hydroxymethyl)piperazin-1-yl)pyridin-3-yl)oxy)pyridin-4-yl)methyl)piperidin-4-yl)methyl)acetamide;
N-((1-((2-((6-(4-aminopiperidin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(3,3-dimethylpiperazin-1-yl)pyridin-3-yl)oxy)pyridin-4-yl)methyl)piperidin-4-yl)methyl)acetamide;
N-((1-((2-((6-(4-amino-3,3-dimethylpiperidin-1-yl)pyridin-3-yl)oxy)-6-(3,5-dichlorophenyl)pyridin-4-yl)methyl)piperidin-4-yl)methyl)acetamide;
N-((1-((2-((6-(2,7-diazaspiro[4.4]nonan-2-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-methylpiperazin-1-yl)pyridin-3-yl)oxy)pyridin-4-yl)methyl)piperidin-4-yl)methyl)acetamide;
2-(1-((2-(3,5-dichlorophenyl)-6-((6-((1S,4S)-5-methyl-2,5-diazabicyclo[2.2.1]heptan-2-yl)pyridin-3-yl)oxy)pyridin-4-yl)methyl)piperidin-4-yl)aceticacid;
2-(1-((2-(3,5-dichlorophenyl)-6-((6-(4-methylpiperazin-1-yl)pyridin-3-yl)oxy)pyridin-4-yl)methyl)piperidin-4-yl)aceticacid;
3-(1-((2-(3,5-dichlorophenyl)-6-((6-(4-methylpiperazin-1-yl)pyridin-3-yl)oxy)pyridin-4-yl)methyl)piperidin-4-yl)-2-methylpropanoicacid;
methyl((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)carbamate;
N-((1-((2-(3,5-dichlorophenyl)-6-((6-(4-(3-(methylsulfonyl)butyl)piperazin-1-yl)pyridin-3-yl)oxy)pyridin-4-yl)methyl)piperidin-4-yl)methyl)acetamide;
1-((1-((2-(3,5-dichlorophenyl)-6-((6-(4-(3-(methylsulfonyl)butyl)piperazin-1-yl)pyridin-3-yl)oxy)pyridin-4-yl)methyl)piperidin-4-yl)methyl)-3-methylurea;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)aceticacid;
2-(1-((2-(3,5-dichlorophenyl)-6-((6-(4-(2-methoxyethyl)piperazin-1-yl)pyridin-3-yl)oxy)pyridin-4-yl)methyl)piperidin-4-yl)aceticacid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)butanoicacid;
N-((1-((2-(3,5-dichlorophenyl)-6-((6-(4-(2-methoxyethyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)acetamide;
2-(1-((2-(3,5-dichlorophenyl)-6-((6-(4-(3-sulfamoylpropyl)piperazin-1-yl)pyridin-3-yl)oxy)pyridin-4-yl)methyl)piperidin-4-yl)aceticacid;
1-((1-((2-(3,5-dichlorophenyl)-6-((6-(4-(2-methoxyethyl)piperazin-1-yl)pyridin-3-yl)oxy)pyridin-4-yl)methyl)piperidin-4-yl)methyl)-3-methylurea;
1-((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyridin-2-yl)piperazin-1-yl)propane-1-sulfonamide;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)acetamide;
3-(4-(5-((4-((4-(acetamidomethyl)piperidin-1-yl)methyl)-6-(3,5-dichlorophenyl)pyridin-2-yl)oxy)-3-fluoropyridin-2-yl)piperazin-1-yl)propanoicacid;
3-(4-(5-((6-(3,5-dichlorophenyl)-4-((4-((3-methylureido)methyl)piperidin-1-yl)methyl)pyridin-2-yl)oxy)-3-fluoropyridin-2-yl)piperazin-1-yl)propanoicacid;
3-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)propanoicacid;
3-(4-(5-((6-(3,5-dichlorophenyl)-4-((4-(2-(methylsulfonyl)ethyl)piperidin-1-yl)methyl)pyridin-2-yl)oxy)pyridin-2-yl)piperazin-1-yl)propanoicacid;
3-(4-(5-((6-(3,5-dichlorophenyl)-4-((4-(2-sulfamoylethyl)piperidin-1-yl)methyl)pyridin-2-yl)oxy)pyridin-2-yl)piperazin-1-yl)propanoicacid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)butanoicacid;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethanesulfonicacid;
2-((4-(5-((4-((4-(acetamidomethyl)piperidin-1-yl)methyl)-6-(3,5-dichlorophenyl)pyridin-2-yl)oxy)pyridin-2-yl)piperazin-1-yl)methyl)butanoicacid;
N-((1-((2-(3,5-dichlorophenyl)-6-((6-(4-(2-sulfamoylethyl)piperazin-1-yl)pyridin-3-yl)oxy)pyridin-4-yl)methyl)piperidin-4-yl)methyl)acetamide;
1-((1-((2-(3,5-dichlorophenyl)-6-((6-(4-(2-(methylsulfonyl)ethyl)piperazin-1-yl)pyridin-3-yl)oxy)pyridin-4-yl)methyl)piperidin-4-yl)methyl)-3-methylurea;
(R)-2-(1-((2-(3,5-dichlorophenyl)-6-((6-(4-(2-hydroxypropyl)piperazin-1-yl)pyridin-3-yl)oxy)pyridin-4-yl)methyl)piperidin-4-yl)aceticacid;
2-(1-((2-(3,5-dichlorophenyl)-6-((6-(4-(2-hydroxyethyl)piperazin-1-yl)pyridin-3-yl)oxy)pyridin-4-yl)methyl)piperidin-4-yl)aceticacid;
(R)-(1-((2-(3,5-dichlorophenyl)-6-((6-(4-(2-hydroxypropyl)piperazin-1-yl)pyridin-3-yl)oxy)pyridin-4-yl)methyl)piperidin-4-yl)methylmethylcarbamate;
(R)-1-((1-((2-(3,5-dichlorophenyl)-6-((6-(4-(2-hydroxypropyl)piperazin-1-yl)pyridin-3-yl)oxy)pyridin-4-yl)methyl)piperidin-4-yl)methyl)-3-methylurea;
3-(1-((2-(3,5-dichlorophenyl)-6-((6-(4-(2-hydroxyethyl)piperazin-1-yl)pyridin-3-yl)oxy)pyridin-4-yl)methyl)piperidin-4-yl)propanoicacid;
2-(1-((6-(3,5-dichlorophenyl)-3-fluoro-2-((6-(4-methylpiperazin-1-yl)pyridin-3-yl)oxy)pyridin-4-yl)methyl)piperidin-4-yl)aceticacid;
2-(1-((6-(3,5-dichlorophenyl)-3-fluoro-2-((2-methyl-6-(4-methylpiperazin-1-yl)pyridin-3-yl)oxy)pyridin-4-yl)methyl)piperidin-4-yl)aceticacid;
methyl3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoate; or
2-(1-((6-(3,5-dichlorophenyl)-3-methyl-2-((6-(4-methylpiperazin-1-yl)pyridin-3-yl)oxy)pyridin-4-yl)methyl)piperidin-4-yl)aceticacid;
or a pharmaceutically acceptable salt thereof.

48. The method, compound, pharmaceutical composition, or use of any one of claims 1 to 29, 32 to 42, 46, or 47, wherein the compound, or pharmaceutically acceptable salt thereof, is of the formula:

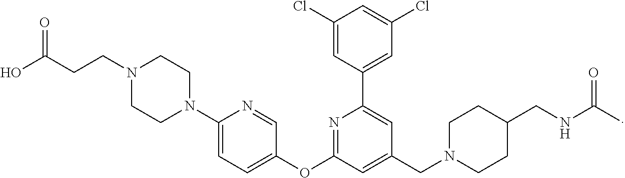

49. A method of identifying a compound useful for treating or preventing a viral infection resulting from a coronaviridae family virus, the method comprising:
(i) providing a test compound or a pharmaceutically acceptable salt thereof;
(ii) contacting a cell with the compound, wherein the cell expresses the pro-(S) protein of the virus; and
(iii) determining if the pro-(S) protein has been processed by furin or an endogenous furin-like enzyme.
